(19) United States
(12) Patent Application Publication
(10) Pub. No.: US 2025/0163056 A1
(43) Pub. Date: May 22, 2025

(54) NONMUSCLE MYOSIN II INHIBITORS

(71) Applicant: The University of Florida Research Foundation, Inc., Gainesville, FL (US)

(72) Inventors: Courtney Anne Miller, Jupiter, FL (US); Patrick Robert Griffin, Jupiter, FL (US); Theodore Mark Kamenecka, Palm Beach Gardens, FL (US); Gavin Rumbaugh, Jupiter, FL (US); Matthew Surman, Albany, NY (US); Steve Young, Lansdale, PA (US); Steven Duddy, Ann Arbor, MI (US); Laszlo Radnai, Stuart, FL (US)

(21) Appl. No.: 18/929,149

(22) Filed: Oct. 28, 2024

Related U.S. Application Data
(63) Continuation of application No. 18/169,660, filed on Feb. 15, 2023.
Application No. 18/169,660 is a continuation of application No. 17/250,203, filed on Dec. 14, 2020, now Pat. No. 11,649,234.
(60) Provisional application No. 62/685,158, filed on Jun. 14, 2018.

(51) Int. Cl. C07D 471/04; C07D 471/14; C07D 495/14; C07D 519/00

(52) U.S. Cl. CPC C07D 471/04; C07D 471/14; C07D 495/14; C07D 519/00

(57) ABSTRACT

The invention can provide compounds, analogs of blebbistatin, effective and selective inhibitors of nonmuscle myosin II relative to cardiac myosin II. Compounds can be used in the method of treating a disease, disorder, or medical condition in a patient, comprising modulating myosin II ATPase, such as treatment of substance abuse relapse disorder, or of renal disease, cancer and metastasis, benign prostate hyperplasia, hemostasis or thrombosis, nerve injury including retinal damage, lung fibrosis, liver fibrosis, arthrofibrosis, wound healing, spinal cord injury, periodontitis, glaucoma and immune-related diseases including multiple sclerosis; or wherein the disease, disorder, or medical condition comprises addiction including abuse of or addiction to anything classified as a Substance-Related or Addictive Disorder in the Diagnostic and Statistical Manual of Mental Disorders (DSM), such as, but not limited to, cocaine, opioids, amphetamines, ethanol, cannabis/marijuana, nicotine, and activities including gambling.

Compounds are of general formula

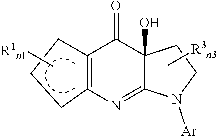

with substituents as defined herein.

### CROSS-REFERENCE TO RELATED APPLICATIONS

The subject patent application is a continuation of U.S. patent application Ser. No. 18/169,660 filed on Feb. 15, 2023, which is a continuation of U.S. patent application Ser. No. 17/250,203 filed on Dec. 14, 2020, which is U.S. national stage filing under 35 U.S.C. § 371 from International Application No. PCT/US2019/036897, filed on Jun. 13, 2019, and published as WO 2019/241469 on Dec. 19, 2019, and claims the benefit of priority to U.S. Provisional Patent Application No. 62/685,158, filed Jun. 14, 2018, the benefit of priority of each application of which is claimed herein, and which applications and publication are hereby incorporated herein by reference in their entirety.

### STATEMENT OF GOVERNMENT SUPPORT

This invention was made with government support under grant number NS096833 awarded by the National Institutes of Health. The government has certain rights in the invention.

### BACKGROUND

Substance abuse disorder is a chronic, relapsing disorder with no relapse preventing pharmacotherapies available for any drug of abuse. This is a major treatment challenge, as deeply engrained drug seeking behaviors persist long after the cessation of drug use. Numerous associations form between the drug's central and peripheral effects and components of the environment present at the time of drug use, which can range from the obvious (e.g. drug paraphernalia) to the more abstract (e.g. music, snow, gum)¹. The associations become highly motivating on their own, serving as rapid triggers to seek out the drug. Perhaps most troubling, drug-associated stimuli retain their ability to motivate drug seeking behavior after successful rehabilitation and prolonged drug-free periods, because of the persistence of associative memories.

### SUMMARY

The invention is directed, in various embodiments, to compounds and methods useful for inhibition of nonmuscle myosin II, which can be selective inhibition of the nonmuscle myosin II with respect to cardiac myosin II. The compounds are analogs of (S)-blebbistatin (“Blebb”).

In various embodiments, the invention can provide a compound of formula (I)

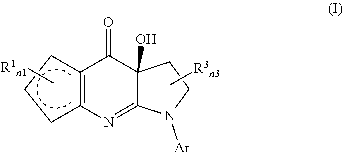

wherein
   the ring bearing R¹ is a 5-membered, 6-membered, or 7-membered aryl or heteroaryl ring system comprising 0, 1, or 2 heteroatoms selected from the group consisting of S, O, N, and NR¹; wherein the ring can be fused with an aryl, heteroaryl, cycloalkyl, or heterocyclyl ring; wherein R¹ can be disposed on any one or more rings of a multiring system;
   R¹ is independently at each occurrence (C1-C4)alkyl, (C1-C4)alkoxyl; (C1-C4)alkoxycarbonyl, (C1-C4)haloalkyl, cyano, nitro, or halo; n1=0, 1, 2, or 3;
   Ar is a monocyclic or bicyclic aryl or heteroaryl ring system, wherein any aryl or heteroaryl thereof can be substituted with 0, 1, 2 or 3 R²;
   R² is independently at each occurrence (C1-C4)alkyl, (C1-C4)alkoxyl), (C1-C4)alkoxycarbonyl, (C1-C4)haloalkyl, hydroxymethyl, R₂NCH₂ wherein R is H or alkyl, cyano, nitro, or halo;
   R³ is independently at each occurrence (C1-C4)alkyl, halo, or (C1-C4)haloalkyl; n3=0, 1, 2, 3, or 4;
   provided that the compound is not blebbistatin or a compound of any of formulas

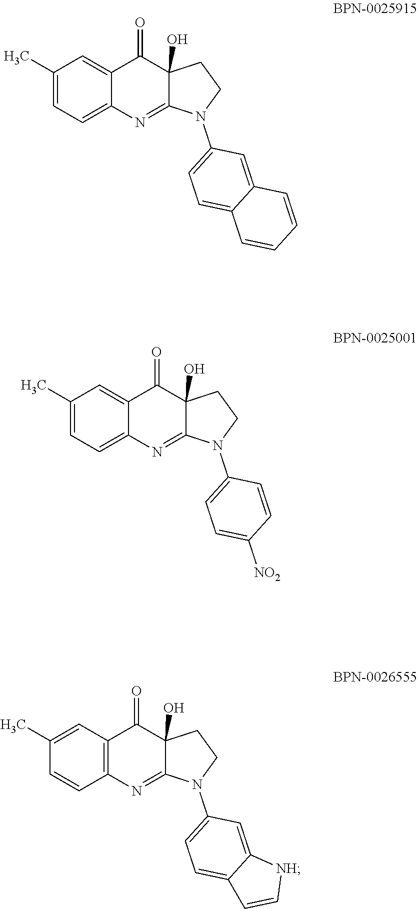 [SMILES: Cc1ccc2c(c1)C(=O)[C@]1(O)CCN(c3ccc4ccccc4c3)C1=N2 ; Cc1ccc2c(c1)C(=O)[C@]1(O)CCN(c3ccc([N+](=O)[O-])cc3)C1=N2 ; Cc1ccc2c(c1)C(=O)[C@]1(O)CCN(c3ccc4cc[nH]c4c3)C1=N2]

or a pharmaceutically acceptable salt thereof.

For example, the ring bearing R¹ can be phenyl, pyridyl, or thienyl.

More specifically, the compound of formula (I) can be a compound of formula (IIa) or (IIb)

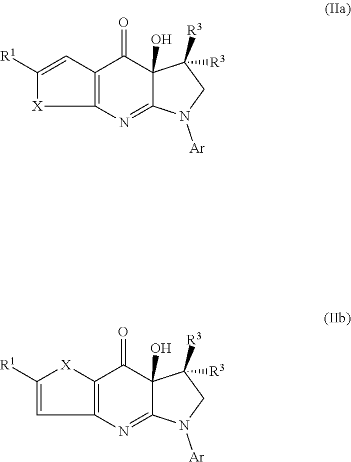

wherein
X is S, or X is a group of formula

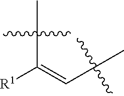

herein wavy lines indicate points of bonding;
   R¹ is independently at each occurrence H, (C1-C4)alkyl, CF₃, or halo;
provided that when X is a group of formula

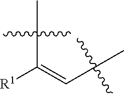

and Ar is unsubstituted phenyl,
at least one R¹ group is other than H or (C1-C4)alkyl;
   Ar is aryl or heteroaryl, wherein any aryl or heteroaryl thereof can be substituted with 0, 1, 2 or 3 R²;
   R² is independently at each occurrence halo, cyano, nitro, CF₃, (C1-C4)alkyl, or (C1-C4)alkoxyl);
   R³ is independently at each occurrence H or CH₃;
   or a pharmaceutically acceptable salt thereof.

For example, for the compound of formula (II), X can be S.

In other embodiments, for the compound of formula (II), X can be a group of formula

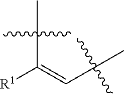

wherein wavy lines indicate points of bonding. In these embodiments, at least one R¹ group of formula (I) can be methyl, halo or CF₃.

In other embodiments of formula (I), Ar can be a group of formula

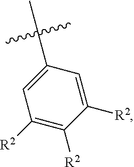

wherein a wavy line indicates a point of bonding.

In other embodiments, Ar can be a pyrazolyl, thiophenyl, isoquinolinyl, benzoxazolyl, quinazolinyl, isoxazolyl, cinnolinyl, quinoxalinyl, benzisoxazolyl, benzothiadiazolyl, pyrazolopyridinyl, imidazopyridinyl, thieopyridinyl, dihydrobenzoxazinyl, triazolopyridinyl, dihydropyridoxazinyl, tetrahydrobenzoxazepinyl, dihydrobenzodioxinyl, dihydrobenzothiazinyl, tetrahydroquinolinyl, tetrahydronaphthyl, or chromanyl, ring system, any of which can be unsubstituted or substituted with 1, 2, or 3 R².

In various specific embodiments of a compound of formula (I) of the invention, the compound can be any one of the compounds depicted in Table 3, with the exception of blebbistatin itself and the three compounds excluded by proviso as described above.

In various embodiments, the invention can provide a method of inhibiting nonmuscle myosin II, comprising contact the nonmuscle myosin II with an effective amount or concentration of a compound of formula (I)

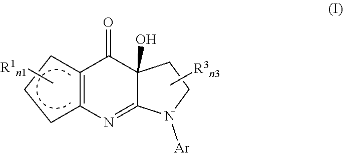

wherein
   the ring bearing R¹ is a 5-membered, 6-membered, or 7-membered aryl or heteroaryl ring system comprising 0, 1, or 2 heteroatoms selected from the group consisting of S, O, N, and NR¹; wherein the ring can be fused with an aryl, heteroaryl, cycloalkyl, or heterocyclyl ring; wherein R¹ can be disposed on any one or more rings of a multiring system;
   R¹ is independently at each occurrence (C1-C4)alkyl, (C1-C4)alkoxyl; (C1-C4)alkoxycarbonyl, (C1-C4)haloalkyl, cyano, nitro, or halo; n1=0, 1, 2, or 3;
   Ar is a monocyclic or bicyclic aryl or heteroaryl ring system, wherein any aryl or heteroaryl thereof can be substituted with 0, 1, 2 or 3 R²;
   R² is independently at each occurrence (C1-C4)alkyl, (C1-C4)alkoxyl), (C1-C4)alkoxycarbonyl, (C1-C4)haloalkyl, hydroxymethyl, dialkylaminomethyl, cyano, nitro, or halo;
   R³ is independently at each occurrence (C1-C4)alkyl, halo, or (C1-C4)haloalkyl; n3=0, 1, 2, 3, or 4;
   provided that the compound is not blebbistatin;
   or a pharmaceutically acceptable salt thereof.

Compounds BPN-0025915, BPN-0025001, and BPN-0026555 (see Table 3) are included in the set of compounds useful for practice of an embodiment of the methods of the invention.

In other embodiments, the invention can provide a method of treatment of substance use relapse in a patient, comprising administering to the patient an effective dose of a compound of formula (I).

In various embodiments, a compound useful for practice of a method of the invention can include:
a compound of formula (I) wherein the ring bearing R¹ is phenyl, pyridyl, or thienyl;
a compound of formula (I) being of formula (IIa) or (IIb)

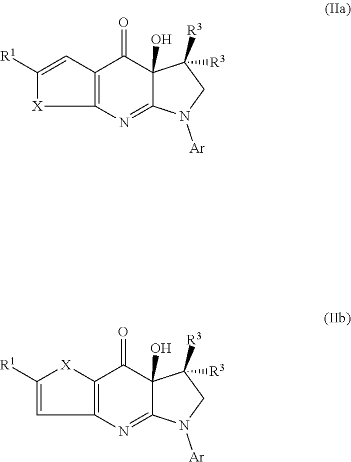

wherein
X is S, or X is a group of formula

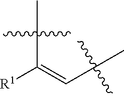

wherein wavy lines indicate points of bonding;
R¹ is independently at each occurrence H, (C1-C4)alkyl, CF₃, or halo;
   provided that when X is a group of formula

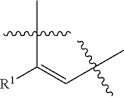

and Ar is unsubstituted phenyl, at least one R¹ group is other than H or (C1-C4)alkyl;
Ar is aryl or heteroaryl, wherein any aryl or heteroaryl thereof can be substituted with 0, 1, 2 or 3 R²;
R² is independently at each occurrence halo, cyano, nitro, CF₃, (C1-C4)alkyl, or (C1-C4)alkoxyl);
R³ is independently at each occurrence H or CHs;
or a pharmaceutically acceptable salt thereof.

In various embodiments of a compound of formula (II) for practice of a method of the invention, X can be S; or X can be a group of formula

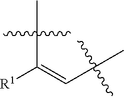

wherein wavy lines indicate points of bonding. In these embodiments, at least one R¹ group of formula (I) can be methyl, halo or CF₃.

In other embodiments of a compound of formula (I) useful for practice of a method of the invention, Ar can be a group of formula

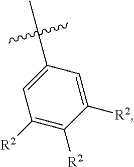

wherein a wavy line indicates a point of bonding.

In other embodiments, Ar can be a pyrazolyl, thiophenyl, isoquinolinyl, benzoxazolyl, quinazolinyl, isoxazolyl, cinnolinyl, quinoxalinyl, benzisoxazolyl, benzothiadiazolyl, pyrazolopyridinyl, imidazopyridinyl, thieopyridinyl, dihydrobenzoxazinyl, triazolopyridinyl, dihydropyridoxazinyl, tetrahydrobenzoxazepinyl, dihydrobenzodioxinyl, dihydrobenzothiazinyl, tetrahydroquinolinyl, tetrahydronaphthyl, or chromanyl, ring system, any of which can be unsubstituted or substituted with 1, 2, or 3 R².

For practice of a method of the invention, in various embodiments a specific example of a compound of formula (I) can be any of the compounds shown in Table 3 other than blebbistatin itself.

In various embodiments, the compound can be more effective, on a molar basis, in inhibition of nonmuscle myosin II relative to myosin II derived from cardiac muscle.

In another aspect, described herein provides a method of treating a disease, disorder, or medical condition in a patient, comprising modulating myosin II ATPase, wherein the modulating of myosin II ATPase comprises administering to the patient at least one compound of Formula (I) or Formula (II) or a pharmaceutically acceptable salt thereof, in a dose, at a frequency, and for a duration to provide a beneficial effect to the patient. In various embodiments, the disease, disorder, or medical condition is selected from the group consisting of renal disease², cancer and metastasis, including hepatocarcinoma³, pancreatic adenocarcinoma⁴, breast cancer⁵⁻⁶, lung carcinoma⁷, glioblastoma⁸⁻¹¹, benign prostate hyperplasia¹², hemostasis or thrombosis¹³⁻¹⁵, nerve injury¹⁶ including retinal damage¹⁷, lung fibrosis¹⁸, liver fibrosis¹⁹, arthrofibrosis²⁰, wound healing²¹⁻²³, spinal cord injury²⁴, periodontitis²⁵, glaucoma²⁶⁻²⁷ and immune-related diseases including multiple sclerosis²⁸. In certain embodiments, the disease, disorder, or medical condition is selected from the group consisting of viral infection including herpes virus²⁹⁻³³, high blood pressure³⁴, pulmonary hypertension³⁵, chronic respiratory diseases³⁶, cardiovascular disease³⁷, erectile disfunctions³⁸, thrombotic disorders¹³, overactive bladder³⁹, cardiomyopathies⁴⁰, spasms⁴¹⁻⁴⁶, skeletal myopathies⁴⁷, and psychiatric disorders, including substance use⁴⁸, anxiety disorders (e.g. phobias)⁴⁹ and stress disorders (e.g. posttraumatic stress disorder)⁵⁰. Addiction can include abuse of or addiction to anything classified as a Substance-Related or Addictive Disorder in the Diagnostic and Statistical Manual of Mental Disorders (DSM), such as, but not limited to, cocaine, opioids, amphetamines⁵¹⁻⁵², ethanol, cannabis/marijuana, nicotine, and activities (e.g. gambling)⁵³.

### BRIEF DESCRIPTION OF THE FIGURES

FIG. 1(A,B,C): Long-lasting disruption of METH (methamphetamine)-associated memory by intra-amygdala administration of (S)-blebbistatin (“Blebb”) (90 ng/ul).

FIGS. 2A, 2B, 2C, 2D: Post-consolidation loss of NMIIB (A-B) produces a lasting disruption of METH-associated memory (C-D). *P<0.05.

FIG. 3(A,B,C,D): Systemic NMII inhibition selectively targets the storage of (A) a METH-associated memory, (B) having no effect on an auditory fear memory. Similarly, spine density associated with a METH-associated memory (C), but not a fear memory (D), is reduced.

FIG. 4: Brain and plasma distribution of blebbistatin after IP dosing at 10 mg/kg in mice.

FIG. 5: Structure of (S)-blebbistatin, and compound numbering scheme.

FIG. 6(A,B,C,D,E): Nonmuscle myosin II inhibition disrupts polydrug-associated memories when the drugs are administered on alternating days (A) Schematic of the experimental design. (B) Vehicle-treated mice demonstrated a significant METH/NIC CPP during Test 1, but not during Test 2. (C) Blebb-treated mice did not demonstrate a significant METH/NIC CPP during either test. (D) Veh-treated mice demonstrated a significant METH/MOR CPP during Test 1 and 2. (E) Blebb-treated mice did not demonstrate a significant METH/MOR CPP during either test. METH/NIC: Veh n=10; METH/NIC: Blebb n=10; METH/MOR: Veh n=19; METH/MOR: Blebb n=19; * p<0.05, ** p<0.01, *** p<0.001. Error bars represent±s.e.m.

FIG. 7: Cardiac safety Characterization for Selected Blebbistatin Analogs: Echocardiography assessment in vivo with IV drug infusion

### DETAILED DESCRIPTION

The physical storage site of memory is dendritic spines⁵⁴. Spines are the small, highly dynamic postsynaptic structures found at the majority of forebrain excitatory synapses. At the time of learning, spines undergo structural and functional changes critical to the formation of lasting memories⁵⁵⁻⁵⁶. The workhorse of this spine plasticity is actin polymerization, the process of linking actin monomers into complex, branched filaments (F-actin)⁵⁷⁻⁵⁸. Disrupting F-actin dynamics at the time of learning prevents the formation of long-term memories⁵⁹⁻⁶³. However, actin rapidly stabilizes after synaptic stimulation, such that the cytoskeleton and associated memory become impervious to disruption by actin depolymerizing agents, such as Latrunculin A (LatA), within minutes of stimulation^{59, 62-64}. In stark contrast to this, we discovered that the F-actin supporting METH-associated memories in the basolateral amygdala complex (BLC), a subregion of the amygdala (AMY) and the brain's emotional memory center and a hub for drug-associated memories, remains dynamic long after learning. This revealed an unexpected weakness of pathogenic drug associations, providing a mechanism to selectively target the storage of METH-associated memories⁶⁵.

Importantly, other types of memories are not disrupted by LatA and a single home cage treatment is sufficient to produce an immediate and long-lasting disruption of memory-induced drug seeking. Thus, it is possible to selectively manipulate drug-associated memories without retrieval⁶⁵.

Given these results, actin depolymerization would seem to be a promising therapeutic target. However, β-actin, the isoform implicated in neuronal plasticity⁶⁶⁻⁶⁹, is ubiquitously expressed throughout the body and critical for a multitude of processes, such as cell division and cardiac function. Therefore, we turned our focus to nonmuscle myosin II (NMII), a direct regulator of the synaptic actin cytoskeleton. NMII is a molecular motor we have shown to be a critical, temporally restricted player in synaptic actin polymerization and fear memory^{59, 63}. (S)-Blebbistatin, the active enantiomer of blebbistatin, is the first and, until recently, the only inhibitor known to have activity against nonmuscle myosin IIs⁷⁰⁻⁷³.

The terms blebblistatin and Blebb as used throughout refer to (S)-blebbistatin. The blebbistatin analogs of the present invention are all likewise (S)-enantiomers. Blebbistatin is commercially available (e.g. Tocris, Sigma) at >98% purity. Using blebbistatin, we have recapitulated all of the LatA findings including a single treatment being sufficient to prevent drug seeking for at least one month in an animal relapse model^{52,74}.

Justification for the proposed discovery and development work is based on several key discoveries made over the past few years with regards to NMII as a target for drug seeking induced by METH-associated memories^{65,74}.

In 2013, we published the finding that depolymerization of actin in the AMY disrupts the storage of METH-associated memories in a selective, retrieval-independent manner⁶⁵. Because of the limited clinical potential of actin depolymerizing agents, we turned to NMII, a molecular motor we previously found to be a key regulator of memory, synaptic plasticity (long-term potentiation [LTP]) and actin polymerization in dendritic spines^{59 63}. Through a series of experiments in a recently published studies, we detailed NMII's potential as a therapeutic target for SUD relapse^{52,74-76}. For instance, through a single intra-BLC or IP administration of blebbistatin, we have found that METH-associated memory (conditioned place preference [CPP]assay) is disrupted in adult and adolescent male and female mice and rats (inactive enantiomer used as control with intra-BLC drug delivery or racemic blebbistatin used with systemic drug delivery due to solubility limitations)^{52, 65}.

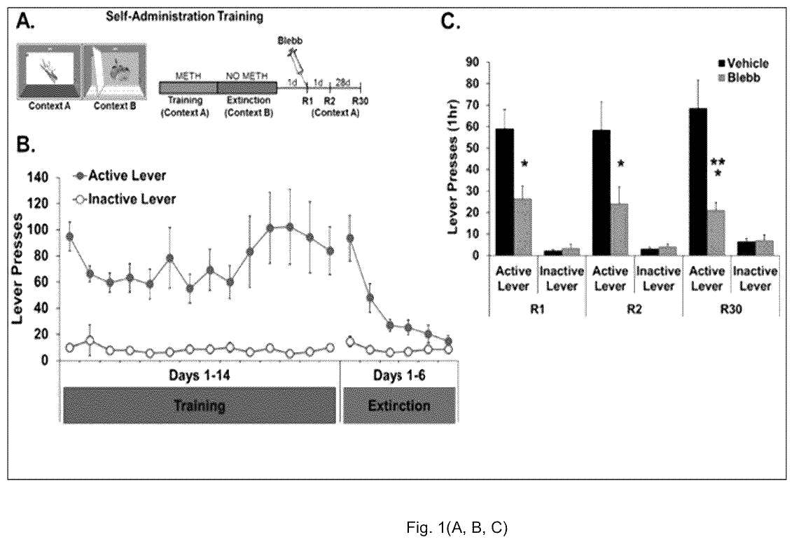

Further, blebbistatin prevents context-induced reinstatement of METH seeking (FIG. 1A; P<0.05-0.005), a gold standard animal model of relapse^{52, 74}. FIG. 1B shows lever pressing for METH during the self-administration training period in Context A, and subsequent extinction of lever pressing in Context B, when METH is removed. FIG. 1C shows lever pressing during the METH-free reinstatement sessions on Days 1, 2 and 30, when animals are returned to the METH-paired environment (Context A). Reinstatement testing on Day 1 was performed 30 minutes after a single intra-BLC administration of blebbistatin or the inactive enantiomer. No blebbistatin was given prior to Reinstatement sessions on Days 2 or 30, indicating the long-lasting memory disruption produced by a single administration of blebbistatin (at least one month), with no spontaneous renewal of the memory.

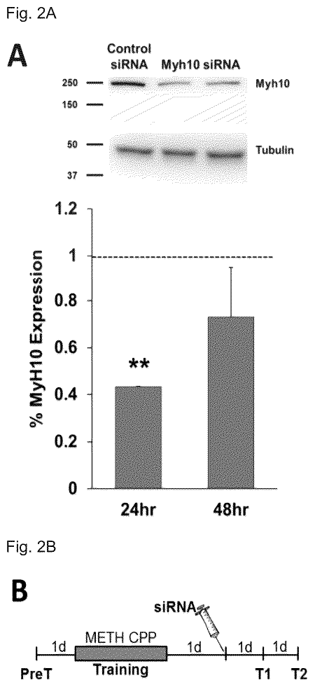

Blebbistatin has been shown to inhibit all classes of myosin IIs 7-79. Myosin IIs are made of light chains, which are interchangeable across classes, and heavy chains, which are unique to each myosin II isoform. The heavy chain contains actin and ATP binding sites, as well as the force generating motor head that moves actin. According to RNA-Seq data from the Miller lab, none of the skeletal muscle heavy chain isoforms are expressed in the BLC of adult mice. However, two of the cardiac muscle myosin II's, Myh7 and Myh7b, the smooth muscle myosin II (Myh11) and all three nonmuscle myosin heavy chain isoforms (Myh14, Myh10, Myh9) are, with Myh10 expression being the highest by several fold. All six myosin IIs are found at the synapse. Therefore, we employed a genetic method of acute, focal knockdown of MYH10 in the BLC and found its post-training loss (FIG. 2A) to be sufficient to disrupt an established METH-associated memory (FIG. 2B-C)⁷⁴. Interestingly, while protein expression data is not available, myosin II transcript levels in the human AMY (Allen Brain Atlas) suggests that a focus on the NMII class as a whole, as opposed to solely NMIIB, may be wise from a drug development perspective, as mRNA for NMIIA, B and C are expressed at equal levels. It is important to note that the NMIIs are a highly conserved class of actin-based molecular motors. For instance, M. musculus Myh10 has 99.1% sequence homology to H. sapiens.

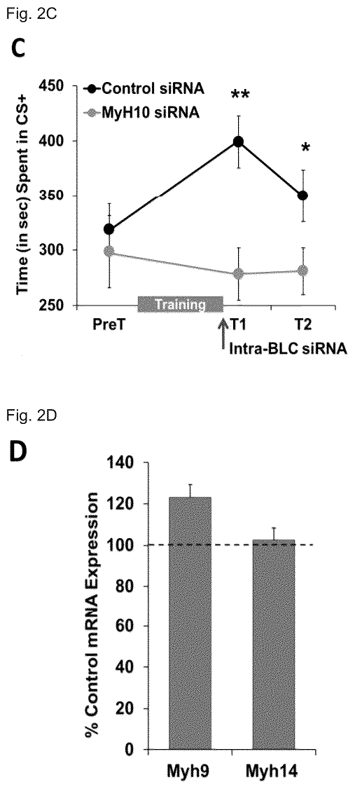

The potential for systemic NMII inhibition to selectively target METH-associated memories in the way that intra-AMY administration does was also investigated. Results with IP administered Blebb indicate that it crosses the blood brain barrier at a high enough concentration to disrupt a METH-associated memory (IP, 10 mg/kg [brain levels=90 μM]; FIG. 3A). Yet, the effect remains selective, having no similarly immediate effect on a fear memory (FIG. 3B), or memories associated with food reward⁷⁵. IP administered Blebbistatin also disrupts reconsolidation of memories for cocaine, nicotine and mephedrone, a result with additional potential therapeutic relevance in treating relapse⁷⁵⁻⁷⁶. In addition, the BLC spine density increase that accompanies a METH-associated memory is reversed by Blebbistatin (FIG. 3C), without altering the spine density increase associated with a fear memory (FIG. 3D)^{65, 74}.

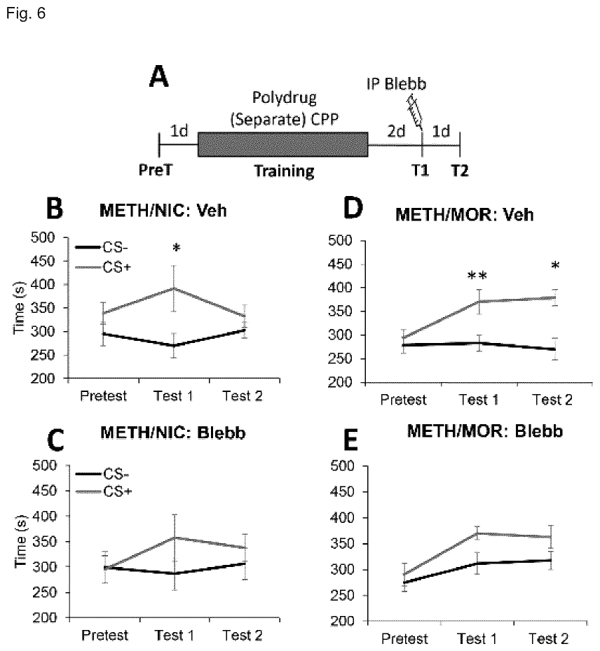

The vast majority of individuals with substance use disorder are polydrug users. For example, most METH users also smoke. We recently made an unexpected discovery with direct relevance to polydrug use⁷⁶. When animals are treated with both METH and a drug that is impervious to Blebb on its own (nicotine or morphine), either concurrently or on alternating days, the nicotine and morphine memories become susceptible to immediate disruption by Blebb, akin to the effect on METH alone memories (FIG. 6). Thus, memories for previously impervious drugs of abuse become susceptible to NMII inhibition when METH associations are also formed. Importantly, this occurs when associative training is performed with METH and the other drug of abuse being administered simultaneously or separately, on different days. We are currently expanding our analysis to other commonly abused drugs, including other opioids, such as heroin, alcohol, cocaine and synthetic cathinones.

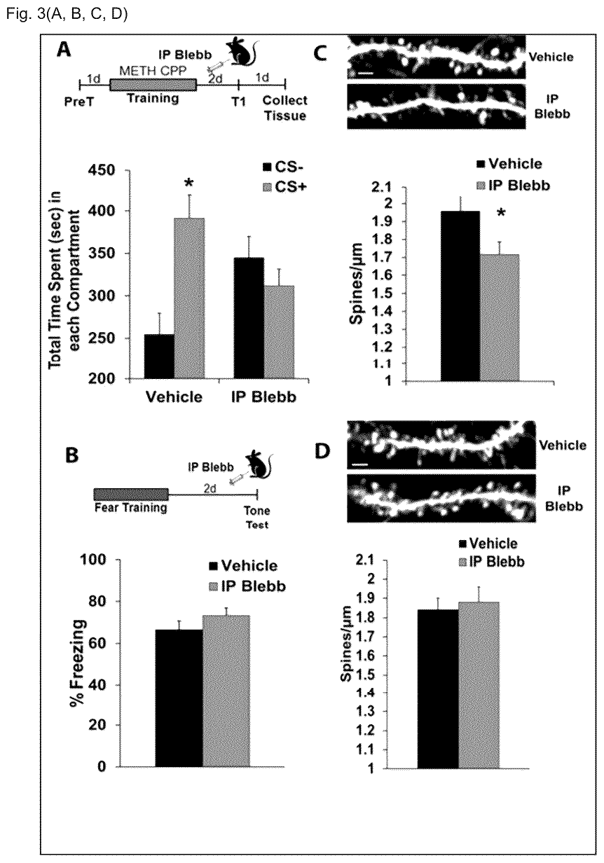

In addition to its efficacy in animal relapse models, several properties make Blebb an excellent scaffold for medicinal chemistry. Paramount among these are the molecule's small size, high brain penetration, rapid clearance from plasma and brain (short-acting is sufficient and reduces unwanted peripheral and central effects; FIG. 4). In addition, we have observed no effect of systemically administered racemic Blebb in a broad panel of rodent behaviors⁷⁴.

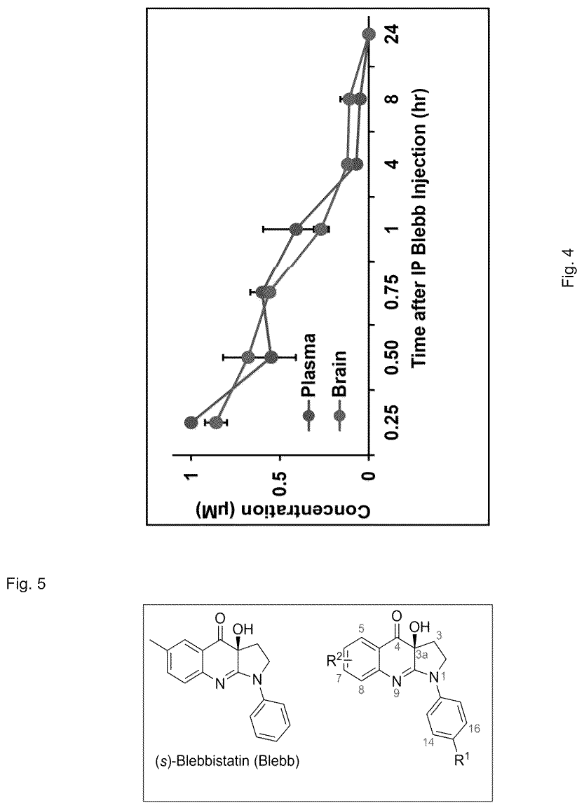

Blebb, identified in an HTS campaign in the early 2000s via an ATPase assay⁷⁰, and its few derivatives (FIG. 5) were until very recently, the only small molecules reported to have inhibitory activity toward NMII. BDM (2,3-butanedione monoxime)⁸⁰ some N-benzylsulphonamides and hydroxycoumarines⁸¹ inhibit some myosin IIs, but they are either not active against NMII and/or lack selectivity and thus, cannot be used as viable probes, even for in vitro studies. Blebb has been widely used as an in vitro probe since its discovery. Due to photoinactivation upon exposure to blue light⁷³, analogs made through simple modifications, including a nitro-derivative, have been developed to establish the photostability.⁸²⁻⁸⁴ More discussion of these compounds can be found in the next section.

Blebb is the (s)-configuration of Blebbistatin (FIG. 5). Since its discovery, its biochemical and cell potency have been assayed against many myosin ATPases^{70, 73, 77, 85-88} and in a number of cells/tissues.^{77, 86-92} The biochemical IC₅₀ values reported for Blebb in various myosin IIs are 1.8-9 μM⁷⁷. Specific IC₅₀ values in cell-based assays have not been reported, but Blebb has been shown to be effective across a wide concentration range from ˜900 nM to 90 μM,^{79, 86-87, 93-94}, indicating that Blebb is cell penetrant, consistent with our own data. Limited SAR has been performed around Blebb's core structure⁷⁰, likely due to the therapeutic potentials of NMII inhibitors having only been realized in the past few years. Modifications and optimization to Blebb are needed in order to obtain more selective NMII inhibitors to serve as viable drug candidates.

The current class of blebbistatin analogs have been synthesized as generally described in Synthetic Schemes 1-7 (below) and are characterized in vitro in Table 1. Chemical structures of compounds of the invention listed in Table 1 are shown in Table 3, below. Synthetic Scheme 1 is an overview of a synthetic method that can be used in preparation of the specific examples and the structures read upon by the generic structural claim, and Synthetic Schemes 2-7 are examples of different embodiments of synthetic methods for certain of the specific examples and the structures read upon by the generic structural claim.

Table 1 lists properties of compounds of the invention, analogs of blebbistatin having an inhibitory selectivity for nonmuscle myosin II relative to cardiac myosin II of greater than 3.0. Compounds having significantly less inhibitory activity (higher Ki) for cardiac myosin II can exhibit lower cardiac toxicity of the compound relative to blebbistatin, as discussed below.

Table 1 lists the selective inhibitory compounds, defined by this ratio of Ki/EC50, the structures of which are shown in Table 3, according to a unique compound identifier also shown with the corresponding chemical structure in the Figures. All compounds of the invention are of the same absolute configuration as (S)-blebbistatin, and are based on the parent ring structure

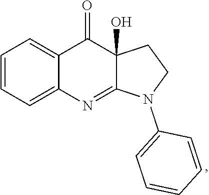 [SMILES: O=C1c2ccccc2N=C2N(c3ccccc3)CC[C@@]12O]

(S)-3a-hydroxy-1-phenyl-1,2,3,3a-tetrahydro-4H-pyrrolo[2,3-b]quinolin-4-one. The phenyl rings in (S)-blebbistatin can be substituted, and can be replaced by various heteroaryl rings, unsubstituted or substituted, in certain of the analogs, compounds of the invention.

The top row of data are provided for blebbistatin itself for purposes of comparison. Blebbistatin has a ratio (CMMII/NMII) of 0.789, showing that the Ki value of the parent compound with respect to inhibition of cardiac myosin II is lower than is the EC50 value of nonmuscle myosin II, i.e., the compound has more potent inhibitory activity of the cardiac form of myosin II than it does towards the nonmuscle form of myosin II. All the compounds of the invention or compounds for practice of methods of the invention, indicated in Table 1, have at least a three-fold higher Ki value for inhibition of cardiac myosin II relative to the EC50 value of the compound for inhibition of nonmuscle myosin II.

The first column of Table 1 provides a unique numerical identifier of the compound, the structure of each of which is indicated in Table 3. The second, third, and fourth columns of Table 1 provide data concerning the photostability of the indicated compound. In comparison with blebbistatin, many compounds used for practice of methods of the invention exhibit a significantly higher photostability than does blebbistatin. These columns (Photostability), provide values for the percentage remaining undegraded of the compound under conditions after 4 and 24 hours illumination, compared to percentage remaining undegraded in the dark under comparable conditions. Blebbistatin is known to be somewhat photo-unstable, and improvement of photostability is a benefit in formulation of pharmaceutical compositions for administration to patients undergoing treatment.

The fifth column of Table 1 provides EC50 values (μM) determined for inhibition of nonmuscle myosin II by the indicated compound. These figures were obtained as described in the bioassays section. The sixth and seventh columns give the Ki values (μM) of inhibition of cardiac myosin II and the ratio of cardiac muscle myosin inhibition relative to non-muscle myosin II. The eighth column gives skeletal myosin II inhibition. For compounds marked as NMII selective (“NMII-sel”), no Ki value for cardiac myosin II could be determined; the compounds had no detectable inhibitory properties versus the cardiac myosin II. The ninth column shows the chiral purity of the sample tested.

TABLE 1
In vitro characterization data for selected blebbistatin analogs

| | | | | | | Ratio | | Chiral |
|---|---|---|---|---|---|---|---|---|
| | Photostability | | | NMII | CMMII | CMMII: | SKMMII | HPLC |
| | 4 | 24 | Dark | EC50 | K_{I} | NMII | K_{I} | Purity |
| Identifier | hours | hours | (24 h) | (μM) | (μM) | Ki/EC50 | (μM) | % |
| Blebbi- | 82.9 | 9.3 | >99 | 1.9 | 1.5 | 0.8 | 0.36 | >99 |
| statin | | | | | | | | |
| BPN- | 90.3 | 12.1 | >99 | 5.2 | 28.0 | 5.4 | 7.00 | 97.0 |
| 0025044 | | | | | | | | |
| BPN- | 96.1 | 48.6 | >99 | 5.1 | 120.0 | 23.5 | 22.00 | >99 |
| 0025059 | | | | | | | | |
| BPN- | 82.8 | 4.7 | 99.0 | 7.5 | 48.0 | 6.4 | 9.70 | >99 |
| 0025100 | | | | | | | | |
| BPN- | 6.6 | 2.6 | >99 | 3.2 | 22.0 | 6.9 | 8.00 | >99 |
| 0025110 | | | | | | | | |
| BPN- | 93.0 | 28.6 | 94.7 | 10.2 | 400.0 | 39.2 | 35.00 | >99 |
| 0025240 | | | | | | | | |
| BPN- | 92.5 | 39.4 | >99 | 4.6 | n/a | NMII-sel | 7.70 | >99 |
| 0025254 | | | | | | | | |
| BPN- | 39.8 | 7.5 | 98.3 | 6.4 | n/a | NMII-sel | 11.00 | 95.4 |
| 0025903 | | | | | | | | |
| BPN- | ND | ND | ND | 3.9 | 18.0 | 4.6 | 1.80 | 91.7 |
| 0025919 | | | | | | | | |
| BPN- | 64.0 | 7.0 | >99 | 48.0 | n/a | NMII-sel | n/a | 87.5 |
| 0026282 | | | | | | | | |
| BPN- | 39.4 | 10.8 | 92.0 | 3.5 | n/a | NMII-sel | 5.00 | 96.7 |
| 0026283 | | | | | | | | |
| BPN- | 51.9 | 8.2 | 94.7 | 1.6 | 9.1 | 5.7 | 0.90 | >99 |
| 0026285 | | | | | | | | |
| BPN- | 98.5 | 90.4 | >99 | 2.2 | 30.0 | 13.6 | 3.50 | >99 |
| 0026324 | | | | | | | | |
| BPN- | 89.0 | 14.0 | 97.8 | 5.3 | n/a | NMII-sel | 13.00 | >99 |
| 0026325 | | | | | | | | |
| BPN- | 85.2 | 18.0 | 93.5 | 2.5 | 13.0 | 5.2 | 1.20 | 98.2 |
| 0026387 | | | | | | | | |
| BPN- | 97.9 | 75.0 | >99 | 3.4 | 11.0 | 3.2 | 1.70 | >99 |
| 0026393 | | | | | | | | |
| BPN- | >99 | 98.7 | >99 | 3.8 | n/a | NMII-sel | 4.80 | 98.25 |
| 0026394 | | | | | | | | |
| BPN- | 81.2 | 9.6 | 97.7 | 3.0 | 50.0 | 16.7 | 3.30 | 99.0 |
| 0026401 | | | | | | | | |
| BPN- | ND | ND | ND | 3.3 | 23.0 | 7.0 | 3.60 | >99 |
| 0026404 | | | | | | | | |
| BPN- | 94.3 | 82.2 | 97.3 | 36.0 | 110.0 | 3.1 | 33.00 | 49.5 |
| 0026413 | | | | | | | | |
| BPN- | 98.6 | 77.1 | >99 | 5.3 | n/a | NMII-sel | 15.00 | 96.5 |
| 0026457 | | | | | | | | |
| BPN- | 80.9 | 15.9 | 93.4 | 6.6 | n/a | NMII-sel | 20.00 | >99 |
| 0026495 | | | | | | | | |
| BPN- | 41.7 | 3.5 | 97.9 | 2.6 | 83.0 | 31.9 | 15.00 | >99 |
| 0026496 | | | | | | | | |
| BPN- | 82.7 | 6.1 | 97.5 | 2.1 | 33.0 | 15.7 | 9.80 | >99 |
| 0026497 | | | | | | | | |
| BPN- | 97.7 | 84.9 | >99 | 0.5 | 5.7 | 12.4 | 0.18 | 92.7 |
| 0026499 | | | | | | | | |
| BPN- | 92.2 | 22.8 | >99 | 2.4 | n/a | NMII-sel | 23.00 | >99 |
| 0026500 | | | | | | | | |
| BPN- | 87.7 | 6.4 | 91.7 | 33.0 | n/a | NMII-sel | 60.00 | 47.7 |
| 0026533 | | | | | | | | |
| BPN- | 51.1 | 10.8 | 93.7 | 5.3 | n/a | NMII-sel | 4.80 | 93.0 |
| 0026544 | | | | | | | | |
| BPN- | 89.4 | 5.1 | >99 | 7.0 | n/a | NMII-sel | 12.00 | 92.1 |
| 0026545 | | | | | | | | |
| BPN- | 81.2 | 12.5 | 93.5 | 8.9 | n/a | NMII-sel | 22.00 | 85.1 |
| 0026546 | | | | | | | | |
| BPN- | 98.6 | >99 | >99 | 5.5 | 18.0 | 3.3 | 4.60 | 98.9 |
| 0026576 | | | | | | | | |
| BPN- | 77.3 | 8.5 | 93.7 | 1.0 | 5.0 | 5.0 | 0.37 | 98.5 |
| 0026579 | | | | | | | | |
| BPN- | 69.6 | 2.3 | 94.2 | 1.0 | 15.0 | 15.0 | 0.29 | >99 |
| 0026605 | | | | | | | | |
| BPN- | 85.9 | 4.1 | 95.5 | 5.9 | n/a | NMII-sel | n/a | 98.6 |
| 0026606 | | | | | | | | |
| BPN- | 98.5 | 84.7 | 98.1 | 6.1 | n/a | NMII-sel | 5.80 | 96.1 |
| 0026607 | | | | | | | | |
| BPN- | 86.7 | <1 | 97.5 | 4.3 | 50.0 | 11.6 | 1.70 | 95.3 |
| 0026608 | | | | | | | | |
| BPN- | ND | ND | ND | 1.5 | 5.5 | 3.7 | 0.43 | 98.5 |
| 0026609 | | | | | | | | |
| BPN- | 92.3 | 17.2 | 96.6 | 4.8 | 18.0 | 3.8 | 5.90 | >99 |
| 0026618 | | | | | | | | |
| BPN- | 77.4 | <1 | 91.0 | 1.6 | 36.0 | 22.5 | 2.50 | 99.0 |
| 0026619 | | | | | | | | |
| BPN- | 94.1 | 27.8 | 92.8 | 6.4 | n/a | NMII-sel | 16.00 | >99 |
| 0026620 | | | | | | | | |
| BPN- | 93.3 | 61.4 | 93.5 | 5.7 | 40.0 | 7.0 | 3.40 | 83.0 |
| 0026621 | | | | | | | | |
| BPN- | 90.5 | 88.2 | 92.2 | 15.0 | 210.0 | 14.0 | 24.00 | >99 |
| 0026624 | | | | | | | | |
| BPN- | 87.9 | 71.9 | 91.6 | 1.7 | 12.0 | 7.1 | 0.37 | 94.3 |
| 0026638 | | | | | | | | |
| BPN- | 89.8 | 49.5 | >99 | 3.3 | 14.0 | 4.2 | 1.10 | >99 |
| 0026639 | | | | | | | | |
| BPN- | 71.1 | 17.8 | 96.8 | 2.0 | 8.0 | 4.0 | 0.53 | 89.6 |
| 0026640 | | | | | | | | |
| BPN- | 64.0 | <1 | 85.1 | 4.8 | 17.0 | 3.5 | 1.90 | >99 |
| 0026643 | | | | | | | | |
| BPN- | 94.3 | 84.3 | 97.7 | 4.7 | 56.0 | 11.9 | 4.50 | 91.3 |
| 0026648 | | | | | | | | |
| BPN- | 80.5 | 5.5 | >99 | 4.9 | n/a | NMII-sel | 4.30 | >99 |
| 0026650 | | | | | | | | |
| BPN- | 73.0 | 5.8 | 97.5 | 3.3 | n/a | NMII-sel | 3.90 | 96.3 |
| 0026651 | | | | | | | | |
| BPN- | 72.1 | 17.9 | 91.1 | 2.6 | 9.0 | 3.5 | 0.93 | 93.2 |
| 0026659 | | | | | | | | |
| BPN- | 87.4 | 7.6 | 93.8 | 8.2 | n/a | NMII-sel | 33.00 | >99 |
| 0026660 | | | | | | | | |
| BPN- | 95.5 | 65.4 | 98.2 | 2.4 | 53.0 | 22.1 | 2.20 | 89.4 |
| 0026661 | | | | | | | | |
| BPN- | 85.4 | 7.5 | >99 | 1.1 | 6.0 | 5.5 | 0.19 | 97.6 |
| 0026662 | | | | | | | | |
| BPN- | 40.7 | 2.0 | 97.3 | 0.8 | 2.8 | 3.5 | 0.12 | >99 |
| 0026663 | | | | | | | | |
| BPN- | 30.3 | <1 | 97.7 | 0.9 | 29.0 | 33.3 | 1.50 | 95.8 |
| 0026664 | | | | | | | | |
| BPN- | 2.7 | <1 | 97.4 | 0.6 | 4.2 | 6.7 | 0.14 | >99 |
| 0026665 | | | | | | | | |
| BPN- | 85.7 | 2.9 | >99 | 1.2 | 6.2 | 5.2 | 0.33 | 98.6 |
| 0026667 | | | | | | | | |
| BPN- | 87.8 | 11.2 | >99 | 5.3 | 25.0 | 4.7 | 1.20 | >99 |
| 0026669 | | | | | | | | |
| BPN- | 34.1 | 1.7 | >99 | 0.6 | 3.5 | 6.0 | 0.27 | 53.0 |
| 0026693 | | | | | | | | |
| BPN- | 40.7 | <1 | >99 | 1.7 | 5.3 | 3.1 | 1.40 | 83.5 |
| 0026708 | | | | | | | | |
| BPN- | 98.7 | 93.0 | 98.2 | 1.2 | 30.6 | 25.5 | 1.30 | 95.7 |
| 0026709 | | | | | | | | |
| BPN- | 6.6 | <1 | >99 | 1.7 | 5.2 | 3.1 | 0.95 | 92.2 |
| 0026710 | | | | | | | | |
| BPN- | 76.3 | 24.1 | 95.4 | 6.0 | n/a | NMII-sel | 4.40 | 95.8 |
| 0026726 | | | | | | | | |
| BPN- | >99 | 94.2 | >99 | 2.9 | 73.0 | 25.2 | 2.60 | 97.7 |
| 0026745 | | | | | | | | |
| BPN- | 58.8 | 10.6 | >99 | 4.5 | 17.0 | 3.8 | 3.60 | 98.3 |
| 0026746 | | | | | | | | |
| BPN- | 88.2 | 22.4 | >99 | 3.0 | 14.0 | 4.7 | 2.60 | 96.7 |
| 0026748 | | | | | | | | |
| BPN- | 87.8 | <5 | >99 | 3.2 | n/a | NMII-sel | 23.00 | >99 |
| 0026749 | | | | | | | | |
| BPN- | >99 | 92.9 | >99 | 3.8 | n/a | NMII-sel | 1.50 | >99 |
| 0026770 | | | | | | | | |
| BPN- | 86.7 | 18.1 | >99 | 2.6 | 8.9 | 3.4 | 1.10 | >99 |
| 0026774 | | | | | | | | |
| BPN- | 95.8 | 59.3 | 91.6 | 7.4 | 23.0 | 3.1 | 5.90 | >99 |
| 0026785 | | | | | | | | |
| BPN- | 87.8 | 66.6 | 96.0 | 11.0 | n/a | NMII-sel | n/a | >99 |
| 0026786 | | | | | | | | |
| BPN- | 82.0 | <1 | >99 | 0.5 | 1.6 | 3.1 | 0.11 | >99 |
| 0026813 | | | | | | | | |
| BPN- | 91.2 | 88.2 | 90.2 | 0.8 | 2.8 | 3.6 | 0.27 | 91.2 |
| 0026815 | | | | | | | | |
| BPN- | 96.6 | 84.8 | 97.3 | 1.8 | 50.0 | 27.8 | 1.70 | >99 |
| 0026819 | | | | | | | | |
| BPN- | 99.0 | 91.8 | >99 | 13.0 | n/a | NMII-sel | n/a | >99 |
| 0026847 | | | | | | | | |
| BPN- | >99 | 98.7 | >99 | 26.0 | 320.0 | 12.3 | 61.00 | 66.7 |
| 0026848 | | | | | | | | |
| BPN- | 98.5 | 91.8 | >99 | 1.0 | 16.0 | 16.0 | 0.77 | >99 |
| 0026849 | | | | | | | | |
| BPN- | 62.4 | 3.8 | 85.1 | 2.6 | 25.0 | 9.6 | 5.60 | 81.8 |
| 0026852 | | | | | | | | |
| BPN- | 91.2 | 88.2 | 90.2 | 12.0 | n/a | NMII-sel | n/a | 98.9 |
| 0026853 | | | | | | | | |
| BPN- | 70.9 | 1.8 | >99 | 1.8 | 11.0 | 6.1 | 0.80 | >99 |
| 0026881 | | | | | | | | |
| BPN- | 98.8 | 96.8 | >99 | 5.8 | 21.0 | 3.6 | 4.50 | >99 |
| 0026882 | | | | | | | | |
| BPN- | >99 | 98.5 | >99 | 2.7 | 19.0 | 7.0 | 4.20 | 87.8 |
| 0026886 | | | | | | | | |
| BPN- | 90.2 | 24.7 | 97.4 | 3.0 | 22.0 | 7.3 | 4.90 | >99 |
| 0027036 | | | | | | | | |
| BPN- | >99 | >99 | >99 | 24.0 | n/a | NMII-sel | 18.00 | 69.1 |
| 0027037 | | | | | | | | |
| BPN- | >99 | 98.7 | >99 | 2.7 | 17.0 | 6.3 | 0.61 | >99 |
| 0027038 | | | | | | | | |
| BPN- | 89.8 | 31.3 | 95.9 | 22.0 | 68.0 | 3.1 | 4.60 | 49.3 |
| 0027039 | | | | | | | | |
| BPN- | 94.8 | 21.4 | 95.7 | 1.2 | 37.0 | 30.8 | 2.60 | 94.1 |
| 0027040 | | | | | | | | |
| BPN- | 92.7 | 13.8 | >99 | 6.7 | n/a | NMII-sel | 10.00 | >99 |
| 0027042 | | | | | | | | |
| BPN- | 97.7 | 72.3 | >99 | 2.9 | n/a | NMII-sel | 15.00 | 89.1 |
| 0027043 | | | | | | | | |
| BPN- | >99 | 95.3 | >99 | 10.0 | n/a | NMII-sel | 20.00 | 45.6 |
| 0027071 | | | | | | | | |
| BPN- | >99 | >99 | >99 | 10.0 | 31.0 | 3.1 | 5.00 | 96.3 |
| 0027072 | | | | | | | | |
| BPN- | 95.8 | 93.4 | 94.5 | 21.0 | 77.0 | 3.7 | 16.00 | >99 |
| 0027074 | | | | | | | | |
| BPN- | 98.1 | 88.1 | >99 | 3.0 | n/a | NMII-sel | 4.80 | >99 |
| 0027080 | | | | | | | | |
| BPN- | 97.0 | 46.3 | 96.9 | 1.1 | 9.0 | 8.2 | 0.29 | 89.8 |
| 0027081 | | | | | | | | |
| BPN- | >99 | 97.0 | >99 | 2.8 | n/a | NMII-sel | 10.00 | >99 |
| 0027108 | | | | | | | | |
| BPN- | 82.5 | 29.4 | >99 | 0.5 | 11.0 | 21.2 | 0.27 | 91.4 |
| 0027109 | | | | | | | | |
| BPN- | 68.6 | 9.2 | 94.4 | 1.2 | 5.5 | 4.6 | 0.68 | 84.2 |
| 0027118 | | | | | | | | |
| BPN- | 75.6 | 6.5 | 98.6 | 2.5 | 19.0 | 7.6 | 2.70 | 70.3 |
| 0027121 | | | | | | | | |
| BPN- | 69.5 | 5.3 | 97.1 | 1.7 | 45.0 | 26.5 | 5.90 | 82.7 |
| 0027122 | | | | | | | | |
| BPN- | 89.9 | 22.5 | >99 | 14.0 | n/a | NMII-sel | 330.00 | >99 |
| 0027159 | | | | | | | | |
| BPN- | 95.5 | 73.0 | >99 | 2.9 | 30.0 | 10.3 | 0.20 | 81.9 |
| 0027160 | | | | | | | | |
| BPN- | 78.6 | 60.2 | 98.3 | 1.1 | 24.0 | 21.8 | 0.24 | 73.7 |
| 0027161 | | | | | | | | |
| BPN- | 98.5 | 95.9 | >99 | 7.9 | 52.0 | 6.6 | 4.50 | 77.2 |
| 0027162 | | | | | | | | |
| BPN- | 95.0 | 44.6 | >99 | 4.2 | 21.0 | 5.0 | 5.90 | 92.4 |
| 0027163 | | | | | | | | |
| BPN- | >99 | 99.0 | >99 | 2.7 | 91.0 | 33.7 | 3.10 | 97.5 |
| 0027181 | | | | | | | | |
| BPN- | 94.9 | 7.1 | >99 | 7.5 | n/a | NMII-sel | 12.00 | 75.2 |
| 0027196 | | | | | | | | |
| BPN- | >99 | 36.4 | >99 | 2.5 | n/a | NMII-sel | 2.40 | 58.8 |
| 0027197 | | | | | | | | |
| BPN- | 89.9 | 21.4 | >99 | 2.9 | 10.5 | 3.6 | 0.88 | 72.8 |
| 0027198 | | | | | | | | |
| BPN- | 46.3 | 1.0 | 85.0 | 2.9 | 33.0 | 11.4 | 0.45 | 79.2 |
| 0027213 | | | | | | | | |
| BPN- | 73.2 | 9.2 | 97.3 | 0.4 | n/a | NMII-sel | 1.00 | >99 |
| 0027215 | | | | | | | | |
| BPN- | 97.2 | 85.0 | >99 | 2.7 | 31.0 | 11.5 | 4.60 | >99 |
| 0027216 | | | | | | | | |
| BPN- | 97.9 | 76.0 | >99 | 7.8 | 160.0 | 20.5 | 21.00 | >99 |
| 0027217 | | | | | | | | |
| BPN- | 90.0 | 4.4 | 95.5 | 6.9 | n/a | NMII-sel | 40.00 | 49.1 |
| 0027218 | | | | | | | | |
| BPN- | 46.2 | 3.8 | 98.3 | 39.0 | n/a | NMII-sel | n/a | 50.0 |
| 0027219 | | | | | | | | |
| BPN- | >99 | 89.1 | >99 | 28.0 | 300.0 | 10.7 | 4.60 | 70.8 |
| 0027228 | | | | | | | | |
| BPN- | >99 | 84.9 | >99 | 5.9 | N/A | NMII-sel | 5.00 | 55.9 |
| 0027236 | | | | | | | | |
| BPN- | 79.8 | 15.8 | 82.7 | 11.0 | 50.0 | 4.5 | 6.70 | 40.8 |
| 0027237 | | | | | | | | |
| BPN- | >99 | >99 | >99 | 4.1 | n/a | NMII-sel | 3.70 | 90.8 |
| 0027238 | | | | | | | | |
| BPN- | 94.1 | 74.6 | 98.0 | 3.8 | n/a | NMII-sel | n/a | 82.3 |
| 0027240 | | | | | | | | |
| BPN- | 91.6 | 87.0 | 97.4 | 13.0 | 100.0 | 7.7 | 7.20 | 54.2 |
| 0027241 | | | | | | | | |
| BPN- | 45.8 | 2.7 | 70.1 | 1.0 | n/a | NMII-sel | 1.00 | 78.1 |
| 0027250 | | | | | | | | |
| BPN- | 82.4 | 42.2 | 98.3 | 1.9 | n/a | NMII-sel | 0.80 | 71.5 |
| 0027255 | | | | | | | | |
| BPN- | 97.0 | 78.0 | 97.4 | 5.9 | 40.0 | 6.8 | 4.20 | 96.7 |
| 0027257 | | | | | | | | |
| BPN- | 91.9 | 26.3 | 97.0 | 1.7 | 50.0 | 29.4 | 1.00 | 95.8 |
| 0027289 | | | | | | | | |
| BPN- | 92.7 | 64.6 | >99 | 10.0 | 80.0 | 8.0 | 4.20 | >99 |
| 0027330 | | | | | | | | |
| BPN- | 97.7 | 98.7 | 98.1 | 4.8 | 33.0 | 6.9 | 2.00 | >99 |
| 0027346 | | | | | | | | |
| BPN- | 96.9 | 86.4 | >99 | 8.7 | n/a | NMII-sel | 0.82 | 68.8 |
| 0027374 | | | | | | | | |
| BPN- | 90.4 | 2.8 | 97.0 | 5.9 | 46.0 | 7.8 | 6.70 | 89.5 |
| 0027376 | | | | | | | | |
| BPN- | 95.2 | 83.9 | 97.5 | 5.2 | 30.0 | 5.8 | 3.50 | 96.3 |
| 0027393 | | | | | | | | |
| BPN- | 92.2 | 57.2 | 98.7 | 4.1 | 14.0 | 3.4 | 1.10 | >99 |
| 0027394 | | | | | | | | |
| BPN- | 85.5 | 23.5 | >99 | 1.8 | 9.5 | 5.3 | 1.10 | 74.1 |
| 0027396 | | | | | | | | |
| BPN- | 75.7 | 1.6 | >99 | 53.0 | 200.0 | 3.8 | 7.50 | 84.0 |
| 0027406 | | | | | | | | |
| BPN- | 91.5 | 41.8 | >99 | 3.4 | 25.0 | 7.4 | 1.60 | 96.8 |
| 0027410 | | | | | | | | |
| BPN- | 81.8 | 6.4 | 98.8 | 6.3 | 51.0 | 8.1 | 1.10 | 74.7 |
| 0027411 | | | | | | | | |
| BPN- | 90.8 | 67.7 | 95.5 | 5.8 | 21.0 | 3.6 | 2.70 | >99 |
| 0027412 | | | | | | | | |
| BPN- | 97.2 | 71.4 | 91.8 | 50.0 | 230.0 | 4.6 | 12.00 | 64.4 |
| 0027441 | | | | | | | | |
| BPN- | 83.3 | 77.5 | 98.3 | 4.0 | n/a | NMII-sel | 1.20 | 62.6 |
| 0027468 | | | | | | | | |
| BPN- | 58.8 | 44.3 | >99 | 2.0 | n/a | NMII-sel | 0.68 | 90.6 |
| 0027469 | | | | | | | | |
| BPN- | 49.4 | 20.0 | 96.4 | 0.8 | n/a | NMII-sel | 0.90 | >99 |
| 0027488 | | | | | | | | |
| BPN- | 76.1 | 8.5 | >99 | 0.8 | 26.0 | 33.3 | 0.94 | 85.0 |
| 0027489 | | | | | | | | |
| BPN- | 80.8 | 6.8 | 98.5 | 0.7 | n/a | NMII-sel | 0.41 | 97.0 |
| 0027490 | | | | | | | | |
| BPN- | 54.0 | 6.7 | 97.1 | 2.3 | 24.0 | 10.4 | 0.68 | 84.9 |
| 0027491 | | | | | | | | |
| BPN- | 95.1 | 66.9 | >99 | 2.9 | n/a | NMII-sel | 2.60 | 89.5 |
| 0027492 | | | | | | | | |
| BPN- | 63.5 | <1.0 | 88.4 | 4.2 | n/a | NMII-sel | 4.70 | Inconclusive |
| 0027494 | | | | | | | | |
| BPN- | 92.7 | 77.6 | 89.4 | 9.3 | 70.0 | 7.5 | 9.40 | 50.7 |
| 0028550 | | | | | | | | |
| BPN- | 87.7 | 62.2 | 91.8 | 1.2 | 26.0 | 21.7 | 2.50 | 71.1 |
| 0028552 | | | | | | | | |
| BPN- | 87.6 | 35.9 | 97.6 | 9.7 | 120.0 | 12.4 | 9.00 | 91.6 |
| 0028554 | | | | | | | | |
| BPN- | 4.7 | <1 | 93.6 | 0.8 | 4.6 | 6.1 | 1.50 | 64.2 |
| 0028555 | | | | | | | | |
| BPN- | 93.2 | 71.0 | 89.4 | 15.0 | 200.0 | 13.3 | 11.00 | 50.8 |
| 0028616 | | | | | | | | |
| BPN- | 84.4 | 84.6 | 81.8 | 5.3 | 300.0 | 56.6 | 8.90 | 46.3 |
| 0028625 | | | | | | | | |
| BPN- | >99 | 97.1 | 98.2 | 4.5 | n/a | NMII-sel | 1.30 | >99 |
| 0028646 | | | | | | | | |
| BPN- | 52.5 | <1 | 98.4 | 1.3 | 4.4 | 3.4 | 0.26 | 82.7 |
| 0028648 | | | | | | | | |
| BPN- | 96.7 | 73.1 | 98.4 | 4.2 | 30.0 | 7.1 | 2.70 | 62.9 |
| 0028649 | | | | | | | | |
| BPN- | 88.7 | 62.6 | 93.4 | 9.1 | 40.0 | 4.4 | 12.00 | 70.0 |
| 0028650 | | | | | | | | |
| BPN- | 86.2 | 22.8 | 98.5 | 8.3 | 80.0 | 9.6 | 1.30 | >99 |
| 0028652 | | | | | | | | |
| BPN- | 55.3 | 11.2 | 98.0 | 1.9 | 16.0 | 8.4 | 1.70 | 82.0 |
| 0028694 | | | | | | | | |
| BPN- | 88.4 | 25.4 | 97.3 | 8.0 | 60.0 | 7.5 | 1.90 | 98.4 |
| 0028697 | | | | | | | | |
| BPN- | 37.6 | 7.4 | >99 | 5.2 | 40 | 7.7 | 2.2 | 81.1 |
| 0028731 | | | | | | | | |
| BPN- | 82.1 | 14.8 | 96.6 | 8.7 | 27 | 3.1 | 6.8 | 74.8 |
| 0028733 | | | | | | | | |
| BPN- | 76 | <1 | 96.3 | 3 | 15 | 5.0 | 2.9 | 86.1 |
| 0028734 | | | | | | | | |
| BPN- | 64 | 2 | 95.3 | 13 | 90 | 6.9 | 1.5 | 91.1 |
| 0028736 | | | | | | | | |
| BPN- | 90.9 | 55.3 | 93.3 | 51 | 210 | 4.1 | 40 | 91 |
| 0028758 | | | | | | | | |
| BPN- | 87.9 | 83 | 87.6 | 3.7 | 16 | 4.3 | 0.94 | 40.3 |
| 0028760 | | | | | | | | |
| BPN- | 92.7 | 64 | 95.3 | 0.63 | 2.4 | 3.8 | 1 | >99 |
| 0028790 | | | | | | | | |
| BPN- | 80.9 | 62.7 | 94.2 | 7.2 | N/A | NMII-sel | 3 | 85.2 |
| 0028820 | | | | | | | | |
| BPN- | 89.8 | 29.1 | 98.5 | 0.96 | 12 | 12.5 | 0.77 | 84.1 |
| 0028821 | | | | | | | | |
| BPN- | 98.8 | 94.7 | 99 | 2.6 | N/A | NMII-sel | 2.8 | 91.2 |
| 0028863 | | | | | | | | |
| BPN- | 97.4 | 88.4 | 98.4 | 7 | N/A | NMII-sel | 12 | 65.4 |
| 0028864 | | | | | | | | |
| BPN- | 77.8 | 2.9 | 97.8 | 10 | N/A | NMII-sel | 400 | 82.8 |
| 0028866 | | | | | | | | |
| BPN- | 69.9 | 1 | 98.7 | 2.8 | N/A | NMII-sel | 1.3 | 88.2 |
| 0028867 | | | | | | | | |
| BPN- | 46.7 | 43.3 | 98.9 | 0.76 | 24 | 31.6 | 0.23 | 88.5 |
| 0028868 | | | | | | | | |
| BPN- | 82.8 | 5.4 | 98.4 | 9 | 300 | 33.3 | 110 | 52.4 |
| 0028897 | | | | | | | | |
| BPN- | 93.4 | 58.1 | 96.4 | 3 | 95 | 31.7 | 1.9 | >99 |
| 0028898 | | | | | | | | |
| BPN- | 70.8 | 29.7 | 97.8 | 1.5 | N/A | NMII-sel | 3.8 | 86.1 |
| 0028899 | | | | | | | | |
| BPN- | 54.2 | 7.8 | >99 | 2 | 11 | 5.5 | 0.64 | 97.2 |
| 0028918 | | | | | | | | |
| BPN- | 48.4 | <1 | 98.5 | 2.8 | N/A | NMII-sel | 1.4 | 95.4 |
| 0028919 | | | | | | | | |
| BPN- | 36.6 | 2.5 | 98.4 | 3.2 | N/A | NMII-sel | 3.9 | 97.5 |
| 0028920 | | | | | | | | |
| BPN- | 2.8 | 3.1 | 97.2 | 0.75 | 8.4 | 11.2 | 1 | 95.9 |
| 0028921 | | | | | | | | |
| BPN- | 86.1 | 27.7 | 94.8 | 1.1 | 9.7 | 8.8 | 0.77 | 95.3 |
| 0028922 | | | | | | | | |
| BPN- | 77.5 | 1 | 98.1 | 3.5 | 30 | 8.6 | 6 | >99 |
| 0028923 | | | | | | | | |
| BPN- | 44.7 | <1 | 97.7 | 1.7 | 20 | 11.8 | 2.6 | 88 |
| 0028924 | | | | | | | | |
| BPN- | 85.9 | 24.7 | 93.7 | 0.8 | 5.8 | 7.3 | 0.7 | 99 |
| 0028925 | | | | | | | | |

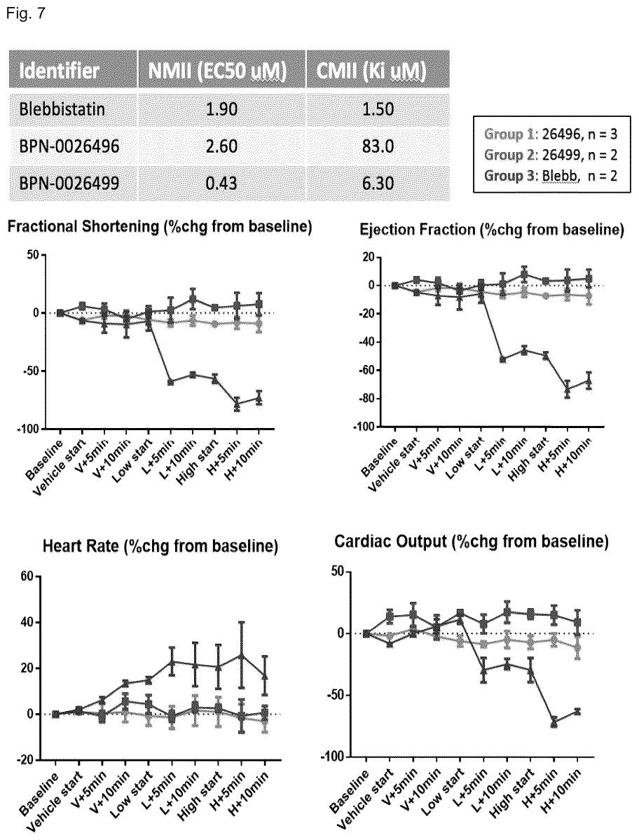

Table 2 and FIG. 7, below, provide data summarizing two different cardiac safety assays. The first (results shown in Table 2) assesses the effects of our compounds on the contractility of cardiomyocytes. As the cardiac Ki increases, the contractility effects dramatically decrease. However, unlike in the adult heart, the hiPS cardiomyocytes used in this assay express NMII, which likely accounts for the residual contractility effects seen at the highest dose tested. For that reason, we moved to in vivo echocardiograms, which assess contractility in live animals (FIG. 7). Similar to our results with the cardiomyocytes, increasing the cardiac Ki protects against the substantial contractility effects observed with Blebbistatin, regardless of NMII potency. Accordingly, the compounds of the invention and compounds used for practice of methods of the invention can produce less cardiac toxicity than does blebbistatin.

TABLE 2
Cardiac safety Characterization for Selected Blebbistatin Analogs I:
Assessment of beating parameters in hiPS-derived cardiomyocyte.

| | | | | Qualitative impact on | |
|---|---|---|---|---|---|
| | | | | Complete inhibition of | Change in |
| | | | | Qualitative impact on | amplitude |
| | NMII | CMMII | CMMII/NMII | contractility (e.g. Full = | at 1 uM @ |
| Compound | EC50 | Ki | Ratio | contraction) | 15 min |
| Blebbistatin | 1.2 uM | 0.67 uM | 0.56 | NOTE: Baseline | −57.9 ± 12.8% |
| | | | | 0.1 uM: No 0.3 uM: Partial | |
| | | | | 1.0u M: Full 3.0 uM: Full | |
| BPN- | 5.1 uM | 47.2 uM | 9.25 | NOTE: Reduced potency, | +1.1 ± 0.1% |
| 0025059 | | | | improved selectivity, solubility, | |
| | | | | photostability | |
| | | | | 0.1 uM-1.0 uM: No effect; 3.0 uM: | |
| | | | | Small effect | |
| BPN- | 0.78 uM | 0.48 uM | 0.62 | NOTE: Improved potency, | −81.8 ± 86.9% |
| 0025060 | | | | selectivity slightly improved, | |
| | | | | worse solubility/photostab | |
| | | | | Similar to Blebb | |
| BPN- | 7.5 uM | 26.3 uM | 3.51 | NOTE: Reduced potency, but | −7.8 ± 1.3% |
| 0025100 | | | | reasonable improvement in | |
| | | | | selectivity and solubility | |
| | | | | 0.1 uM-1.0 uM: No effect; 3.0 uM: | |
| | | | | Small effect | |
| BPN- | 2.6 uM | 1.97 uM | 0.76 | NOTE: Slightly reduced potency, | −38.9 ± 2.4% |
| 0025002 | | | | but slight improvement in | |
| | | | | selectivity | |
| | | | | Similar to Blebb | |
| BPN- | 26.8 uM | 1.74 uM | 0.07 | NOTE: Reduced potency and | −47.6 ± 5.2% |
| 0025046 | | | | large shift towards cardiac | |
| | | | | selectivity | |
| | | | | Similar to Blebb | |
| BPN- | Inactive | Inactive | N/A | NOTE: Inactive in cytokinesis, | +2.0 ± 2.8% |
| 0025098 | | | | improved solubility (86 uM vs | |
| | | | | Blebb = 9.3 uM) | |
| | | | | No effect at any dose | |

TABLE 3
Structures of Compounds for Methods of Invention

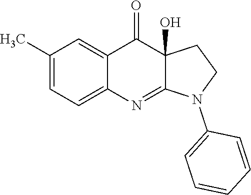 [SMILES: Cc1ccc2c(c1)C(=O)[C@]1(O)CCN(c3ccccc3)C1=N2]

BPN-0024998 (blebbistatin, control)

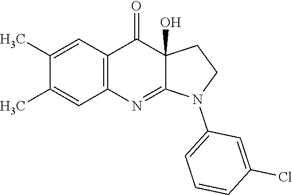 [SMILES: Cc1cc2c(cc1C)C(=O)[C@]1(O)CCN(c3cccc(Cl)c3)C1=N2]

BPN-0026500

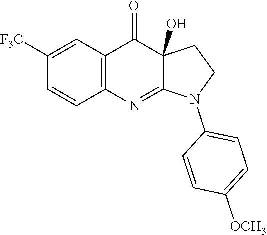 [SMILES: COc1ccc(N2CC[C@@]3(O)C(=O)c4cc(C(F)(F)F)ccc4N=C23)cc1]

BPN-0026393

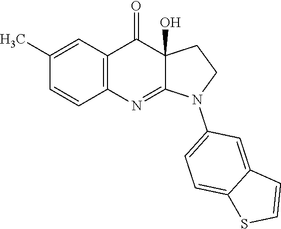 [SMILES: Cc1ccc2c(c1)C(=O)[C@]1(O)CCN(c3ccc4sccc4c3)C1=N2]

BPN-0026651

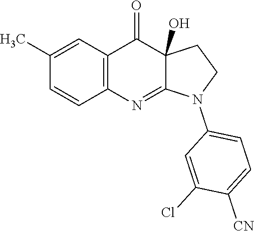 [SMILES: Cc1ccc2c(c1)C(=O)[C@]1(O)CCN(c3ccc(C#N)c(Cl)c3)C1=N2]

BPN-0026394/002

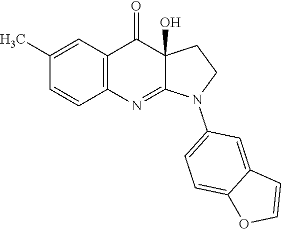 [SMILES: Cc1ccc2c(c1)C(=O)[C@]1(O)CCN(c3ccc4occc4c3)C1=N2]

BPN-0026283

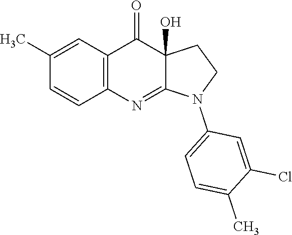 [SMILES: Cc1ccc2c(c1)C(=O)[C@]1(O)CCN(c3ccc(C)c(Cl)c3)C1=N2]

BPN-0025254

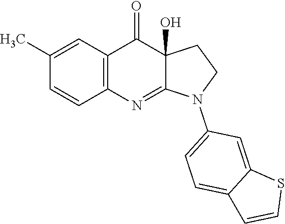 [SMILES: Cc1ccc2c(c1)C(=O)[C@]1(O)CCN(c3ccc4ccsc4c3)C1=N2]

BPN-0026650

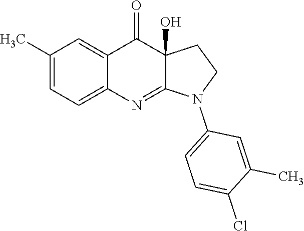

BPN-0026325

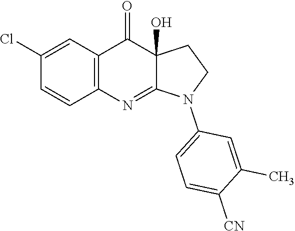 [SMILES: Cc1cc(N2CC[C@@]3(O)C(=O)c4cc(Cl)ccc4N=C23)ccc1C#N]

BPN-0026457

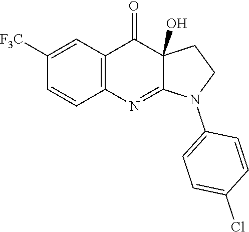 [SMILES: O=C1c2cc(C(F)(F)F)ccc2N=C2N(c3ccc(Cl)cc3)CC[C@@]12O]

BPN-0026544

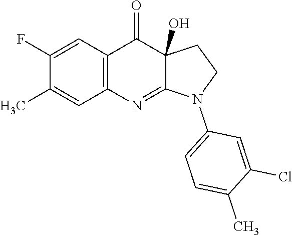 [SMILES: Cc1cc2c(cc1F)C(=O)[C@]1(O)CCN(c3ccc(C)c(Cl)c3)C1=N2]

BPN-0026606

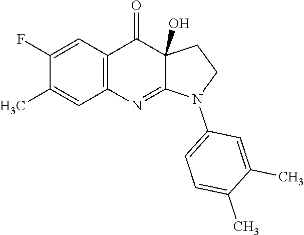 [SMILES: Cc1ccc(N2CC[C@@]3(O)C(=O)c4cc(F)c(C)cc4N=C23)cc1C]

BPN-0026607

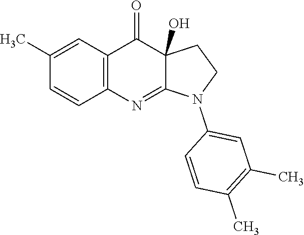

BPN-0025903

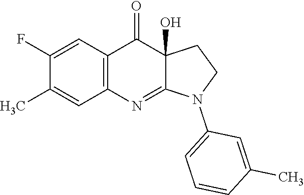 [SMILES: Cc1cccc(N2CC[C@@]3(O)C(=O)c4cc(F)c(C)cc4N=C23)c1]

BPN-0026620

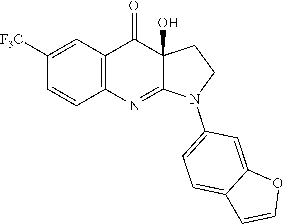 [SMILES: O=C1c2cc(C(F)(F)F)ccc2N=C2N(c3ccc4ccoc4c3)CC[C@@]12O]

BPN-0026495

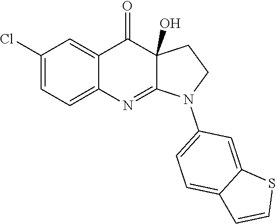 [SMILES: O=C1c2cc(Cl)ccc2N=C2N(c3ccc4ccsc4c3)CC[C@@]12O]

BPN-0026545

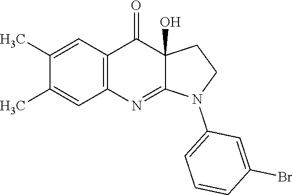 [SMILES: Cc1cc2c(cc1C)C(=O)[C@]1(O)CCN(c3cccc(Br)c3)C1=N2]

BPN-0026660

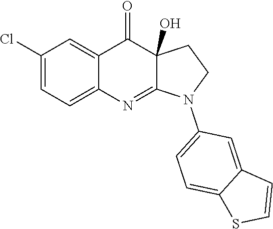 [SMILES: O=C1c2cc(Cl)ccc2N=C2N(c3ccc4sccc4c3)CC[C@@]12O]

BPN-0026546

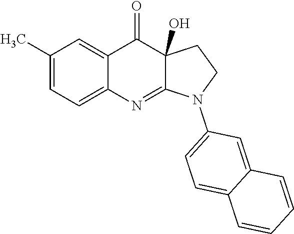 [SMILES: Cc1ccc2c(c1)C(=O)[C@]1(O)CCN(c3ccc4ccccc4c3)C1=N2]

BPN-0025915

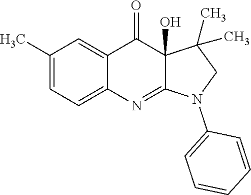 [SMILES: Cc1ccc2c(c1)C(=O)[C@@]1(O)C(=N2)N(c2ccccc2)CC1(C)C]

BPN-0026282

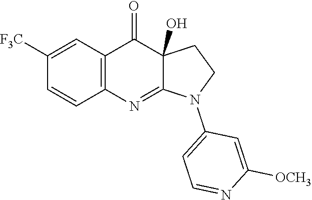 [SMILES: COc1cc(N2CC[C@@]3(O)C(=O)c4cc(C(F)(F)F)ccc4N=C23)ccn1]

BPN-0026533

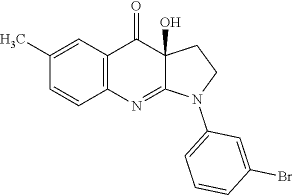 [SMILES: Cc1ccc2c(c1)C(=O)[C@]1(O)CCN(c3cccc(Br)c3)C1=N2]

BPN-0025240

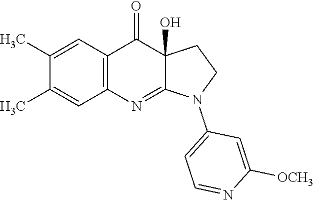 [SMILES: COc1cc(N2CC[C@@]3(O)C(=O)c4cc(C)c(C)cc4N=C23)ccn1]

BPN-0026709

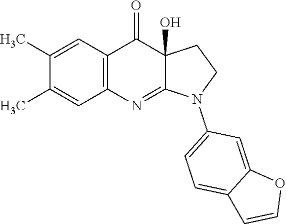 [SMILES: Cc1cc2c(cc1C)C(=O)[C@]1(O)CCN(c3ccc4ccoc4c3)C1=N2]

BPN-0026664

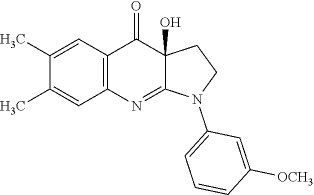 [SMILES: COc1cccc(N2CC[C@@]3(O)C(=O)c4cc(C)c(C)cc4N=C23)c1]

BPN-0026496

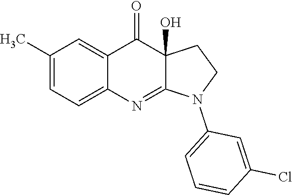 [SMILES: Cc1ccc2c(c1)C(=O)[C@]1(O)CCN(c3cccc(Cl)c3)C1=N2]

BPN-0025059

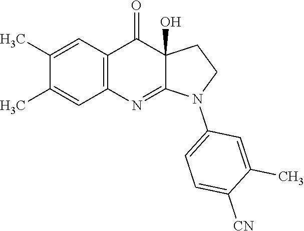

BPN-0026661

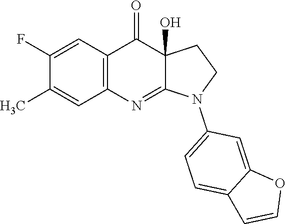 [SMILES: Cc1cc2c(cc1F)C(=O)[C@]1(O)CCN(c3ccc4ccoc4c3)C1=N2]

BPN-0026619

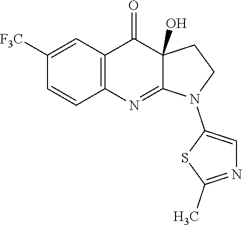 [SMILES: Cc1ncc(N2CC[C@@]3(O)C(=O)c4cc(C(F)(F)F)ccc4N=C23)s1]

BPN-0026404

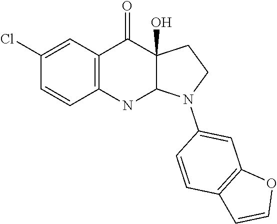 [SMILES: O=C1c2cc(Cl)ccc2NC2N(c3ccc4ccoc4c3)CC[C@@]12O]

BPN-0026401

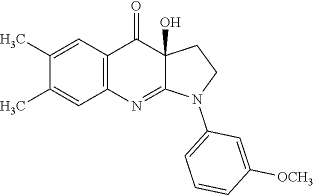 [SMILES: COc1cccc(N2CC[C@@]3(O)C(=O)c4cc(C)c(C)cc4N=C23)c1]

BPN-0026497

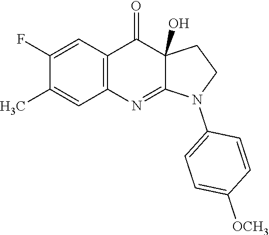 [SMILES: COc1ccc(N2CC[C@@]3(O)C(=O)c4cc(F)c(C)cc4N=C23)cc1]

BPN-0026605

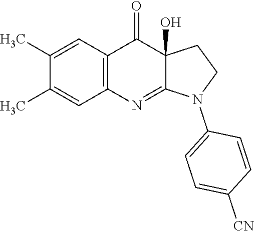 [SMILES: Cc1cc2c(cc1C)C(=O)[C@]1(O)CCN(c3ccc(C#N)cc3)C1=N2]

BPN-0026499

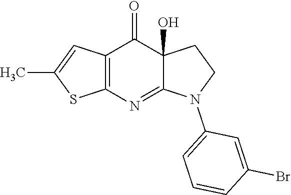 [SMILES: Cc1cc2c(s1)N=C1N(c3cccc(Br)c3)CC[C@@]1(O)C2=O]

BPN-0026624

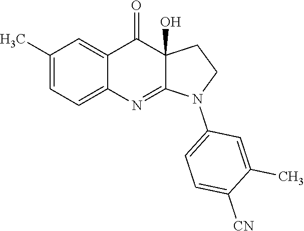

BPN-0026324

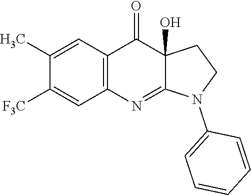 [SMILES: Cc1cc2c(cc1C(F)(F)F)N=C1N(c3ccccc3)CC[C@@]1(O)C2=O]

BPN-0026648

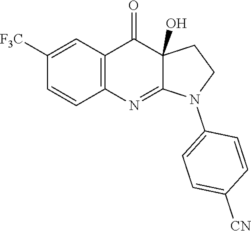 [SMILES: N#Cc1ccc(N2CC[C@@]3(O)C(=O)c4cc(C(F)(F)F)ccc4N=C23)cc1]

BPN-0026608

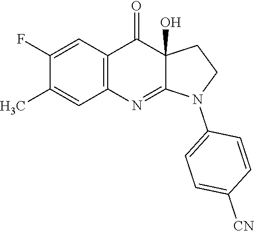 [SMILES: Cc1cc2c(cc1F)C(=O)[C@]1(O)CCN(c3ccc(C#N)cc3)C1=N2]

BPN-0026638

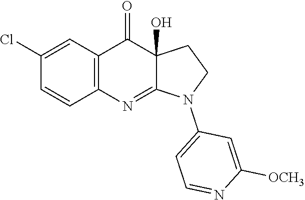 [SMILES: COc1cc(N2CC[C@@]3(O)C(=O)c4cc(Cl)ccc4N=C23)ccn1]

BPN-0026621

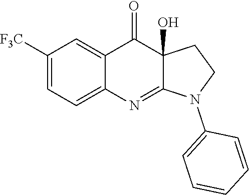 [SMILES: O=C1c2cc(C(F)(F)F)ccc2N=C2N(c3ccccc3)CC[C@@]12O]

BPN-0025110

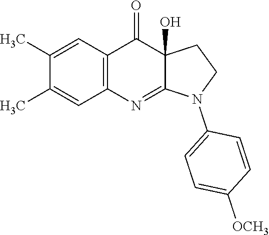 [SMILES: COc1ccc(N2CC[C@@]3(O)C(=O)c4cc(C)c(C)cc4N=C23)cc1]

BPN-0026665

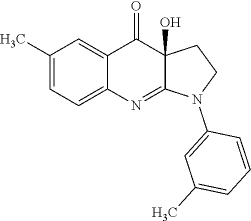 [SMILES: Cc1cccc(N2CC[C@@]3(O)C(=O)c4cc(C)ccc4N=C23)c1]

BPN-0025100

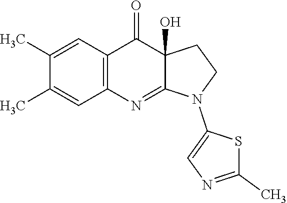

BPN-0026693

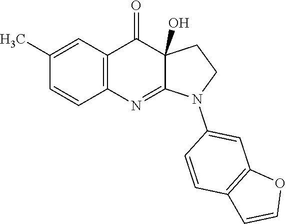 [SMILES: Cc1ccc2c(c1)C(=O)[C@]1(O)CCN(c3ccc4ccoc4c3)C1=N2]

BPN-0026285

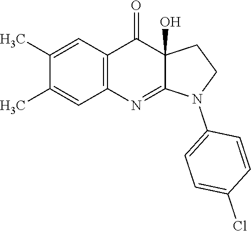 [SMILES: Cc1cc2c(cc1C)C(=O)[C@]1(O)CCN(c3ccc(Cl)cc3)C1=N2]

BPN-0026662

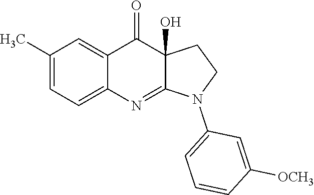 [SMILES: COc1cccc(N2CC[C@@]3(O)C(=O)c4cc(C)ccc4N=C23)c1]

BPN-0025044

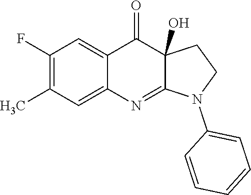 [SMILES: Cc1cc2c(cc1F)C(=O)[C@]1(O)CCN(c3ccccc3)C1=N2]

BPN-0026387

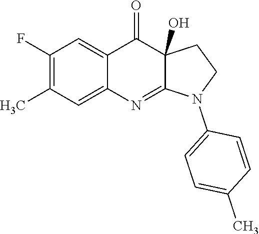 [SMILES: Cc1ccc(N2CC[C@@]3(O)C(=O)c4cc(F)c(C)cc4N=C23)cc1]

BPN-0026579

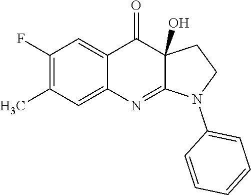 [SMILES: Cc1cc2c(cc1F)C(=O)[C@]1(O)CCN(c3ccccc3)C1=N2]

BPN-0026669

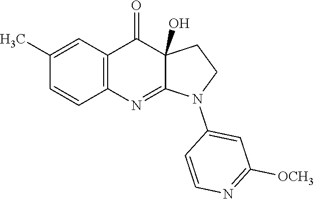 [SMILES: COc1cc(N2CC[C@@]3(O)C(=O)c4cc(C)ccc4N=C23)ccn1]

BPN-0025919

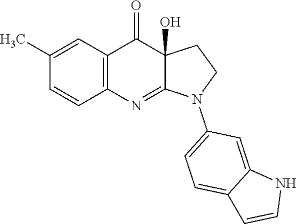 [SMILES: Cc1ccc2c(c1)C(=O)[C@]1(O)CCN(c3ccc4cc[nH]c4c3)C1=N2]

BPN-0026555

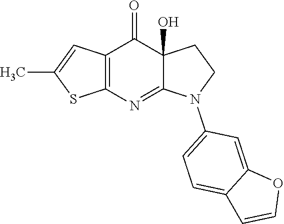 [SMILES: Cc1cc2c(s1)N=C1N(c3ccc4ccoc4c3)CC[C@@]1(O)C2=O]

BPN-0026639

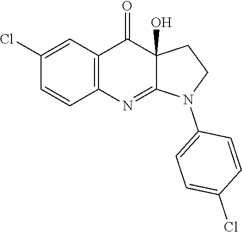 [SMILES: O=C1c2cc(Cl)ccc2N=C2N(c3ccc(Cl)cc3)CC[C@@]12O]

BPN-0026640

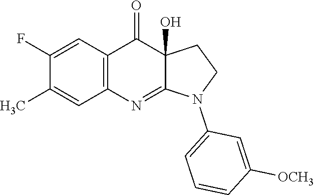 [SMILES: COc1cccc(N2CC[C@@]3(O)C(=O)c4cc(F)c(C)cc4N=C23)c1]

BPN-0026618

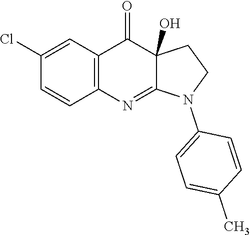 [SMILES: Cc1ccc(N2CC[C@@]3(O)C(=O)c4cc(Cl)ccc4N=C23)cc1]

BPN-0026609

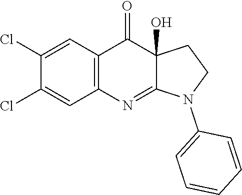 [SMILES: O=C1c2cc(Cl)c(Cl)cc2N=C2N(c3ccccc3)CC[C@@]12O]

BPN-0026643

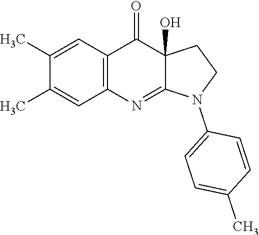 [SMILES: Cc1ccc(N2CC[C@@]3(O)C(=O)c4cc(C)c(C)cc4N=C23)cc1]

BPN-0026663

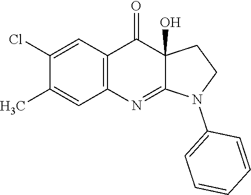 [SMILES: Cc1cc2c(cc1Cl)C(=O)[C@]1(O)CCN(c3ccccc3)C1=N2]

BPN-0026659

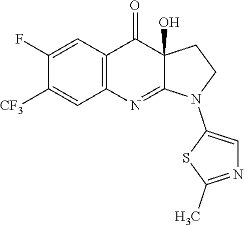 [SMILES: Cc1ncc(N2CC[C@@]3(O)C(=O)c4cc(F)c(C(F)(F)F)cc4N=C23)s1]

BPN-0026667

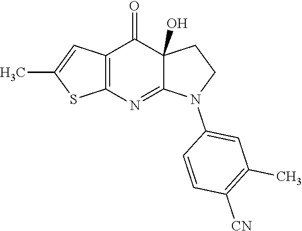

BPN-0026576

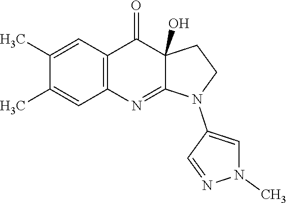

BPN-0026710

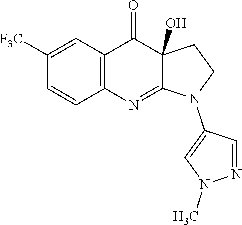 [SMILES: Cn1cc(N2CC[C@@]3(O)C(=O)c4cc(C(F)(F)F)ccc4N=C23)cn1]

BPN-0026413

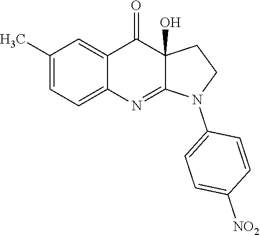 [SMILES: Cc1ccc2c(c1)C(=O)[C@]1(O)CCN(c3ccc([N+](=O)[O-])cc3)C1=N2]

BPN-0025001

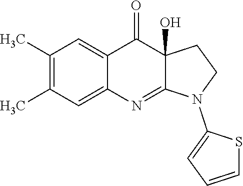 [SMILES: Cc1cc2c(cc1C)C(=O)[C@]1(O)CCN(c3cccs3)C1=N2]

BPN-0026708

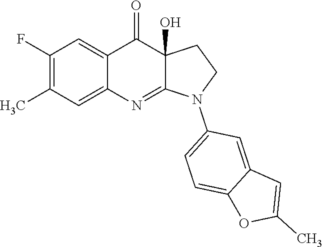 [SMILES: Cc1cc2cc(N3CC[C@@]4(O)C(=O)c5cc(F)c(C)cc5N=C34)ccc2o1]

BPN-0026726

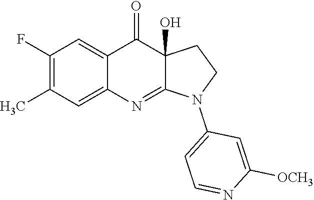 [SMILES: COc1cc(N2CC[C@@]3(O)C(=O)c4cc(F)c(C)cc4N=C23)ccn1]

BPN-0026745

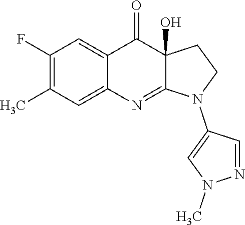 [SMILES: Cc1cc2c(cc1F)C(=O)[C@]1(O)CCN(c3cnn(C)c3)C1=N2]

BPN-0026746

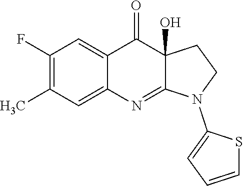 [SMILES: Cc1cc2c(cc1F)C(=O)[C@]1(O)CCN(c3cccs3)C1=N2]

BPN-0026748

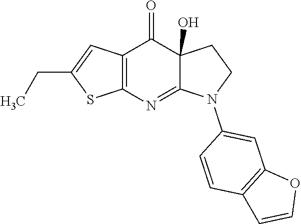 [SMILES: CCc1cc2c(s1)N=C1N(c3ccc4ccoc4c3)CC[C@@]1(O)C2=O]

BPN-0026749

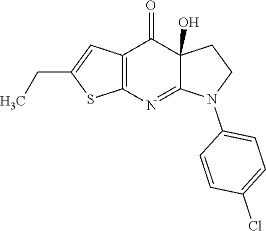 [SMILES: CCc1cc2c(s1)N=C1N(c3ccc(Cl)cc3)CC[C@@]1(O)C2=O]

BPN-0026770

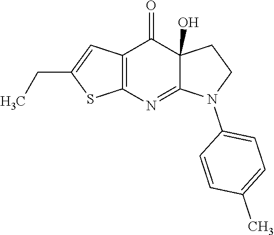 [SMILES: CCc1cc2c(s1)N=C1N(c3ccc(C)cc3)CC[C@@]1(O)C2=O]

BPN-0026774

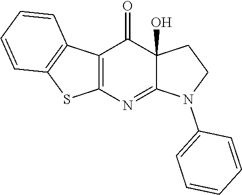 [SMILES: O=C1c2c(sc3ccccc23)N=C2N(c3ccccc3)CC[C@@]12O]

BPN-0026785

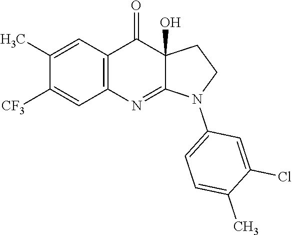 [SMILES: Cc1ccc(N2CC[C@@]3(O)C(=O)c4cc(C)c(C(F)(F)F)cc4N=C23)cc1Cl]

BPN-0026786

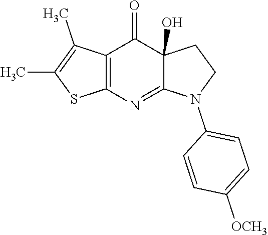 [SMILES: COc1ccc(N2CC[C@@]3(O)C(=O)c4c(sc(C)c4C)N=C23)cc1]

BPN-0026813

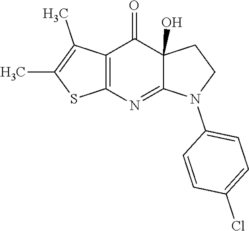 [SMILES: Cc1sc2c(c1C)C(=O)[C@]1(O)CCN(c3ccc(Cl)cc3)C1=N2]

BPN-0026815

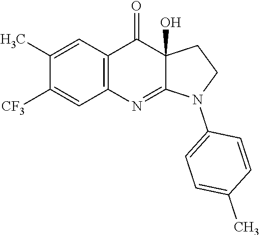 [SMILES: Cc1ccc(N2CC[C@@]3(O)C(=O)c4cc(C)c(C(F)(F)F)cc4N=C23)cc1]

BPN-0026819

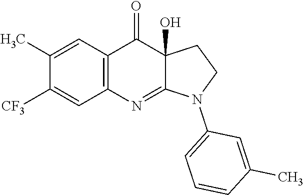

BPN-0026847

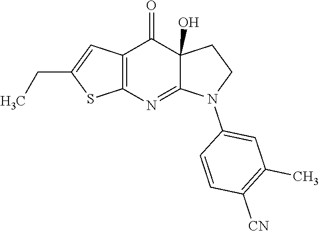 [SMILES: CCc1cc2c(s1)N=C1N(c3ccc(C#N)c(C)c3)CC[C@@]1(O)C2=O]

BPN-0026848

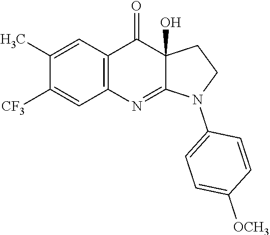 [SMILES: COc1ccc(N2CC[C@@]3(O)C(=O)c4cc(C)c(C(F)(F)F)cc4N=C23)cc1]

BPN-0026849

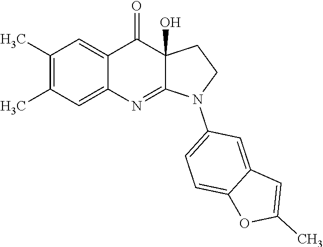

BPN-0026852

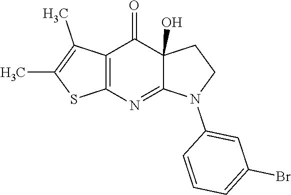 [SMILES: Cc1sc2c(c1C)C(=O)[C@]1(O)CCN(c3cccc(Br)c3)C1=N2]

BPN-0026853

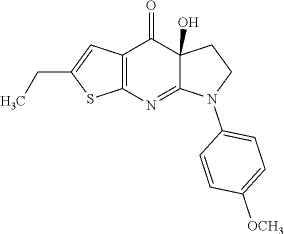 [SMILES: CCc1cc2c(s1)N=C1N(c3ccc(OC)cc3)CC[C@@]1(O)C2=O]

BPN-0026881

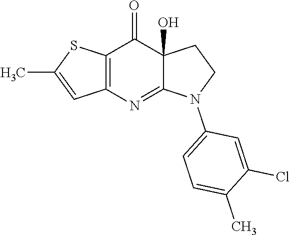 [SMILES: Cc1cc2c(s1)C(=O)[C@]1(O)CCN(c3ccc(C)c(Cl)c3)C1=N2]

BPN-0026882

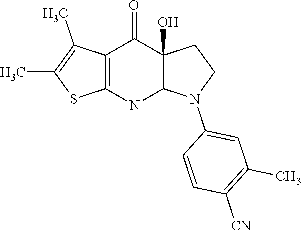

BPN-0026886

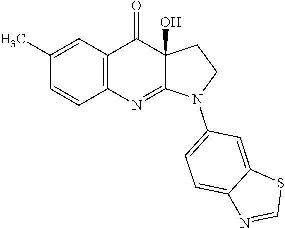 [SMILES: Cc1ccc2c(c1)C(=O)[C@]1(O)CCN(c3ccc4ncsc4c3)C1=N2]

BPN-0027036

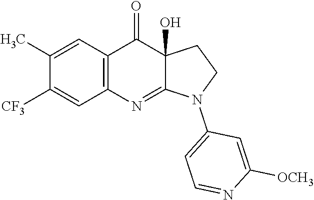 [SMILES: COc1cc(N2CC[C@@]3(O)C(=O)c4cc(C)c(C(F)(F)F)cc4N=C23)ccn1]

BPN-0027037

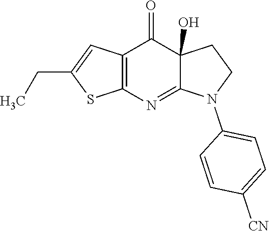 [SMILES: CCc1cc2c(s1)N=C1N(c3ccc(C#N)cc3)CC[C@@]1(O)C2=O]

BPN-0027038

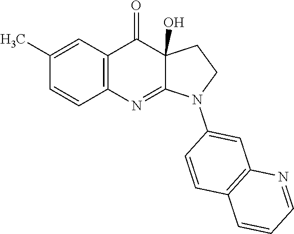 [SMILES: Cc1ccc2c(c1)C(=O)[C@]1(O)CCN(c3ccc4cccnc4c3)C1=N2]

BPN-0027039

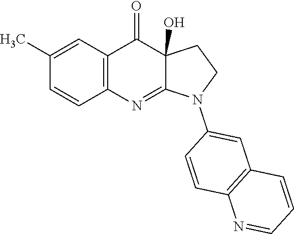 [SMILES: Cc1ccc2c(c1)C(=O)[C@]1(O)CCN(c3ccc4ncccc4c3)C1=N2]

BPN-0027040

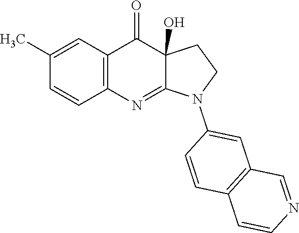 [SMILES: Cc1ccc2c(c1)C(=O)[C@]1(O)CCN(c3ccc4ccncc4c3)C1=N2]

BPN-0027042

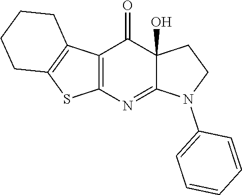 [SMILES: O=C1c2c(sc3c2CCCC3)N=C2N(c3ccccc3)CC[C@@]12O]

BPN-0027043

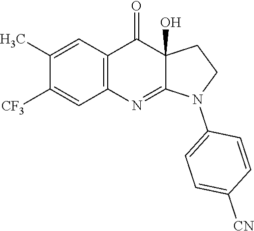 [SMILES: Cc1cc2c(cc1C(F)(F)F)N=C1N(c3ccc(C#N)cc3)CC[C@@]1(O)C2=O]

BPN-0027071

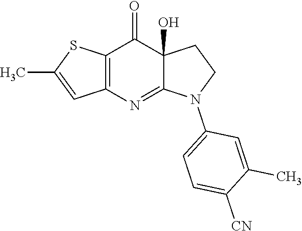

BPN-0027072

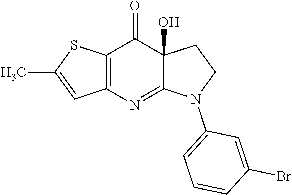 [SMILES: Cc1cc2c(s1)C(=O)[C@]1(O)CCN(c3cccc(Br)c3)C1=N2]

BPN-0027074

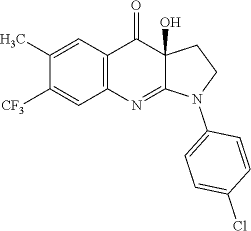 [SMILES: Cc1cc2c(cc1C(F)(F)F)N=C1N(c3ccc(Cl)cc3)CC[C@@]1(O)C2=O]

BPN-0027080

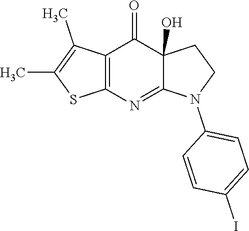 [SMILES: Cc1sc2c(c1C)C(=O)[C@]1(O)CCN(c3ccc(I)cc3)C1=N2]

BPN-0027081

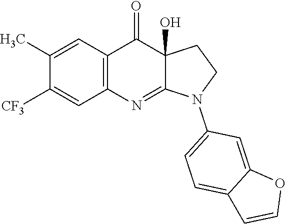 [SMILES: Cc1cc2c(cc1C(F)(F)F)N=C1N(c3ccc4ccoc4c3)CC[C@@]1(O)C2=O]

BPN-0027108

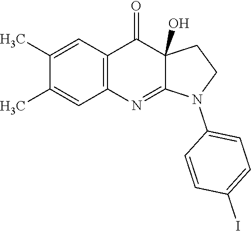 [SMILES: Cc1cc2c(cc1C)C(=O)[C@]1(O)CCN(c3ccc(I)cc3)C1=N2]

BPN-0027109

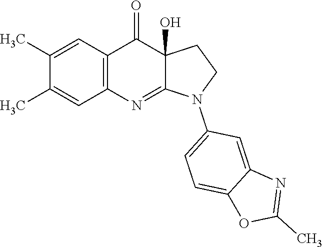

BPN-0027118

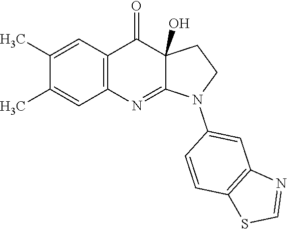 [SMILES: Cc1cc2c(cc1C)C(=O)[C@]1(O)CCN(c3ccc4scnc4c3)C1=N2]

BPN-0027121

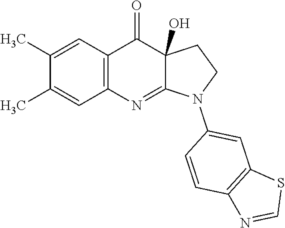 [SMILES: Cc1cc2c(cc1C)C(=O)[C@]1(O)CCN(c3ccc4ncsc4c3)C1=N2]

BPN-0027122

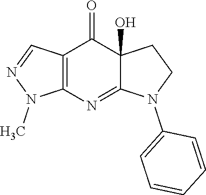 [SMILES: Cn1ncc2c1N=C1N(c3ccccc3)CC[C@@]1(O)C2=O]

BPN-0027159

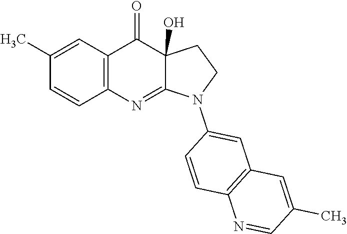

BPN-0027160

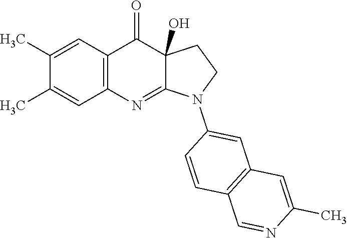

BPN-0027161

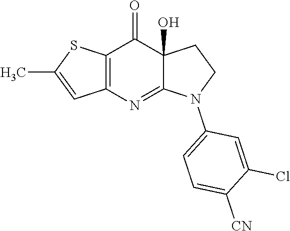 [SMILES: Cc1cc2c(s1)C(=O)[C@]1(O)CCN(c3ccc(C#N)c(Cl)c3)C1=N2]

BPN-0027162

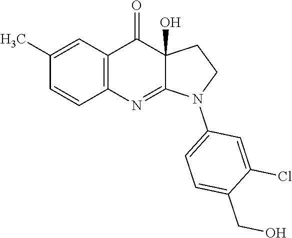 [SMILES: Cc1ccc2c(c1)C(=O)[C@]1(O)CCN(c3ccc(CO)c(Cl)c3)C1=N2]

BPN-0027163

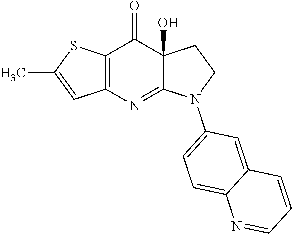 [SMILES: Cc1cc2c(s1)C(=O)[C@]1(O)CCN(c3ccc4ncccc4c3)C1=N2]

BPN-0027181

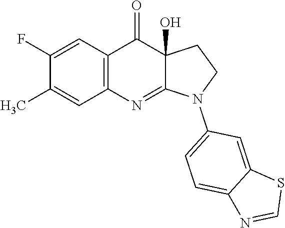 [SMILES: Cc1cc2c(cc1F)C(=O)[C@]1(O)CCN(c3ccc4ncsc4c3)C1=N2]

BPN-0027196

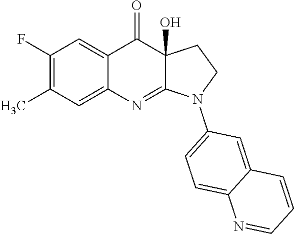 [SMILES: Cc1cc2c(cc1F)C(=O)[C@]1(O)CCN(c3ccc4ncccc4c3)C1=N2]

BPN-0027197

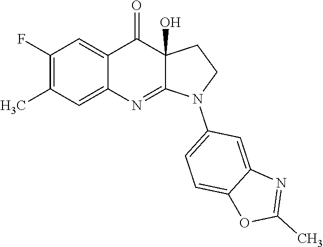 [SMILES: Cc1nc2cc(N3CC[C@@]4(O)C(=O)c5cc(F)c(C)cc5N=C34)ccc2o1]

BPN-0027198

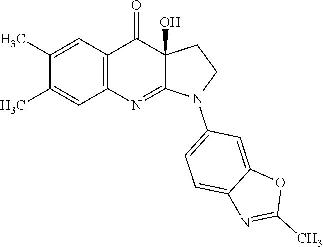

BPN-0027213

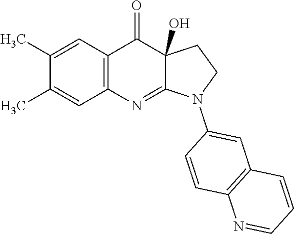 [SMILES: Cc1cc2c(cc1C)C(=O)[C@]1(O)CCN(c3ccc4ncccc4c3)C1=N2]

BPN-0027215

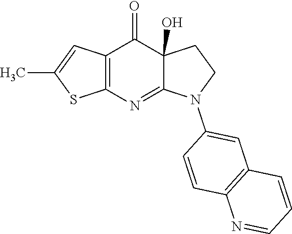 [SMILES: Cc1cc2c(s1)N=C1N(c3ccc4ncccc4c3)CC[C@@]1(O)C2=O]

BPN-0027216

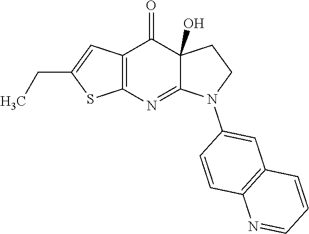 [SMILES: CCc1cc2c(s1)N=C1N(c3ccc4ncccc4c3)CC[C@@]1(O)C2=O]

BPN-0027217

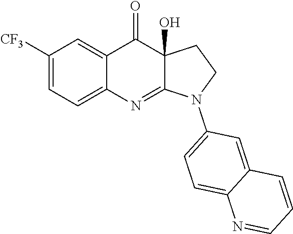 [SMILES: O=C1c2cc(C(F)(F)F)ccc2N=C2N(c3ccc4ncccc4c3)CC[C@@]12O]

BPN-0027218

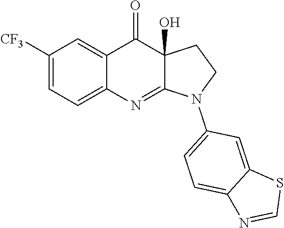 [SMILES: O=C1c2cc(C(F)(F)F)ccc2N=C2N(c3ccc4ncsc4c3)CC[C@@]12O]

BPN-0027219

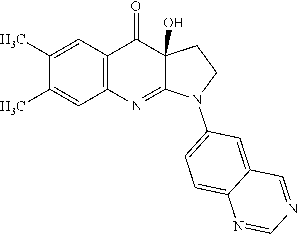 [SMILES: Cc1cc2c(cc1C)C(=O)[C@]1(O)CCN(c3ccc4ncncc4c3)C1=N2]

BPN-0027228

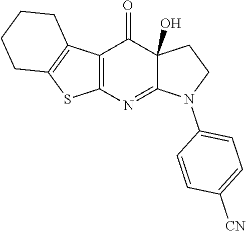 [SMILES: N#Cc1ccc(N2CC[C@@]3(O)C(=O)c4c(sc5c4CCCC5)N=C23)cc1]

BPN-0027236

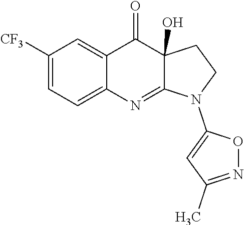 [SMILES: Cc1cc(N2CC[C@@]3(O)C(=O)c4cc(C(F)(F)F)ccc4N=C23)on1]

BPN-0027237

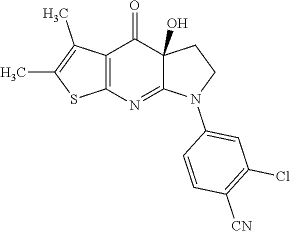 [SMILES: Cc1sc2c(c1C)C(=O)[C@]1(O)CCN(c3ccc(C#N)c(Cl)c3)C1=N2]

BPN-0027238

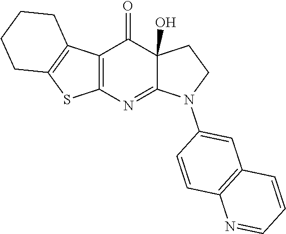 [SMILES: O=C1c2c(sc3c2CCCC3)N=C2N(c3ccc4ncccc4c3)CC[C@@]12O]

BPN-0027240

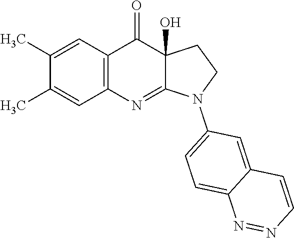 [SMILES: Cc1cc2c(cc1C)C(=O)[C@]1(O)CCN(c3ccc4nnccc4c3)C1=N2]

BPN-0027241

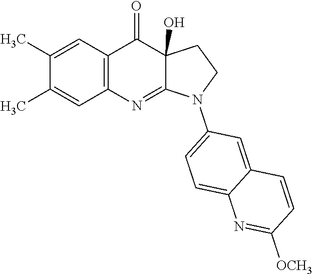 [SMILES: COc1ccc2cc(N3CC[C@@]4(O)C(=O)c5cc(C)c(C)cc5N=C34)ccc2n1]

BPN-0027250

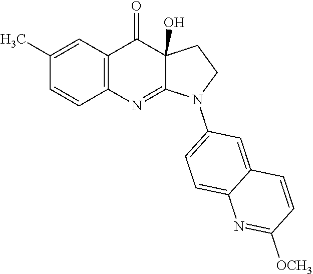 [SMILES: COc1ccc2cc(N3CC[C@@]4(O)C(=O)c5cc(C)ccc5N=C34)ccc2n1]

BPN-0027255

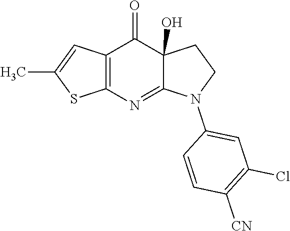 [SMILES: Cc1cc2c(s1)N=C1N(c3ccc(C#N)c(Cl)c3)CC[C@@]1(O)C2=O]

BPN-0027257

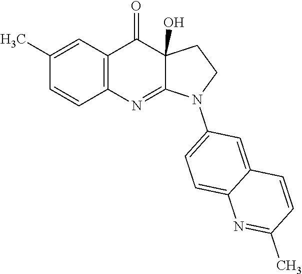 [SMILES: Cc1ccc2c(c1)C(=O)[C@]1(O)CCN(c3ccc4nc(C)ccc4c3)C1=N2]

BPN-0027289

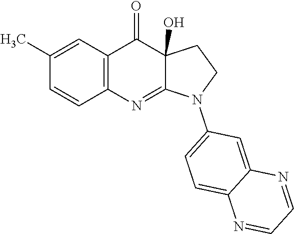 [SMILES: Cc1ccc2c(c1)C(=O)[C@]1(O)CCN(c3ccc4nccnc4c3)C1=N2]

BPN-0027330

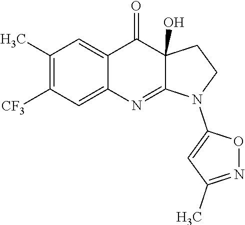 [SMILES: Cc1cc(N2CC[C@@]3(O)C(=O)c4cc(C)c(C(F)(F)F)cc4N=C23)on1]

BPN-0027346

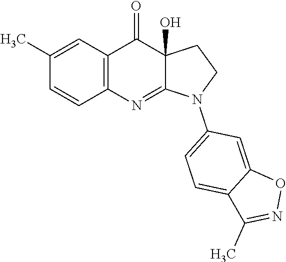 [SMILES: Cc1ccc2c(c1)C(=O)[C@]1(O)CCN(c3ccc4c(C)noc4c3)C1=N2]

BPN-0027374

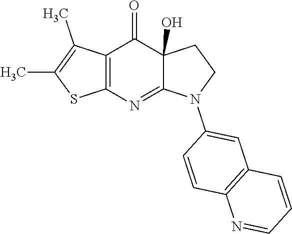 [SMILES: Cc1sc2c(c1C)C(=O)[C@]1(O)CCN(c3ccc4ncccc4c3)C1=N2]

BPN-0027376

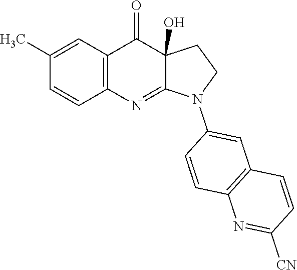 [SMILES: Cc1ccc2c(c1)C(=O)[C@]1(O)CCN(c3ccc4nc(C#N)ccc4c3)C1=N2]

BPN-0027393

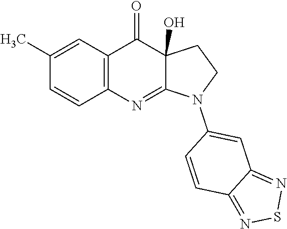 [SMILES: Cc1ccc2c(c1)C(=O)[C@]1(O)CCN(c3ccc4nsnc4c3)C1=N2]

BPN-0027394

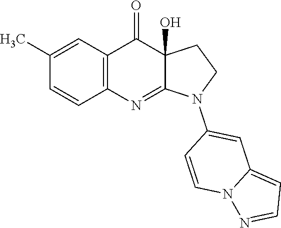 [SMILES: Cc1ccc2c(c1)C(=O)[C@]1(O)CCN(c3ccn4nccc4c3)C1=N2]

BPN-0027396

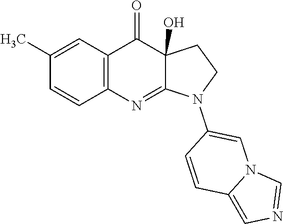 [SMILES: Cc1ccc2c(c1)C(=O)[C@]1(O)CCN(c3ccc4cncn4c3)C1=N2]

BPN-0027406

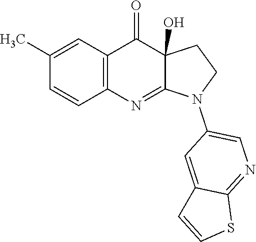 [SMILES: Cc1ccc2c(c1)C(=O)[C@]1(O)CCN(c3cnc4sccc4c3)C1=N2]

BPN-0027410

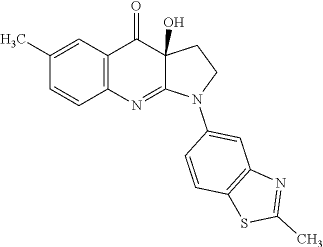

BPN-0027411

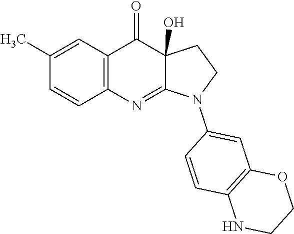 [SMILES: Cc1ccc2c(c1)C(=O)[C@]1(O)CCN(c3ccc4c(c3)OCCN4)C1=N2]

BPN-0027412

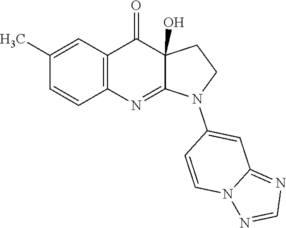 [SMILES: Cc1ccc2c(c1)C(=O)[C@]1(O)CCN(c3ccn4ncnc4c3)C1=N2]

BPN-0027441

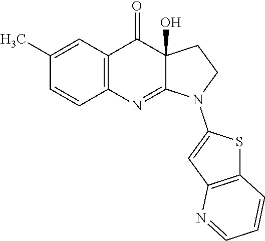 [SMILES: Cc1ccc2c(c1)C(=O)[C@]1(O)CCN(c3cc4ncccc4s3)C1=N2]

BPN-0027468

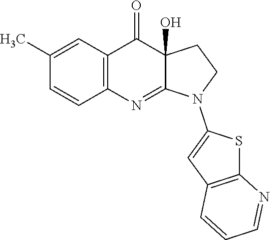 [SMILES: Cc1ccc2c(c1)C(=O)[C@]1(O)CCN(c3cc4cccnc4s3)C1=N2]

BPN-0027469

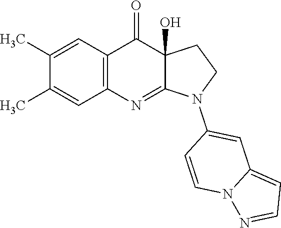 [SMILES: Cc1cc2c(cc1C)C(=O)[C@]1(O)CCN(c3ccn4nccc4c3)C1=N2]

BPN-0027488

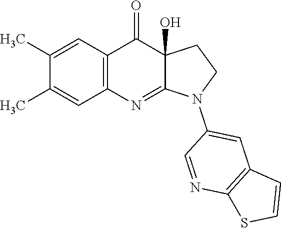 [SMILES: Cc1cc2c(cc1C)C(=O)[C@]1(O)CCN(c3cnc4sccc4c3)C1=N2]

BPN-0027489

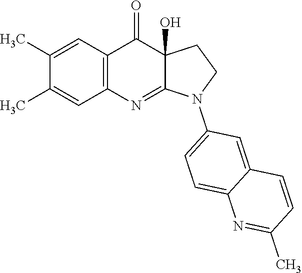 [SMILES: Cc1ccc2cc(N3CC[C@@]4(O)C(=O)c5cc(C)c(C)cc5N=C34)ccc2n1]

BPN-0027490

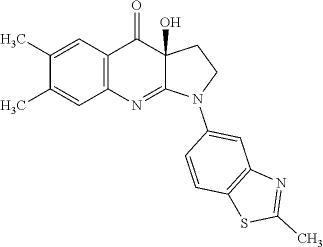

BPN-0027491

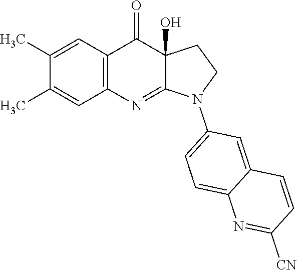 [SMILES: Cc1cc2c(cc1C)C(=O)[C@]1(O)CCN(c3ccc4nc(C#N)ccc4c3)C1=N2]

BPN-0027492

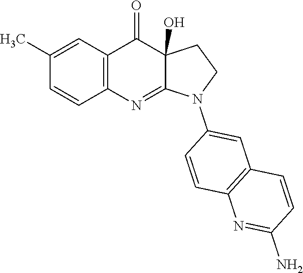 [SMILES: Cc1ccc2c(c1)C(=O)[C@]1(O)CCN(c3ccc4nc(N)ccc4c3)C1=N2]

BPN-0027494

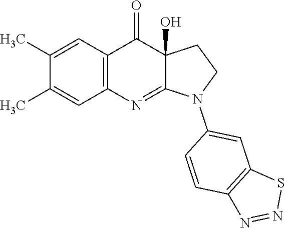 [SMILES: Cc1cc2c(cc1C)C(=O)[C@]1(O)CCN(c3ccc4nnsc4c3)C1=N2]

BPN-0028550

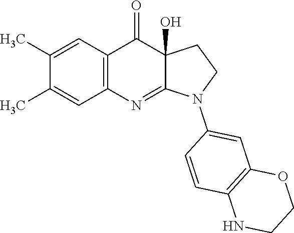 [SMILES: Cc1cc2c(cc1C)C(=O)[C@]1(O)CCN(c3ccc4c(c3)OCCN4)C1=N2]

BPN-0028552

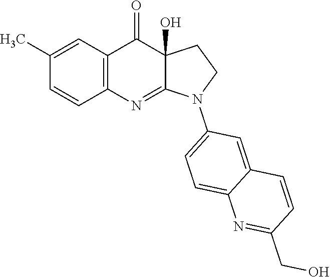 [SMILES: Cc1ccc2c(c1)C(=O)[C@]1(O)CCN(c3ccc4nc(CO)ccc4c3)C1=N2]

BPN-0028554

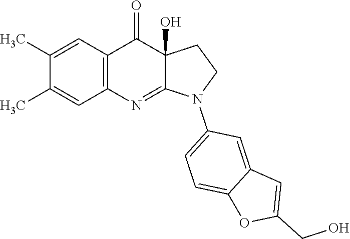 [SMILES: Cc1cc2c(cc1C)C(=O)[C@]1(O)CCN(c3ccc4oc(CO)cc4c3)C1=N2]

BPN-0028555

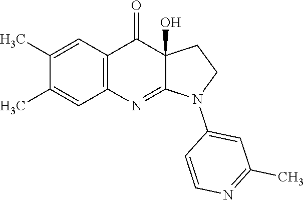

BPN-0028616

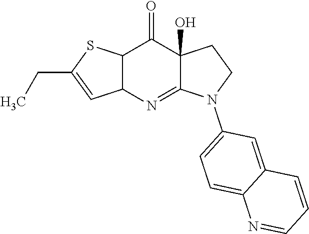 [SMILES: CCC1=CC2N=C3N(c4ccc5ncccc5c4)CC[C@@]3(O)C(=O)C2S1]

BPN-0028625

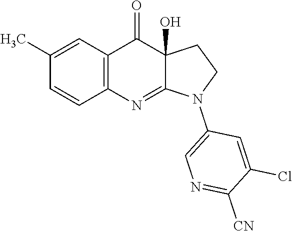 [SMILES: Cc1ccc2c(c1)C(=O)[C@]1(O)CCN(c3cnc(C#N)c(Cl)c3)C1=N2]

BPN-0028646

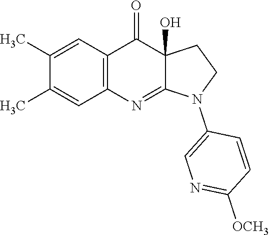 [SMILES: COc1ccc(N2CC[C@@]3(O)C(=O)c4cc(C)c(C)cc4N=C23)cn1]

BPN-0028648

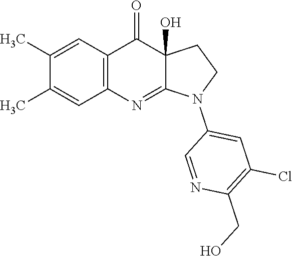 [SMILES: Cc1cc2c(cc1C)C(=O)[C@]1(O)CCN(c3cnc(CO)c(Cl)c3)C1=N2]

BPN-0028649

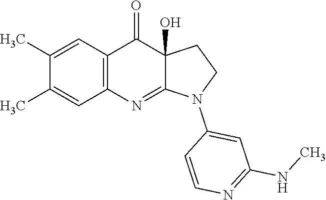

BPN-0028650

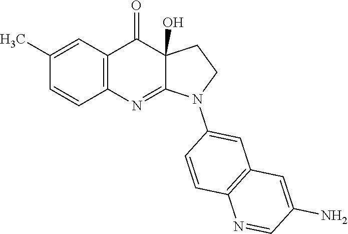 [SMILES: Cc1ccc2c(c1)C(=O)[C@]1(O)CCN(c3ccc4ncc(N)cc4c3)C1=N2]

BPN-0028652

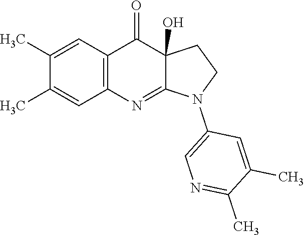

BPN-0028694

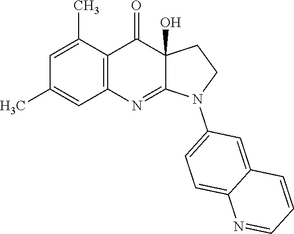 [SMILES: Cc1cc(C)c2c(c1)N=C1N(c3ccc4ncccc4c3)CC[C@@]1(O)C2=O]

BPN-0028697

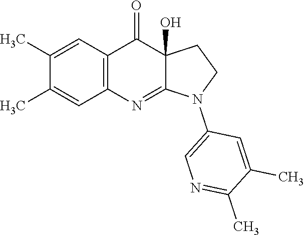

BPN-0028731

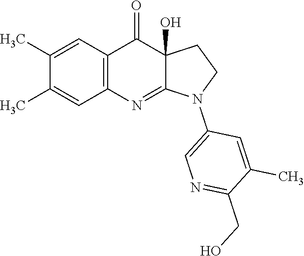

BPN-0028733

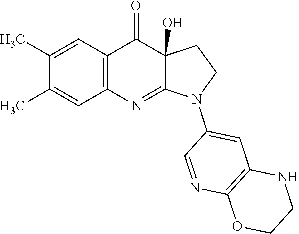 [SMILES: Cc1cc2c(cc1C)C(=O)[C@]1(O)CCN(c3cnc4c(c3)NCCO4)C1=N2]

BPN-0028734

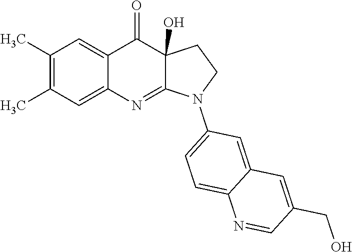 [SMILES: Cc1cc2c(cc1C)C(=O)[C@]1(O)CCN(c3ccc4ncc(CO)cc4c3)C1=N2]

BPN-0028736

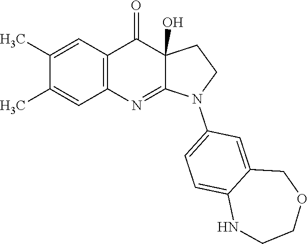 [SMILES: Cc1cc2c(cc1C)C(=O)[C@]1(O)CCN(c3ccc4c(c3)COCCN4)C1=N2]

BPN-0028758

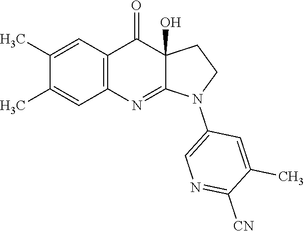

BPN-0028760

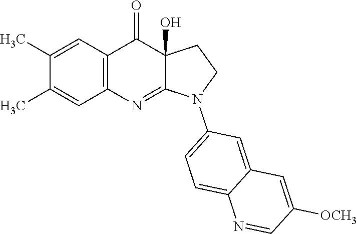 [SMILES: COc1cnc2ccc(N3CC[C@@]4(O)C(=O)c5cc(C)c(C)cc5N=C34)cc2c1]

BPN-0028788

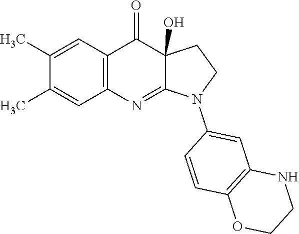 [SMILES: Cc1cc2c(cc1C)C(=O)[C@]1(O)CCN(c3ccc4c(c3)NCCO4)C1=N2]

BPN-0028790

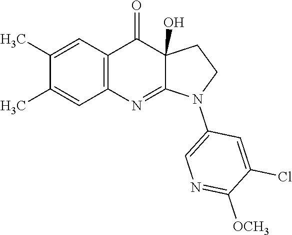 [SMILES: COc1ncc(N2CC[C@@]3(O)C(=O)c4cc(C)c(C)cc4N=C23)cc1Cl]

BPN-0028820

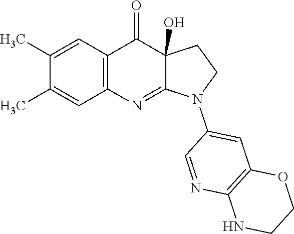 [SMILES: Cc1cc2c(cc1C)C(=O)[C@]1(O)CCN(c3cnc4c(c3)OCCN4)C1=N2]

BPN-0028821

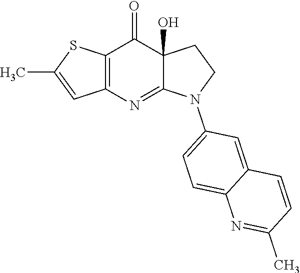 [SMILES: Cc1ccc2cc(N3CC[C@@]4(O)C(=O)c5sc(C)cc5N=C34)ccc2n1]

BPN-0028863

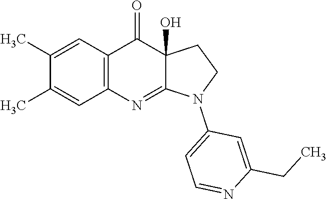 [SMILES: CCc1cc(N2CC[C@@]3(O)C(=O)c4cc(C)c(C)cc4N=C23)ccn1]

BPN-0028864

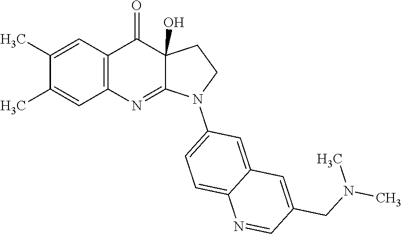

BPN-0028866

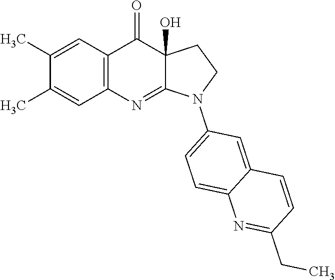 [SMILES: CCc1ccc2cc(N3CC[C@@]4(O)C(=O)c5cc(C)c(C)cc5N=C34)ccc2n1]

BPN-0028867

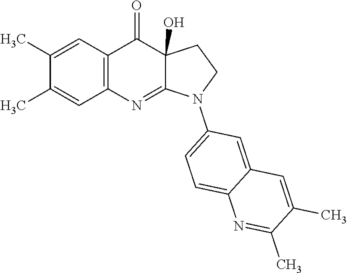

BPN-0028868

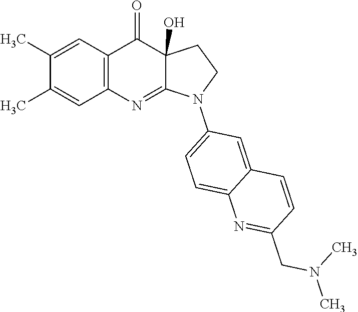

BPN-0028897

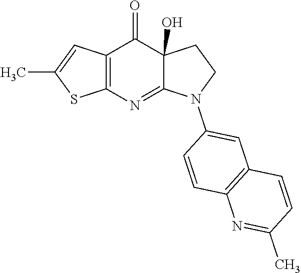 [SMILES: Cc1ccc2cc(N3CC[C@@]4(O)C(=O)c5cc(C)sc5N=C34)ccc2n1]

BPN-0028898

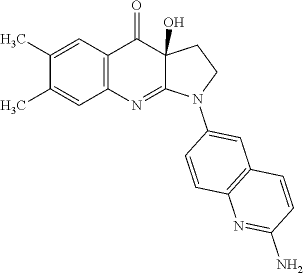 [SMILES: Cc1cc2c(cc1C)C(=O)[C@]1(O)CCN(c3ccc4nc(N)ccc4c3)C1=N2]

BPN-0028899

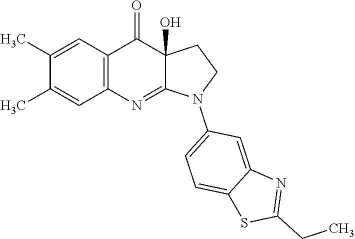 [SMILES: CCc1nc2cc(N3CC[C@@]4(O)C(=O)c5cc(C)c(C)cc5N=C34)ccc2s1]

BPN-0028918

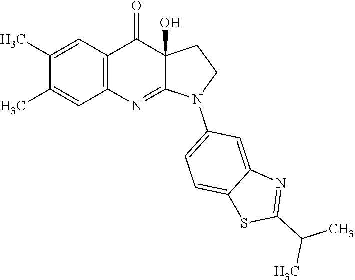 [SMILES: Cc1cc2c(cc1C)C(=O)[C@]1(O)CCN(c3ccc4sc(C(C)C)nc4c3)C1=N2]

BPN-0028919

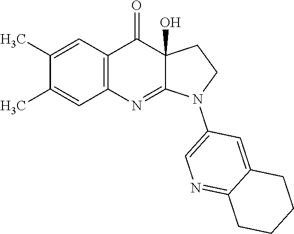 [SMILES: Cc1cc2c(cc1C)C(=O)[C@]1(O)CCN(c3cnc4c(c3)CCCC4)C1=N2]

BPN-0028920

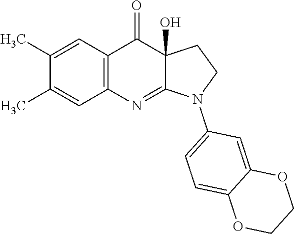 [SMILES: Cc1cc2c(cc1C)C(=O)[C@]1(O)CCN(c3ccc4c(c3)OCCO4)C1=N2]

BPN-0028921

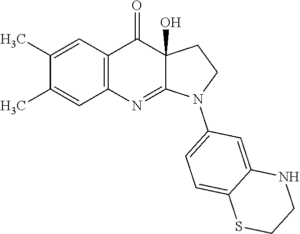 [SMILES: Cc1cc2c(cc1C)C(=O)[C@]1(O)CCN(c3ccc4c(c3)NCCS4)C1=N2]

BPN-0028922

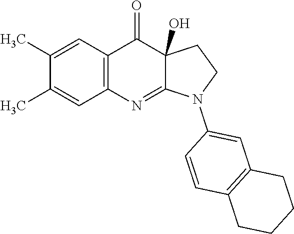 [SMILES: Cc1cc2c(cc1C)C(=O)[C@]1(O)CCN(c3ccc4c(c3)CCCC4)C1=N2]

BPN-0028923

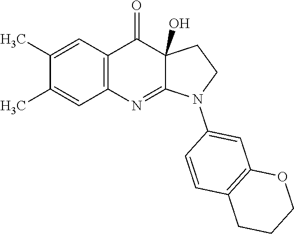 [SMILES: Cc1cc2c(cc1C)C(=O)[C@]1(O)CCN(c3ccc4c(c3)OCCC4)C1=N2]

BPN-0028924

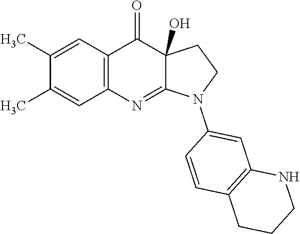 [SMILES: Cc1cc2c(cc1C)C(=O)[C@]1(O)CCN(c3ccc4c(c3)NCCC4)C1=N2]

BPN-0028925

### Blebbistatin Analog Bioassay Methods

### 1. Assaying Cardiac Muscle Myosin II

The cardiac muscle myosin II actin-activated ATPase assay is a biochemical assay. Specifically, it is an NADH (nicotinamide adenine dinucleotide)-coupled ATPase assay that relies on NADH fluorescence as a readout. Cardiac myosin is a mechanochemical energy transducer that hydrolyzes ATP to generate force in the presence of its activator, F-actin. The resulting ADP is regenerated to ATP by pyruvate kinase (PK) that transforms one molecule of phosphoenolpyruvate (PEP) to pyruvate in parallel. Subsequently, pyruvate is reduced to lactate by lactate dehydrogenase (LDH) that, in turn, oxidizes one molecule of NADH to NAD. Therefore, the decrease in NADH concentration as a function of time equals the ATP hydrolysis rate. Bovine cardiac myosin is obtained from a commercial source, Cytoskeleton. PK, LDH, ATP, PEP, and NADH are obtained from Sigma. F-actin is prepared in house from Rabbit Muscle Acetone Powder. The assay is run at 25° C. in 384 well black-wall polystyrene microplates with a total volume of 20 μl per well. NADH fluorescence is monitored for 30 minutes with an EnVision Multimode Plate Reader. The slope of the fluorescence response, which is proportional to the reaction rate, is determined by simple linear regression. Final assay conditions are 300 nM cardiac myosin, 10 μM actin, 40 U/ml LDH, 200 U/ml PK, 220 μM NADH, 1 mM PEP, 1 mM ATP in a buffer containing 10 mM 3-(N-morpholino)propanesulfonic acid (pH=7.0), 2 mM MgCl₂, 0.15 mM ethylene glycol-bis(p-aminoethyl ether)-N,N,N′,N′-tetraacetic acid, 0.1 mg/mL bovine serum albumin, 0.5% (V/V) dimethyl sulfoxide (DMSO) and 1 mM dithiothreitol. Prior to testing the inhibitory activity of the compounds, a two-fold dilution series starting at 10 mM compound concentration is prepared in DMSO. Subsequently, 100 nl is transferred to each well of the measuring plate containing a mixture of myosin, LDH and PK. The enzymatic reaction is started with the addition of a mixture containing ATP, PEP, NADH and actin. The highest final compound concentration is 50 uM. 20 μM para-aminoblebbistatin in 0.5% DMSO serves as the positive control and 0.5% DMSO alone is the negative control. Reaction rates are determined by using the fluorescence responses of a dilution series of NADH included in all plates and plotted as a function of inhibitor concentration. All measurements are carried out in triplicate. Inhibitory constants are determined by fitting the 16 point dose-response data to a quadratic equation corresponding to a simple one-to-one binding model. Small molecules showing no observable inhibition at or below their solubility are reported as inactive.

### 2. Assaying Skeletal Muscle Myosin II

The skeletal muscle myosin II actin-activated ATPase assay is performed the same as for cardiac muscle myosin II with the following exceptions: Rabbit skeletal myosin is obtained from Cytoskeleton and the final assay conditions contain 20 nM skeletal muscle myosin II.

### 3. Assaying Nonmuscle Myosin II

Cytokinesis is a cell-based assay to assess nonmuscle myosin II function, performed using COS7 cells. The assay is run in a 96 well plate, with 2,000 cells plated per well. Total incubation time is 48 hours, with 24 hours of treatment with the compound of interest. Prior to treatment with small molecules, a two-fold dilution series of compound solutions is prepared in DMSO. Starting concentrations are determined based on compound solubility. Compound solutions are further diluted in DMEM medium to a final DMSO concentration of 2% (50-fold dilution). Subsequently, 100 μl diluted solution is transferred to each well of the plate containing 100 μl of cell culture (achieving 1% final DMSO concentration). All measurements are carried out in triplicate. Cells are stained by fluorescein diacetate (6 μM), a cell viability dye, the cell-permeant Hoescht33342 (10 μM) and the membrane-impermeant propidium iodide (4 μM) to label all nuclei and those belonging to dead cells, respectively. Dye solutions are replaced by fresh media after 10 minutes of incubation. An INCell Analyzer 6000 is used for imaging. The signal of interest is the ratio of nuclei to cell numbers, as nonmuscle myosin II inhibition prevents cellular blebbing, resulting in multinucleated cells. Cytotoxicity is also assayed and quantified as the ratio of dead nuclei to total nuclei. 20 μM para-aminoblebbistatin in 1% DMSO serves as the positive control and 1% DMSO alone is the negative control. Half maximal effective concentration (EC₅₀) is determined by fitting the 6-point dose-response data to the Hill equation. Small molecules showing no observable inhibition are reported as inactive.

### 4. Assaying Cardiac Safety (Assay 1)

The spontaneous contractions of cardiomyocytes (CMs) is measured label-free in real time using the ACEA xCELLigence RTCA Cardio instrument. All measurements are performed at 37° C. in a cell culture incubator, allowing for spontaneous beating of CMs at physiological conditions. Cor.4U CMs are seeded at 3×104 cells/well in 180 μl/well maintenance medium. Prior to seeding, a background impedance measurement (plate plus medium) is performed. The contraction of CMs involves cyclic modulation of cell morphology and adhesion inducing a delta change in impedance. From the cyclic changes in impedance a beating frequency, amplitude and effects on beating pattern can be calculated. Time point “0” (baseline) is recorded 10× for 20 seconds at 1 minute intervals. Thereafter, compounds and control (0.1% DMSO) are added to the wells and effects are measured 30 minutes later. Vehicle control is 0.1% DMSO and positive control is 100 nM isoproterenol. Compounds are tested at 0.1, 0.3, 1.0 and 3.0 uM. Data is collected at 5, 10, 15, 20 and 30 minutes, and 1, 4, 6, 12 and 24 hours post-incubation.

### 5. Assaying Cardiac Safety (Assay 2)

Male Sprague-Dawley rats are acclimated for approximately 1 week prior to use. Rats are lightly anesthetized using isoflurane and an intravenous catheter is placed for dosing purposes. Baseline 2-D echocardiograms are collected (standard SAX B-mode and M-mode at the level of the papillary muscle). Each rat receives a total of 3 IV treatments. Vehicle (10% DMSO, 10% Tween 80 and 80% water) is first administered, followed 10 minutes later by two subsequent infusions, separated by 10 minutes. Infusions of the positive control, Blebbistatin, are delivered at 0.5 mg/kg IV. Test compound concentrations vary depending upon the compound and experimental question. However, when available, dose is adjusted based on plasma pharmacokinetics and NMII potency. Standard SAX ECHO images are collected at the initiation of and at 1, 5, and 10 min after the initiation of each treatment. Measurements including fractional shortening, ejection fraction, heart rate and cardiac output are calculated.

Compounds of the invention were prepared according to the Synthetic Schemes 1-7, below, and all compounds disclosed and claimed herein can be prepared according to these Schemes, in conjunction with ordinary skill and knowledge of a synthetic organic chemist and illustrated by the detailed procedures for specific examples further provided below. HPLC Conditions:

### Method A

Column: Waters Symmetry 5 μm C18 (250×4.6 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method a Gradient

| Time | Flow | | |
|---|---|---|---|
| (min) | (mL/min) | % A | % B |
| 0.0 | 1.0 | 98.0 | 2.0 |
| 20.0 | 1.0 | 0.0 | 100.0 |
| 25.0 | 1.0 | 0.0 | 100.0 |

### Method B

Column: YMC ODS-AQ C18 120 Å (150×4.6 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method B Gradient

| Time | Flow | | |
|---|---|---|---|
| (min) | (mL/min) | % A | % B |
| 0.0 | 1.0 | 98.0 | 2.0 |
| 15.0 | 1.0 | 0.0 | 100.0 |
| 19.0 | 1.0 | 0.0 | 100.0 |

### Method C

Column: xBridge 3.5 μm C18 (150×4.6 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method C Gradient

| Time | Flow | | |
|---|---|---|---|
| min) | (mL/min) | % A | % B |
| 0.0 | 1.0 | 95.0 | 5.0 |
| 20.0 | 1.0 | 0.0 | 100.0 |
| 25.0 | 1.0 | 0.0 | 100.0 |

### Method D

Column: YMC ODS-AQ C18 120 Å (150×4.6 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method D Gradient

| Time | Flow | | |
|---|---|---|---|
| (min) | (mL/min) | % A | % B |
| 0.0 | 1.0 | 95.0 | 5.0 |
| 20.0 | 1.0 | 0.0 | 100.0 |
| 25.0 | 1.0 | 0.0 | 100.0 |

### Method E

Column: YMC ODS-AQ C18 120 Å (150×4.6 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method E Gradient

| Time | Flow | | |
|---|---|---|---|
| (min) | (mL/min) | % A | % B |
| 0.0 | 1.0 | 100.0 | 0.0 |
| 5.0 | 1.0 | 100.0 | 0.0 |
| 15.0 | 1.0 | 0.0 | 100.0 |
| 18.0 | 1.0 | 0.0 | 100.0 |

### Method F

Column: Waters Symmetry 5 μm C18 (250×4.6 mm)

Mobile Phase A: Water containing 0.1% v/v Trifluoroacetic Acid

Mobile Phase B: Acetonitrile containing 0.1% v/v Trifluoroacetic Acid

Detection: 290 nm

### Method F Gradient

| Time | Flow | | |
|---|---|---|---|
| (min) | (mL/min) | % A | % B |
| 0.0 | 1.0 | 95.0 | 5.0 |
| 20.0 | 1.0 | 0.0 | 100.0 |
| 25.0 | 1.0 | 0.0 | 100.0 |

### UPLC Conditions:

### Method A

Column: Acquity UPLC BEH 1.7 μm C18 (75×2.1 mm)

Mobile Phase A: Water containing 0.1% v/v Trifluoroacetic Acid

Mobile Phase B: Acetonitrile containing 0.1% v/v Trifluoroacetic Acid

Detection: 254 nm

### Method A Gradient

| Time | Flow | | |
|---|---|---|---|
| (min) | mL/min) | % A | % B |
| 0.0 | 0.5 | 95.0 | 5.0 |
| 6.0 | 0.5 | 0.0 | 100.0 |
| 8.0 | 0.5 | 0.0 | 100.0 |

### Chiral HPLC Conditions:

### Method A

Column: Chiralpak AD 5 μm (250×4.6 mm)

Mobile Phase A: Heptane

Mobile Phase B: i-Propyl Alcohol

Detection: 254 nm

### Method A Gradient

| Time | Flow | | |
|---|---|---|---|
| (min) | (mL/min) | % A | % B |
| 0.0 | 1.0 | 90.0 | 10.0 |
| 5.0 | 1.0 | 90.0 | 10.0 |
| 20.0 | 1.0 | 50.0 | 50.0 |
| 35.0 | 1.0 | 50.0 | 50.0 |

### Method B

Column: Chiralpak AD 5 μm (250×4.6 mm)

Mobile Phase A: Heptane

Mobile Phase B: i-Propyl Alcohol

Detection: 290 nm

### Method B Gradient

| Time | Flow | | |
|---|---|---|---|
| (min) | (mL/min) | % A | % B |
| 0.0 | 1.0 | 90.0 | 10.0 |
| 5.0 | 1.0 | 90.0 | 10.0 |
| 20.0 | 1.0 | 50.0 | 50.0 |
| 35.0 | 1.0 | 50.0 | 50.0 |

### Chiral SFC Conditions:

### Method A

Column: Chiralcel OJ-H 5 μm (100×4.6 mm)

Mobile Phase A: CO₂

Mobile Phase B: i-Propyl Alcohol

Detection: 254 nm

### Method A Gradient

| Time | Flow | | |
|---|---|---|---|
| (min) | (mL/min) | % A | % B |
| 0.0 | 1.5 | 95.0 | 5.0 |
| 8.0 | 1.5 | 60.0 | 40.0 |

### Method B

Column: Chiralcel OJ-H 5 μm (100×4.6 mm)

Mobile Phase A: CO₂

Mobile Phase B: Methanol

Detection: 254 nm

### Method B Gradient

| Time | Flow | | |
|---|---|---|---|
| (min) | (mL/min) | % A | % B |
| 0.0 | 1.5 | 95.0 | 5.0 |
| 8.0 | 1.5 | 60.0 | 40.0 |

### Synthetic Schemes

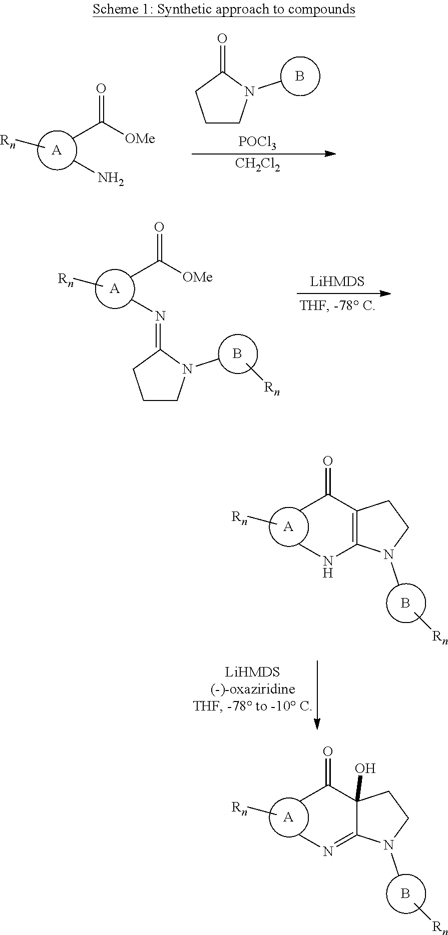

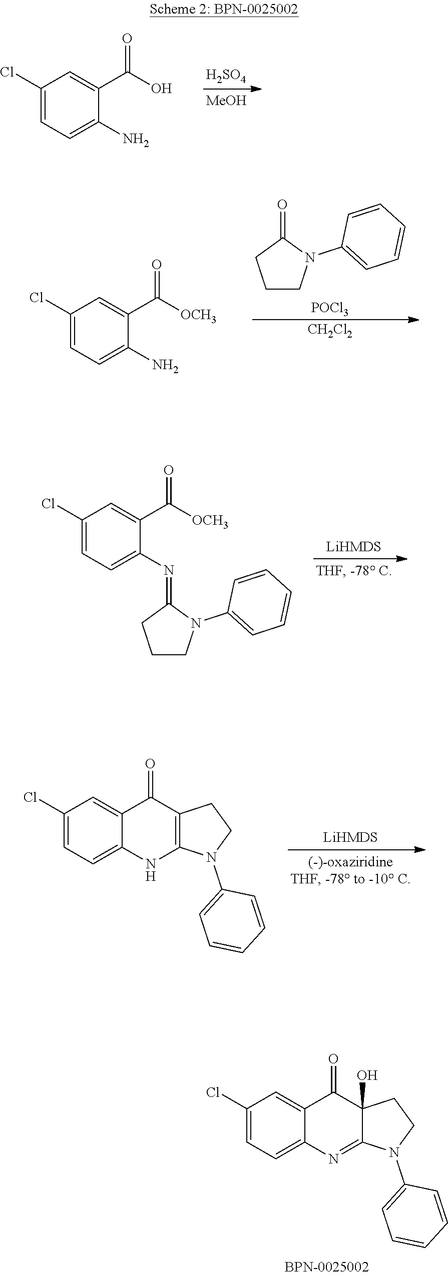

### Preparation of Methyl 2-amino-5-chlorobenzoate

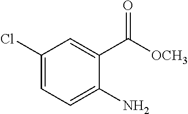 [SMILES: COC(=O)c1cc(Cl)ccc1N]

A solution of 2-amino-5-chlorobenzoic acid (5.00 g, 29.1 mmol) in methanol (75 mL) was treated with concentrated sulfuric acid (7.5 mL) and heated at 60° C. under a nitrogen atmosphere for 72 h. After this time, the reaction mixture was concentrated under reduced pressure to remove the volatiles. The resulting residue was carefully treated with saturated aqueous sodium bicarbonate (150 mL) and extracted with ethyl acetate (3×100 mL). The combined organics were dried over sodium sulfate, filtered, and concentrated under reduced pressure to provide methyl 2-amino-5-chlorobenzoate (5.10 g, 94%) as a brown solid: ¹H NMR (500 MHz, DMSO-d₆) δ 7.63 (d, J=2.5 Hz, 1H), 7.28 (dd, J=8.5, 2.5 Hz, 1H), 6.81 (d, J=8.5 Hz, 1H), 6.77 (br s, 2H), 3.80 (s, 3H).

### Preparation of Methyl 5-chloro-2-((1-phenylpyrrolidin-2-ylidene)amino)benzoate

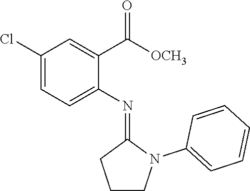 [SMILES: COC(=O)c1cc(Cl)ccc1/N=C1\CCCN1c1ccccc1]

A solution of 1-phenylpyrrolidin-2-one (1.04 g, 6.45 mmol) in methylene chloride (13 mL) was treated with phosphorous oxychloride (0.60 mL, 0.99 g, 6.4 mmol) and stirred under a nitrogen atmosphere at ambient temperature for 3 h. The mixture was treated with a solution of methyl 2-amino-5-chlorobenzoate (1.00 g, 5.39 mmol) in methylene chloride (3 mL) and heated at 50° C. for 16 h. After this time, the reaction mixture was allowed to cool to ambient temperature, diluted with methylene chloride (25 mL), washed with saturated aqueous sodium bicarbonate (2×25 mL) and brine (25 mL), dried over sodium sulfate, filtered, and concentrated under reduced pressure. The crude residue was purified by column chromatography (silica gel, 0-30% ethyl acetate/methylene chloride) to provide methyl 5-chloro-2-((1-phenylpyrrolidin-2-ylidene)amino)benzoate (0.72 g, 41%) as a light tan oil: ESI MS m/z 329 [C₁₈H₁₇ClN₂O₂+H]⁺.

### Preparation of 6-Chloro-1-phenyl-2,3-dihydro-1H-pyrrolo[2,3-b]quinolin-4(9H-one

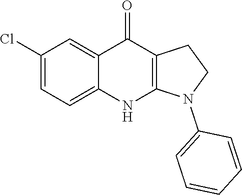 [SMILES: O=c1c2c([nH]c3ccc(Cl)cc13)N(c1ccccc1)CC2]

A solution of methyl 5-chloro-2-((1-phenylpyrrolidin-2-ylidene)amino)benzoate (715 mg, 2.17 mmol) in tetrahydrofuran (10 mL) was cooled in a dry ice/acetone bath under a nitrogen atmosphere and treated dropwise with a 1.0 M solution of lithium bis(trimethylsilyl)amide in tetrahydrofuran (5.4 mL, 5.4 mmol). The mixture was stirred for 3.5 h, during which time the bath temperature increased to ˜0° C. After this time, the mixture was treated with chilled saturated aqueous ammonium chloride (50 mL) and stirred rapidly for 1 h. The resulting solids were isolated by filtration, washed with water and ethyl acetate, and dried under vacuum to provide 6-chloro-1-phenyl-2,3-dihydro-1H-pyrrolo[2,3-b]quinolin-4(9H)-one (435 mg, 67%) as a light orange solid: ¹H NMR (500 MHz, DMSO-d₆) δ 10.77 (s, 1H), 8.05 (d, J=8.0 Hz, 2H), 7.93 (d, J=2.5 Hz, 1H), 7.60 (d, J=8.5 Hz, 1H), 7.47 (dd, J=8.5, 2.5 Hz, 1H), 7.39 (apparent t, J=7.5 Hz, 2H), 7.02 (apparent t, J=7.5 Hz, 1H), 4.11 (t, J=8.0 Hz, 2H), 3.19 (t, J=8.0 Hz, 2H); ESI MS m/z 297 [C₁₇H₁₃ClN₂O+H]⁺.

### Preparation of (S)-6-Chloro-3a-hydroxy-1-phenyl-3,3a-dihydro-1H-pyrrolo[2,3-b]quinolin-4(2H)-one

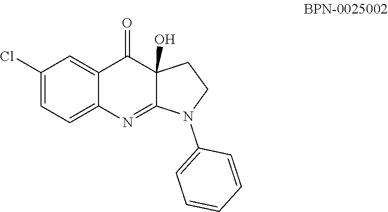 [SMILES: O=C1c2cc(Cl)ccc2N=C2N(c3ccccc3)CC[C@@]12O]

A solution of 6-chloro-1-phenyl-2,3-dihydro-1H-pyrrolo[2,3-b]quinolin-4(9H)-one (101 mg, 0.339 mmol) in tetrahydrofuran (6 mL) was cooed in a dry ice/acetone bath and treated with a 1.0 M solution of lithium bis(trimethylsilyl)amide in tetrahydrofuran (0.41 mL, 0.41 mmol) under a nitrogen atmosphere. After stirring for 45 min, the mixture was treated with a solution of (−)-(8,8-dichlorocamphorylsulfonyl)oxaziridine (245 mg, 0.821 mmol) in tetrahydrofuran (3 mL). The mixture was stirred for 2 h while the bath temperature increased to ˜0° C. The acetone bath was replaced by a wet ice/brine bath, and the mixture was stirred for 3 h. After this time, the mixture was treated with saturated aqueous ammonium iodide (6 mL) followed by saturated aqueous sodium thiosulfate (25 ml) and extracted with ethyl acetate (3×25 mL). The organics were extracted with 0.3 M hydrochloric acid (3×25 mL). The combined acid layers were adjusted to pH ˜8 with 2.0 M aqueous sodium hydroxide and extracted with ethyl acetate (4×25 mL). The organics were dried over sodium sulfate, filtered, and concentrated under reduced pressure. The crude product was recrystallized from hot acetonitrile to provide (S)-6-chloro-3a-hydroxy-1-phenyl-3,3a-dihydro-1H-pyrrolo[2,3-b]quinolin-4(2H)-one (37 mg, 35%) as a yellow solid: mp=199-200° C. decomposed; ¹H NMR (500 MHz, DMSO-d₆) δ 8.06 (dd, J=7.5, 1.0 Hz, 2H), 7.64 (d, J=2.5 Hz, 1H), 7.56 (dd, J=8.5, 2.5 Hz, 1H), 7.44 (apparent t, J=7.0 Hz, 2H), 7.22 (d, J=8.5 Hz, 1H), 7.17 (apparent t, J=7.0 Hz, 1H), 6.96 (s, 1H), 4.14-4.08 (m, 1H), 3.98 (apparent t, J=9.0 Hz, 1H), 2.37-2.31 (m, 1H), 2.26 (dd, J=13.5, 6.0 Hz, 1H); ESI MS m/z 313 [C₁₇H₁₃ClN₂O₂+H]⁺; HPLC (Method A) >99% (AUC), t_{R}=11.17 min; Chiral HPLC (Chiralpak AD, Method A) >99% (AUC), t_{R}=11.17 min.

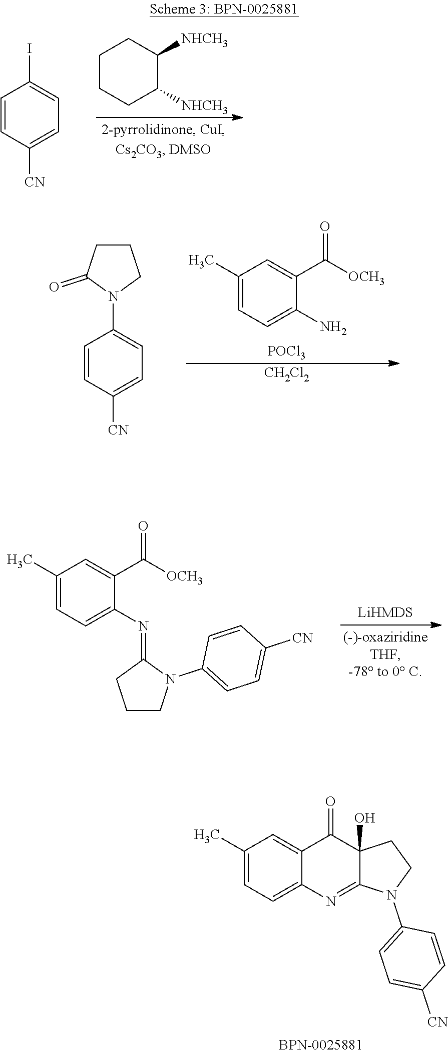

### Preparation of 4-(2-Oxopyrrolidin-1-yl)benzonitrile

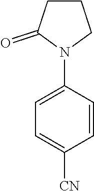 [SMILES: N#Cc1ccc(N2CCCC2=O)cc1]

A solution of 4-iodobenzonitrile (1.00 g, 4.36 mmol) in dimethyl sulfoxide (10 mL) was treated with 2-pyrrolidinone (331 μL, 4.36 mmol), copper iodide (83.0 mg, 0.436 mmol), cesium carbonate (4.26 g, 13.1 mmol) and N,N-dimethyl-(1R,2R)-1,2-cyclohexanediamine (137 μL, 0.872 mmol) and heated at 110° C. under a nitrogen atmosphere for 16 h. After this time, the reaction mixture was allowed to cool to ambient temperature, diluted with water (50 mL), and extracted with ethyl acetate (4×50 mL). The combined organics were washed with water (4×10 mL), dried over sodium sulfate, filtered, and concentrated under reduced pressure. The crude residue was purified by column chromatography (silica gel, 0-100% ethyl acetate/heptane) to provide 4-(2-oxopyrrolidin-1-yl)benzonitrile (514 mg, 63%) as an off-white solid: ¹H NMR (300 MHz, DMSO-d₆) δ 7.90-7.82 (m, 4H), 3.87 (t, J=7.2 Hz, 2H), 2.56 (t, J=3.9 Hz, 2H), 2.12-2.05 (m, 2H).

### Preparation of Methyl 2-((1-(4-Cyanophenyl)pyrrolidin-2-ylidene)amino)-5-methylbenzoate

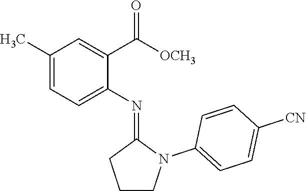 [SMILES: COC(=O)c1cc(C)ccc1/N=C1\CCCN1c1ccc(C#N)cc1]

A solution of 4-(2-oxopyrrolidin-1-yl)benzonitrile (400 mg, 2.14 mmol) in methylene chloride (9 mL) was treated with phosphorous oxychloride (0.30 mL, 3.2 mmol) and stirred under a nitrogen atmosphere at ambient temperature for 16 h. The mixture was treated with a solution of methyl 2-amino-5-methylbenzoate (355 mg, 2.14 mmol) in methylene chloride (2 mL) and heated at 45° C. for 5 d. After this time, the reaction mixture was allowed to cool to ambient temperature, quenched with saturated aqueous sodium bicarbonate (15 mL), and extracted with ethyl acetate. The organics were dried over sodium sulfate, filtered, and concentrated under reduced pressure. The crude residue was dissolved in ethyl acetate and extracted with 0.3 M hydrochloric acid (2×20 mL). The combined acid layers were adjusted to pH ˜11 with 2.0 M aqueous sodium hydroxide and extracted with ethyl acetate (3×25 mL). The organics were dried over sodium sulfate, filtered, and concentrated under reduced pressure to provide methyl 2-((1-(4-cyanophenyl)pyrrolidin-2-ylidene)amino)-5-methylbenzoate (226 mg, 32%) as a yellow oil, which was used without further purification: ESI MS m/z 334 [C₂₀H₁₉N₃O₂+H]⁺.

### Preparation of (S)-4-(3a-Hydroxy-6-methyl-4-oxo-2,3,3a,4-tetrahydro-1H-pyrrolo[2,3-b]quinolin-1-yl)benzonitrile

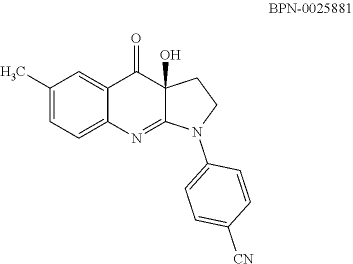 [SMILES: Cc1ccc2c(c1)C(=O)[C@]1(O)CCN(c3ccc(C#N)cc3)C1=N2]

A solution of methyl 2-((1-(4-cyanophenyl)pyrrolidin-2-ylidene)amino)-5-methylbenzoate (226 mg, 0.678 mmol) in tetrahydrofuran (12 mL) was cooled in a dry ice/acetone bath under a nitrogen atmosphere and treated with a 1.0 M solution of lithium bis(trimethylsilyl)amide in tetrahydrofuran (2.03 mL, 2.03 mmol). The acetone bath was replaced by a wet ice/water bath, and the mixture was stirred for 3 h. After stirring for 3 h, the mixture was treated with a solution of (−)-(8,8-dichlorocamphorylsulfonyl)oxaziridine (505 mg, 1.70 mmol) in tetrahydrofuran (8 mL). The mixture was stirred for 2 h at ˜0° C. After this time, the mixture was treated sequentially with saturated aqueous ammonium iodide (0.7 mL), saturated aqueous sodium thiosulfate (2.3 mL), and brine (20 mL). The aqueous layer was extracted with ethyl acetate (3×25 mL). The organics were combined, dried over sodium sulfate, filtered, and concentrated under reduced pressure. The crude residue was purified by column chromatography (silica gel, 0-100% ethyl acetate/heptane) to obtain a yellow gum, which was re-purified by column chromatography (silica gel, 0-80% ethyl acetate/heptane) and recrystallization from hot acetonitrile to provide (S)-4-(3a-hydroxy-6-methyl-4-oxo-2,3,3a,4-tetrahydro-1H-pyrrolo[2,3-b]quinolin-1-yl)benzonitrile (39 mg, 18%) as a yellow solid: mp=222-223° C.; ¹H NMR (500 MHz, DMSO-d₆) δ 8.32 (dd, J=7.0, 2.0 Hz, 2H), 7.88 (dd, J=7.0, 2.0 Hz, 2H), 7.57 (apparent d, J=2.0 Hz, 1H), 7.42 (dd, J=8.5, 1.5 Hz, 1H), 7.20 (d, J=8 Hz, 1H), 6.92 (s, 1H), 4.06-4.02 (m, 2H), 2.32 (s, 3H), 2.29-2.27 (m, 2H); ESI MS m/z 318 [C₁₉H₁₅N₃O₂+H]⁺; HPLC (Method B) >99% (AUC), t_{R}=9.48 min.

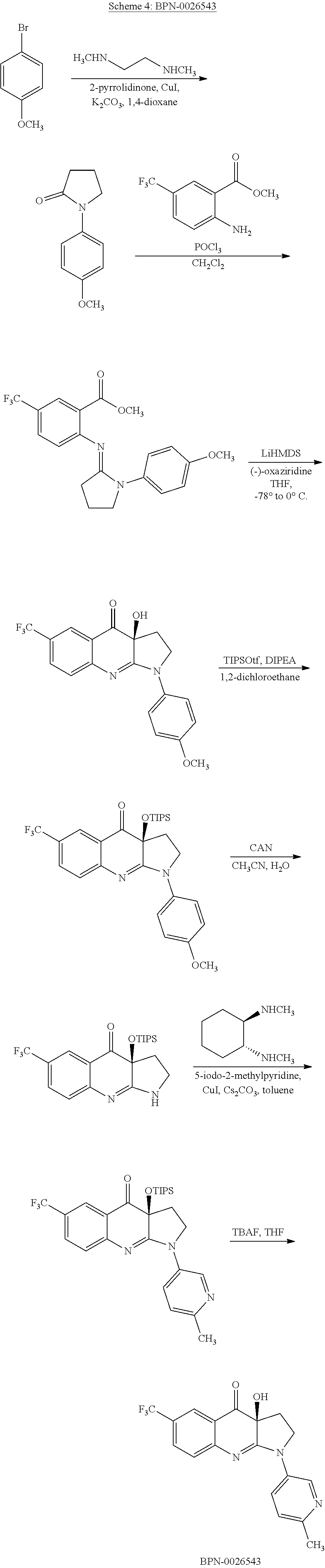

### Preparation of Methyl 2-((1-(4-Methoxyphenyl)pyrrolidin-2-ylidene)amino)-5-(trifluoromethyl)benzoate

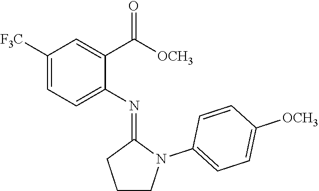 [SMILES: COC(=O)c1cc(C(F)(F)F)ccc1/N=C1\CCCN1c1ccc(OC)cc1]

A solution of 1-(4-methoxyphenyl)pyrrolidin-2-one (2.00 g, 10.5 mmol) in methylene chloride (20 mL) was treated with phosphorous oxychloride (1.46 mL, 15.7 mmol) and stirred under a nitrogen atmosphere at ambient temperature for 7 h. The mixture was treated with a solution of methyl 2-amino-5-(trifluoromethyl)benzoate (3.21 g, 14.6 mmol) in methylene chloride (20 mL) and heated at 45° C. for 2 d. After this time, the reaction mixture was allowed to cool to ambient temperature, quenched with saturated aqueous sodium bicarbonate (40 mL), and extracted with ethyl acetate. The organics were dried over sodium sulfate, filtered, and concentrated under reduced pressure. The crude residue was purified by column chromatography (silica gel, 12-100% ethyl acetate/heptane) to provide methyl 2-((1-(4-methoxyphenyl)pyrrolidin-2-ylidene)amino)-5-(trifluoromethyl)benzoate (2.19 g, 53%) as a clear gum: ¹H NMR (500 MHz, CDCl₃) δ 8.10 (s, 1H), 7.63 (d, J=8.5 Hz, 2H), 7.56 (dd, J=8.5, 1.5 Hz, 1H), 6.90 (d, J=8.5 Hz, 3H), 3.88-3.85 (m, 5H), 3.79 (s, 3H), 2.47 (t, J=7.5 Hz, 2H), 2.11-2.05 (m, 2H).

### Preparation of (S)-3a-Hydroxy-1-(4-methoxyphenyl)-6-(trifluoromethyl)-1,2,3,3a-tetrahydro-4H-pyrrolo[2,3-b]quinolin-4-one

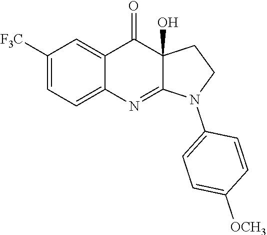 [SMILES: COc1ccc(N2CC[C@@]3(O)C(=O)c4cc(C(F)(F)F)ccc4N=C23)cc1]

A solution of methyl 2-((1-(4-methoxyphenyl)pyrrolidin-2-ylidene)amino)-5-(trifluoromethyl)benzoate (2.19 g, 5.59 mmol) in tetrahydrofuran (35 mL) was cooled in a dry ice/acetone bath under a nitrogen atmosphere and treated with a 1.0 M solution of lithium bis(trimethylsilyl)amide in tetrahydrofuran (16.8 mL, 16.8 mmol). The acetone bath was replaced by a wet ice/water bath, and the mixture was stirred for 1 h. After this time, the mixture was treated with a solution of (−)-(8,8-dichlorocamphorylsulfonyl)oxaziridine (4.17 g, 14.0 mmol) in tetrahydrofuran (20 mL). The mixture was stirred for 1 h at ˜0° C. After this time, the mixture was treated sequentially with saturated aqueous ammonium iodide (20 mL), saturated aqueous sodium thiosulfate (37 mL), and brine (50 mL). The aqueous layer was extracted with ethyl acetate (3×50 mL). The organics were combined, dried over sodium sulfate, filtered, and concentrated under reduced pressure. The crude residue was triturated in hot acetonitrile to provide (S)-3a-hydroxy-1-(4-methoxyphenyl)-6-(trifluoromethyl)-1,2,3,3a-tetrahydro-4H-pyrrolo[2,3-b]quinolin-4-one (1.26 g, 60%) as a yellow-brown solid: ¹H NMR (500 MHz, DMSO-d₆) δ 7.93 (dd, J=7.0, 2.5 Hz, 2H), 7.90 (d, J=1.5 Hz, 1H), 7.81 (dd, J=8.5, 2.5 Hz, 1H), 7.29 (d, J=8.0 Hz, 1H), 7.03 (dd, J=7.0, 2.5 Hz, 2H), 6.99 (s, 1H), 4.16-4.12 (m, 1H), 3.98-3.94 (m, 1H), 3.79 (s, 3H), 2.42-2.35 (m, 1H), 2.28-2.25 (m, 1H).

### Preparation of (S)-1-(4-Methoxyphenyl)-6-(trifluoromethyl)-3a-((triisopropylsilyl)oxy)-1,2,3,3a-tetrahydro-4H-pyrrolo[2,3-b]quinolin-4-one

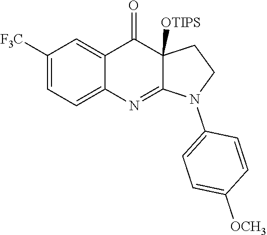

A solution of (S)-3a-hydroxy-1-(4-methoxyphenyl)-6-(trifluoromethyl)-1,2,3,3a-tetrahydro-4H-pyrrolo[2,3-b]quinolin-4-one (730 mg, 1.94 mmol) in 1,2-dichloroethane (30 mL) was treated with N,N-diisopropylethylamine (1.35 mL, 7.76 mmol) and triisopropylsilyl trifluoromethanesulfonate (1.56 mL, 5.82 mmol) and stirred under a nitrogen atmosphere at 90° C. for 16 h. After this time, the reaction mixture was allowed to cool to ambient temperature. The mixture was treated with cold deionized water (17 mL) followed by saturated aqueous ammonium chloride (40 mL). The aqueous layer was extracted with ethyl acetate (3×50 mL). The organics were dried over sodium sulfate, filtered, and concentrated under reduced pressure. The crude residue was purified by column chromatography (silica gel, 0-60% ethyl acetate/heptane) to provide (S)-1-(4-methoxyphenyl)-6-(trifluoromethyl)-3a-((triisopropylsilyl)oxy)-1,2,3,3a-tetrahydro-4H-pyrrolo[2,3-b]quinolin-4-one (870 mg, 84%) as a yellow solid: ¹H NMR (500 MHz, DMSO-d₆) δ 7.93-7.88 (m, 3H), 7.82 (d, J=8.5 Hz, 1H), 7.30 (d, J=8.5 Hz, 1H), 7.04 (d, J=9.0 Hz, 2H), 4.10-4.05 (m, 2H), 3.79 (s, 3H), 2.57-2.50 (m, 1H), 2.33-2.30 (m, 1H), 0.86-0.80 (m, 21H).

### Preparation of (S)-6-(Trifluoromethyl)-3a-((triisopropylsilyl)oxy)-1,2,3,3a-tetrahydro-4H-pyrrolo[2,3-b]quinolin-4-one

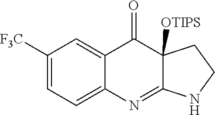

A solution of (S)-1-(4-methoxyphenyl)-6-(trifluoromethyl)-3a-((triisopropylsilyl)oxy)-1,2,3,3a-tetrahydro-4H-pyrrolo[2,3-b]quinolin-4-one (870 mg, 1.63 mmol) in acetonitrile (24 mL) was cooled in a wet ice/water bath under a nitrogen atmosphere and treated dropwise with a solution of ammonium cerium(IV) nitrate (3.58 g, 6.53 mmol) in deionized water (6 mL) and stirred for 2 h at ˜0° C. The mixture was treated with an additional solution of ammonium cerium(IV) nitrate (1.79 g, 3.27 mmol) in deionized water (3 mL) and acetonitrile (12 mL) and stirred for 3 h at ˜0° C. After this time, the mixture was treated with sodium thiosulfate pentahydrate (3.67 g) in deionized water (6 mL) and acetonitrile (25 mL) followed by saturated aqueous sodium bicarbonate (50 mL) to form a slurry. The solid was removed by filtration through diatomaceous earth and rinsed with ethyl acetate. The filtrate was extracted with ethyl acetate (3×50 mL). The organics were combined and washed with saturated sodium bicarbonate, water, and brine. The organics were dried over sodium sulfate, filtered, and concentrated under reduced pressure. The crude residue was purified by column chromatography (silica gel, 12-100% ethyl acetate/heptane) to provide (S)-6-(trifluoromethyl)-3a-((triisopropylsilyl)oxy)-1,2,3,3a-tetrahydro-4H-pyrrolo[2,3-b]quinolin-4-one (358 mg, 52%) as a yellow solid: ¹H NMR (300 MHz, DMSO-d₆) δ 7.84 (s, 1H), 7.80 (d, J=8.7 Hz, 1H), 7.15 (d, J=8.7 Hz, 1H), 3.70 (br s, 2H), 2.39-2.34 (m, 1H), 2.17-2.16 (m, 1H), 0.90-0.81 (m, 21H); NH proton not observed.

### Preparation of (S)-1-(6-Methylpyridin-3-yl)-6-(trifluoromethyl)-3a-((triisopropylsilyl)oxy)-1,2,3,3a-tetrahydro-4H-pyrrolo[2,3-b]quinolin-4-one

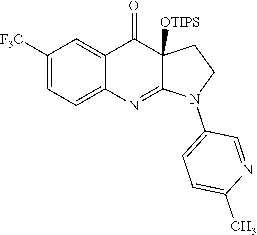

A solution of (S)-6-(trifluoromethyl)-3a-((triisopropylsilyl)oxy)-1,2,3,3a-tetrahydro-4H-pyrrolo[2,3-b]quinolin-4-one (125 mg, 0.293 mmol) in toluene (3 mL) was treated with 5-iodo-2-methylpyridine (96 mg, 0.44 mmol), copper iodide (6.0 mg, 0.029 mmol), cesium carbonate (286 mg, 0.879 mmol), and N,N′dimethyl-(1R,2R)-1,2-cyclohexanediamine (9 μL, 0.06 mmol) and heated at 105° C. in a sealed vial for 16 h. After this time, the reaction mixture was allowed to cool to ambient temperature. The solids were removed by filtration and washed with ethyl acetate. The filtrate was concentrated under reduced pressure. The crude residue was purified by column chromatography (silica gel, 12-100% ethyl acetate/heptane) to provide (S)-1-(6-methylpyridin-3-yl)-6-(trifluoromethyl)-3a-((triisopropylsilyl)oxy)-1,2,3,3a-tetrahydro-4H-pyrrolo[2,3-b]quinolin-4-one (143 mg, 94%) as a yellow gum: ESI MS m/z 518 [C₂₇H₃₄F₃N₃O₂Si+H]⁺.

### Preparation of provide (S)-3a-Hydroxy-1-(6-methylpyridin-3-yl)-6-(trifluoromethyl)-1,2,3,3a-tetrahydro-4H-pyrrolo[2,3-b]quinolin-4-one

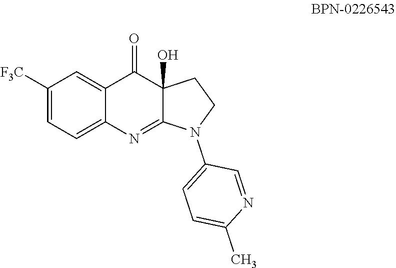 [SMILES: Cc1ccc(N2CC[C@@]3(O)C(=O)c4cc(C(F)(F)F)ccc4N=C23)cn1]

A solution of (S)-1-(6-methylpyridin-3-yl)-6-(trifluoromethyl)-3a-((triisopropylsilyl)oxy)-1,2,3,3a-tetrahydro-4H-pyrrolo[2,3-b]quinolin-4-one (143 mg, 0.276 mmol) in tetrahydrofuran (5 mL) under a nitrogen atmosphere was treated with a 1.0 M solution of tetrabutylammonium fluoride in tetrahydrofuran (0.829 mL, 0.829 mmol), and the mixture was stirred for 1 h. After this time, the mixture was concentrated under reduced pressure. The crude residue was purified by column chromatography (silica gel, 0-20% methanol/methylene chloride) to provide (S)-3a-hydroxy-1-(6-methylpyridin-3-yl)-6-(trifluoromethyl)-1,2,3,3a-tetrahydro-4H-pyrrolo[2,3-b]quinolin-4-one (84 mg, 84%) as a yellow solid: mp=237-240° C. decomposed; ¹H NMR (300 MHz, DMSO-d₆) δ 9.09 (d, J=2.4 Hz, 1H), 8.39 (dd, J=8.7, 2.7 Hz, 1H), 7.93 (s, 1H), 7.87 (d, J=8.4 Hz, 1H), 7.36 (dd, J=8.4, 4.8 Hz, 2H), 7.08 (s, 1H), 4.19-4.12 (m, 1H), 4.06-4.00 (m, 1H), 2.32-2.27 (m, 2H), CHs protons obscured by solvent; ESI MS m/z 362 [C₁₈H₁₄F₃N₃O₂+H]⁺; HPLC (Method C) >99% (AUC), t_{R}=12.49 min; Chiral HPLC (Chiralpak AD, Method A) 49.7% (AUC), t_{R}=16.24 min.

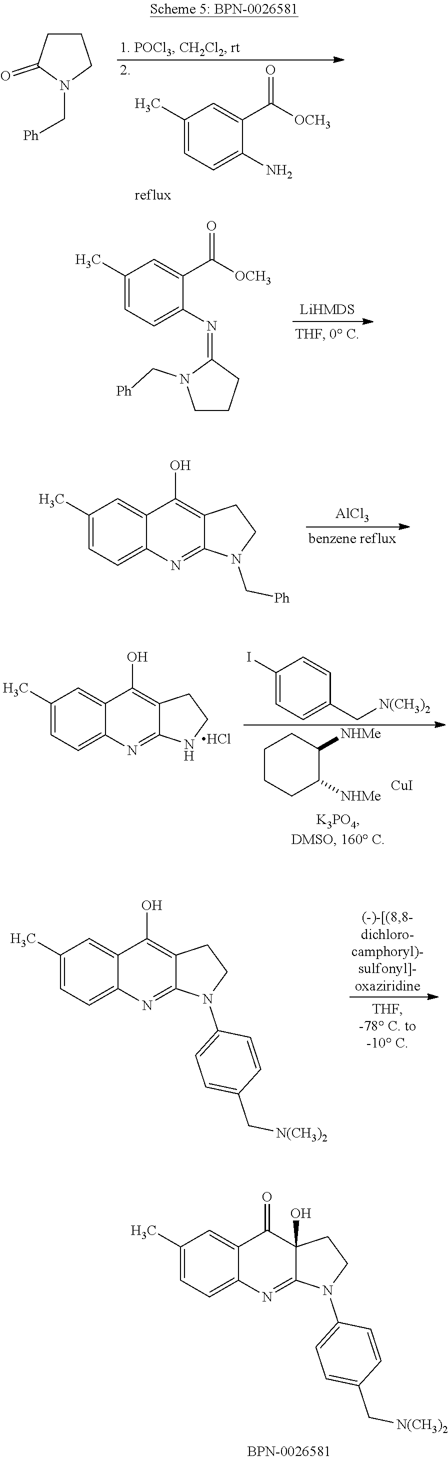

### Preparation of Methyl 2-((1-benzylpyrrolidin-3-ylidene)amino)-5-methylbenzoate

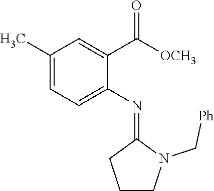 [SMILES: COC(=O)c1cc(C)ccc1/N=C1\CCCN1Cc1ccccc1]

A solution of 1-benzylpyrrolidin-2-one (6.30 g, 35.9 mmol) in methylene chloride (200 mL) was treated with phosphorous oxychloride (4.50 mL, 49.2 mmol) and stirred under a nitrogen atmosphere at ambient temperature for 4 h. The mixture was treated with a solution of methyl 2-amino-5-methylbenzoate (5.30 g, 32.1 mmol) in methylene chloride (20 mL) and heated at reflux for 48 h. After this time, the reaction mixture was allowed to cool to ambient temperature and concentrated under reduced pressure. The residue was diluted with ethyl acetate, washed with saturated aqueous sodium bicarbonate and then water. The organic layer was extracted with 0.4 M hydrochloric acid, and the aqueous extract was basified to pH ˜12 by adding sodium hydroxide. The mixture was extracted with ethyl acetate, dried over sodium sulfate, filtered, and concentrated under reduced pressure to provide methyl 2-((1-benzylpyrrolidin-3-ylidene)amino)-5-methylbenzoate (8.40 g, 81%) as a pale yellow oil: ESI MS m/z 323 [C₂₀H₂₂N₂O₂+H]⁺.

### Preparation of 1-Benzyl-6-methyl-2,3-dihydro-1H-pyrrolo[2,3-b]quinolin-4-ol

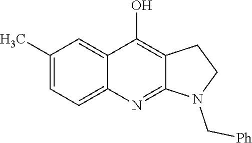 [SMILES: Cc1ccc2nc3c(c(O)c2c1)CCN3Cc1ccccc1]

A solution of methyl 2-((1-benzylpyrrolidin-3-ylidene)amino)-5-methylbenzoate (8.40 g, 26.1 mmol) in tetrahydrofuran (200 mL) under a nitrogen atmosphere at 0° C. was treated dropwise with a 1.0 M solution of lithium bis(trimethylsilyl)amide in tetrahydrofuran (55 mL, 55 mmol) and stirred for 3 h. After this time, a saturated solution of ammonium chloride (100 mL) was added, and the mixture was stirred for 30 min. The precipitate was collected by filtration, washed with water and diethyl ether and dried under high vacuum to provide 1-benzyl-6-methyl-2,3-dihydro-1H-pyrrolo[2,3-b]quinolin-4-ol (3.18 g, 42%) as an off-white solid: ESI MS m/z 291 [C₁₉H₁₈N₂O+H]⁺.

### Preparation of 6-Methyl-2,3-dihydro-1H-pyrrolo[2,3-b]quinolin-4-ol hydrochloride

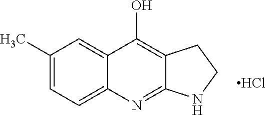

A mixture of 1-benzyl-6-methyl-2,3-dihydro-1H-pyrrolo[2,3-b]quinolin-4-ol (6.30 g, 21.7 mmol) and aluminum trichloride (12.8 g, 96.0 mmol) in benzene (140 mL) was stirred at reflux for 3 h. After this time, the mixture was cooled to room temperature and poured into stirred ice/water (250 mL). The mixture was stirred for 15 min and then the precipitate was collected by filtration, washed with diethyl ether and dried under high vacuum to provide 6-methyl-2,3-dihydro-1H-pyrrolo[2,3-b]quinolin-4-ol hydrochloride (5.1 g, quantitative): ESI MS m/z 201 [C₁₂H₁₂N₂O+H]⁺.

### Preparation of 1-(4-((Dimethylamino)methyl)phenyl)-6-methyl-2,3-dihydro-1H-pyrrolo[2,3-b]quinolin-4-ol

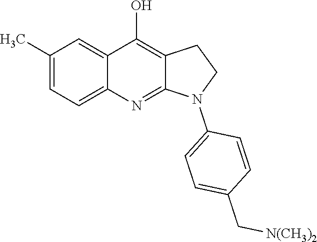 [SMILES: Cc1ccc2nc3c(c(O)c2c1)CCN3c1ccc(CN(C)C)cc1]

A solution of 6-methyl-2,3-dihydro-1H-pyrrolo[2,3-b]quinolin-4-ol hydrochloride (260 mg, 1.10 mmol) in dimethyl sulfoxide (4 mL) was treated with 1-(4-iodophenyl)-N,N-dimethylmethanamine (460 mg, 1.77 mmol), copper iodide (40 mg, 0.21 mmol), tripotassium phosphate (650 mg, 3.06 mmol), and trans-N,N′-dimethyl-1,2-cyclohexanediamine (58 mg, 0.41 mmol). The resulting mixture was heated in a microwave at 160° C. for 4 h. After this time, the reaction mixture was filtered through diatomaceous earth using 3/1 methylene chloride/methanol as eluent. The filtrate was concentrated under reduced pressure, and the residue was purified by column chromatography (silica gel, 0-80% (methylene chloride/methanol/ammonium hydroxide 80/18/2), methylene chloride) to provide 1-(4-((dimethylamino)methyl)phenyl)-6-methyl-2,3-dihydro-1H-pyrrolo[2,3-b]quinolin-4-ol (200 mg, 54%): ESI MS m/z 334 [C₂₁H₂₃N₃O+H]⁺.

### Preparation of (S)-1-(4-((Dimethylamino)methyl)phenyl)-3a-hydroxy-6-methyl-3,3a-dihydro-1H-pyrrolo[2,3-b]quinolin-4(2H)-one

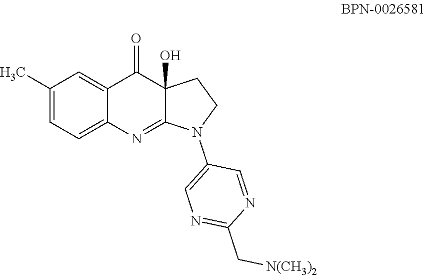 [SMILES: Cc1ccc2c(c1)C(=O)[C@]1(O)CCN(c3cnc(CN(C)C)nc3)C1=N2]

A solution of 1-(4-((dimethylamino)methyl)phenyl)-6-methyl-2,3-dihydro-1H-pyrrolo[2,3-b]quinolin-4-ol (200 mg, 0.60 mmol) in tetrahydrofuran (8 mL) at −78° C. was treated dropwise with a 1.0 M solution of lithium bis(trimethylsilyl)amide in tetrahydrofuran (1.0 mL, 1.0 mmol), followed by (−)-(8,8-dichlorocamphorylsulfonyl)oxaziridine (600 mg, 2.01 mmol) in one portion. The mixture was stirred while allowing the temperature to raise to −10° C. over 2 h. After this time, the mixture was treated with saturated aqueous ammonium iodide (4 mL) and stirred for 20 min. Saturated aqueous sodium thiosulfate (10 ml) was added, and the mixture was stirred for 20 min and then extracted with ethyl acetate. The organics were extracted with 0.8 M hydrochloric acid, and the aqueous extract was basified to pH ˜12 by adding sodium hydroxide. The mixture was extracted with ethyl acetate, dried over sodium sulfate, filtered, and concentrated under reduced pressure. The residue was crystallized from hot acetonitrile to provide (S)-1-(4-((dimethylamino)methyl)phenyl)-3a-hydroxy-6-methyl-3,3a-dihydro-1H-pyrrolo[2,3-b]quinolin-4(2H)-one (26 mg, 12%) as a yellow solid: mp=194-195° C.; ¹H NMR (500 MHz, DMSO-d₆) δ 8.01 (d, J=8.7 Hz, 1H), 7.52 (d, J=1.5 Hz, 1H), 7.37 (d, J=8.2, 1.7 Hz, 1H), 7.32 (d, J=8.6 Hz, 1H), 7.10 (d, J=8.1 Hz, 1H), 6.80 (s, 1H), 4.08-4.02 (m, 1H), 3.97-3.93 (m, 1H), 3.37 (s, 2H), 2.30 (s, 3H), 2.27-2.23 (m, 2H), 2.14 (s, 6H); ESI MS m/z 350 [C₂₁H₂₃N₃O₂+H]⁺; HPLC (Method C) 96.4% (AUC), t_{R}=9.73 min; Chiral HPLC (Chiralpak AD, Method A) 94.5% (AUC), t_{R}=15.86 min.

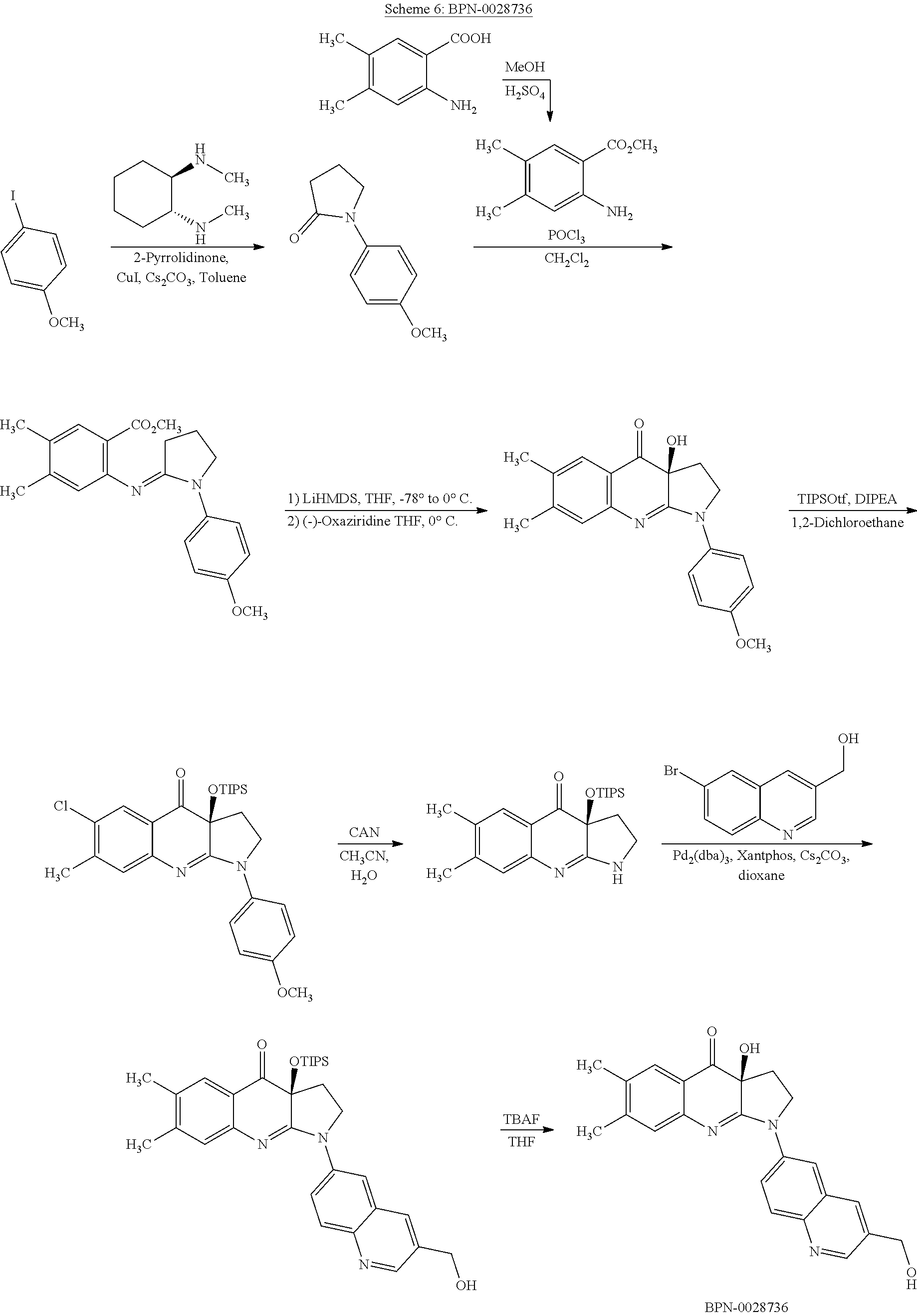

### Preparation of 1-(4-Methoxyphenyl)pyrrolidin-2-one

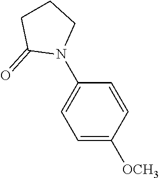 [SMILES: COc1ccc(N2CCCC2=O)cc1]

A solution of 1-iodo-4-methoxybenzene (8.00 g, 33.3 mmol) in toluene (50 mL) was treated with 2-pyrrolidinone (3.8 mL, 4.3 g, 50 mmol), copper iodide (638 mg, 3.35 mmol), cesium carbonate (26.1 g, 80.0 mmol) and (1R,2R)-N¹,N²-dimethylcyclohexane-1,2-diamine (1.1 mL, 0.99 g, 7.0 mmol) and heated at 110° C. under a nitrogen atmosphere for 22 h. After this time, the reaction mixture was allowed to cool to ambient temperature, filtered through celite, and the filtrate was concentrated under reduced pressure. The crude residue was purified by column chromatography (silica gel, 12-100% ethyl acetate/heptane) to provide 1-(4-methoxyphenyl)pyrrolidin-2-one (6.07 g, 95%) as a white solid: ¹H NMR (300 MHz, DMSO-d₆) δ 7.54 (d, J=9.3 Hz, 2H), 6.93 (d, J=9.0 Hz, 2H), 3.78 (t, J=7.2 Hz, 2H), 3.74 (s, 3H), 2.45 (t, J=8.1 Hz, 2H), 2.09-1.99 (m, 2H); ESI MS m/z 192 [C₁₁H₁₃NO₂+H]⁺.

### Preparation of Methyl 2-Amino-4,5-dimethylbenzoate

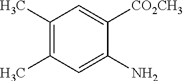

A solution of 2-amino-4,5-dimethylbenzoic acid (10.00 g, 60.54 mmol) in methanol (180 mL) was treated with concentrated sulfuric acid (18 mL) and heated at 60° C. under a nitrogen atmosphere for 64.5 h. After this time, the reaction mixture was concentrated under reduced pressure to remove the volatiles. The resulting residue was carefully treated with saturated aqueous sodium bicarbonate (800 mL) and extracted with ethyl acetate (2×300 mL). The combined organics were dried over sodium sulfate, filtered, and concentrated under reduced pressure to provide methyl 2-amino-4,5-dimethylbenzoate (10.14 g, 94%) as a light brown solid: ¹H NMR (300 MHz, DMSO-d₆) δ 7.44 (s, 1H), 6.56 (s, 1H), 6.38 (s, 2H), 3.75 (s, 3H), 2.12 (s, 3H), 2.07 (s, 3H); ESI MS m/z 180 [C₁₀H₁₃NO₂+H]⁺.

### Preparation of Methyl 2-((1-(4-Methoxyphenyl)pyrrolidin-2-ylidene)amino)-4,5-dimethylbenzoate

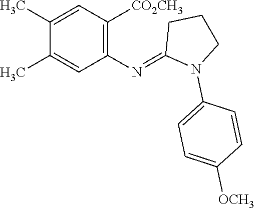 [SMILES: COC(=O)c1cc(C)c(C)cc1/N=C1\CCCN1c1ccc(OC)cc1]

A solution of 1-(4-methoxyphenyl)pyrrolidin-2-one (16.34 g, 85.45 mmol) in 1,2-dichloroethane (100 mL) was treated with phosphorous oxychloride (12 mL, 20 g, 130 mmol) and stirred under a nitrogen atmosphere at ambient temperature for 5.25 h. The mixture was treated with a solution of methyl 2-amino-4,5-dimethylbenzoate (15.36 g, 85.70 mmol) in 1,2-dichloroethane (100 mL) and refluxed at 80° C. for 67 h. After this time, the reaction mixture was allowed to cool to ambient temperature and was treated with sodium bicarbonate (200 mL). The organic and aqueous layers were separated, and the aqueous layer was washed with ethyl acetate. The combined organics were dried over sodium sulfate, filtered, and concentrated under reduced pressure. The residue was dissolved in ethyl acetate (200 mL) and extracted with 0.3 M hydrochloric acid. The combined acid layers were adjusted to pH ˜11 with 2.0 M aqueous sodium hydroxide and extracted with and ethyl acetate. The combined organics were dried over sodium sulfate, filtered, and concentrated under reduced pressure to provide methyl 2-((1-(4-methoxyphenyl)pyrrolidin-2-ylidene)amino)-4,5-dimethylbenzoate (25.93 g, 86%) as a brown oil: ¹H NMR (300 MHz, DMSO-d₆) δ 7.75-7.70 (m, 2H), 7.51 (s, 1H), 6.94-6.88 (m, 2H), 6.58 (s, 1H), 3.79 (apparent t, J=6.9 Hz, 2H), 3.73-3.70 (m, 6H), 2.34 (apparent t, J=7.8 Hz, 2H), 2.19-2.18 (m, 6H), 1.97-1.90 (m, 2H); ESI MS m/z 353 [C₂₁H₂₄N₂O₃+H]⁺.

### Preparation of (S)-3a-Hydroxy-1-(4-methoxyphenyl)-6,7-dimethyl-1,2,3,3a-tetrahydro-4H-pyrrolo[2,3-b]quinolin-4-one

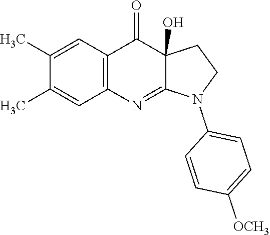 [SMILES: COc1ccc(N2CC[C@@]3(O)C(=O)c4cc(C)c(C)cc4N=C23)cc1]

A solution of methyl 2-((1-(4-methoxyphenyl)pyrrolidin-2-ylidene)amino)-4,5-dimethylbenzoate (1.87 g, 5.31 mmol) in tetrahydrofuran (10 mL) was cooled in a dry ice/acetone bath under a nitrogen atmosphere and treated dropwise with a 1.0 M solution of lithium bis(trimethylsilyl)amide in tetrahydrofuran (16 mL, 16 mmol). The mixture was stirred for 1 h, during which time the bath temperature increased to ˜0° C. After 1 h, the mixture was treated with a solution of (−)-(8,8-dichlorocamphorylsulfonyl)oxaziridine (3.17 mg, 10.6 mmol) in tetrahydrofuran (10 mL) and stirred for 1 h. After this time, the mixture was treated with saturated aqueous ammonium iodide (6 mL) followed by saturated aqueous sodium thiosulfate (12 ml) and brine (40 mL). The organic and aqueous layers were separated, and the aqueous layer was washed with ethyl acetate. The combined organics were dried over sodium sulfate, filtered, and concentrated under reduced pressure. The residue was dissolved in ethyl acetate (25 mL) and extracted with 0.3 M hydrochloric acid (4×40 mL). The combined acid layers were adjusted to pH ˜8 with 2.0 M aqueous sodium hydroxide and extracted with ethyl acetate (3×100 mL). The organics were dried over sodium sulfate, filtered, and concentrated under reduced pressure. The crude product was purified by recrystallization from hot ethanol to provide (S)-3a-hydroxy-1-(4-methoxyphenyl)-6,7-dimethyl-1,2,3,3a-tetrahydro-4H-pyrrolo[2,3-b]quinolin-4-one (422 mg, 24%) as a yellow solid: mp=199-200° C.; ¹H NMR (500 MHz, DMSO-d₆) δ 7.98-7.95 (m, 2H), 7.46 (s, 1H), 7.00-6.98 (m, 3H), 6.72 (s, 1H), 4.06-4.01 (m, 1H), 3.91-3.88 (m, 1H), 3.77 (s, 3H), 2.25 (s, 3H), 2.23-2.21 (m, 5H); ESI MS m/z 337 [C₂₀H₂₀N₂O₃+H]⁺; HPLC (Method B) >99% (AUC), t_{R}=8.71 min; Chiral HPLC (Chiralpak AD, Method A) >99% (AUC), t_{R}=18.75 min.

### Preparation of (S)-1-(4-Methoxyphenyl)-6,7-dimethyl-3a-((triisopropylsilyl)oxy)-1,2,3,3a-tetrahydro-4H-pyrrolo[2,3-b]quinolin-4-one

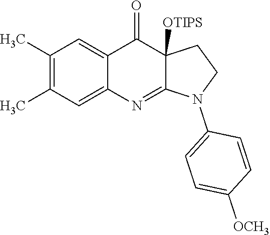

A solution of (S)-3a-hydroxy-1-(4-methoxyphenyl)-6,7-dimethyl-1,2,3,3a-tetrahydro-4H-pyrrolo[2,3-b]quinolin-4-one (725 mg, 2.16 mmol) in 1,2-dichloroethane (25 mL) was treated with N,N-diisopropylethylamine (1.5 mL, 1.1 g, 8.6 mmol) and triisopropylsilyl trifluoromethanesulfonate (1.8 mL, 2.0 g, 6.7 mmol) and stirred under a nitrogen atmosphere at 80° C. for 21 h. After this time, the reaction mixture was allowed to cool to ambient temperature. The mixture was treated with cold deionized water (25 mL), followed by saturated aqueous ammonium chloride (25 mL) and the organic and aqueous layers were separated. The aqueous layer was washed with ethyl acetate (2×25 mL). The organics were dried over sodium sulfate, filtered, and concentrated under reduced pressure. The crude residue was purified by column chromatography (silica gel, 5-40% ethyl acetate/heptane) to provide (S)-1-(4-methoxyphenyl)-6,7-dimethyl-3a-((triisopropylsilyl)oxy)-1,2,3,3a-tetrahydro-4H-pyrrolo[2,3-b]quinolin-4-one (690 mg, 65%) as an yellow oil: ¹H NMR (300 MHz, DMSO-d₆) δ 7.96 (d, J=9.0 Hz, 2H), 7.46 (s, 1H), 7.02-6.99 (m, 3H), 4.02-3.96 (m, 2H), 3.77 (s, 3H), 2.33-2.26 (m, 5H), 2.20 (s, 3H), 0.83-0.81 (m, 21H); ESI MS m/z 493 [C₂₉H₄₀N₂O₃Si+H]⁺.

### Preparation of (S)-6,7-Dimethyl-3a-((triisopropylsilyl)oxy)-1,2,3,3a-tetrahydro-4H-pyrrolo[2,3-b]quinolin-4-one

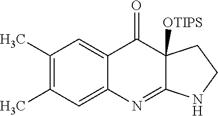

A solution of (S)-1-(4-methoxyphenyl)-6,7-dimethyl-3a-((triisopropylsilyl)oxy)-1,2,3,3a-tetrahydro-4H-pyrrolo[2,3-b]quinolin-4-one (1.24 g, 2.52 mmol) in acetonitrile (20 mL) was cooled in a wet ice/water bath and treated dropwise with a solution of ammonium cerium(IV) nitrate (5.52 g, 10.1 mmol) in deionized water (10 mL) and stirred under a nitrogen atmosphere at ˜0° C. for 30 min. After this time, the mixture was treated with aqueous sodium thiosulfate (15 mL) and saturated aqueous sodium bicarbonate (15 mL). The mixture was filtered through celite and the solids were washed with ethyl acetate. The filtrate was extracted with ethyl acetate (2×100 mL). The combined organics were washed with saturated sodium bicarbonate, water, and brine. The organics were dried over sodium sulfate, filtered, and concentrated under reduced pressure. The crude residue was purified by column chromatography (silica gel, 5-100% ethyl acetate/heptane, 0-20% methanol/ethyl acetate) to provide (S)-6,7-dimethyl-3a-((triisopropylsilyl)oxy)-1,2,3,3a-tetrahydro-4H-pyrrolo[2,3-b]quinolin-4-one (589 mg, 61%) as a yellow-orange solid: ¹H NMR (300 MHz, DMSO-d₆) δ 7.41 (s, 1H), 6.77 (s, 1H), 3.70-3.61 (m, 2H), 2.21-2.16 (m, 8H), 0.86-0.84 (m, 21H), NH proton not observed; ESI MS m/z 387 [C₂₂H₃₄N₂O₂Si+H]⁺.

### Preparation of (S)-1-(3-(Hydroxymethyl)quinolin-6-yl)-6,7-dimethyl-3a-((triisopropylsilyl)oxy)-1,2,3,3a-tetrahydro-4H-pyrrolo[2,3-b]quinolin-4-one

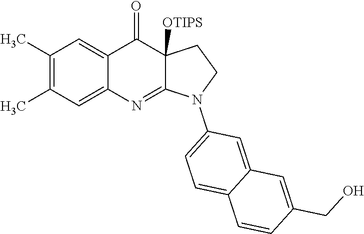

A solution of (S)-6,7-dimethyl-3a-((triisopropylsilyl)oxy)-1,2,3,3a-tetrahydro-4H-pyrrolo[2,3-b]quinolin-4-one (100 mg, 0.260 mmol), (6-bromoquinolin-3-yl)methanol (105 mg, 0.440 mmol), 4,5-bis(diphenylphosphino)-9,9-dimethylxanthene (xantphos) (90 mg, 0.16 mmol), and cesium carbonate (186 mg, 0.570 mmol) in 1,4-dioxane (6 mL) was degassed with argon for 15 min. Tris(dibenzylideneacetone)dipalladium(0) (25 mg, 0.026 mmol) was added, and the resulting mixture was heated at 105° C. for 3 h. After this time, the reaction mixture was filtered through diatomaceous earth using methylene chloride as an eluent. The filtrate was concentrated under reduced pressure, and the residue was purified by column chromatography (silica gel, 0-100% ethyl acetate/hexanes) to provide (S)-1-(3-(hydroxymethyl)quinolin-6-yl)-6,7-dimethyl-3a-((triisopropylsilyl)oxy)-1,2,3,3a-tetrahydro-4H-pyrrolo[2,3-b]quinolin-4-one (43 mg, 30%): ESI MS m/z 544 [C₃₂H₄₁N₃O₃Si+H]⁺.

### Preparation of (S)-3a-Hydroxy-1-(3-(hydroxymethyl)quinolin-6-yl)-6,7-dimethyl-1,2,3,3a-tetrahydro-4H-pyrrolo[2,3-b]quinolin-4-one

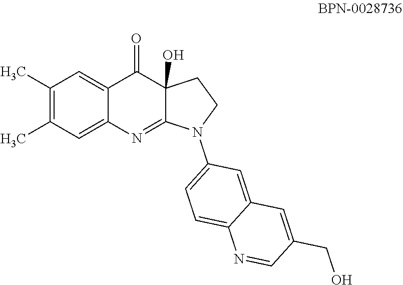 [SMILES: Cc1cc2c(cc1C)C(=O)[C@]1(O)CCN(c3ccc4ncc(CO)cc4c3)C1=N2]

A solution of (S)-1-(3-(hydroxymethyl)quinolin-6-yl)-6,7-dimethyl-3a-((triisopropylsilyl)oxy)-1,2,3,3a-tetrahydro-4H-pyrrolo[2,3-b]quinolin-4-one (43 mg, 0.08 mmol) in tetrahydrofuran (3 mL) under a nitrogen atmosphere was treated with a 1.0 M solution of tetrabutylammonium fluoride in tetrahydrofuran (0.10 mL, 0.10 mmol), and the mixture was stirred for 4 h. After this time, the mixture was concentrated under reduced pressure. The crude residue was purified by column chromatography (silica gel, 0-20% methanol/methylene chloride) to provide (S)-3a-hydroxy-1-(3-(hydroxymethyl)quinolin-6-yl)-6,7-dimethyl-1,2,3,3a-tetrahydro-4H-pyrrolo[2,3-b]quinolin-4-one as a yellow solid: ¹H NMR (300 MHz, DMSO-d₆) δ 8.78 (s, 1H), 8.70 (d, J=9.6 Hz, 1H), 8.49 (s, 1H), 8.23 (s, 1H), 8.05 (d, J=9.0 Hz, 1H), 7.52 (s, 1H), 7.15 (s, 1H), 6.87 (s, 1H), 5.47 (m, 1H), 4.73 (d, J=4.2 Hz, 2H) 4.19-4.13 (m, 2H), 2.31-2.24 (m, 8H); ESI MS m/z 388 [C₂₃H₂₁N₃O₃+H]⁺; UPLC (Method A) 98.3% (AUC), t_{R}=10.24 min; Chiral HPLC (Chiralpak AD, Method A) 91.1% (AUC), t_{R}=19.45 min.

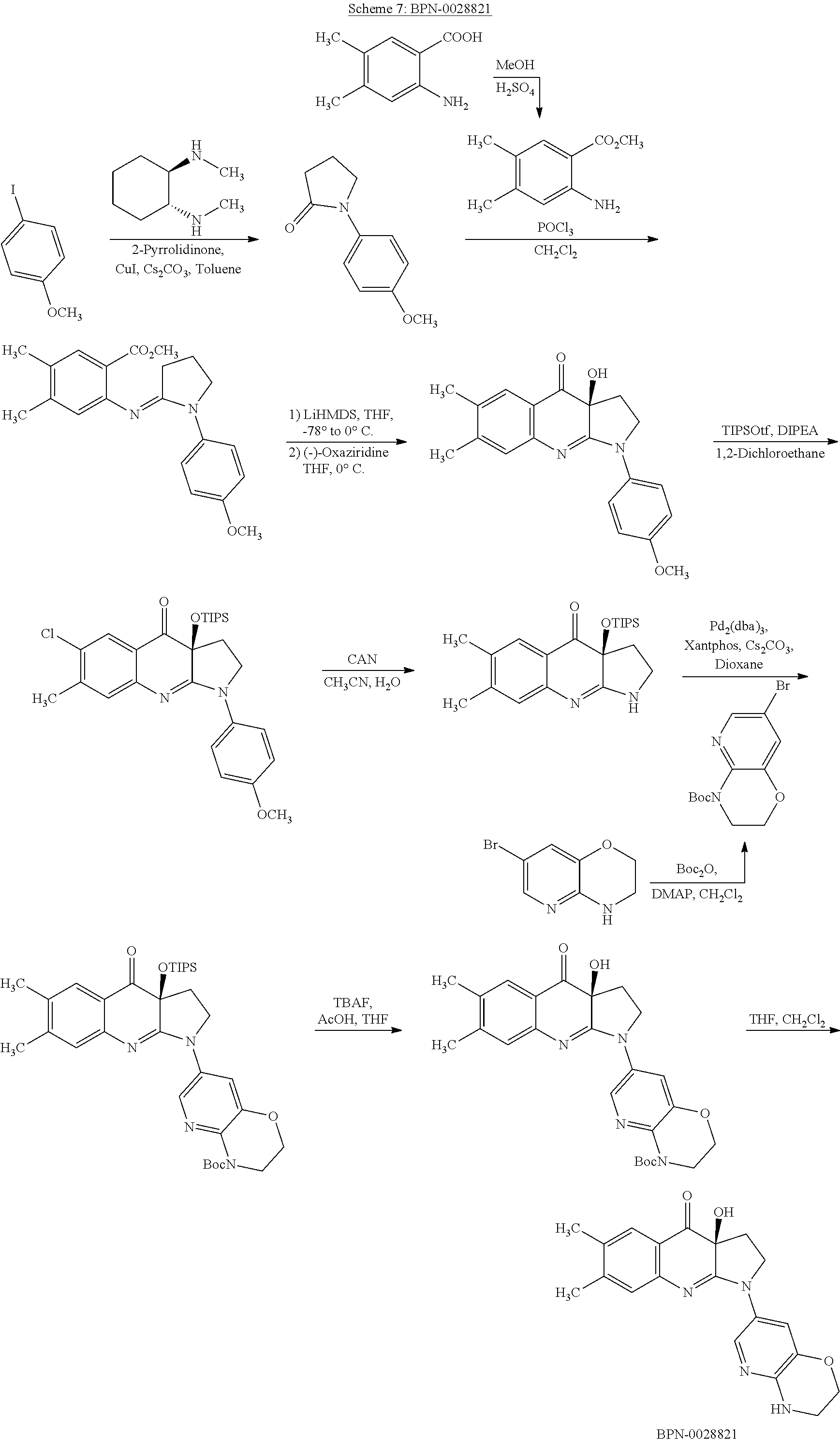

### Preparation of 1-(4-Methoxyphenyl)pyrrolidin-2-one

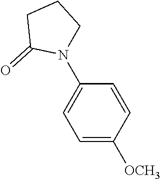 [SMILES: COc1ccc(N2CCCC2=O)cc1]

A solution of 1-iodo-4-methoxybenzene (8.00 g, 33.3 mmol) in toluene (50 mL) was treated with 2-pyrrolidinone (3.8 mL, 4.3 g, 50 mmol), copper iodide (638 mg, 3.35 mmol), cesium carbonate (26.1 g, 80.0 mmol) and (1R,2R)-N¹,N²-dimethylcyclohexane-1,2-diamine (1.1 mL, 0.99 g, 7.0 mmol) and heated at 110° C. under a nitrogen atmosphere for 22 h. After this time, the reaction mixture was allowed to cool to ambient temperature, filtered through celite, and the filtrate was concentrated under reduced pressure. The crude residue was purified by column chromatography (silica gel, 12-100% ethyl acetate/heptane) to provide 1-(4-methoxyphenyl)pyrrolidin-2-one (6.07 g, 95%) as a white solid: ¹H NMR (300 MHz, DMSO-d₆) δ 7.54 (d, J=9.3 Hz, 2H), 6.93 (d, J=9.0 Hz, 2H), 3.78 (t, J=7.2 Hz, 2H), 3.74 (s, 3H), 2.45 (t, J=8.1 Hz, 2H), 2.09-1.99 (m, 2H); ESI MS m/z 192 [C₁₁H₁₃NO₂+H]⁺.

### Preparation of Methyl 2-Amino-4,5-dimethylbenzoate

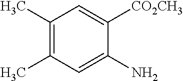

A solution of 2-amino-4,5-dimethylbenzoic acid (10.00 g, 60.54 mmol) in methanol (180 mL) was treated with concentrated sulfuric acid (18 mL) and heated at 60° C. under a nitrogen atmosphere for 64.5 h. After this time, the reaction mixture was concentrated under reduced pressure to remove the volatiles. The resulting residue was carefully treated with saturated aqueous sodium bicarbonate (800 mL) and extracted with ethyl acetate (2×300 mL). The combined organics were dried over sodium sulfate, filtered, and concentrated under reduced pressure to provide methyl 2-amino-4,5-dimethylbenzoate (10.14 g, 94%) as a light brown solid: ¹H NMR (300 MHz, DMSO-d₆) δ 7.44 (s, 1H), 6.56 (s, 1H), 6.38 (s, 2H), 3.75 (s, 3H), 2.12 (s, 3H), 2.07 (s, 3H); ESI MS m/z 180 [C₁₀H₁₃NO₂+H]⁺.

### Preparation of Methyl 2-((1-(4-Methoxyphenyl)pyrrolidin-2-ylidene)amino)-4,5-dimethylbenzoate

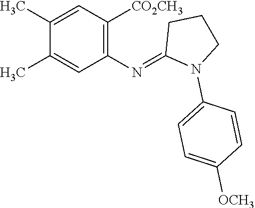 [SMILES: COC(=O)c1cc(C)c(C)cc1/N=C1\CCCN1c1ccc(OC)cc1]

A solution of 1-(4-methoxyphenyl)pyrrolidin-2-one (16.34 g, 85.45 mmol) in 1,2-dichloroethane (100 mL) was treated with phosphorous oxychloride (12. mL, 20. g, 130 mmol) and stirred under a nitrogen atmosphere at ambient temperature for 5.25 h. The mixture was treated with a solution of methyl 2-amino-4,5-dimethylbenzoate (15.36 g, 85.70 mmol) in 1,2-dichloroethane (100 mL) and refluxed at 80° C. for 67 h. After this time, the reaction mixture was allowed to cool to ambient temperature and was treated with saturated aqueous sodium bicarbonate (200 mL). The organic and aqueous layers were separated, and the aqueous layer was washed with ethyl acetate. The combined organics were dried over sodium sulfate, filtered, and concentrated under reduced pressure. The residue was dissolved in ethyl acetate (200 mL) and extracted with 0.3 M hydrochloric acid. The combined acid layers were adjusted to pH ˜11 with 2.0 M aqueous sodium hydroxide and extracted with ethyl acetate. The combined organics were dried over sodium sulfate, filtered, and concentrated under reduced pressure to provide methyl 2-((1-(4-methoxyphenyl)pyrrolidin-2-ylidene)amino)-4,5-dimethylbenzoate (25.93 g, 86%) as a brown oil: ¹H NMR (300 MHz, DMSO-d₆) δ 7.75-7.70 (m, 2H), 7.51 (s, 1H), 6.94-6.88 (m, 2H), 6.58 (s, 1H), 3.79 (apparent t, J=6.9 Hz, 2H), 3.73-3.70 (m, 6H), 2.34 (apparent t, J=7.8 Hz, 2H), 2.19-2.18 (m, 6H), 1.97-1.90 (m, 2H); ESI MS m/z 353 [C₂₁H₂₄N₂O₃+H]⁺.

### Preparation of (S)-3a-Hydroxy-1-(4-methoxyphenyl)-6,7-dimethyl-1,2,3,3a-tetrahydro-4H-pyrrolo[2,3-b]quinolin-4-one

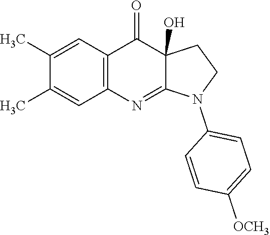 [SMILES: COc1ccc(N2CC[C@@]3(O)C(=O)c4cc(C)c(C)cc4N=C23)cc1]

A solution of methyl 2-((1-(4-methoxyphenyl)pyrrolidin-2-ylidene)amino)-4,5-dimethylbenzoate (1.87 g, 5.31 mmol) in tetrahydrofuran (10 mL) was cooled in a dry ice/acetone bath under a nitrogen atmosphere and treated dropwise with a 1.0 M solution of lithium bis(trimethylsilyl)amide in tetrahydrofuran (16 mL, 16 mmol). The mixture was stirred for 1 h, during which time the bath temperature increased to ˜0° C. After 1 h, the mixture was treated with a solution of (−)-(8,8-dichlorocamphorylsulfonyl)oxaziridine (3.17 mg, 10.6 mmol) in tetrahydrofuran (10 mL) and stirred for 1 h. After this time, the mixture was treated with saturated aqueous ammonium iodide (6 mL) followed by saturated aqueous sodium thiosulfate (12 ml) and brine (40 mL). The organic and aqueous layers were separated, and the aqueous layer was washed with ethyl acetate. The combined organics were dried over sodium sulfate, filtered, and concentrated under reduced pressure. The residue was dissolved in ethyl acetate (25 mL) and extracted with 0.3 M hydrochloric acid (4×40 mL). The combined acid layers were adjusted to pH ˜8 with 2.0 M aqueous sodium hydroxide and extracted with ethyl acetate (3×100 mL). The organics were dried over sodium sulfate, filtered, and concentrated under reduced pressure. The crude product was purified by recrystallization from hot ethanol to provide (S)-3a-hydroxy-1-(4-methoxyphenyl)-6,7-dimethyl-1,2,3,3a-tetrahydro-4H-pyrrolo[2,3-b]quinolin-4-one (422 mg, 24%) as a yellow solid: mp=199-200° C.; ¹H NMR (500 MHz, DMSO-d₆) δ 7.98-7.95 (m, 2H), 7.46 (s, 1H), 7.00-6.98 (m, 3H), 6.72 (s, 1H), 4.06-4.01 (m, 1H), 3.91-3.88 (m, 1H), 3.77 (s, 3H), 2.25 (s, 3H), 2.23-2.21 (m, 5H); ESI MS m/z 337 [C₂₀H₂₀N₂O₃+H]⁺; HPLC (Method B) >99% (AUC), t_{R}=8.71 min; Chiral HPLC (Chiralpak AD, Method A) >99% (AUC), t_{R}=18.75 min.

### Preparation of (S)-1-(4-Methoxyphenyl)-6,7-dimethyl-3a-((triisopropylsilyl)oxy)-1,2,3,3a-tetrahydro-4H-pyrrolo[2,3-b]quinolin-4-one

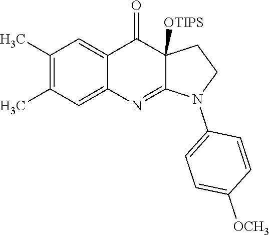

A solution of (S)-3a-hydroxy-1-(4-methoxyphenyl)-6,7-dimethyl-1,2,3,3a-tetrahydro-4H-pyrrolo[2,3-b]quinolin-4-one (725 mg, 2.16 mmol) in 1,2-dichloroethane (25 mL) was treated with N,N-diisopropylethylamine (1.5 mL, 1.1 g, 8.6 mmol) and triisopropylsilyl trifluoromethanesulfonate (1.8 mL, 2.0 g, 6.7 mmol) and stirred under a nitrogen atmosphere at 80° C. for 21 h. After this time, the reaction mixture was allowed to cool to ambient temperature. The mixture was treated with cold deionized water (25 mL), followed by saturated aqueous ammonium chloride (25 mL), and the organic and aqueous layers were separated. The aqueous layer was washed with ethyl acetate (2×25 mL). The organics were dried over sodium sulfate, filtered, and concentrated under reduced pressure. The crude residue was purified by column chromatography (silica gel, 5-40% ethyl acetate/heptane) to provide (S)-1-(4-methoxyphenyl)-6,7-dimethyl-3a-((triisopropylsilyl)oxy)-1,2,3,3a-tetrahydro-4H-pyrrolo[2,3-b]quinolin-4-one (690 mg, 65%) as an yellow oil: ¹H NMR (300 MHz, DMSO-d₆) δ 7.96 (d, J=9.0 Hz, 2H), 7.46 (s, 1H), 7.02-6.99 (m, 3H), 4.02-3.96 (m, 2H), 3.77 (s, 3H), 2.33-2.26 (m, 5H), 2.20 (s, 3H), 0.83-0.81 (m, 21H); ESI MS m/z 493 [C₂₉H₄₀N₂O₃Si+H]⁺.

### Preparation of (S)-6,7-Dimethyl-3a-((triisopropylsilyl)oxy)-1,2,3,3a-tetrahydro-4H-pyrrolo[2,3-b]quinolin-4-one

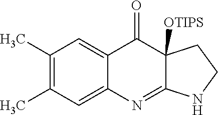

A solution of (S)-1-(4-methoxyphenyl)-6,7-dimethyl-3a-((triisopropylsilyl)oxy)-1,2,3,3a-tetrahydro-4H-pyrrolo[2,3-b]quinolin-4-one (1.24 g, 2.52 mmol) in acetonitrile (20 mL) was cooled in a wet ice/water bath and treated dropwise with a solution of ammonium cerium(IV) nitrate (5.52 g, 10.1 mmol) in deionized water (10 mL) and stirred under a nitrogen atmosphere at ˜0° C. for 30 min. After this time, the mixture was treated with aqueous sodium thiosulfate (15 mL) and saturated aqueous sodium bicarbonate (15 mL). The mixture was filtered through celite, and the solids were washed with ethyl acetate. The filtrate was extracted with ethyl acetate (2×100 mL). The combined organics were washed with saturated sodium bicarbonate, water, and brine. The organics were dried over sodium sulfate, filtered, and concentrated under reduced pressure. The crude residue was purified by column chromatography (silica gel, 5-100% ethyl acetate/heptane, 0-20% methanol/ethyl acetate) to provide (S)-6,7-dimethyl-3a-((triisopropylsilyl)oxy)-1,2,3,3a-tetrahydro-4H-pyrrolo[2,3-b]quinolin-4-one (589 mg, 61%) as a yellow-orange solid: ¹H NMR (300 MHz, DMSO-d₆) δ 7.41 (s, 1H), 6.77 (s, 1H), 3.70-3.61 (m, 2H), 2.21-2.16 (m, 8H), 0.86-0.84 (m, 21H), NH proton not observed; ESI MS m/z 387 [C₂₂H₃₄N₂O₂Si+H]⁺.

### Preparation of tert-Butyl 7-Bromo-2,3-dihydro-4H-pyrido[3,2-b][1,4]oxazine-4-carboxylate

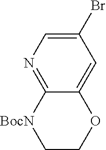 [SMILES: CC(C)(C)OC(=O)N1CCOc2cc(Br)cnc21]

A solution of 7-bromo-3,4-dihydro-2H-pyrido[3,2-b][1,4]oxazine (305 mg, 1.42 mmol) and 4-dimethylaminopyridine (19 mg, 0.16 mmol) in dichloromethane (20 mL) was cooled in a wet ice/water bath and treated dropwise with a solution of 2M di-tert-butyl dicarbonate in dichloromethane (0.78 mL, 1.6 mmol). After 10 min, the reaction was warmed to room temperature and stirred for 24 h. After this time, water (20 mL) was added, and the organic and aqueous layers were separated. The organics were washed with brine, dried over sodium sulfate, filtered, and concentrated under reduced pressure to provide tert-butyl 7-bromo-2,3-dihydro-4H-pyrido[3,2-b][1,4]oxazine-4-carboxylate (353 mg, 79%) as a pink solid: ¹H NMR (500 MHz, DMSO-d₆) δ 8.03 (d, J=2.2 Hz, 1H), 7.57 (d, J=2.2 Hz, 1H), 4.26-4.24 (m, 2H), 3.84-3.82 (m, 2H), 1.46 (s, 9H).

### Preparation of tert-Butyl (S)-7-(6,7-Dimethyl-4-oxo-3a-((triisopropylsilyl)oxy)-2,3,3a,4-tetrahydro-1H-pyrrolo[2,3-b]quinolin-1-yl)-2,3-dihydro-4H-pyrido[3,2-b][1,4]oxazine-4-carboxylate

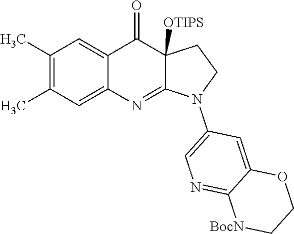

A solution of (S)-6,7-dimethyl-3a-((triisopropylsilyl)oxy)-1,2,3,3a-tetrahydro-4H-pyrrolo[2,3-b]quinolin-4-one (202 mg, 0.522 mmol), tert-butyl 7-bromo-2,3-dihydro-4H-pyrido[3,2-b][1,4]oxazine-4-carboxylate (250. mg, 0.793 mmol), and cesium carbonate (172 mg, 0.578 mmol) in 1,4-dioxane (10 mL) was degassed with argon for 10 min. Tris(dibenzylideneacetone)dipalladium(0) (61 mg, 0.067 mmol) and 4,5-bis(diphenylphosphino)-9,9-dimethylxanthene (xantphos) (188 mg, 0.325 mmol) were added and the resulting mixture was heated at 90° C. for 23.5 h in a sealed vial. After this time, the reaction mixture was filtered through diatomaceous earth using ethyl acetate as an eluent. The filtrate was concentrated under reduced pressure, and the residue was purified by column chromatography (silica gel, 12-100% ethyl acetate/hexanes) to provide tert-butyl (S)-7-(6,7-dimethyl-4-oxo-3a-((triisopropylsilyl)oxy)-2,3,3a,4-tetrahydro-1H-pyrrolo[2,3-b]quinolin-1-yl)-2,3-dihydro-4H-pyrido[3,2-b][1,4]oxazine-4-carboxylate (133 mg, 41%) as an orange solid: ESI MS m/z 621 [C₃₄H₄₈N₄O₅Si+H]⁺.

### Preparation of tert-Butyl (S)-7-(3a-Hydroxy-6,7-dimethyl-4-oxo-2,3,3a,4-tetrahydro-1H-pyrrolo[2,3-b]quinolin-1-yl)-2,3-dihydro-4H-pyrido[3,2-b][1,4]oxazine-4-carboxylate

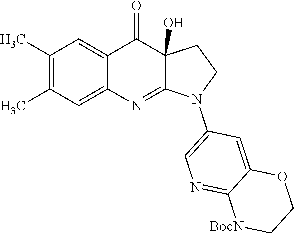 [SMILES: Cc1cc2c(cc1C)C(=O)[C@]1(O)CCN(c3cnc4c(c3)OCCN4C(=O)OC(C)(C)C)C1=N2]

A solution of tert-butyl (S)-7-(6,7-dimethyl-4-oxo-3a-((triisopropylsilyl)oxy)-2,3,3a,4-tetrahydro-1H-pyrrolo[2,3-b]quinolin-1-yl)-2,3-dihydro-4H-pyrido[3,2-b][1,4]oxazine-4-carboxylate (133 mg, 0.214 mmol) in tetrahydrofuran (5 mL) under a nitrogen atmosphere was treated with a 1.0 M solution of tetrabutylammonium fluoride in tetrahydrofuran (0.64 mL, 0.64 mmol) and acetic acid (0.07 mL, 0.07 g, 1 mmol), and the mixture was stirred. After 17 h, additional 1.0 M solution of tetrabutylammonium fluoride in tetrahydrofuran (0.21 mL, 0.21 mmol) was added, and the reaction continued to stir. After 12 h, additional 1.0 M solution of tetrabutylammonium fluoride in tetrahydrofuran (0.21 mL, 0.21 mmol) was added, and the reaction continued to stir. After 4 h, additional 1.0 M solution of tetrabutylammonium fluoride in tetrahydrofuran (0.21 mL, 0.21 mmol) was added, and the reaction continued to stir for 3 h. After this time, the mixture was concentrated under reduced pressure. The crude residue was purified by column chromatography (silica gel, 2-20% methanol/methylene chloride). The semi-crude product was dissolved in ethyl acetate (20 mL) and extracted with 0.3 M hydrochloric acid. The combined acid layers were adjusted to pH ˜8 with 2.0 M aqueous sodium hydroxide and extracted with ethyl acetate. The organics were dried over sodium sulfate, filtered, and concentrated under reduced pressure to provide a mixture of tert-butyl (S)-7-(3a-hydroxy-6,7-dimethyl-4-oxo-2,3,3a,4-tetrahydro-1H-pyrrolo[2,3-b]quinolin-1-yl)-2,3-dihydro-4H-pyrido[3,2-b][1,4]oxazine-4-carboxylate and (S)-1-(3,4-dihydro-2H-pyrido[3,2-b][1,4]oxazin-7-yl)-3a-hydroxy-6,7-dimethyl-1,2,3,3a-tetrahydro-4H-pyrrolo[2,3-b]quinolin-4-one (59 mg): ESI MS m/z 465 [C₂₅H₂₈N₄O₅+H]⁺; ESI MS m/z 365 [C₂₀H₂₀N₄O₃+H]⁺.

### Preparation of (S)-1-(3,4-Dihydro-2H-pyrido[3,2-b][1,4]oxazin-7-yl)-3a-hydroxy-6,7-dimethyl-1,2,3,3a-tetrahydro-4H-pyrrolo[2,3-b]quinolin-4-one

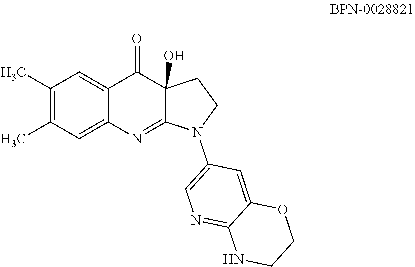 [SMILES: Cc1cc2c(cc1C)C(=O)[C@]1(O)CCN(c3cnc4c(c3)OCCN4)C1=N2]

A mixture of tert-butyl (S)-7-(3a-hydroxy-6,7-dimethyl-4-oxo-2,3,3a,4-tetrahydro-1H-pyrrolo[2,3-b]quinolin-1-yl)-2,3-dihydro-4H-pyrido[3,2-b][1,4]oxazine-4-carboxylate and (S)-1-(3,4-dihydro-2H-pyrido[3,2-b][1,4]oxazin-7-yl)-3a-hydroxy-6,7-dimethyl-1,2,3,3a-tetrahydro-4H-pyrrolo[2,3-b]quinolin-4-one (59 mg) was dissolved in dichloromethane (5 mL) and treated with trifluoroacetic acid (0.01 mL, 0.01 g, 0.1 mmol). The reaction mixture was stirred under a nitrogen atmosphere at room temperature for 3.5 h. After this time, the mixture was diluted with dichloromethane (5 mL) and treated with sodium bicarbonate (10 mL). The organic and aqueous layers were separated, and the organics were dried over sodium sulfate, filtered, and concentrated under reduced pressure. The residue was crystallized from hot acetonitrile to provide (S)-1-(3,4-dihydro-2H-pyrido[3,2-b][1,4]oxazin-7-yl)-3a-hydroxy-6,7-dimethyl-1,2,3,3a-tetrahydro-4H-pyrrolo[2,3-b]quinolin-4-one (34 mg, 74%) as an orange solid: mp=231-232° C.; ¹H NMR (500 MHz, DMSO-d₆) δ 8.03 (d, J=2.4 Hz, 1H), 7.93 (d, J=2.2 Hz, 1H), 7.45 (s, 1H), 6.96 (s, 1H), 6.70 (s, 1H), 6.67 (s, 1H), 4.15-4.13 (m, 2H), 4.00-3.95 (m, 1H), 3.87-3.83 (m, 1H), 3.42-3.39 (m, 2H), 2.25 (s, 3H), 2.20-2.19 (m, 5H); ESI MS m/z 365 [C₂₀H₂₀N₄O₃+H]⁺; UPLC (Method A) 99.0% (AUC), t_{R}=2.66 min; Chiral HPLC (Chiralpak AD, Method A) 84.1% (AUC), t_{R}=20.76 min.

### Preparation of (S)-1-(Benzo[b]thiophen-6-yl)-6-chloro-3a-hydroxy-1,2,3,3a-tetrahydro-4H-pyrrolo[2,3-b]quinolin-4-one

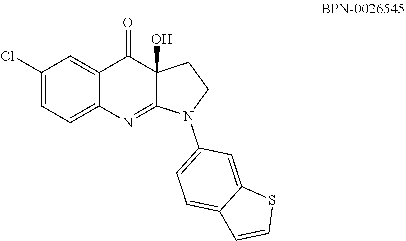 [SMILES: O=C1c2cc(Cl)ccc2N=C2N(c3ccc4ccsc4c3)CC[C@@]12O]

(S)-1-(Benzo[b]thiophen-6-yl)-6-chloro-3a-hydroxy-1,2,3,3a-tetrahydro-4H-pyrrolo[2,3-b]quinolin-4-one was prepared as an orange-red solid according to Synthetic Scheme 3: mp=221-229° C.; ¹H NMR (300 MHz, DMSO-d₆) δ 8.72 (d, J=1.7, 1H), 8.15 (dd, J=8.8, 2.0 Hz, 1H), 7.92 (d, J=8.2 Hz, 1H), 7.73 (d, J=5.4 Hz, 1H), 7.65 (d, J=2.6, 1H), 7.58 (dd, J=8.6, 2.6 Hz, 1H), 7.45 (d, J=5.4 Hz, 1H), 7.28 (d, J=8.5 Hz, 1H), 7.01 (s, 1H), 4.22-4.04 (m, 2H), 2.36-2.27 (m, 2H); ESI MS m/z 369 [C₁₉H₁₃ClN₂O₂S+H]⁺; HPLC (Method C) >99% (AUC), t_{R}=13.20 min; Chiral HPLC (Chiralpak AD, Method A) 92.1% (AUC), t_{R}=17.57 min.

### Preparation of (S)-1-(Benzo[b]thiophen-5-yl)-6-chloro-3a-hydroxy-1,2,3,3a-tetrahydro-4H-pyrrolo[2,3-b]quinolin-4-one

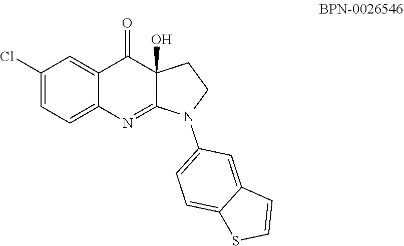 [SMILES: O=C1c2cc(Cl)ccc2N=C2N(c3ccc4sccc4c3)CC[C@@]12O]

(S)-1-(Benzo[b]thiophen-5-yl)-6-chloro-3a-hydroxy-1,2,3,3a-tetrahydro-4H-pyrrolo[2,3-b]quinolin-4-one was prepared as a yellow solid according to Synthetic Scheme 3: mp=204-208° C.; ¹H NMR (300 MHz, DMSO-d₆) δ 8.16 (d, J=2.1 Hz, 1H), 8.15 (d, J=2.0 Hz, 1H), 8.05 (d, J=8.9 Hz, 1H), 7.82 (d, J=5.4, 1H), 7.65 (dd, J=2.5, 2.6 Hz, 1H), 7.58 (d, J=3.7 Hz, 1H), 7.50 (d, J=5.4 Hz, 1H), 7.25 (d, J=8.6 Hz, 1H), 7.01 (s, 1H), 4.20-4.05 (m, 2H), 2.50-2.27 (m, 2H); ESI MS m/z 369 [C₁₉H₁₃ClN₂O₂S+H]⁺; HPLC (Method C) >99% (AUC), t_{R}=13.15 min; Chiral HPLC (Chiralpak AD, Method A) 85.1% (AUC), t_{R}=18.25 min.

### Preparation of (S)-6-Chloro-3a-hydroxy-1-(p-tolyl)-1,2,3,3a-tetrahydro-4H-pyrrolo[2,3-b]quinolin-4-one

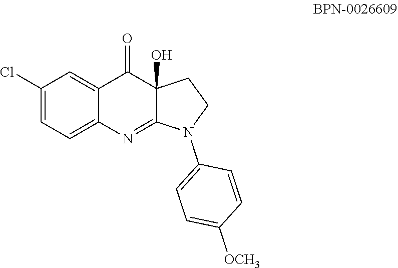 [SMILES: COc1ccc(N2CC[C@@]3(O)C(=O)c4cc(Cl)ccc4N=C23)cc1]

(S)-6-Chloro-3a-hydroxy-1-(p-tolyl)-1,2,3,3a-tetrahydro-4H-pyrrolo[2,3-b]quinolin-4-one was prepared as an orange solid according to Synthetic Scheme 2: mp=217-220° C.; ¹H NMR (300 MHz, DMSO-d₆) δ 7.93 (d, J=8.5 Hz, 2H), 7.63 (d, J=2.5 Hz, 1H), 7.55 (dd, J=3.7 Hz, 1H), 7.22 (m, 3H), 6.96 (s, 1H), 4.02 (m, 2H), 2.31 (s, 3H), 2.27 (m, 2H); ESI MS m/z 327 [C₁₈H₁₅ClN₂O₂+H]⁺; HPLC (Method C) 97.0% (AUC), t_{R}=12.45 min; Chiral HPLC (Chiralpak AD, Method A) 98.5% (AUC), t_{R}=14.31 min.

### Preparation of (S)-6-Chloro-1-(4-chlorophenyl)-3a-hydroxy-3,3a-dihydro-1H-pyrrolo[2,3-b]quinolin-4(2H)-one

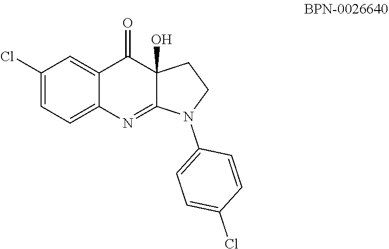 [SMILES: O=C1c2cc(Cl)ccc2N=C2N(c3ccc(Cl)cc3)CC[C@@]12O]

(S)-6-Chloro-1-(4-chlorophenyl)-3a-hydroxy-3,3a-dihydro-1H-pyrrolo[2,3-b]quinolin-4(2H)-one was prepared as a bright yellow solid according to Synthetic Scheme 2: mp=212-216° C.; ¹H NMR (300 MHz, DMSO-d₆) δ 8.15-8.10 (m, 2H), 7.65 (d, J=2.6 Hz, 1H), 7.59 (dd, J=8.5, 2.6 Hz, 1H), 7.52-7.47 (m, 2H), 7.24 (d, J=8.5 Hz, 1H), 7.00 (s, 1H), 4.13-3.94 (m, 2H), 2.37-2.22 (m, 2H); ESI MS m/z 347 [C₁₇H₁₂Cl₂N₂O₂+H]⁺; HPLC (Method C) 98.2% (AUC), t_{R}=13.45 min; Chiral HPLC (Chiralpak AD, Method A) 89.6% (AUC), t_{R}=19.74 min.

### Preparation of (S)-6-Chloro-3a-hydroxy-7-methyl-1-phenyl-3,3a-dihydro-1H-pyrrolo[2,3-b]quinolin-4(2H)-one

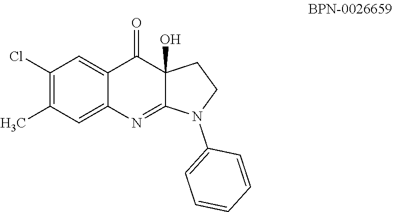 [SMILES: Cc1cc2c(cc1Cl)C(=O)[C@]1(O)CCN(c3ccccc3)C1=N2]

(S)-6-Chloro-3a-hydroxy-7-methyl-1-phenyl-3,3a-dihydro-1H-pyrrolo[2,3-b]quinolin-4(2H)-one was prepared as a light yellow solid according to Synthetic Scheme 2: mp=219-223° C.; ¹H NMR (300 MHz, DMSO-d₆) δ 8.07 (d, J=8.0 Hz, 2H), 7.63 (s, 1H), 7.44 (apparent t, J=7.9 Hz, 2H), 7.24-7.15 (m, 2H), 6.94 (s, 1H), 4.14-3.94 (m, 2H), 2.37-2.21 (m, 5H); ESI MS m/z 327 [C₁₈H₁₅ClN₂O₂+H]⁺; HPLC (Method C) 99.0% (AUC), t_{R}=12.69 min; Chiral HPLC (Chiralpak AD, Method A) 93.2% (AUC), t_{R}=18.38 min.

### Preparation of (S)-3a-Hydroxy-1-(3-methoxyphenyl)-6-methyl-3,3a-dihydro-1H-pyrrolo[2,3-b]quinolin-4(2H)-one

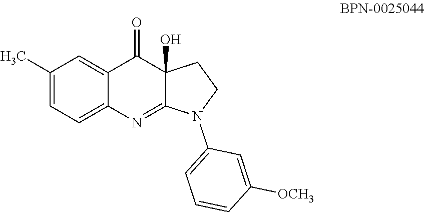 [SMILES: COc1cccc(N2CC[C@@]3(O)C(=O)c4cc(C)ccc4N=C23)c1]

(S)-3a-Hydroxy-1-(3-methoxyphenyl)-6-methyl-3,3a-dihydro-1H-pyrrolo[2,3-b]quinolin-4(2H)-one was prepared as a yellow solid according to Synthetic Scheme 3: mp=196-197° C.; ¹H NMR (500 MHz, DMSO-d₆) δ 7.99 (apparent t, J=2.1 Hz, 1H), 7.53 (d, J=1.2 Hz, 1H), 7.48-7.46 (m, 1H), 7.38 (dd, J=8.1, 2.0 Hz, 1H), 7.32 (apparent t, J=8.2 Hz, 1H), 7.12 (d, J=8.1 Hz, 1H), 6.81 (s, 1H), 6.73 (dd, J=8.2, 2.2 Hz, 1H), 4.06-4.01 (m, 1H), 3.97-3.94 (m, 1H), 3.80 (s, 3H), 2.30 (s, 3H), 2.26-2.24 (m, 2H); ESI MS m/z 323 [C₁₉H₁₈N₂O₃+H]⁺; HPLC (Method C) >99% (AUC), t_{R}=12.05 min; Chiral HPLC (Chiralpak AD, Method A) 97.0% (AUC), t_{R}=15.07 min.

### Preparation of (S)-1-(3-Chlorophenyl)-3a-hydroxy-6-methyl-3,3a-dihydro-1H-pyrrolo[2,3-b]quinolin-4(2H)-one

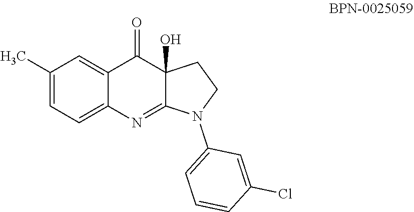 [SMILES: Cc1ccc2c(c1)C(=O)[C@]1(O)CCN(c3cccc(Cl)c3)C1=N2]

(S)-1-(3-Chlorophenyl)-3a-hydroxy-6-methyl-3,3a-dihydro-1H-pyrrolo[2,3-b]quinolin-4(2H)-one was prepared as an orange solid according to Synthetic Scheme 2: mp=199-200° C.; ¹H NMR (500 MHz, DMSO-d₆) δ 8.39 (apparent t, J=2.1 Hz, 1H), 7.93 (ddd, J=8.4, 2.2, 0.7 Hz, 1H), 7.55 (d, J=1.6 Hz, 1H), 7.45 (apparent t, J=8.2 Hz, 1H), 7.41-7.39 (m, 1H), 7.19 (ddd, J=7.9, 1.9, 0.6 Hz, 1H), 7.15 (d, J=8.1 Hz, 1H), 6.86 (s, 1H), 4.07-3.96 (m, 2H), 2.31-2.25 (m, 5H); ESI MS m/z 327 [C₁₈H₁₅ClN₂O₂+H]⁺; HPLC (Method C) >99% (AUC), t_{R}=12.18 min; Chiral HPLC (Chiralpak AD, Method A) >99% (AUC), t_{R}=14.59 min.

### Preparation of (S)-3a-Hydroxy-6-methyl-1-(m-tolyl)-3,3a-dihydro-1H-pyrrolo[2,3-b]quinolin-4(2H)-one

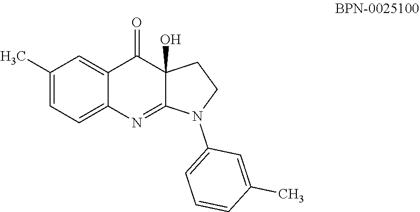

(S)-3a-Hydroxy-6-methyl-1-(m-tolyl)-3,3a-dihydro-1H-pyrrolo[2,3-b]quinolin-4(2H)-one was prepared as an orange solid according to Synthetic Scheme 3: mp=192-193° C.; ¹H NMR (500 MHz, DMSO-d₆) δ 7.94 (dd, J=8.2, 2.0 Hz, 1H), 7.84 (s, 1H), 7.52 (d, J=2.0 Hz, 1H), 7.38-7.36 (m, 1H), 7.30 (apparent t, J=7.9 Hz, 1H), 7.11 (d, J=8.1 Hz, 1H), 6.96 (d, J=7.5 Hz, 1H), 6.79 (s, 1H), 4.07-4.02 (m, 1H), 3.96-3.93 (m, 1H), 2.36 (s, 3H), 2.30 (s, 3H), 2.26-2.23 (m, 2H); ESI MS m/z 307 [C₁₉H₁₈N₂O₂+H]⁺; HPLC (Method C) >99% (AUC), t_{R}=14.48 min; Chiral HPLC (Chiralpak AD, Method A) >99% (AUC), t_{R}=12.51 min.

### Preparation of (S)-1-(3-Bromophenyl)-3a-hydroxy-6-methyl-3,3a-dihydro-1H-pyrrolo[2,3-b]quinolin-4(2H)-one

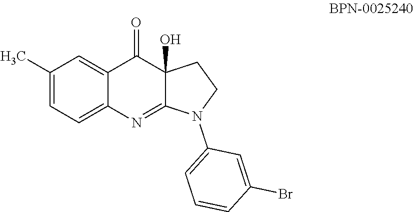 [SMILES: Cc1ccc2c(c1)C(=O)[C@]1(O)CCN(c3cccc(Br)c3)C1=N2]

(S)-1-(3-Bromophenyl)-3a-hydroxy-6-methyl-3,3a-dihydro-1H-pyrrolo[2,3-b]quinolin-4(2H)-one was prepared as a yellow solid according to Synthetic Scheme 3: mp=195-196° C.; ¹H NMR (500 MHz, DMSO-d₆) δ 8.53 (apparent t, J=2.0 Hz, 1H), 7.96 (ddd, J=8.3, 2.2, 0.9 Hz, 1H), 7.55 (d, J=1.8 Hz, 1H), 7.41-7.37 (m, 2H), 7.32 (ddd, J=7.9, 1.8, 0.9 Hz, 1H), 7.14 (d, J=8.1 Hz, 1H), 6.87 (s, 1H), 4.06-3.95 (m, 2H), 2.31 (s, 3H), 2.28-2.24 (m, 2H); ESI MS m/z 371 [C₁₈H₁₅BrN₂O₂+H]⁺; HPLC (Method C) 96.2% (AUC), t_{R}=12.72 min; Chiral HPLC (Chiralpak AD, Method A) >99% (AUC), t_{R}=13.18 min.

### Preparation of (S)-1-(3-Chloro-4-methylphenyl)-3a-hydroxy-6-methyl-3,3a-dihydro-1H-pyrrolo[2,3-b]quinolin-4(2H)-one

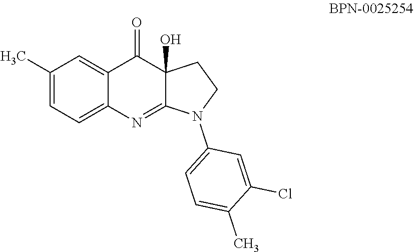 [SMILES: Cc1ccc2c(c1)C(=O)[C@]1(O)CCN(c3ccc(C)c(Cl)c3)C1=N2]

(S)-1-(3-Chloro-4-methylphenyl)-3a-hydroxy-6-methyl-3,3a-dihydro-1H-pyrrolo[2,3-b]quinolin-4(2H)-one was prepared as a yellow solid according to Synthetic Scheme 3: mp=213-214° C.; ¹H NMR (500 MHz, DMSO-d₆) δ 8.37 (d, J=2.4 Hz, 1H), 7.82 (dd, J=8.4, 2.3 Hz, 1H), 7.54 (d, J=1.6 Hz, 1H), 7.39 (d, J=8.3 Hz, 2H), 7.12 (d, J=8.1 Hz, 1H), 6.84 (s, 1H), 4.05-3.93 (m, 2H), 2.33-2.31 (m, 6H), 2.27-2.22 (m, 2H); ESI MS m/z 341 [C₁₉H₁₇ClN₂O₂+H]⁺; HPLC (Method C) >99% (AUC), t_{R}=2.30 min; Chiral HPLC (Chiralpak AD, Method A) >99% (AUC), t_{R}=14.28 min.

### Preparation of (S)-1-(3,4-Dimethylphenyl)-3a-hydroxy-6-methyl-3,3a-dihydro-1H-pyrrolo[2,3-b]quinolin-4(2H)-one

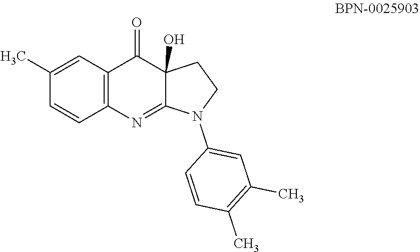

(S)-1-(3,4-Dimethylphenyl)-3a-hydroxy-6-methyl-3,3a-dihydro-1H-pyrrolo[2,3-b]quinolin-4(2H)-one was prepared as a yellow solid according to Synthetic Scheme 3: mp=197-199° C.; ¹H NMR (500 MHz, DMSO-d₆) δ 7.83 (dd, J=8.5, 2.0 Hz, 1H), 7.78 (d, J=1.5 Hz, 1H), 7.51 (d, J=1.0 Hz, 1H), 7.35 (dd, J=7.5, 1.5 Hz, 1H), 7.17 (d, J=8.5 Hz, 1H), 7.09 (d, J=8.0 Hz, 1H), 6.77 (s, 1H), 4.05-4.00 (m, 1H), 3.94-3.90 (m, 1H), 2.29-2.22 (m, 11H); ESI MS m/z 321 [C₂₀H₂₀N₂O₂+H]⁺; HPLC (Method F) 98.4% (AUC), t_{R}=14.99 min; Chiral HPLC (Chiralpak AD, Method A) 95.4% (AUC), t_{R}=13.05 min.

### Preparation of (S)-1-(Benzofuran-6-yl)-3a-hydroxy-6-methyl-3,3a-dihydro-1H-pyrrolo[2,3-b]quinolin-4(2H)-one

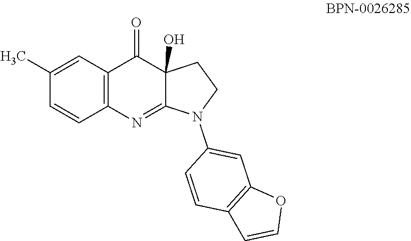 [SMILES: Cc1ccc2c(c1)C(=O)[C@]1(O)CCN(c3ccc4ccoc4c3)C1=N2]

(S)-1-(Benzofuran-6-yl)-3a-hydroxy-6-methyl-3,3a-dihydro-1H-pyrrolo[2,3-b]quinolin-4(2H)-one was prepared as a red-orange solid according to Synthetic Scheme 3: mp=204-205° C.; ¹H NMR (500 MHz, DMSO-d₆) δ 8.54 (s, 1H), 7.98 (d, J=2.5 Hz, 1H), 7.88 (dd, J=8.5, 2.0 Hz, 1H), 7.68 (d, J=8.5 Hz, 1H), 7.54 (d, J=1.5 Hz, 1H), 7.38 (dd, J=8.5, 2.0 Hz, 1H), 7.15 (d, J=8.0 Hz, 1H), 6.95 (dd, J=2.0, 2.0 Hz, 1H), 6.84 (s, 1H), 4.16-4.11 (m, 1H), 4.05-4.01 (m, 1H), 2.33-2.25 (m, 5H); ESI MS m/z 333 [C₂₀H₁₆N₂O₃+H]⁺; HPLC (Method C) >99% (AUC), t_{R}=12.24 min; Chiral HPLC (Chiralpak AD, Method A) >99% (AUC), t_{R}=17.78 min.

### Preparation of (S)-1-(4-Chloro-3-methylphenyl)-3a-hydroxy-6-methyl-3,3a-dihydro-1H-pyrrolo[2,3-b]quinolin-4(2H)-one

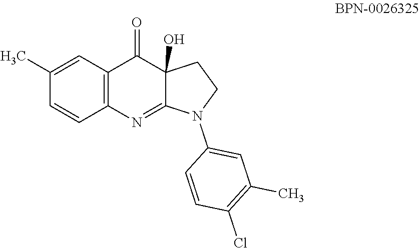

(S)-1-(4-Chloro-3-methylphenyl)-3a-hydroxy-6-methyl-3,3a-dihydro-1H-pyrrolo[2,3-b]quinolin-4(2H)-one was prepared as a yellow solid according to Synthetic Scheme 3: mp=203-204° C.; ¹H NMR (500 MHz, DMSO-d₆) δ 8.06 (dd, J=9.0, 3.0 Hz, 1H), 8.01 (d, J=3.0 Hz, 1H), 7.53 (d, J=2.0 Hz, 1H), 7.44 (d, J=8.5 Hz, 1H), 7.38 (dd, J=8.5, 2.0 Hz, 1H), 7.14 (d, J=8.0 Hz, 1H), 6.83 (s, 1H), 4.05-4.00 (m, 1H), 3.97-3.93 (m, 1H), 2.38 (s, 3H), 2.31 (s, 3H), 2.27-2.24 (m, 2H); ESI MS m/z 341 [C₁₉H₁₇ClN₂O₂+H]⁺; HPLC (Method C) >99% (AUC), t_{R}=13.07 min; Chiral HPLC (Chiralpak AD, Method A) >99% (AUC), t_{R}=13.68 min.

### Preparation of (S)-1-(Benzofuran-6-yl)-6-chloro-3a-hydroxy-3,3a-dihydro-1H-pyrrolo[2,3-b]quinolin-4(2H)-one

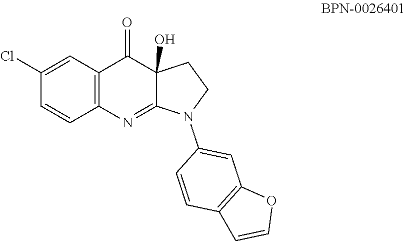 [SMILES: O=C1c2cc(Cl)ccc2N=C2N(c3ccc4ccoc4c3)CC[C@@]12O]

(S)-1-(Benzofuran-6-yl)-6-chloro-3a-hydroxy-3,3a-dihydro-1H-pyrrolo[2,3-b]quinolin-4(2H)-one was prepared as a yellow-orange solid according to Synthetic Scheme 3: mp=209-211° C.; ¹H NMR (500 MHz, DMSO-d₆) δ 8.50 (s, 1H), 8.00 (d, J=2.0 Hz, 1H), 7.87 (dd, J=9.0, 2.0 Hz, 1H), 7.69 (d, J=9.0 Hz, 1H), 7.65 (d, J=2.5 Hz, 1H), 7.57 (dd, J=8.5, 2.5 Hz, 1H), 7.26 (d, J=8.5 Hz, 1H), 6.98 (s, 1H), 6.96 (dd, J=2.5, 1.0 Hz, 1H), 4.20-4.16 (m, 1H), 4.07-4.03 (m, 1H), 2.38-2.34 (m, 1H), 2.30-2.26 (m, 1H); ESI MS m/z 353 [C₁₉H₁₃ClN₂O₃+H]⁺; HPLC (Method B) >99% (AUC), t_{R}=9.12 min; Chiral HPLC (Chiralpak AD, Method A) 99.0% (AUC), t_{R}=17.23 min.

### Preparation of (S)-3a-Hydroxy-6,7-dimethyl-1-(m-tolyl)-3,3a-dihydro-1H-pyrrolo[2,3-b]quinolin-4(2H)-one

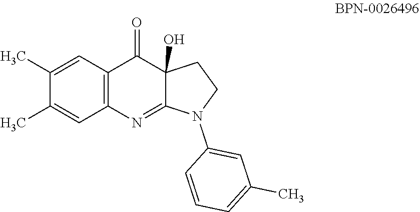

(S)-3a-Hydroxy-6,7-dimethyl-1-(m-tolyl)-3,3a-dihydro-1H-pyrrolo[2,3-b]quinolin-4(2H)-one was prepared as an orange solid according to Synthetic Scheme 3: mp=196-198° C.; ¹H NMR (500 MHz, DMSO-d₆) δ 7.95 (d, J=8.5 Hz, 1H), 7.83 (s, 1H), 7.48 (s, 1H), 7.30 (apparent t, J=8.0 Hz, 1H), 7.03 (s, 1H), 6.96 (d, J=7.5 Hz, 1H), 6.74 (s, 1H), 4.06-4.01 (m, 1H), 3.95-3.92 (m, 1H), 2.36 (s, 3H), 2.27 (s, 3H), 2.24-2.20 (m, 5H); ESI MS m/z 321 [C₂₀H₂₀N₂O₂+H]⁺; HPLC (Method C) >99% (AUC), t_{R}=12.62 min; Chiral HPLC (Chiralpak AD, Method A) >99% (AUC), t_{R}=13.47 min.

### Preparation of (S)-3a-Hydroxy-1-(3-methoxyphenyl)-6,7-dimethyl-3,3a-dihydro-1H-pyrrolo[2,3-b]quinolin-4(2H)-one

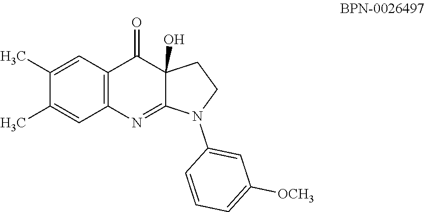 [SMILES: COc1cccc(N2CC[C@@]3(O)C(=O)c4cc(C)c(C)cc4N=C23)c1]

(S)-3a-Hydroxy-1-(3-methoxyphenyl)-6,7-dimethyl-3,3a-dihydro-1H-pyrrolo[2,3-b]quinolin-4(2H)-one was prepared as an yellow solid according to Synthetic Scheme 3: mp=176-178° C.; ¹H NMR (500 MHz, DMSO-d₆) δ 7.97 (apparent t, J=2.0 Hz, 1H), 7.48 (dd, J=8.0, 2.5 Hz, 2H), 7.31 (apparent t, J=8.5 Hz, 1H), 7.04 (s, 1H), 6.76 (s, 1H), 6.73 (dd, J=8.0, 2.0 Hz, 1H), 4.05-4.00 (m, 1H), 3.96-3.93 (m, 1H), 3.80 (s, 3H), 2.27 (s, 3H), 2.24-2.21 (m, 5H); ESI MS m/z 337 [C₂₀H₂₀N₂O₃+H]⁺; HPLC (Method C) >99% (AUC), t_{R}=12.41 min; Chiral HPLC (Chiralpak AD, Method A) >99% (AUC), t_{R}=16.05 min.

### Preparation of (S)-4-(3a-Hydroxy-6,7-dimethyl-4-oxo-2,3,3a,4-tetrahydro-1H-pyrrolo[2,3-b]quinolin-1-yl)benzonitrile

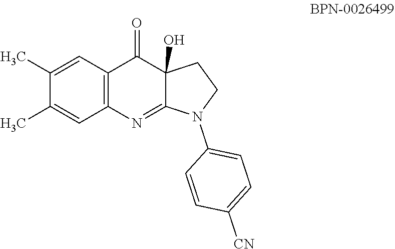 [SMILES: Cc1cc2c(cc1C)C(=O)[C@]1(O)CCN(c3ccc(C#N)cc3)C1=N2]

(S)-4-(3a-hydroxy-6,7-dimethyl-4-oxo-2,3,3a,4-tetrahydro-1H-pyrrolo[2,3-b]quinolin-1-yl)benzonitrile was prepared as a yellow-brown solid according to Synthetic Scheme 3: mp=229-231° C.; ¹H NMR (500 MHz, DMSO-d₆) δ 8.32 (d, J=9.0 Hz, 2H), 7.87 (dd, J=7.5, 2.0 Hz, 2H), 7.52 (s, 1H), 7.13 (s, 1H), 6.86 (s, 1H), 4.07-3.99 (m, 2H), 2.29-2.24 (m, 8H); ESI MS m/z 332 [C₂₀H₁₇N₃O₂+H]⁺; HPLC (Method C) >99% (AUC), t_{R}=12.60 min; Chiral HPLC (Chiralpak AD, Method A) 97.2% (AUC), t_{R}=19.40 min.

### Preparation of (S)-1-(3-Chlorophenyl)-3a-hydroxy-6,7-dimethyl-3,3a-dihydro-1H-pyrrolo[2,3-b]quinolin-4(2H)-one

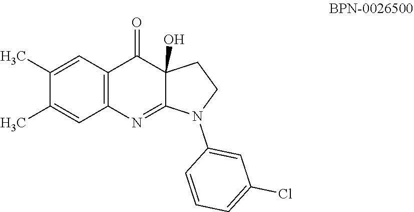 [SMILES: Cc1cc2c(cc1C)C(=O)[C@]1(O)CCN(c3cccc(Cl)c3)C1=N2]

(S)-1-(3-Chlorophenyl)-3a-hydroxy-6,7-dimethyl-3,3a-dihydro-1H-pyrrolo[2,3-b]quinolin-4(2H)-one was prepared as a yellow solid according to Synthetic Scheme 3: mp=203-204° C.; ¹H NMR (500 MHz, DMSO-d₆) δ 8.40 (apparent t, J=2.0 Hz, 1H), 7.92 (d, J=7.5 Hz, 1H), 7.50 (s, 1H), 7.44 (apparent t, J=8.5 Hz, 1H), 7.19 (d, J=7.5 Hz, 1H), 7.01 (s, 1H), 6.80 (s, 1H), 4.06-4.00 (m, 1H), 3.97-3.95 (m, 1H), 2.29 (s, 3H), 2.25-2.23 (m, 5H); ESI MS m/z 341 [C₁₉H₁₇ClN₂O₂+H]⁺; HPLC (Method C) >99% (AUC), t_{R}=12.89 min; Chiral HPLC (Chiralpak AD, Method A) >99% (AUC), t_{R}=13.96 min.

### Preparation of (S)-4-(4a-Hydroxy-2-methyl-4-oxo-5,6-dihydro-4H-pyrrolo[2,3-b]thieno[3,2-e]pyridin-7(4aH)-yl)-2-methylbenzonitrile

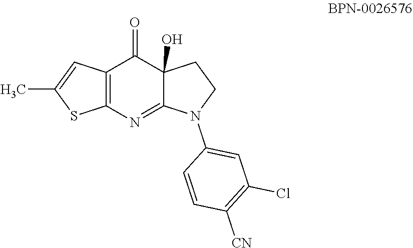 [SMILES: Cc1cc2c(s1)N=C1N(c3ccc(C#N)c(Cl)c3)CC[C@@]1(O)C2=O]

(S)-4-(4a-Hydroxy-2-methyl-4-oxo-5,6-dihydro-4H-pyrrolo[2,3-b]thieno[3,2-e]pyridin-7(4aH)-yl)-2-methylbenzonitrile was prepared as an orange solid according to Synthetic Scheme 3: mp=226-227° C.; ¹H NMR (500 MHz, DMSO-d₆) δ 8,17 (dd, J=9.0, 2.5 Hz, 1H), 7.90 (dd, J=2.0 Hz, 1H), 7.83 (d, J=9.0 Hz, 1H), 6.97 (s, 1H), 6.84 (d, J=1.0 Hz, 1H), 4.15-4.06 (m, 2H), 2.52 (s, 3H), 2.37 (d, J=1.0, 3H), 2.25-2.21 (m, 2H); ESI MS m/z 338 [C₁₈H₁₅N₃O₂S+H]⁺; HPLC (Method C) >99% (AUC), t_{R}=15.23 min; Chiral HPLC (Chiralpak AD, Method A) 98.9% (AUC), t_{R}=19.02 min.

### Preparation of (S)-6-Chloro-3a-hydroxy-1-(2-methoxypyridin-4-yl)-3,3a-dihydro-1H-pyrrolo[2,3-b]quinolin-4(2H)-one

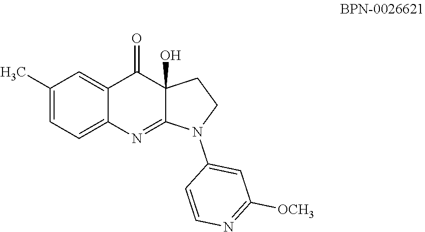 [SMILES: COc1cc(N2CC[C@@]3(O)C(=O)c4cc(C)ccc4N=C23)ccn1]

(S)-6-Chloro-3a-hydroxy-1-(2-methoxypyridin-4-yl)-3,3a-dihydro-1H-pyrrolo[2,3-b]quinolin-4(2H)-one was prepared as a yellow solid according to Synthetic Scheme 6: mp=230-231° C.; ¹H NMR (500 MHz, DMSO-d₆) δ 8.14 (d, J=6.0 Hz, 1H), 7.72 (dd, J=6.0, 2.0 Hz, 1H), 7.68 (d, J=3.0 Hz, 1H), 7.63 (dd, J=8.5, 2.5 Hz, 1H), 7.56 (d, J=2.0 Hz, 1H), 7.34 (d, J=8.5 Hz, 1H), 7.04 (s, 1H), 4.02-4.00 (m, 2H), 3.87 (s, 3H), 2.39-2.32 (m, 1H), 2.26-2.23 (m, 1H); ESI MS m/z 344 [C₁₇H₁₄ClN₃O₃+H]⁺; HPLC (Method C) 96.0% (AUC), t_{R}=13.01 min; Chiral HPLC (Chiralpak AD, Method A) 83.0% (AUC), t_{R}=14.71 min.

### Preparation of (S)-7-(3-Bromophenyl)-4a-hydroxy-2-methyl-4a,5,6,7-tetrahydro-4H-pyrrolo[2,3-b]thieno[3,2-e]pyridin-4-one

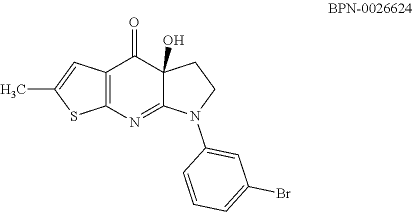 [SMILES: Cc1cc2c(s1)N=C1N(c3cccc(Br)c3)CC[C@@]1(O)C2=O]

(S)-7-(3-Bromophenyl)-4a-hydroxy-2-methyl-4a,5,6,7-tetrahydro-4H-pyrrolo[2,3-b]thieno[3,2-e]pyridin-4-one was prepared as a red-orange solid according to Synthetic Scheme 3: mp=236-238° C.; ¹H NMR (500 MHz, DMSO-d₆) δ 8.26 (apparent t, J=2.0 Hz, 1H), 7.90 (apparent dt, J=8.0, 1.5 Hz, 1H), 7.43-7.37 (m, 2H), 6.92 (s, 1H), 6.81 (d, J=1.0, 1H), 4.15-4.10 (m, 1H), 4.05-4.01 (m, 1H), 2.36 (d, J=1.0, 3H), 2.22-2.19 (m, 2H); ESI MS m/z 377 [C₁₆H₁₃BrN₂O₂S+H]⁺; HPLC (Method C) 94.0% (AUC), t_{R}=16.31 min; Chiral HPLC (Chiralpak AD, Method A) >99% (AUC), t_{R}=16.36 min.

### Preparation of (S)-7-(Benzofuran-6-yl)-4a-hydroxy-2-methyl-4a,5,6,7-tetrahydro-4H-pyrrolo[2,3-b]thieno[3,2-e]pyridin-4-one

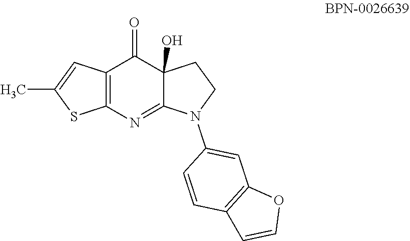 [SMILES: Cc1cc2c(s1)N=C1N(c3ccc4ccoc4c3)CC[C@@]1(O)C2=O]

(S)-7-(Benzofuran-6-yl)-4a-hydroxy-2-methyl-4a,5,6,7-tetrahydro-4H-pyrrolo[2,3-b]thieno[3,2-e]pyridin-4-one was prepared as a red solid according to Synthetic Scheme 3: mp=247-248° C.; ¹H NMR (500 MHz, DMSO-d₆) δ 8.29 (s, 1H), 8.02 (d, J=2.5 Hz, 1H), 7.81 (dd, J=8.5, 2.0 Hz, 1H), 7.71 (d, J=8.5 Hz, 1H), 7.97 (dd, J=2.0, 1.0 Hz, 1H), 6.90 (s, 1H), 6.79 (d, J=1.0, 1H), 4.25-4.20 (m, 1H), 4.11-4.08 (m, 1H), 2.35 (d, J=1.0, 3H), 2.26-2.22 (m, 2H); ESI MS m/z 339 [C₁₈H₁₄N₂O₃S+H]⁺; HPLC (Method B) >99% (AUC), t_{R}=10.20 min; Chiral HPLC (Chiralpak AD, Method A) >99% (AUC), t_{R}=22.00 min.

### Preparation of (S)-1-(3-Bromophenyl)-3a-hydroxy-6,7-dimethyl-3,3a-dihydro-1H-pyrrolo[2,3-b]quinolin-4(2H)-one

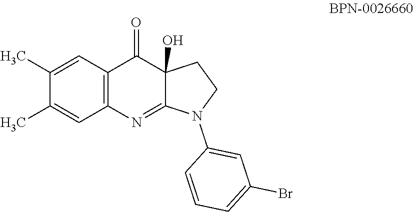 [SMILES: Cc1cc2c(cc1C)C(=O)[C@]1(O)CCN(c3cccc(Br)c3)C1=N2]

(S)-1-(3-Bromophenyl)-3a-hydroxy-6,7-dimethyl-3,3a-dihydro-1H-pyrrolo[2,3-b]quinolin-4(2H)-one was prepared as a yellow solid according to Synthetic Scheme 3: mp=223-224° C.; ¹H NMR (500 MHz, DMSO-d₆) δ 8.52 (apparent t, J=2.0 Hz, 1H), 7.96 (ddd, J=8.3, 2.2, 1.0 Hz, 1H), 7.50 (s, 1H), 7.38 (apparent t, J=8.1 Hz, 1H), 7.32 (ddd, J=7.9, 1.7, 0.9 Hz, 1H), 7.05 (s, 1H), 6.81 (s, 1H), 4.05-4.00 (m, 1H), 3.98-3.94 (m, 1H), 2.28 (s, 3H), 2.25-2.23 (m, 5H); ESI MS m/z 385 [C₁₉H₁₇BrN₂O₂+H]⁺; HPLC (Method C) 93.1% (AUC), t_{R}=13.27 min; Chiral HPLC (Chiralpak AD, Method A) >99% (AUC), t_{R}=14.35 min.

### Preparation of (S)-4-(3a-Hydroxy-6,7-dimethyl-4-oxo-2,3,3a,4-tetrahydro-1H-pyrrolo[2,3-b]quinolin-1-yl)-2-methylbenzonitrile

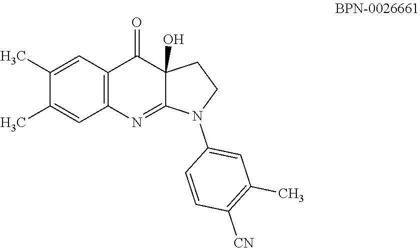

(S)-4-(3a-Hydroxy-6,7-dimethyl-4-oxo-2,3,3a,4-tetrahydro-1H-pyrrolo[2,3-b]quinolin-1-yl)-2-methylbenzonitrile was prepared as a yellow-brown solid according to Synthetic Scheme 3: mp=240-241° C.; ¹H NMR (500 MHz, DMSO-d₆) δ 8.28 (dd, J=8.8, 2.2 Hz, 1H), 8.10 (d, J=1.9 Hz, 1H), 7.79 (d, J=8.7 Hz, 1H), 7.52 (s, 1H), 7.13 (s, 1H), 7.85 (s, 1H), 4.03-4.00 (m, 2H), 2.53 (s, 3H), 2.29 (s, 3H), 2.27-2.24 (m, 5H); ESI MS m/z 346 [C₂₁H₁₉N₃O₂+H]⁺; HPLC (Method C) 98.6% (AUC), t_{R}=13.14 min; Chiral HPLC (Chiralpak AD, Method A) 88.1% (AUC), t_{R}=17.66 min.

### Preparation of (S)-1-(4-Chlorophenyl)-3a-hydroxy-6,7-dimethyl-3,3a-dihydro-1H-pyrrolo[2,3-b]quinolin-4(2H)-one

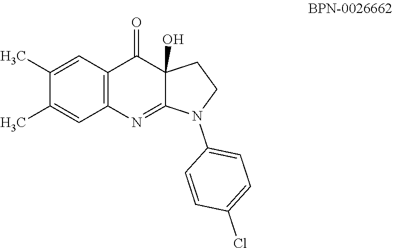 [SMILES: Cc1cc2c(cc1C)C(=O)[C@]1(O)CCN(c3ccc(Cl)cc3)C1=N2]

(S)-1-(4-Chlorophenyl)-3a-hydroxy-6,7-dimethyl-3,3a-dihydro-1H-pyrrolo[2,3-b]quinolin-4(2H)-one was prepared as a yellow solid according to Synthetic Scheme 3: mp=214-216° C.; ¹H NMR (500 MHz, DMSO-d₆) δ 8.15-8.12 (m, 2H), 7.49-7.46 (m, 3H), 7.06 (s, 1H), 6.79 (s, 1H), 4.05-4.02 (m, 1H), 3.96-3.92 (m, 1H), 2.27 (s, 3H), 2.25-2.22 (m, 5H); ESI MS m/z 341 [C₁₉H₁₇ClN₂O₂+H]⁺; HPLC (Method C) >99% (AUC), t_{R}=13.06 min; Chiral HPLC (Chiralpak AD, Method A) 97.6% (AUC), t_{R}=16.42 min.

### Preparation of (S)-3a-Hydroxy-6,7-dimethyl-1-(p-tolyl)-3,3a-dihydro-1H-pyrrolo[2,3-b]quinolin-4(2H)-one

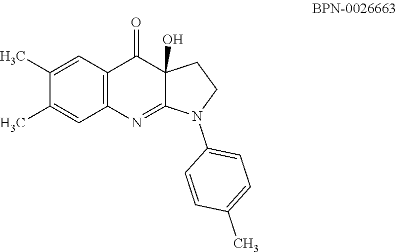 [SMILES: Cc1ccc(N2CC[C@@]3(O)C(=O)c4cc(C)c(C)cc4N=C23)cc1]

(S)-3a-Hydroxy-6,7-dimethyl-1-(p-tolyl)-3,3a-dihydro-1H-pyrrolo[2,3-b]quinolin-4(2H)-one was prepared as a yellow solid according to Synthetic Scheme 3: mp=219-220° C.; ¹H NMR (500 MHz, DMSO-d₆) δ 7.96-3.94 (m, 2H), 7.47 (s, 1H), 7.22 (d, J=8.3 Hz, 2H), 7.02 (s, 1H), 6.73 (s, 1H), 4.05-4.00 (m, 1H), 3.93-3.89 (m, 1H), 2.31 (s, 3H), 2.26 (s, 3H), 2.24-2.21 (m, 5H); ESI MS m/z 321 [C₂₀H₂₀N₂O₂+H]⁺; HPLC (Method B) >99% (AUC), t_{R}=8.97 min; Chiral HPLC (Chiralpak AD, Method A) >99% (AUC), t_{R}=15.61 min.

### Preparation of (S)-1-(Benzofuran-6-yl)-3a-hydroxy-6,7-dimethyl-1,2,3,3a-tetrahydro-4H-pyrrolo[2,3-b]quinolin-4-one

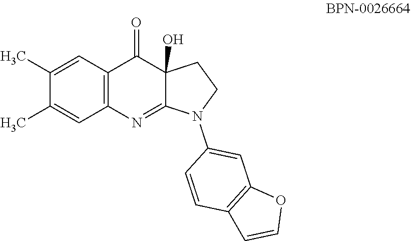 [SMILES: Cc1cc2c(cc1C)C(=O)[C@]1(O)CCN(c3ccc4ccoc4c3)C1=N2]

(S)-1-(Benzofuran-6-yl)-3a-hydroxy-6,7-dimethyl-1,2,3,3a-tetrahydro-4H-pyrrolo[2,3-b]quinolin-4-one was prepared as a red solid according to Synthetic Scheme 3: mp=213-214° C.; ¹H NMR (500 MHz, DMSO-d₆) δ 8.56 (s, 1H), 7.98 (d, J=2.2 Hz, 1H), 7.88 (dd, J=8.6, 2.0 Hz, 1H), 7.67 (d, J=8.6 Hz, 1H), 7.49 (s, 1H), 7.09 (s, 1H), 6.50 (dd, J=2.2, 0.9 Hz, 1H), 6.79 (s, 1H), 4.15-4.10 (m, 1H), 4.04-4.00 (m, 1H), 2.28-2.25 (m, 5H), 2.23 (s, 3H); ESI MS m/z 347 [C₂₁H₁₈N₂O₃+H]⁺; HPLC (Method B) >99% (AUC), t_{R}=9.10 min; Chiral HPLC (Chiralpak AD, Method A) 95.8% (AUC), t_{R}=19.08 min.

### Preparation of (S)-3a-Hydroxy-1-(4-methoxyphenyl)-6,7-dimethyl-1,2,3,3a-tetrahydro-4H-pyrrolo[2,3-b]quinolin-4-one

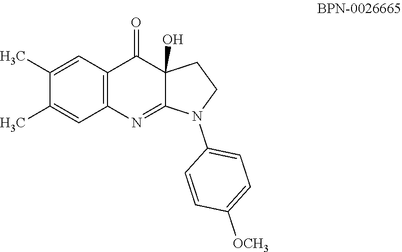 [SMILES: COc1ccc(N2CC[C@@]3(O)C(=O)c4cc(C)c(C)cc4N=C23)cc1]

(S)-3a-Hydroxy-1-(4-methoxyphenyl)-6,7-dimethyl-1,2,3,3a-tetrahydro-4H-pyrrolo[2,3-b]quinolin-4-one was prepared as a yellow solid according to Synthetic Scheme 3: mp=199-200° C.; ¹H NMR (500 MHz, DMSO-d₆) δ 7.98-7.95 (m, 2H), 7.46 (s, 1H), 7.00-6.98 (m, 3H), 6.72 (s, 1H), 4.06-4.01 (m, 1H), 3.91-3.88 (m, 1H), 3.77 (s, 3H), 2.25 (s, 3H), 2.23-2.21 (m, 5H); ESI MS m/z 337 [C₂₀H₂₀N₂O₃+H]⁺; HPLC (Method B) >99% (AUC), t_{R}=8.71 min; Chiral HPLC (Chiralpak AD, Method A) >99% (AUC), t_{R}=18.75 min.

### Preparation of (S)-3a-Hydroxy-6,7-dimethyl-1-(2-methylthiazol-5-yl)-1,2,3,3a-tetrahydro-4H-pyrrolo[2,3-b]quinolin-4-one

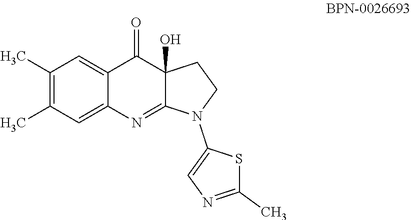

(S)-3a-hydroxy-6,7-dimethyl-1-(2-methylthiazol-5-yl)-1,2,3,3a-tetrahydro-4H-pyrrolo[2,3-b]quinolin-4-one was prepared as an orange-yellow solid according to Synthetic Scheme 6: mp=234-235° C.; ¹H NMR (500 MHz, DMSO-d₆) δ 7.49 (s, 1H), 7.44 (s, 1H), 7.05 (s, 1H), 6.87 (s, 1H), 4.03-3.94 (m, 2H), 2.60 (s, 3H), 2.31-2.27 (m, 5H), 2.22 (s, 3H); ESI MS m/z 328 [C₁₇H₁₇N₃O₂S+H]⁺; HPLC (Method C) >99% (AUC), t_{R}=12.84 min; Chiral HPLC (Chiralpak AD, Method A) 53.0% (AUC), t_{R}=15.14 min.

### Preparation of (S)-3a-Hydroxy-6,7-dimethyl-1-(thiophen-2-yl)-1,2,3,3a-tetrahydro-4H-pyrrolo[2,3-b]quinolin-4-one

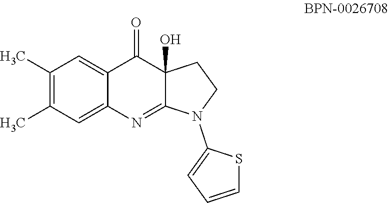 [SMILES: Cc1cc2c(cc1C)C(=O)[C@]1(O)CCN(c3cccs3)C1=N2]

(S)-3a-Hydroxy-6,7-dimethyl-1-(thiophen-2-yl)-1,2,3,3a-tetrahydro-4H-pyrrolo[2,3-b]quinolin-4-one was prepared as an orange solid according to Synthetic Scheme 4: mp=229-231° C.; ¹H NMR (500 MHz, DMSO-d₆) δ 7.49 (s, 1H), 7.14 (dd, J=5.5, 1.4 Hz, 1H), 7.05 (s, 1H), 6.96 (dd, J=5.4, 3.8 Hz, 1H), 6.83-6.82 (m, 2H), 4.07-3.97 (m, 2H), 2.37-2.35 (m, 2H), 2.28 (s, 3H), 2.22 (s, 3H); ESI MS m/z 313 [C₁₇H₁₆N₂O₂S+H]⁺; HPLC (Method B) >99% (AUC), t_{R}=13.17 min; Chiral HPLC (Chiralpak AD, Method A) 83.5% (AUC), t_{R}=17.17 min.

### Preparation of (S)-3a-Hydroxy-1-(2-methoxypyridin-4-yl)-6,7-dimethyl-1,2,3,3a-tetrahydro-4H-pyrrolo[2,3-b]quinolin-4-one

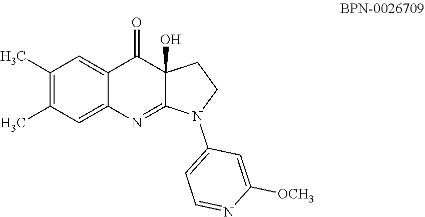 [SMILES: COc1cc(N2CC[C@@]3(O)C(=O)c4cc(C)c(C)cc4N=C23)ccn1]

(S)-3a-Hydroxy-1-(2-methoxypyridin-4-yl)-6,7-dimethyl-1,2,3,3a-tetrahydro-4H-pyrrolo[2,3-b]quinolin-4-one was prepared as a yellow solid according to Synthetic Scheme 4: mp=218-220° C.; ¹H NMR (500 MHz, DMSO-d₆) δ 8.12 (d, J=5.9 Hz, 1H), 7.69 (dd, J=5.9, 2.0 Hz, 1H), 7.59 (d, J=1.8 Hz, 1H), 7.52 (s, 1H), 7.14 (s, 1H), 6.86 (s, 1H), 3.97-3.94 (m, 2H), 3.87 (s, 3H), 2.30 (s, 3H), 2.26-2.23 (m, 5H); ESI MS m/z 338 [C₁₉H₁₉N₃O₃+H]⁺; HPLC (Method B) >99% (AUC), t_{R}=9.04 min; Chiral HPLC (Chiralpak AD, Method A) 90.6% (AUC), t_{R}=15.27 min.

### Preparation of (S)-3a-Hydroxy-6,7-dimethyl-1-(1-methyl-1H-pyrazol-4-yl)-1,2,3,3a-tetrahydro-4H-pyrrolo[2,3-b]quinolin-4-one

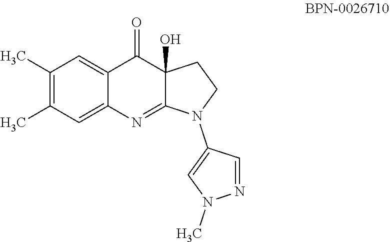 [SMILES: Cc1cc2c(cc1C)C(=O)[C@]1(O)CCN(c3cnn(C)c3)C1=N2]

(S)-3a-Hydroxy-6,7-dimethyl-1-(1-methyl-1H-pyrazol-4-yl)-1,2,3,3a-tetrahydro-4H-pyrrolo[2,3-b]quinolin-4-one was prepared as a yellow solid according to Synthetic Scheme 4: mp=226-227° C.; ¹H NMR (500 MHz, DMSO-d₆) δ 7.25 (s, 1H), 7.81 (s, 1H), 7.44 (s, 1H), 7.03 (s, 1H), 6.69 (s, 1H), 3.92-3.87 (m, 4H), 3.81-3.78 (m, 1H), 2.29-2.22 (m, 5H), 2.20 (s, 3H); ESI MS m/z 311 [C₁₇H₁₈N₄O₂+H]⁺; HPLC (Method B) >99% (AUC), t_{R}=7.50 min; Chiral HPLC (Chiralpak AD, Method A) 92.2% (AUC), t_{R}=18.21 min.

### Preparation of (S)-7-(Benzofuran-6-yl)-4a-hydroxy-2-ethyl-4a,5,6,7-tetrahydro-4H-pyrrolo[2,3-b]thieno[3,2-e]pyridin-4-one

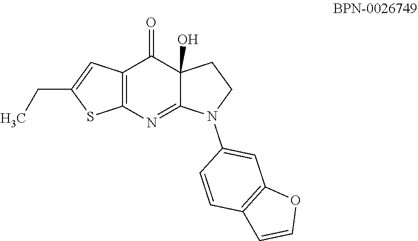 [SMILES: CCc1cc2c(s1)N=C1N(c3ccc4ccoc4c3)CC[C@@]1(O)C2=O]

(S)-7-(Benzofuran-6-yl)-4a-hydroxy-2-ethyl-4a,5,6,7-tetrahydro-4H-pyrrolo[2,3-b]thieno[3,2-e]pyridin-4-one was prepared as a red-orange solid according to Synthetic Scheme 3: mp=233-235° C.; ¹H NMR (500 MHz, DMSO-d₆) δ 8.30 (s, 1H), 8.02 (d, J=2.2 Hz, 1H), 7.81 (dd, J=8.6, 1.9 Hz, 1H), 7.71 (d, J=8.6 Hz, 1H), 6.97 (dd, J=2.0, 0.8 Hz, 1H), 6.91 (s, 1H), 6.82 (s, 1H), 4.26-4.21 (m, 1H), 4.11-4.08 (m, 1H), 2.70 (q, J=7.5, 2H), 2.28-2.20 (m, 2H), 1.22 (t, J=7.5 Hz, 3H); ESI MS m/z 353 [C₁₉H₁₆N₂O₃S+H]⁺; HPLC (Method B) >99% (AUC), t_{R}=10.51 min; Chiral HPLC (Chiralpak AD, Method A) >99% (AUC), t_{R}=21.69 min.

### Preparation of (S)-7-(4-Chlorophenyl)-4a-hydroxy-2-ethyl-4a,5,6,7-tetrahydro-4H-pyrrolo[2,3-b]thieno[3,2-e]pyridin-4-one

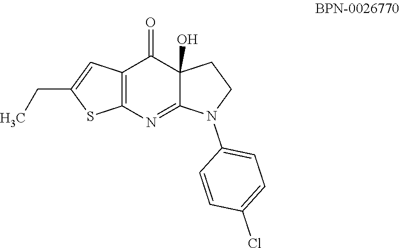 [SMILES: CCc1cc2c(s1)N=C1N(c3ccc(Cl)cc3)CC[C@@]1(O)C2=O]

(S)-7-(4-Chlorophenyl)-4a-hydroxy-2-ethyl-4a,5,6,7-tetrahydro-4H-pyrrolo[2,3-b]thieno[3,2-e]pyridin-4-one was prepared as a red-orange solid according to Synthetic Scheme 3: mp=235-236° C.; ¹H NMR (500 MHz, DMSO-d₆) δ 8.00-7.97 (m, 2H), 7.53-7.50 (m, 2H), 6.91 (s, 1H), 6.82 (apparent t, J=1.0 Hz, 1H), 4.16-4.11 (m, 1H), 4.03-4.00 (m, 1H), 2.70 (q, J=7.4 Hz, 2H), 2.23-2.20 (m, 2H), 1.21 (t, J=7.5 Hz, 3H); ESI MS m/z 347 [C₁₇H₁₅ClN₂O₂S+H]⁺; HPLC (Method B) >99% (AUC), t_{R}=11.20 min; Chiral HPLC (Chiralpak AD, Method A) >99% (AUC), t_{R}=18.88 min.

### Preparation of (S)-2-Ethyl-4a-hydroxy-7-(p-tolyl)-4a,5,6,7-tetrahydro-4H-pyrrolo[2,3-b]thieno[3,2-e]pyridin-4-one

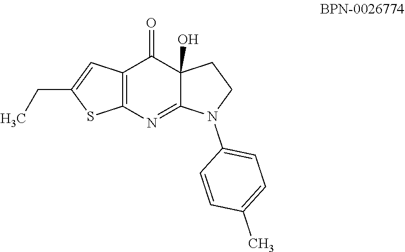 [SMILES: CCc1cc2c(s1)N=C1N(c3ccc(C)cc3)CC[C@@]1(O)C2=O]

(S)-2-Ethyl-4a-hydroxy-7-(p-tolyl)-4a,5,6,7-tetrahydro-4H-pyrrolo[2,3-b]thieno[3,2-e]pyridin-4-one was prepared as a red solid according to Synthetic Scheme 3: mp=248-249° C.; ¹H NMR (500 MHz, DMSO-d₆) δ 7.81-7.79 (m, 2H), 7.26-7.24 (m, 2H), 6.85 (s, 1H), 6.79 (apparent t, J=1.1 Hz, 1H), 4.15-4.10 (m, 1H), 4.01-3.98 (m, 1H), 2.71-2.66 (m, 2H), 2.31 (s, 3H), 2.25-2.15 (m, 2H), 2.21 (t, J=7.5 Hz, 3H); ESI MS m/z 327 [C₁₈H₁₈N₂O₂S+H]⁺; HPLC (Method B) >99% (AUC), t_{R}=10.30 min; Chiral HPLC (Chiralpak AD, Method A) >99% (AUC), t_{R}=18.53 min.

### Preparation of (S)-4a-Hydroxy-7-(4-methoxyphenyl)-2,3-dimethyl-4a,5,6,7-tetrahydro-4H-pyrrolo[2,3-b]thieno[3,2-e]pyridin-4-one

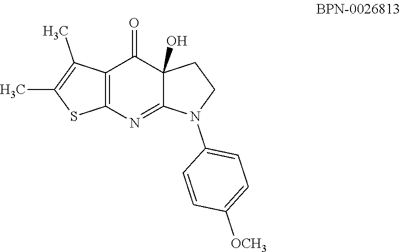 [SMILES: COc1ccc(N2CC[C@@]3(O)C(=O)c4c(sc(C)c4C)N=C23)cc1]

(S)-4a-Hydroxy-7-(4-methoxyphenyl)-2,3-dimethyl-4a,5,6,7-tetrahydro-4H-pyrrolo[2,3-b]thieno[3,2-e]pyridin-4-one was prepared as an orange solid according to Synthetic Scheme 3: mp=232-233° C.; ¹H NMR (500 MHz, DMSO-d₆) δ 7.83-7.81 (m, 2H), 7.02-7.00 (m, 2H), 6.80 (s, 1H), 4.15-4.09 (m, 1H), 3.99-3.95 (m, 1H), 3.77 (s, 3H), 2.20-2.17 (m, 8H); ESI MS m/z 343 [C₁₈H₁₈N₂O₃S+H]⁺; HPLC (Method B) >99% (AUC), t_{R}=9.49 min; Chiral HPLC (Chiralpak AD, Method A) 85.2% (AUC), t_{R}=18.94 min.

### Preparation of (S)-7-(4-Chlorophenyl)-4a-hydroxy-2,3-dimethyl-4a,5,6,7-tetrahydro-4H-pyrrolo[2,3-b]thieno[3,2-e]pyridin-4-one

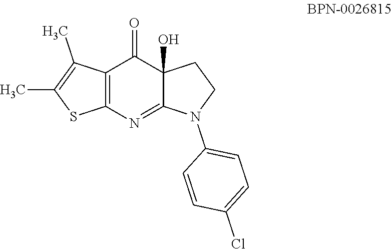 [SMILES: Cc1sc2c(c1C)C(=O)[C@]1(O)CCN(c3ccc(Cl)cc3)C1=N2]

(S)-7-(4-Chlorophenyl)-4a-hydroxy-2,3-dimethyl-4a,5,6,7-tetrahydro-4H-pyrrolo[2,3-b]thieno[3,2-e]pyridin-4-one was prepared as a yellow-orange solid according to Synthetic Scheme 3: mp=257-258° C.; ¹H NMR (500 MHz, DMSO-d₆) δ 7.99-7.98 (m, 2H), 7.51-7.49 (m, 2H), 6.88 (s, 1H), 4.14-4.09 (m, 1H), 4.03-4.00 (m, 1H), 2.22-2.19 (m, 8H); ESI MS m/z 347 [C₁₇H₁₅ClN₂O₂S+H]⁺; HPLC (Method B) >99% (AUC), t_{R}=11.17 min; Chiral HPLC (Chiralpak AD, Method A) 91.2% (AUC), t_{R}=16.03 min.

### Preparation of (S)-4-(2-Ethyl-4a-hydroxy-4-oxo-4,4a,5,6-tetrahydro-7H-pyrrolo[2,3-b]thieno[3,2-e]pyridin-7-yl)-2-methylbenzonitrile

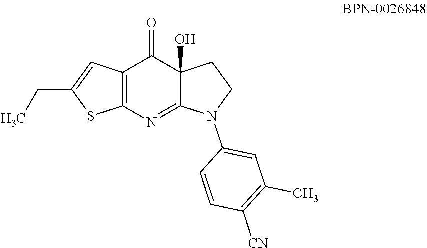 [SMILES: CCc1cc2c(s1)N=C1N(c3ccc(C#N)c(C)c3)CC[C@@]1(O)C2=O]

(S)-4-(2-Ethyl-4a-hydroxy-4-oxo-4,4a,5,6-tetrahydro-7H-pyrrolo[2,3-b]thieno[3,2-e]pyridin-7-yl)-2-methylbenzonitrile was prepared as an orange solid according to Synthetic Scheme 3: ¹H NMR (500 MHz, DMSO-d₆) δ 8.16 (dd, J=8.6, 2.2 Hz, 1H), 7.91 (d, J=1.7 Hz, 1H), 7.83 (d, J=8.7 Hz, 1H), 6.98 (s, 1H), 6.86 (apparent t, J=1.0 Hz, 1H), 4.13-4.06 (m, 2H), 2.74-2.70 (m, 2H), 2.24-2.21 (m, 2H), 1.22 (t, J=7.5 Hz, 3H), 3H obscured by solvent peak; ESI MS m/z 352 [C₁₉H₁₇N₃O₂S+H]⁺; HPLC (Method B) >99% (AUC), t_{R}=11.33 min; Chiral HPLC (Chiralpak AD, Method A) 66.7% (AUC), t_{R}=19.14 min.

### Preparation of (S)-7-(3-Bromophenyl)-4a-hydroxy-2,3-dimethyl-4a,5,6,7-tetrahydro-4H-pyrrolo[2,3-b]thieno[3,2-e]pyridin-4-one

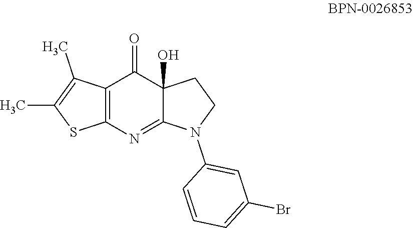 [SMILES: Cc1sc2c(c1C)C(=O)[C@]1(O)CCN(c3cccc(Br)c3)C1=N2]

(S)-7-(3-Bromophenyl)-4a-hydroxy-2,3-dimethyl-4a,5,6,7-tetrahydro-4H-pyrrolo[2,3-b]thieno[3,2-e]pyridin-4-one was prepared as a yellow solid according to Synthetic Scheme 3: mp=259-260° C.; ¹H NMR (500 MHz, DMSO-d₆) δ 8.27 (apparent t, J=1.9 Hz, 1H), 7.90 (ddd, J=8.0, 2.0, 1.3 Hz, 1H), 7.42-7.36 (m, 2H), 6.90 (s, 1H), 4.14-4.09 (m, 1H), 4.04-4.01 (m, 1H), 2.31-2.19 (m, 8H); ESI MS m/z 391 [C₁₇H₁₅BrN₂O₂S+H]⁺; HPLC (Method C) 90.9% (AUC), t_{R}=18.45 min; Chiral HPLC (Chiralpak AD, Method A) 98.9% (AUC), t_{R}=14.50 min.

### Preparation of (S)-2-Ethyl-4a-hydroxy-7-(4-methoxyphenyl)-4a,5,6,7-tetrahydro-4H-pyrrolo[2,3-b]thieno[3,2-e]pyridin-4-one

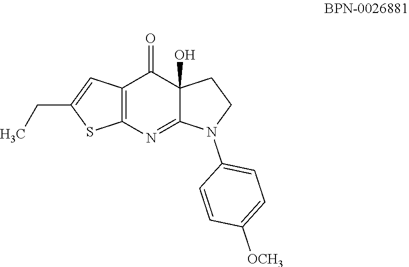 [SMILES: CCc1cc2c(s1)N=C1N(c3ccc(OC)cc3)CC[C@@]1(O)C2=O]

(S)-2-Ethyl-4a-hydroxy-7-(4-methoxyphenyl)-4a,5,6,7-tetrahydro-4H-pyrrolo[2,3-b]thieno[3,2-e]pyridin-4-one was prepared as an orange solid according to Synthetic Scheme 3: mp=228-229° C.; ¹H NMR (500 MHz, DMSO-d₆) δ 7.83-7.80 (m, 2H), 7.03-7.00 (m, 2H), 6.83 (s, 1H), 6.78 (apparent t, J=1.2 Hz, 1H), 4.16-4.11 (m, 1H), 3.99-3.96 (m, 1H), 3.77 (s, 3H), 2.68 (q, J=7.4 Hz, 2H), 2.25-2.15 (m, 2H), 1.20 (t, J=7.5 Hz, 3H); ESI MS m/z 343 [C₁₈H₁₈N₂O₃S+H]⁺; HPLC (Method B) >99% (AUC), t_{R}=10.28 min; Chiral HPLC (Chiralpak AD, Method A) >99% (AUC), t_{R}=22.29 min.

### Preparation of (S)-4-(4a-Hydroxy-2,3-dimethyl-4-oxo-4,4a,5,6-tetrahydro-7H-pyrrolo[2,3-b]thieno[3,2-e]pyridin-7-yl)-2-methylbenzonitrile

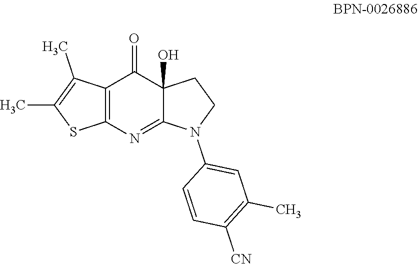

(S)-4-(4a-Hydroxy-2,3-dimethyl-4-oxo-4,4a,5,6-tetrahydro-7H-pyrrolo[2,3-b]thieno[3,2-e]pyridin-7-yl)-2-methylbenzonitrile was prepared as an orange solid according to Synthetic Scheme 3: mp=245-246° C.; ¹H NMR (500 MHz, DMSO-d₆) δ 8.17 (dd, J=8.7, 1.9 Hz, 1H), 7.89 (d, J=1.9 Hz, 1H), 7.82 (d, J=8.6 Hz, 1H), 6.95 (s, 1H), 4.14-4.05 (m, 2H), 2.24-2.21 (m, 8H), 3H obscured by solvent peak; ESI MS m/z 352 [C₁₉H₁₇N₃O₂S+H]⁺; HPLC (Method B) 98.6% (AUC), t_{R}=12.07 min; Chiral HPLC (Chiralpak AD, Method A) 87.8% (AUC), t_{R}=17.22 min.

### Preparation of (S)-4-(2-Ethyl-4a-hydroxy-4-oxo-4,4a,5,6-tetrahydro-7H-pyrrolo[2,3-b]thieno[3,2-e]pyridin-7-yl)benzonitrile

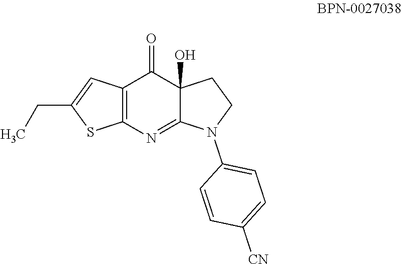 [SMILES: CCc1cc2c(s1)N=C1N(c3ccc(C#N)cc3)CC[C@@]1(O)C2=O]

(S)-4-(2-Ethyl-4a-hydroxy-4-oxo-4,4a,5,6-tetrahydro-7H-pyrrolo[2,3-b]thieno[3,2-e]pyridin-7-yl)benzonitrile was prepared as an orange solid according to Synthetic Scheme 3: mp=238-239° C.; ¹H NMR (500 MHz, DMSO-d₆) δ 8.19-8.17 (m, 2H), 7.92-7.90 (m, 2H), 6.99 (s, 1H), 6.87 (apparent t, J=1.0 Hz, 1H), 4.17-4.08 (m, 2H), 2.75-2.70 (m, 2H), 2.26-2.22 (m, 2H), 1.22 (t, J=7.5 Hz, 3H); ESI MS m/z 338 [C₁₈H₁₅N₃O₂S+H]⁺; HPLC (Method B) >99% (AUC), t_{R}=11.89 min; Chiral HPLC (Chiralpak AD, Method A) >99% (AUC), t_{R}=21.29 min.

### Preparation of (S)-3a-Hydroxy-6-methyl-1-(quinolin-7-yl)-1,2,3,3a-tetrahydro-4H-pyrrolo[2,3-b]quinolin-4-one

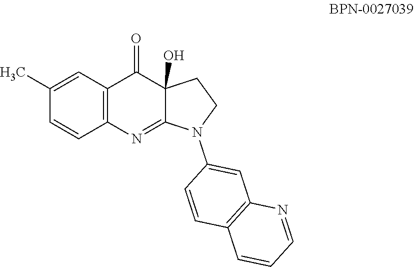 [SMILES: Cc1ccc2c(c1)C(=O)[C@]1(O)CCN(c3ccc4cccnc4c3)C1=N2]

(S)-3a-Hydroxy-6-methyl-1-(quinolin-7-yl)-1,2,3,3a-tetrahydro-4H-pyrrolo[2,3-b]quinolin-4-one was prepared as a yellow solid according to Synthetic Scheme 4: mp=242-243° C.; ¹H NMR (500 MHz, DMSO-d₆) δ 8.89 (dd, J=4.3, 1.8 Hz, 1H), 8.59 (d, J=2.2 Hz, 1H), 8.56 (dd, J=9.0, 2.3 Hz, 1H), 8.33 (dd, J=8.2, 1.1 Hz, 1H), 8.02 (d, J=9.0 Hz, 1H), 7.57 (d, J=1.5 Hz, 1H), 7.46 (dd, J=8.2, 4.2 Hz, 1H), 7.42 (dd, J=8.2, 1.9 Hz, 1H), 7.21 (d, J=8.0 Hz, 1H), 6.91 (s, 1H), 4.23-4.14 (m, 2H), 2.34-2.29 (m, 5H); ESI MS m/z 344 [C₂₁H₁₇N₃O₂+H]⁺; HPLC (Method B) >99% (AUC), t_{R}=10.08 min; Chiral HPLC (Chiralpak AD, Method A) 49.3% (AUC), t_{R}=21.31 min.

### Preparation of (S)-3a-Hydroxy-6-methyl-1-(quinolin-6-yl)-1,2,3,3a-tetrahydro-4H-pyrrolo[2,3-b]quinolin-4-one

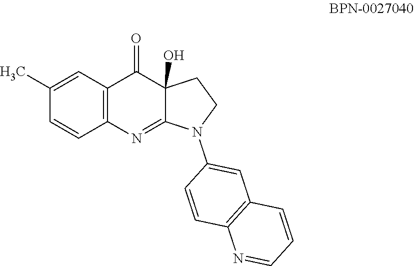 [SMILES: Cc1ccc2c(c1)C(=O)[C@]1(O)CCN(c3ccc4ncccc4c3)C1=N2]

(S)-3a-Hydroxy-6-methyl-1-(quinolin-6-yl)-1,2,3,3a-tetrahydro-4H-pyrrolo[2,3-b]quinolin-4-one was prepared as a yellow solid according to Synthetic Scheme 3: mp=236-238° C.; ¹H NMR (500 MHz, DMSO-d₆) δ 8.83 (dd, J=4.2, 1.7 Hz, 1H), 8.77 (dd, J=9.3, 2.6 Hz, 1H), 8.47 (d, J=2.5 Hz, 1H), 8.36 (dd, J=8.6, 0.9 Hz, 1H), 8.07 (d, J=9.3 Hz, 1H), 7.56 (d, J=1.7 Hz, 1H), 7.53 (dd, J=8.3, 4.2 Hz, 1H), 7.42 (dd, J=8.4, 1.9 Hz, 1H), 7.23 (d, J=8.0 Hz, 1H), 6.91 (s, 1H), 4.21-4.12 (m, 2H), 2.34-2.30 (m, 5H); ESI MS m/z 344 [C₂₁H₁₇N₃O₂+H]⁺; HPLC (Method B) >99% (AUC), t_{R}=6.83 min; Chiral HPLC (Chiralpak AD, Method A) 94.1% (AUC), t_{R}=19.50 min.

### Preparation of (S)-3a-Hydroxy-1-(isoquinolin-7-yl)-6-methyl-1,2,3,3a-tetrahydro-4H-pyrrolo[2,3-b]quinolin-4-one

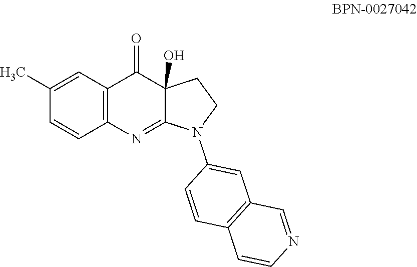 [SMILES: Cc1ccc2c(c1)C(=O)[C@]1(O)CCN(c3ccc4ccncc4c3)C1=N2]

(S)-3a-Hydroxy-1-(isoquinolin-7-yl)-6-methyl-1,2,3,3a-tetrahydro-4H-pyrrolo[2,3-b]quinolin-4-one was prepared as a yellow solid according to Synthetic Scheme 4: mp=217-218° C.; ¹H NMR (500 MHz, DMSO-d₆) δ 9.32 (s, 1H), 8.77 (dd, J=9.1, 2.3 Hz, 1H), 8.63 (d, J=2.0 Hz, 1H), 8.44 (d, J=5.7 Hz, 1H), 8.03 (d, J=9.1 Hz, 1H), 7.80 (d, J=5.7 Hz, 1H), 7.57 (d, J=1.5 Hz, 1H), 7.42 (dd, J=8.2, 1.9 Hz, 1H), 7.24 (d, J=8.1 Hz, 1H), 6.91 (s, 1H), 4.22-4.13 (m, 2H), 2.34-2.31 (m, 5H); ESI MS m/z 344 [C₂₁H₁₇N₃O₂+H]⁺; HPLC (Method B) >99% (AUC), t_{R}=9.72 min; Chiral HPLC (Chiralpak AD, Method A) >99% (AUC), t_{R}=20.95 min.

### Preparation of (S)-4a-Hydroxy-7-(4-iodophenyl)-2,3-dimethyl-4a,5,6,7-tetrahydro-4H-pyrrolo[2,3-b]thieno[3,2-e]pyridin-4-one

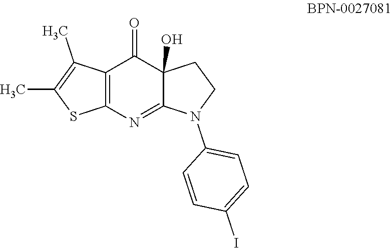 [SMILES: Cc1sc2c(c1C)C(=O)[C@]1(O)CCN(c3ccc(I)cc3)C1=N2]

(S)-4a-Hydroxy-7-(4-iodophenyl)-2,3-dimethyl-4a,5,6,7-tetrahydro-4H-pyrrolo[2,3-b]thieno[3,2-e]pyridin-4-one was prepared as an orange solid according to Synthetic Scheme 3: mp=251-252° C.; ¹H NMR (500 MHz, DMSO-d₆) δ 7.78 (s, 4H), 6.88 (s, 1H), 4.12-4.07 (m, 1H), 4.01-3.96 (m, 1H), 2.22-2.18 (m, 8H); ESI MS m/z 439 [C₁₇H₁₅IN₂O₂S+H]⁺; HPLC (Method B) >99% (AUC), t_{R}=11.15 min; Chiral HPLC (Chiralpak AD, Method A) 89.8% (AUC), t_{R}=17.96 min.

### Preparation of (S)-3a-Hydroxy-l-(4-iodophenyl)-6,7-dimethyl-1,2,3,3a-tetrahydro-4H-pyrrolo[2,3-b]quinolin-4-one

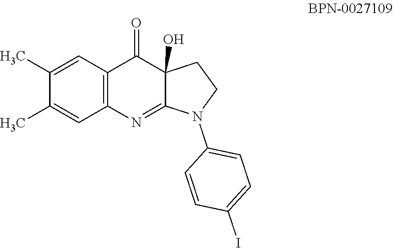 [SMILES: Cc1cc2c(cc1C)C(=O)[C@]1(O)CCN(c3ccc(I)cc3)C1=N2]

(S)-3a-Hydroxy-1-(4-iodophenyl)-6,7-dimethyl-1,2,3,3a-tetrahydro-4H-pyrrolo[2,3-b]quinolin-4-one was prepared as a yellow solid according to Synthetic Scheme 3: mp=240-241° C.; ¹H NMR (500 MHz, DMSO-d₆) δ 7.95-7.93 (m, 2H), 7.75-7.73 (m, 2H), 7.49 (s, 1H), 7.05 (s, 1H), 6.79 (s, 1H), 4.01-3.96 (m, 1H), 3.94-3.89 (m, 1H), 2.27 (s, 3H), 2.25-2.22 (m, 5H); ESI MS m/z 433 [C₁₉H₁₇IN₂O₂+H]⁺; HPLC (Method B) >99% (AUC), t_{R}=8.61 min; Chiral HPLC (Chiralpak AD, Method A) 91.4% (AUC), t_{R}=18.01 min.

### Preparation of (S)-1-(Benzo[d]thiazol-5-yl)-3a-hydroxy-6,7-dimethyl-1,2,3,3a-tetrahydro-4H-pyrrolo[2,3-b]quinolin-4-one

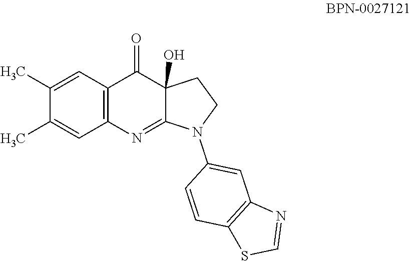 [SMILES: Cc1cc2c(cc1C)C(=O)[C@]1(O)CCN(c3ccc4scnc4c3)C1=N2]

(S)-1-(Benzo[d]thiazol-5-yl)-3a-hydroxy-6,7-dimethyl-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 9.42 (s, 1H), 8.85 (d, J=2.1 Hz, 1H), 8.28 (dd, J=8.9, 2.2 Hz, 1H), 8.19 (d, J=8.9 Hz, 1H), 7.51 (s, 1H), 7.10 (s, 1H), 6.82 (s, 1H), 4.19-4.14 (m, 1H), 4.09-4.06 (m, 1H), 2.30-2.28 (m, 5H), 2.23 (s, 3H); ESI MS m/z 364 [C₂₀H₁₇N₃O₂S+H]⁺; HPLC (Method B) 95.3% (AUC), t_{R}=8.01 min; Chiral HPLC (Chiralpak AD, Method A) 70.3% (AUC), t_{R}=27.20 min.

### Preparation of (S)-1-(Benzo[d]thiazol-6-yl)-3a-hydroxy-6,7-dimethyl-1,2,3,3a-tetrahydro-4H-pyrrolo[2,3-b]quinolin-4-one

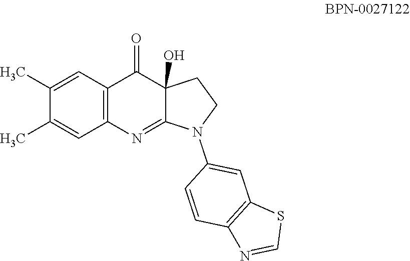 [SMILES: Cc1cc2c(cc1C)C(=O)[C@]1(O)CCN(c3ccc4ncsc4c3)C1=N2]

(S)-1-(Benzo[d]thiazol-6-yl)-3a-hydroxy-6,7-dimethyl-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 9.32 (s, 1H), 8.90 (d, J=2.3 Hz, 1H), 8.35 (dd, J=9.0, 2.3 Hz, 1H), 8.12 (d, J=9.0 Hz, 1H), 7.51 (s, 1H), 7.13 (s, 1H), 6.82 (s, 1H), 4.17-4.12 (m, 1H), 4.07-4.04 (m, 1H), 2.30-2.29 (m, 5H), 2.23 (s, 3H); ESI MS m/z 364 [C₂₀H₁₇N₃O₂S+H]⁺; HPLC (Method B) 97.5% (AUC), t_{R}=7.92 min; Chiral HPLC (Chiralpak AD, Method A) 83.3% (AUC), t_{R}=21.14 min.

### Preparation of (S)-3a-Hydroxy-6-methyl-1-(3-methylquinolin-6-yl)-1,2,3,3a-tetrahydro-4H-pyrrolo[2,3-b]quinolin-4-one

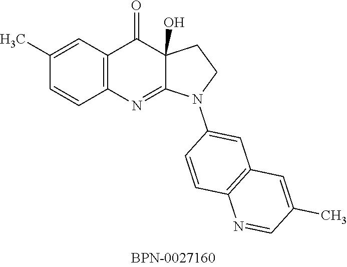

(S)-3a-Hydroxy-6-methyl-1-(3-methylquinolin-6-yl)-1,2,3,3a-tetrahydro-4H-pyrrolo[2,3-b]quinolin-4-one was prepared as a yellow solid according to Synthetic Scheme 4: mp=263-264° C.; ¹H NMR (500 MHz, DMSO-d₆) δ 8.69 (d, J=2.1 Hz, 1H), 8.63 (dd, J=9.3, 2.5 Hz, 1H), 8.43 (d, J=2.5 Hz, 1H), 8.12 (s, 1H), 8.01 (d, J=9.3 Hz, 1H), 7.56 (d, J=1.6 Hz, 1H), 7.41 (dd, J=8.4, 2.0 Hz, 1H), 7.22 (d, J=8.1 Hz, 1H), 6.89 (s, 1H), 4.20-4.10 (m, 2H), 2.32-2.31 (m, 5H), 3H obscured by solvent peak; ESI MS m/z 358 [C₂₂H₁₉N₃O₂+H]⁺; HPLC (Method C) 97.1% (AUC), t_{R}=11.24 min; Chiral HPLC (Chiralpak AD, Method A) 81.9% (AUC), t_{R}=18.76 min.

### Preparation of (S)-3a-Hydroxy-6,7-dimethyl-1-(3-methylisoquinolin-6-yl)-1,2,3,3a-tetrahydro-4H-pyrrolo[2,3-b]quinolin-4-one

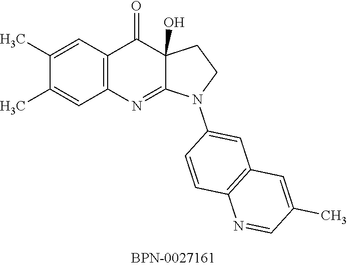

(S)-3a-Hydroxy-6,7-dimethyl-1-(3-methylisoquinolin-6-yl)-1,2,3,3a-tetrahydro-4H-pyrrolo[2,3-b]quinolin-4-one was prepared as a yellow solid according to Synthetic Scheme 4: mp=265-267° C.; ¹H NMR (500 MHz, DMSO-d₆) δ 8.69 (d, J=2.1 Hz, 1H), 8.63 (dd, J=9.3, 2.6 Hz, 1H), 8.43 (d, J=2.5 Hz, 1H), 8.12 (s, 1H), 8.01 (d, J=9.2 Hz, 1H), 7.51 (s, 1H), 7.14 (s, 1H), 6.84 (s, 1H), 4.19-4.09 (m, 2H), 2.32-2.30 (m, 5H), 2.24 (s, 3H), 3H obscured by solvent peak; ESI MS m/z 372 [C₂₃H₂₁N₃O₂+H]⁺; HPLC (Method B) >99% (AUC), t_{R}=7.61 min; Chiral HPLC (Chiralpak AD, Method A) 73.7% (AUC), t_{R}=19.44 min.

### Preparation of (S)-3a-Hydroxy-6,7-dimethyl-1-(quinolin-6-yl)-1,2,3,3a-tetrahydro-4H-pyrrolo[2,3-b]quinolin-4-one

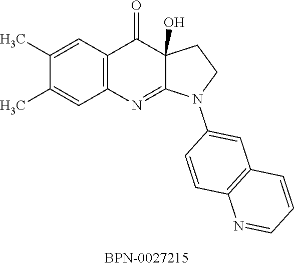 [SMILES: Cc1cc2c(cc1C)C(=O)[C@]1(O)CCN(c3ccc4ncccc4c3)C1=N2]

(S)-3a-Hydroxy-6,7-dimethyl-1-(quinolin-6-yl)-1,2,3,3a-tetrahydro-4H-pyrrolo[2,3-b]quinolin-4-one was prepared as a yellow solid according to Synthetic Scheme 4: mp=246-247° C.; ¹H NMR (500 MHz, DMSO-d₆) δ 8.83 (dd, J=4.2, 1.7 Hz, 1H), 8.79 (dd, J=9.3, 2.6 Hz, 1H), 8.45 (d, J=2.5 Hz, 1H), 8.36 (d, J=7.6 Hz, 1H), 8.06 (d, J=9.3 Hz, 1H), 7.54-7.52 (m, 2H), 7.15 (s, 1H), 6.85 (s, 1H), 4.20-4.09 (m, 2H), 2.33-2.30 (m, 5H), 2.24 (s, 3H); ESI MS m/z 358 [C₂₂H₁₉N₃O₂+H]⁺; HPLC (Method B) >97.9% (AUC), t_{R}=8.10 min; Chiral HPLC (Chiralpak AD, Method A) 61.1% (AUC), t_{R}=20.30 min.

### Preparation of (S)-4a-Hydroxy-2-methyl-7-(quinolin-6-yl)-4a,5,6,7-tetrahydro-4H-pyrrolo[2,3-b]thieno[3,2-e]pyridin-4-one

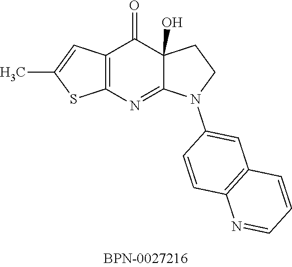 [SMILES: Cc1cc2c(s1)N=C1N(c3ccc4ncccc4c3)CC[C@@]1(O)C2=O]

(S)-4a-Hydroxy-2-methyl-7-(quinolin-6-yl)-4a,5,6,7-tetrahydro-4H-pyrrolo[2,3-b]thieno[3,2-e]pyridin-4-one was prepared as a red-orange solid according to Synthetic Scheme 3: mp=242-243° C.; ¹H NMR (500 MHz, DMSO-d₆) δ 8.86 (dd, J=4.2, 1.6 Hz, 1H), 8.60 (dd, J=9.3, 2.6 Hz, 1H), 8.37 (d, J=7.6 Hz, 1H), 8.31 (d, J=2.5 Hz, 1H), 8.09 (d, J=9.3 Hz, 1H), 7.55 (dd, J=8.3, 4.2 Hz, 1H), 6.97 (s, 1H), 6.82 (d, J=1.3 Hz, 1H), 4.30-4.25 (m, 1H), 4.21-4.18 (m, 1H), 2.37-2.36 (m, 3H), 2.30-2.27 (m, 2H); ESI MS m/z 350 [C₁₉H₁₅N₃O₂S+H]⁺; HPLC (Method C) 98.7% (AUC), t_{R}=11.45 min; Chiral HPLC (Chiralpak AD, Method A) >99% (AUC), t_{R}=21.33 min.

### Preparation of (S)-2-Ethyl-4a-hydroxy-7-(quinolin-6-yl)-4a,5,6,7-tetrahydro-4H-pyrrolo[2,3-b]thieno[3,2-e]pyridin-4-one

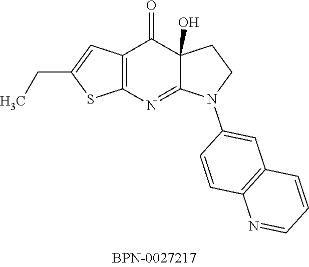 [SMILES: CCc1cc2c(s1)N=C1N(c3ccc4ncccc4c3)CC[C@@]1(O)C2=O]

(S)-4a-Hydroxy-2-methyl-7-(quinolin-6-yl)-4a,5,6,7-tetrahydro-4H-pyrrolo[2,3-b]thieno[3,2-e]pyridin-4-one was prepared as an orange-brown solid according to Synthetic Scheme 3: mp=217-219° C.; ¹H NMR (500 MHz, DMSO-d₆) δ 8.86 (dd, J=4.2, 1.6 Hz, 1H), 8.59 (dd, J=9.3, 2.6 Hz, 1H), 8.37 (d, J=8.5 Hz, 1H), 8.32 (d, J=2.4 Hz, 1H), 8.09 (d, J=9.3 Hz, 1H), 7.55 (dd, J=8.3, 4.2 Hz, 1H), 6.97 (s, 1H), 6.85 (s, 1H), 4.30-4.25 (m, 1H), 4.21-4.18 (m, 1H), 2.72 (q, J=7.5 Hz, 2H), 2.31-2.27 (m, 2H), 2.23 (t, J=7.5 Hz, 3H); ESI MS m/z 364 [C₂₀H₁₇N₃O₂S+H]⁺; HPLC (Method C) >99% (AUC), t_{R}=12.26 min; Chiral HPLC (Chiralpak AD, Method A) >99% (AUC), t_{R}=20.87 min.

### Preparation of (S)-4-(3a-Hydroxy-4-oxo-2,3,3a,4,5,6,7,8-octahydro-1H-benzo[4,5]thieno[2,3-b]pyrrolo[3,2-e]pyridin-1-yl)benzonitrile

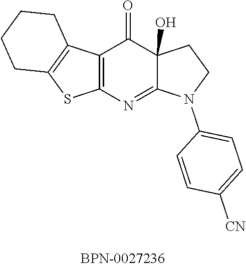 [SMILES: N#Cc1ccc(N2CC[C@@]3(O)C(=O)c4c(sc5c4CCCC5)N=C23)cc1]

(S)-4-(3a-Hydroxy-4-oxo-2,3,3a,4,5,6,7,8-octahydro-1H-benzo[4,5]thieno[2,3-b]pyrrolo[3,2-e]pyridin-1-yl)benzonitrile was prepared as a yellow-brown solid according to Synthetic Scheme 3: ¹H NMR (500 MHz, DMSO-d₆) δ 8.18-8.16 (m, 2H), 7.92-7.90 (m, 2H), 6.96 (s, 1H), 4.16-4.08 (m, 2H), 2.24-2.21 (m, 3H), 1.84-1.65 (m, 7H); ESI MS m/z 364 [C₂₀H₁₇N₃O₂S+H]⁺; HPLC (Method B) 95.4% (AUC), t_{R}=10.52 min; Chiral HPLC (Chiralpak AD, Method A) 55.9% (AUC), t_{R}=19.88 min.

### Preparation of (S)-2-Chloro-4-(4a-hydroxy-2,3-dimethyl-4-oxo-4,4a,5,6-tetrahydro-7H-pyrrolo[2,3-b]thieno[3,2-e]pyridin-7-yl)benzonitrile

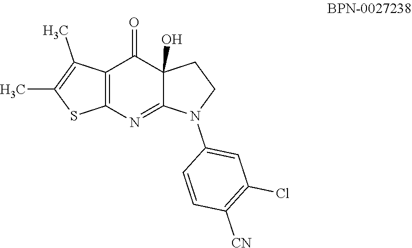 [SMILES: Cc1sc2c(c1C)C(=O)[C@]1(O)CCN(c3ccc(C#N)c(Cl)c3)C1=N2]

(S)-2-Chloro-4-(4a-hydroxy-2,3-dimethyl-4-oxo-4,4a,5,6-tetrahydro-7H-pyrrolo[2,3-b]thieno[3,2-e]pyridin-7-yl)benzonitrile was prepared as an orange solid according to Synthetic Scheme 3: mp=275-276° C.; ¹H NMR (500 MHz, DMSO-d₆) δ 8.38 (d, J=2.2 Hz, 1H), 8.13 (dd, J=8.8, 2.2 Hz, 1H), 8.02 (d, J=8.8 Hz, 1H), 7.01 (s, 1H), 4.13-4.10 (m, 2H), 2.26 (s, 3H), 2.14-2.22 (m, 5H); ESI MS m/z 372 [C₁₈H₁₄ClN₃O₂S+H]⁺; HPLC (Method B) 96.7% (AUC), t_{R}=10.69 min; Chiral HPLC (Chiralpak AD, Method A) 90.8% (AUC), t_{R}=20.01 min.

### Preparation of (S)-3a-Hydroxy-1-(quinolin-6-yl)-1,2,3,3a,5,6,7,8-octahydro-4H-benzo[4,5]thieno[2,3-b]pyrrolo[3,2-e]pyridin-4-one

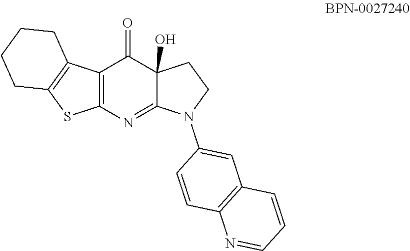 [SMILES: O=C1c2c(sc3c2CCCC3)N=C2N(c3ccc4ncccc4c3)CC[C@@]12O]

(S)-3a-Hydroxy-1-(quinolin-6-yl)-1,2,3,3a,5,6,7,8-octahydro-4H-benzo[4,5]thieno[2,3-b]pyrrolo[3,2-e]pyridin-4-one was prepared as an orange solid according to Synthetic Scheme 3: mp=245-246° C.; ¹H NMR (500 MHz, DMSO-d₆) δ 8.86 (dd, J=4.2, 1.7 Hz, 1H), 8.62 (dd, J=9.3, 2.6 Hz, 1H), 8.37 (dd, J=8.3, 1.1 Hz, 1H), 8.30 (d, J=2.6 Hz, 1H), 8.09 (d, J=9.2 Hz, 1H), 7.54 (dd, J=8.3, 4.2 Hz, 1H), 6.95 (s, 1H), 4.29-4.24 (m, 1H), 4.21-4.18 (m, 1H), 2.65-2.57 (m, 4H), 2.31-2.25 (m, 2H), 1.79-1.70 (m, 4H); ESI MS m/z 390 [C₂₂H₁₉N₃O₂S+H]⁺; HPLC (Method B) 97.8% (AUC), t_{R}=9.06 min; Chiral HPLC (Chiralpak AD, Method A) 82.3% (AUC), t_{R}=19.47 min.

### Preparation of (S)-2-Chloro-4-(4a-hydroxy-2-methyl-4-oxo-4,4a,5,6-tetrahydro-7H-pyrrolo[2,3-b]thieno[3,2-e]pyridin-7-yl)benzonitrile

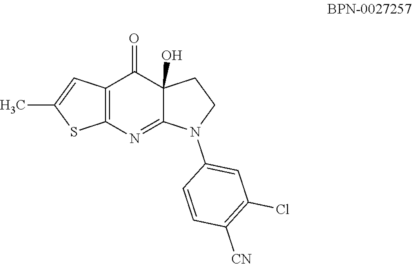 [SMILES: Cc1cc2c(s1)N=C1N(c3ccc(C#N)c(Cl)c3)CC[C@@]1(O)C2=O]

(S)-2-Chloro-4-(4a-hydroxy-2-methyl-4-oxo-4,4a,5,6-tetrahydro-7H-pyrrolo[2,3-b]thieno[3,2-e]pyridin-7-yl)benzonitrile was prepared as an orange solid according to Synthetic Scheme 3: ¹H NMR (500 MHz, DMSO-d₆) δ 8.37 (d, J=2.2 Hz, 1H), 8.15 (dd, J=8.8, 2.2 Hz, 1H), 8.02 (d, J=8.8 Hz, 1H), 7.03 (s, 1H), 6.87 (d, J=1.3 Hz, 1H), 4.14-4.10 (m, 2H), 2.38 (d, J=1.2 Hz, 3H), 2.25-2.22 (m, 2H); ESI MS m/z 358 [C₁₇H₁₂ClN₃O₂S+H]⁺; HPLC (Method C) 98.3% (AUC), t_{R}=16.02 min; Chiral HPLC (Chiralpak AD, Method A) 96.7% (AUC), t_{R}=20.06 min.

### Preparation of (S)-4a-Hydroxy-2,3-dimethyl-7-(quinolin-6-yl)-4a,5,6,7-tetrahydro-4H-pyrrolo[2,3-b]thieno[3,2-e]pyridin-4-one

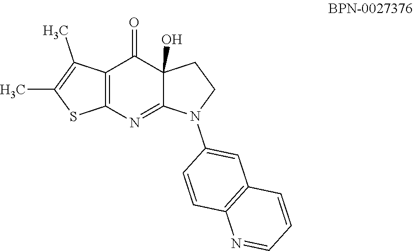 [SMILES: Cc1sc2c(c1C)C(=O)[C@]1(O)CCN(c3ccc4ncccc4c3)C1=N2]

(S)-4a-Hydroxy-2,3-dimethyl-7-(quinolin-6-yl)-4a,5,6,7-tetrahydro-4H-pyrrolo[2,3-b]thieno[3,2-e]pyridin-4-one was prepared as an orange-brown solid according to Synthetic Scheme 3: mp=219-224° C. ¹H NMR (500 MHz, DMSO-d₆) δ 8.85 (dd, J=4.1, 1.6 Hz, 1H), 8.61 (dd, J=9.1, 2.6 Hz, 1H), 8.37 (d, J=8.6 Hz, 1H), 8.30 (d, J=2.7 Hz, 1H), 8.09 (d, J=9.5 Hz, 1H), 7.54 (dd, J=8.3, 4.2 Hz, 1H), 6.94 (s, 1H), 4.28-4.23 (m, 1H), 4.21-4.18 (m, 1H), 2.25-2.23 (m, 8H); ESI MS m/z 364 [C₂₀H₁₇N₃O₂S+H]⁺; HPLC (Method C) 95.8% (AUC), t_{R}=12.69 min; Chiral HPLC (Chiralpak AD, Method A) 89.5% (AUC), t_{R}=19.48 min.

### Preparation of (S)-6-(3a-Hydroxy-6-methyl-4-oxo-2,3,3a,4-tetrahydro-1H-pyrrolo[2,3-b]quinolin-1-yl)quinoline-2-carbonitrile

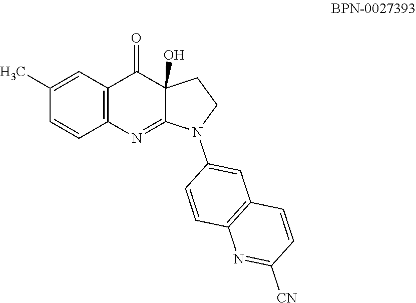 [SMILES: Cc1ccc2c(c1)C(=O)[C@]1(O)CCN(c3ccc4nc(C#N)ccc4c3)C1=N2]

(S)-6-(3a-Hydroxy-6-methyl-4-oxo-2,3,3a,4-tetrahydro-1H-pyrrolo[2,3-b]quinolin-1-yl)quinoline-2-carbonitrile was prepared as a yellow-orange solid according to Synthetic Scheme 6: mp=271-273° C.; ¹H NMR (500 MHz, DMSO-d₆) δ 9.01 (dd, J=9.4, 2.5 Hz, 1H), 8.64-8.60 (m, 2H), 8.19 (d, J=9.4 Hz, 1H), 8.01 (d, J=8.4 Hz, 1H), 7.58 (d, J=1.3 Hz, 1H), 7.45 (dd, J=8.0, 2.1 Hz, 1H), 7.29 (d, J=8.0 Hz, 1H), 6.96 (s, 1H), 4.19-4.16 (m, 2H), 2.35-2.31 (m, 5H); ESI MS m/z 369 [C₂₂H₁₆N₄O₂+H]⁺; HPLC (Method C) 99.0% (AUC), t_{R}=13.28 min; Chiral HPLC (Chiralpak AD, Method A) 96.3% (AUC), t_{R}=22.01 min.

### Preparation of (S)-1-(Benzo[c][1,2,5]thiadiazol-5-yl)-3a-hydroxy-6-methyl-1,2,3,3a-tetrahydro-4H-pyrrolo[2,3-b]quinolin-4-one

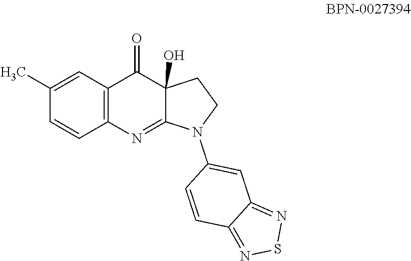 [SMILES: Cc1ccc2c(c1)C(=O)[C@]1(O)CCN(c3ccc4nsnc4c3)C1=N2]

(S)-1-(Benzo[c][1,2,5]thiadiazol-5-yl)-3a-hydroxy-6-methyl-1,2,3,3a-tetrahydro-4H-pyrrolo[2,3-b]quinolin-4-one was prepared as an orange-yellow solid according to Synthetic Scheme 6: mp=231-232° C.; ¹H NMR (500 MHz, DMSO-d₆) δ 8.80 (dd, J=9.7, 2.2 Hz, 1H), 8.60 (d, J=2.1 Hz, 1H), 8.13 (d, J=9.6 Hz, 1H), 7.58 (d, J=1.7 Hz, 1H), 7.43 (dd, J=8.2, 1.9 Hz, 1H), 7.23 (d, J=8.0 Hz, 1H), 6.95 (s, 1H), 4.23-4.13 (m, 2H), 2.34-2.29 (m, 5H); ESI MS m/z 351 [C₁₈H₁₄N₄O₂S+H]⁺; HPLC (Method C) 98.8% (AUC), t_{R}=12.54 min; Chiral HPLC (Chiralpak AD, Method A) >99% (AUC), t_{R}=18.44 min.

### Preparation of (S)-3a-Hydroxy-6-methyl-1-(thieno[2,3-b]pyridin-5-yl)-1,2,3,3a-tetrahydro-4H-pyrrolo[2,3-b]quinolin-4-one

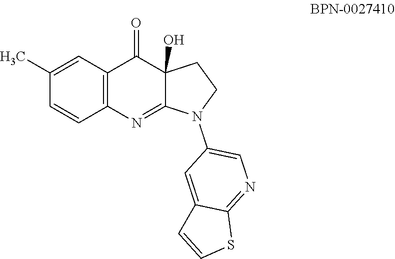 [SMILES: Cc1ccc2c(c1)C(=O)[C@]1(O)CCN(c3cnc4sccc4c3)C1=N2]

(S)-3a-Hydroxy-6-methyl-1-(thieno[2,3-b]pyridin-5-yl)-1,2,3,3a-tetrahydro-4H-pyrrolo[2,3-b]quinolin-4-one was prepared as a yellow solid according to Synthetic Scheme 6: mp=208-210° C.; ¹H NMR (500 MHz, DMSO-d₆) δ 9.33 (d, J=2.5 Hz, 1H), 8.94 (d, J=2.5 Hz, 1H), 7.93 (d, J=5.9 Hz, 1H), 7.56 (d, J=1.7 Hz, 1H), 7.50 (d, J=5.9 Hz, 1H), 7.41 (dd, J=8.1, 2.1 Hz, 1H), 7.18 (d, J=8.1 Hz, 1H), 6.91 (s, 1H), 4.20-4.15 (m, 1H), 4.09-4.06 (m, 1H), 2.34-2.26 (m, 5H); ESI MS m/z 350 [C₁₉H₁₅N₃O₂S+H]⁺; UPLC (Method A) 97.9% (AUC), t_{R}=2.96 min; Chiral HPLC (Chiralpak AD, Method A) 96.8% (AUC), t_{R}=19.56 min.

### Preparation of (S)-3a-Hydroxy-6-methyl-1-(2-methylbenzo[d]thiazol-5-yl)-1,2,3,3a-tetrahydro-4H-pyrrolo[2,3-b]quinolin-4-one

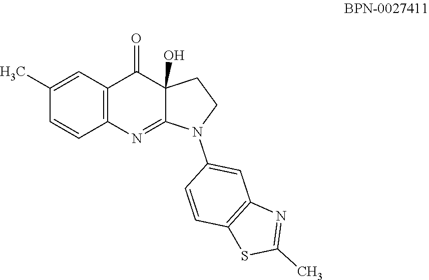

(S)-3a-Hydroxy-6-methyl-1-(2-methylbenzo[d]thiazol-5-yl)-1,2,3,3a-tetrahydro-4H-pyrrolo[2,3-b]quinolin-4-one was prepared as a yellow solid according to Synthetic Scheme 6: mp=258-259° C.; ¹H NMR (500 MHz, DMSO-d₆) δ 8.71 (d, J=2.1 Hz, 1H), 8.12 (dd, J=8.9, 2.2 Hz, 1H), 8.04 (d, J=8.8 Hz, 1H), 7.55 (d, J=1.3 Hz, 1H), 7.39 (dd, J=8.2, 1.9 Hz, 1H), 7.16 (d, J=8.1 Hz, 1H), 6.87 (s, 1H), 4.18-4.13 (m, 1H), 4.06-4.03 (m, 1H), 2.81 (s, 3H), 2.31-2.26 (m, 5H); ESI MS m/z 364 [C₂₀H₁₇N₃O₂S+H]⁺; UPLC (Method A) >99% (AUC), t_{R}=3.00 min; Chiral HPLC (Chiralpak AD, Method A) 74.7% (AUC), t_{R}=21.78 min.

### Preparation of (S)-1-(3,4-Dihydro-2H-benzo[b][1,4]oxazin-7-yl)-3a-hydroxy-6-methyl-1,2,3,3a-tetrahydro-4H-pyrrolo[2,3-b]quinolin-4-one

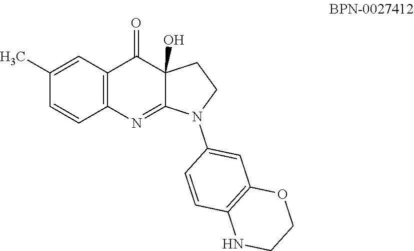 [SMILES: Cc1ccc2c(c1)C(=O)[C@]1(O)CCN(c3ccc4c(c3)OCCN4)C1=N2]

(S)-1-(3,4-Dihydro-2H-benzo[b][1,4]oxazin-7-yl)-3a-hydroxy-6-methyl-1,2,3,3a-tetrahydro-4H-pyrrolo[2,3-b]quinolin-4-one was prepared as an orange solid according to Synthetic Scheme 4: mp=207-208° C.; ¹H NMR (500 MHz, DMSO-d₆) δ 7.53 (d, J=2.4 Hz, 1H), 7.48 (d, J=1.6 Hz, 1H), 7.32 (dd, J=8.1, 2.0 Hz, 1H), 7.21 (dd, J=8.7, 2.5 Hz, 1H), 7.02 (d, J=8.1 Hz, 1H), 6.71 (s, 1H), 6.58 (d, J=8.6 Hz, 1H), 5.73 (s, 1H), 4.15-4.13 (m, 2H), 3.99-3.94 (m, 1H), 3.85-3.81 (m, 1H), 2.28 (s, 3H), 2.21-2.18 (m, 2H), 2H obscured by solvent peak; ESI MS m/z 350 [C₂₀H₁₉N₃O₃+H]⁺; UPLC (Method A) 96.6% (AUC), t_{R}=2.96 min; Chiral HPLC (Chiralpak AD, Method A) >99% (AUC), t_{R}=23.12 min.

### Preparation of (S)-1-([1,2,4]Triazolo[1,5-a]pyridin-7-yl)-3a-hydroxy-6-methyl-1,2,3,3a-tetrahydro-4H-pyrrolo[2,3-b]quinolin-4-one

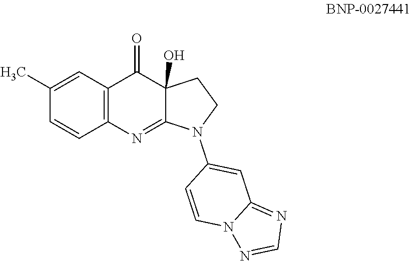 [SMILES: Cc1ccc2c(c1)C(=O)[C@]1(O)CCN(c3ccn4ncnc4c3)C1=N2]

(S)-1-([1,2,4]Triazolo[1,5-a]pyridin-7-yl)-3a-hydroxy-6-methyl-1,2,3,3a-tetrahydro-4H-pyrrolo[2,3-b]quinolin-4-one was prepared as a yellow solid according to Synthetic Scheme 6: mp=260-261° C.; ¹H NMR (500 MHz, DMSO-d₆) δ 8.95 (d, J=7.7 Hz, 1H), 8.43 (s, 1H), 8.35 (d, J=2.2 Hz, 1H), 8.29 (dd, J=7.6, 2.4 Hz, 1H), 7.59 (d, J=1.6 Hz, 1H), 7.45 (dd, J=8.3, 2.0 Hz, 1H), 7.25 (d, J=8.0 Hz, 1H), 6.97 (s, 1H), 4.15-4.10 (m, 2H), 2.35-2.27 (m, 5H); ESI MS m/z 334 [C₁₈H₁₅N₅O₂+H]⁺; UPLC (Method A) 98.0% (AUC), t_{R}=2.85 min; Chiral HPLC (Chiralpak AD, Method A) 64.4% (AUC), t_{R}=20.26 min.

### Preparation of (S)-1-(2-Aminoquinolin-6-yl)-3a-hydroxy-6-methyl-1,2,3,3a-tetrahydro-4H-pyrrolo[2,3-b]quinolin-4-one

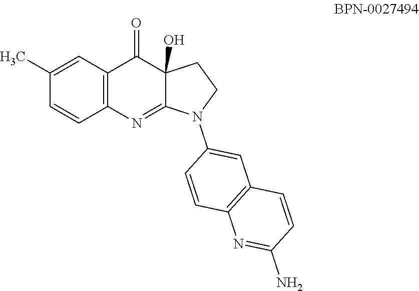 [SMILES: Cc1ccc2c(c1)C(=O)[C@]1(O)CCN(c3ccc4nc(N)ccc4c3)C1=N2]

(S)-1-(2-Aminoquinolin-6-yl)-3a-hydroxy-6-methyl-1,2,3,3a-tetrahydro-4H-pyrrolo[2,3-b]quinolin-4-one was prepared as a yellow-orange solid according to Synthetic Scheme 6: mp=261-262° C.; ¹H NMR (500 MHz, DMSO-d₆) δ 8.36 (dd, J=9.1, 2.6 Hz, 1H), 8.14 (d, J=2.5 Hz, 1H), 7.90 (d, J=8.9 Hz, 1H), 7.53 (d, J=2.0 Hz, 1H), 7.49 (d, J=9.2 Hz, 1H), 7.37 (dd, J=8.5, 1.8 Hz, 1H), 7.13 (d, J=8.1 Hz, 1H), 6.82 (s, 1H), 6.78 (d, J=8.9 Hz, 1H), 6.38 (s, 2H), 4.15-4.10 (m, 1H), 4.04-4.01 (m, 1H), 2.30-2.27 (m, 5H); ESI MS m/z 359 [C₂₁H₁₈N₄O₂+H]⁺; UPLC (Method A) >99% (AUC), t_{R}=2.42 min.

### Preparation of (S)-3-Chloro-5-(3a-hydroxy-6-methyl-4-oxo-2,3,3a,4-tetrahydro-1H-pyrrolo[2,3-b]quinolin-1-yl)picolinonitrile

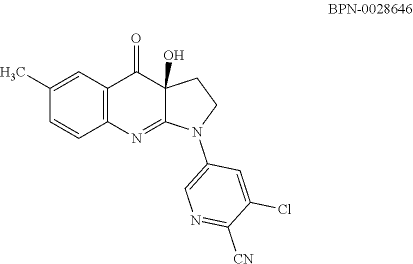 [SMILES: Cc1ccc2c(c1)C(=O)[C@]1(O)CCN(c3cnc(C#N)c(Cl)c3)C1=N2]

(S)-3-Chloro-5-(3a-hydroxy-6-methyl-4-oxo-2,3,3a,4-tetrahydro-1H-pyrrolo[2,3-b]quinolin-1-yl)picolinonitrile was prepared as a yellow solid according to Synthetic Scheme 6: mp=243-245° C.; ¹H NMR (500 MHz, DMSO-d₆) δ 9.42 (d, J=2.3 Hz, 1H), 9.01 (d, J=2.3 Hz, 1H), 7.60 (d, J=1.5 Hz, 1H), 7.46 (ddd, J=8.0, 2.2, 0.7 Hz, 1H), 7.28 (d, J=8.1 Hz, 1H), 7.02 (s, 1H), 4.14-4.04 (m, 2H), 2.34-2.29 (m, 5H); ESI MS m/z 352 [C₁₈H₁₃ClN₄O₂+H]⁺; UPLC (Method A) >99% (AUC), t_{R}=4.64 min; Chiral HPLC (Chiralpak AD, Method A) >99% (AUC), t_{R}=17.22 min.

### Preparation of (S)-1-(3-Aminoquinolin-6-yl)-3a-hydroxy-6-methyl-1,2,3,3a-tetrahydro-4H-pyrrolo[2,3-b]quinolin-4-one

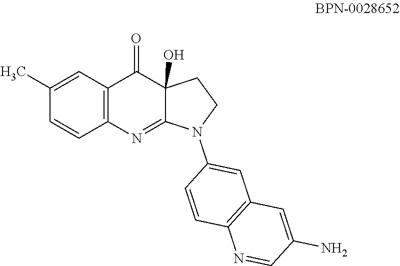 [SMILES: Cc1ccc2c(c1)C(=O)[C@]1(O)CCN(c3ccc4ncc(N)cc4c3)C1=N2]

(S)-1-(3-Aminoquinolin-6-yl)-3a-hydroxy-6-methyl-1,2,3,3a-tetrahydro-4H-pyrrolo[2,3-b]quinolin-4-one was prepared as a yellow solid according to Synthetic Scheme 6: mp=243-244° C.; ¹H NMR (500 MHz, DMSO-d₆) δ 8.36 (d, J=2.7 Hz, 1H), 8.28 (dd, J=9.2, 2.5 Hz, 1H), 8.08 (d, J=2.5 Hz, 1H), 7.79 (d, J=9.2 Hz, 1H), 7.55 (d, J=1.8 Hz, 1H), 7.39 (dd, J=8.1, 2.2 Hz, 1H), 7.16 (d, J=8.1 Hz, 1H), 7.12 (d, J=2.6 Hz, 1H), 6.85 (s, 1H), 5.66 (s, 2H), 4.17-4.12 (m, 1H), 4.09-4.05 (m, 1H), 2.31-2.28 (m, 5H); ESI MS m/z 358 [C₂₁H₁₈N₄O₂+H]⁺; UPLC (Method A) >99% (AUC), t_{R}=2.44 min; Chiral HPLC (Chiralpak AD, Method A) >99% (AUC), t_{R}=21.10 min.

### Preparation of (S)-1-(5,6-Dimethylpyridin-3-yl)-3a-hydroxy-6,7-dimethyl-1,2,3,3a-tetrahydro-4H-pyrrolo[2,3-b]quinolin-4-one

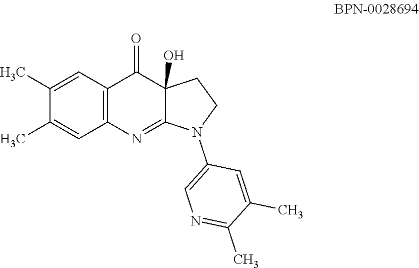

(S)-1-(5,6-Dimethylpyridin-3-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=250-251° C.; ¹H NMR (500 MHz, DMSO-d₆) δ 8.96 (d, J=2.5 Hz, 1H), 8.23 (d, J=2.3 Hz, 1H), 7.48 (s, 1H), 7.05 (s, 1H), 6.79 (s, 1H), 4.05-4.00 (m, 1H), 3.99-3.93 (m, 1H), 2.42 (s, 3H), 2.30 (s, 3H), 2.27-2.22 (m, 8H); ESI MS m/z 336 [C₂₀H₂₁N₃O₂+H]⁺; UPLC (Method A) 98.4% (AUC), t_{R}=3.01 min; Chiral HPLC (Chiralpak AD, Method A) 82.0% (AUC), t_{R}=15.59 min.

### Preparation of (S)-1-(2,3-Dihydro-1H-pyrido[2,3-b][1,4]oxazin-7-yl)-3a-hydroxy-6,7-dimethyl-1,2,3,3a-tetrahydro-4H-pyrrolo[2,3-b]quinolin-4-one

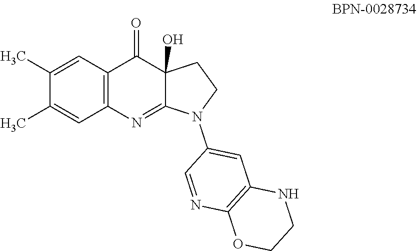 [SMILES: Cc1cc2c(cc1C)C(=O)[C@]1(O)CCN(c3cnc4c(c3)NCCO4)C1=N2]

(S)-1-(2,3-Dihydro-1H-pyrido[2,3-b][1,4]oxazin-7-yl)-3a-hydroxy-6,7-dimethyl-1,2,3,3a-tetrahydro-4H-pyrrolo[2,3-b]quinolin-4-one was prepared as a red-orange solid according to Synthetic Scheme 4: mp=225-230° C.; ¹H NMR (500 MHz, DMSO-d₆) δ 7.86 (d, J=2.4 Hz, 1H), 7.82 (d, J=2.4 Hz, 1H), 7.46 (s, 1H), 6.99 (s, 1H), 6.73 (s, 1H), 6.23 (s, 1H), 4.25 (t, J=4.5 Hz, 2H), 3.98-3.94 (m, 1H), 3.87-3.84 (m, 1H), 2.26-2.21 (m, 10H); ESI MS m/z 365 [C₂₀H₂₀N₄O₃+H]⁺; UPLC (Method A) 98.3% (AUC), t_{R}=2.72 min; Chiral HPLC (Chiralpak AD, Method A) 86.1% (AUC), t_{R}=18.80 min.

### Preparation of (S)-3a-Hydroxy-6,7-dimethyl-1-(1,2,3,5-tetrahydrobenzo[e][1,4]oxazepin-7-yl)-1,2,3,3a-tetrahydro-4H-pyrrolo[2,3-b]quinolin-4-one

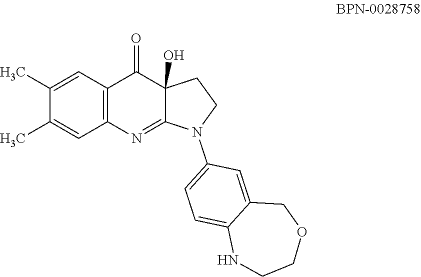 [SMILES: Cc1cc2c(cc1C)C(=O)[C@]1(O)CCN(c3ccc4c(c3)COCCN4)C1=N2]

(S)-3a-Hydroxy-6,7-dimethyl-1-(1,2,3,5-tetrahydrobenzo[e][1,4]oxazepin-7-yl)-1,2,3,3a-tetrahydro-4H-pyrrolo[2,3-b]quinolin-4-one was prepared as an orange solid according to Synthetic Scheme 4: ¹H NMR (500 MHz, DMSO-d₆) δ 7.78 (dd, J=8.6, 2.6 Hz, 1H), 7.74 (d, J=2.6 Hz, 1H), 7.45 (s, 1H), 6.99 (s, 1H), 6.93 (d, J=8.6 Hz, 1H), 6.68 (s, 1H), 5.68 (apparent t, J=3.7 Hz, 1H), 4.50 (s, 2H), 4.02-3.97 (m, 1H), 3.89-3.85 (m, 1H), 3.72-3.70 (m, 2H), 3.02-2.99 (m, 2H), 2.25 (s, 3H), 2.22-2.18 (m, 5H); ESI MS m/z 378 [C₂₂H₂₃N₃O₃+H]⁺; UPLC (Method A) 96.3% (AUC), t_{R}=2.97 min; Chiral HPLC (Chiralpak AD, Method A) 91.0% (AUC), t_{R}=23.89 min.

### Preparation of (S)-1-(3,4-Dihydro-2H-benzo[b][1,4]oxazin-6-yl)-3a-hydroxy-6,7-dimethyl-1,2,3,3a-tetrahydro-4H-pyrrolo[2,3-b]quinolin-4-one

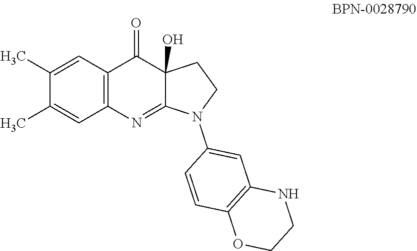 [SMILES: Cc1cc2c(cc1C)C(=O)[C@]1(O)CCN(c3ccc4c(c3)NCCO4)C1=N2]

(S)-1-(3,4-Dihydro-2H-benzo[b][1,4]oxazin-6-yl)-3a-hydroxy-6,7-dimethyl-1,2,3,3a-tetrahydro-4H-pyrrolo[2,3-b]quinolin-4-one was prepared as a yellow-orange solid according to Synthetic Scheme 4: mp=200-203° C.; ¹H NMR (500 MHz, DMSO-d₆) δ 7.44 (s, 1H), 7.35 (d, J=2.6 Hz, 1H), 7.03 (dd, J=8.7, 2.7 Hz, 1H), 6.97 (s, 1H), 6.67-6.64 (m, 1H), 5.88 (s, 1H), 4.12-4.10 (m, 2H), 3.98-3.93 (m, 1H), 3.83-3.79 (m, 1H), 2.25 (s, 3H), 2.20-2.14 (m, 5H), 3H obscured by solvent peak; ESI MS m/z 364 [C₂₁H₂₁N₃O₃+H]⁺; UPLC (Method A) >99% (AUC), t_{R}=3.14 min; Chiral HPLC (Chiralpak AD, Method A) >99% (AUC), t_{R}=20.43 min.

### Preparation of (S)-1-(5-Chloro-6-methoxypyridin-3-yl)-3a-hydroxy-6,7-dimethyl-1,2,3,3a-tetrahydro-4H-pyrrolo[2,3-b]quinolin-4-one

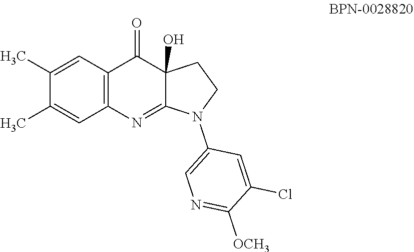 [SMILES: COc1ncc(N2CC[C@@]3(O)C(=O)c4cc(C)c(C)cc4N=C23)cc1Cl]

(S)-1-(5-Chloro-6-methoxypyridin-3-yl)-3a-hydroxy-6,7-dimethyl-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 8.85 (d, J=2.5 Hz, 1H), 8.70 (d, J=2.56 Hz, 1H), 7.49 (s, 1H), 7.05 (s, 1H), 6.82 (s, 1H), 4.05-4.02 (m, 1H), 3.97-3.96 (m, 4H), 2.27 (s, 3H), 2.26-2.24 (m, 2H), 2.22 (s, 3H); ESI MS m/z 372 [C₁₉H₁₈ClN₃O₃+H]⁺; UPLC (Method A) 98.2% (AUC), t_{R}=3.42 min; Chiral HPLC (Chiralpak AD, Method A) 85.2% (AUC), t_{R}=13.81 min.

### Preparation of (S)-4a-Hydroxy-2-methyl-7-(2-methylquinolin-6-yl)-4a,5,6,7-tetrahydro-4H-pyrrolo[2,3-b]thieno[3,2-e]pyridin-4-one

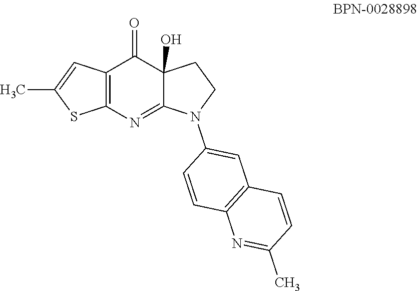 [SMILES: Cc1ccc2cc(N3CC[C@@]4(O)C(=O)c5cc(C)sc5N=C34)ccc2n1]

(S)-4a-Hydroxy-2-methyl-7-(2-methylquinolin-6-yl)-4a,5,6,7-tetrahydro-4H-pyrrolo[2,3-b]thieno[3,2-e]pyridin-4-one was prepared as a red solid according to Synthetic Scheme 3: mp=247-249° C.; ¹H NMR (500 MHz, DMSO-d₆) δ 8.53 (d, J=10.5 Hz, 1H), 8.26-8.24 (m, 2H), 7.99 (d, J=9.5 Hz, 1H), 7.43 (d, J=9.1 Hz, 1H), 6.95 (s, 1H), 6.82 (s, 1H), 4.27-4.24 (m, 1H), 4.19-3.17 (m, 1H), 2.66 (s, 3H), 2.29-2.28 (m, 2H), 3H obscured by solvent peak; ESI MS m/z 364 [C₂₀H₁₇N₃O₂S+H]⁺; UPLC (Method A) 97.5% (AUC), t_{R}=2.80 min; Chiral HPLC (Chiralpak AD, Method A) >99% (AUC), t_{R}=18.84 min.

### Preparation of (S)-1-(2-Aminoquinolin-6-yl)-3a-hydroxy-6,7-dimethyl-1,2,3,3a-tetrahydro-4H-pyrrolo[2,3-b]quinolin-4-one

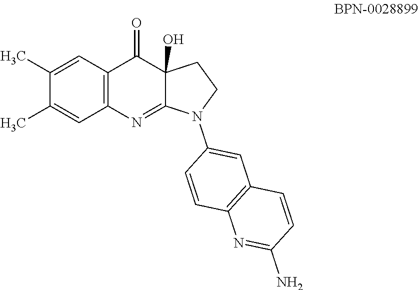 [SMILES: Cc1cc2c(cc1C)C(=O)[C@]1(O)CCN(c3ccc4nc(N)ccc4c3)C1=N2]

(S)-1-(2-Aminoquinolin-6-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=282-283° C.; ¹H NMR (500 MHz, DMSO-d₆) δ 8.38 (dd, J=9.1, 2.5 Hz, 1H), 8.13 (d, J=2.5 Hz, 1H), 7.91 (d, J=9.1 Hz, 1H), 7.50-7.48 (m, 2H), 7.05 (s, 1H), 6.79-6.76 (m, 2H), 6.37 (s, 2H), 4.15-4.09 (m, 1H), 4.04-4.00 (m, 1H), 2.27-2.25 (m, 5H), 2.22 (s, 3H); ESI MS m/z 373 [C₂₂H₂₀N₄O₂+H]⁺; UPLC (Method A) >99% (AUC), t_{R}=2.58 min; Chiral HPLC (Chiralpak AD, Method A) 86.1% (AUC), t_{R}=23.54 min.

### Preparation of (S)-3a-Hydroxy-6,7-dimethyl-1-(5,6,7,8-tetrahydronaphthalen-2-yl)-1,2,3,3a-tetrahydro-4H-pyrrolo[2,3-b]quinolin-4-one

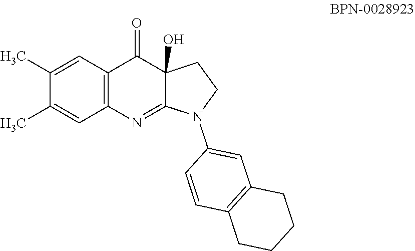 [SMILES: Cc1cc2c(cc1C)C(=O)[C@]1(O)CCN(c3ccc4c(c3)CCCC4)C1=N2]

(S)-3a-Hydroxy-6,7-dimethyl-1-(5,6,7,8-tetrahydronaphthalen-2-yl)-1,2,3,3a-tetrahydro-4H-pyrrolo[2,3-b]quinolin-4-one was prepared as a yellow solid according to Synthetic Scheme 6: mp=211-212° C.; ¹H NMR (500 MHz, DMSO-d₆) δ 7.84-7.82 (m, 1H), 7.64-7.63 (m, 1H), 7.46 (s, 1H), 7.08 (d, J=8.5 Hz, 1H), 6.99 (s, 1H), 6.71 (s, 1H), 4.03-3.98 (m, 1H), 3.91-3.88 (m, 1H), 2.77-2.74 (m, 2H), 2.72-2.69 (m, 2H), 2.26 (s, 3H), 2.22-2.19 (m, 5H), 1.76-1.74 (m, 4H); ESI MS m/z 361 [C₂₃H₂₄N₂O₂+H]⁺; UPLC (Method A) 96.8% (AUC), t_{R}=3.88 min; Chiral HPLC (Chiralpak AD, Method A) >99% (AUC), t_{R}=13.95 min.

### Preparation of (S)-1-(Chroman-7-yl)-3a-hydroxy-6,7-dimethyl-1,2,3,3a-tetrahydro-4H-pyrrolo[2,3-b]quinolin-4-one

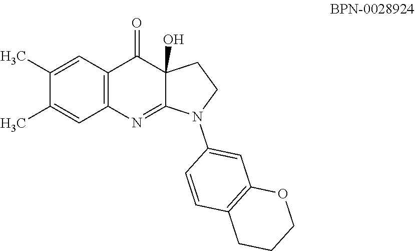 [SMILES: Cc1cc2c(cc1C)C(=O)[C@]1(O)CCN(c3ccc4c(c3)OCCC4)C1=N2]

(S)-1-(Chroman-7-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=219-221° C.; ¹H NMR (500 MHz, DMSO-d₆) δ 7.63 (d, J=2.3 Hz, 1H), 7.47 (s, 1H), 7.41 (dd, J=8.4, 2.3 Hz, 1H), 7.07 (d, J=8.4 Hz, 1H), 7.02 (s, 1H), 6.72 (s, 1H), 4.15-4.13 (m, 2H), 4.01-3.95 (m, 1H), 3.90-3.87 (m, 1H), 2.74-2.71 (m, 2H), 2.27 (s, 3H), 2.21-2.18 (m, 5H), 1.95-1.90 (m, 2H); ESI MS m/z 363 [C₂₂H₂₂N₂O₃+H]⁺; UPLC (Method A) >99% (AUC), t_{R}=3.46 min; Chiral HPLC (Chiralpak AD, Method A) 88.0% (AUC), t_{R}=19.01 min.

### Preparation of (S)-3a-Hydroxy-6,7-dimethyl-1-(1,2,3,4-tetrahydroquinolin-7-yl)-1,2,3,3a-tetrahydro-4H-pyrrolo[2,3-b]quinolin-4-one

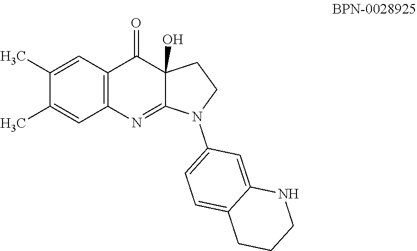 [SMILES: Cc1cc2c(cc1C)C(=O)[C@]1(O)CCN(c3ccc4c(c3)NCCC4)C1=N2]

(S)-3a-Hydroxy-6,7-dimethyl-1-(1,2,3,4-tetrahydroquinolin-7-yl)-1,2,3,3a-tetrahydro-4H-pyrrolo[2,3-b]quinolin-4-one was prepared as a yellow solid according to Synthetic Scheme 6: mp=220-221° C.; ¹H NMR (500 MHz, DMSO-d₆) δ 7.45 (s, 1H), 7.19 (d, J=2.2 Hz, 1H), 7.02 (dd, J=8.2, 2.3 Hz, 1H), 6.99 (s, 1H), 6.83 (d, J=8.2 Hz, 1H), 6.68 (s, 1H), 5.73 (s, 1H), 3.97-3.92 (m, 1H), 3.83-3.80 (m, 1H), 3.19-3.17 (m, 2H), 2.65-2.63 (m, 2H), 2.25 (s, 3H), 2.20-2.16 (m, 5H), 1.82-1.77 (m, 2H); ESI MS m/z 362 [C₂₂H₂₃N₃O₂+H]⁺; UPLC (Method A) 98.26% (AUC), t_{R}=3.07 min; Chiral HPLC (Chiralpak AD, Method A) 99.0% (AUC), t_{R}=20.30 min.

### Preparation of (S)-3a-Hydroxy-1-phenyl-6-(trifluoromethyl)-3,3a-dihydro-1H-pyrrolo[2,3-b]quinolin-4(2H)-one

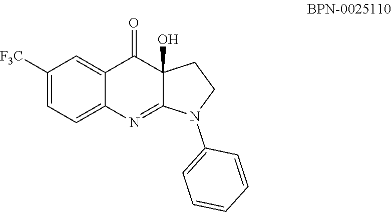 [SMILES: O=C1c2cc(C(F)(F)F)ccc2N=C2N(c3ccccc3)CC[C@@]12O]

(S)-3a-Hydroxy-1-phenyl-6-(trifluoromethyl)-3,3a-dihydro-1H-pyrrolo[2,3-b]quinolin-4(2H)-one was prepared as a yellow solid according to Synthetic Scheme 2: mp=222-223° C.; ¹H NMR (500 MHz, DMSO-d₆) δ 8.06 (dd, J=8.5, 1.0 Hz, 2H), 7.93 (d, J=2.5 Hz, 1H), 7.84 (dd, J=8.5, 2.5 Hz, 1H), 7.46 (apparent dt, J=7.0, 2.0 Hz, 2H), 7.36 (d, J=8.5 Hz, 1H), 7.21 (apparent t, J=7.0 Hz, 1H), 7.04 (s, 1H), 4.20-4.14 (m, 1H), 4.02 (apparent t, J=9.0 Hz, 1H), 2.43-2.36 (m, 1H), 2.28 (dd, J=13.5, 6.0 Hz, 1H); ESI MS m/z 347 [C₁₈H₁₃F₃N₂O₂+H]⁺; Chiral HPLC (Chiralpak AD, Method A) >99% (AUC), t_{R}=10.74 min.

### Preparation of (S)-3a-Hydroxy-3,3,6-trimethyl-1-phenyl-3,3a-dihydro-1H-pyrrolo[2,3-b]quinolin-4(2H)-one

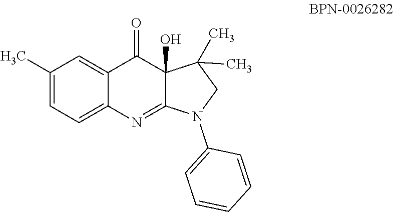 [SMILES: Cc1ccc2c(c1)C(=O)[C@@]1(O)C(=N2)N(c2ccccc2)CC1(C)C]

(S)-3a-Hydroxy-3,3,6-trimethyl-1-phenyl-3,3a-dihydro-1H-pyrrolo[2,3-b]quinolin-4(2H)-one was prepared as a yellow solid according to Synthetic Scheme 3: mp=177-178° C.; ¹H NMR (500 MHz, DMSO-d₆) δ 8.03 (dd, J=8.5, 1.0 Hz, 2H), 7.56 (d, J=2.0 Hz, 1H), 7.43-7.39 (m, 3H), 7.15-7.12 (m, 2H), 6.88 (s, 1H), 3.85 (d, J=9.5 Hz, 1H), 3.54 (d, J=9.5 Hz, 1H), 2.30 (s, 3H), 1.40 (s, 3H), 1.00 (s, 3H); ESI MS m/z 321 [C₂₀H₂₀N₂O₂+H]⁺; HPLC (Method C) >99% (AUC), t_{R}=13.15 min; Chiral HPLC (Chiralpak AD, Method A) 87.5% (AUC), t_{R}=10.04 min.

### Preparation of (S)-6-Fluoro-3a-hydroxy-7-methyl-1-phenyl-3,3a-dihydro-1H-pyrrolo[2,3-b]quinolin-4(2H)-one

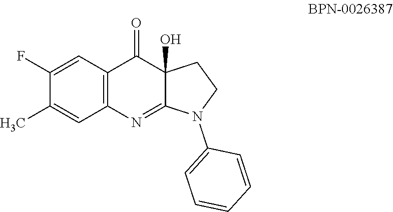 [SMILES: Cc1cc2c(cc1F)C(=O)[C@]1(O)CCN(c3ccccc3)C1=N2]

(S)-6-Fluoro-3a-hydroxy-7-methyl-1-phenyl-3,3a-dihydro-1H-pyrrolo[2,3-b]quinolin-4(2H)-one was prepared as an orange solid according to Synthetic Scheme 2: mp=211-213° C.; ¹H NMR (500 MHz, DMSO-d₆) δ 8.07 (dd, J=8.5, 1.0 Hz, 2H), 7.43 (apparent dt, J=7.5, 2.0 Hz, 2H), 7.36 (d, J=9.5 Hz, 1H), 7.18-7.14 (m, 2H), 6.87 (s, 1H), 4.10-4.05 (m, 1H), 3.98-2.95 (m, 1H), 2.29-2.25 (m, 5H); ESI MS m/z 311 [C₁₈H₁₅FN₂O₂+H]⁺; HPLC (Method C) 95.0% (AUC), t_{R}=11.66 min; Chiral HPLC (Chiralpak AD, Method A) 98.2% (AUC), t_{R}=17.81 min.

### Preparation of (S)-3a-Hydroxy-1-phenyl-3,3a,5,6,7,8-hexahydro-1H-benzo[4,5]thieno[2,3-b]pyrrolo[3,2-e]pyridin-4(2H)-one

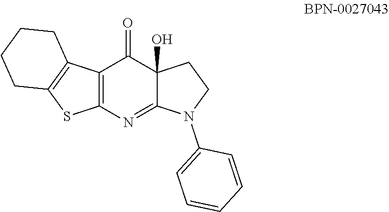 [SMILES: O=C1c2c(sc3c2CCCC3)N=C2N(c3ccccc3)CC[C@@]12O]

(S)-3a-Hydroxy-1-phenyl-3,3a,5,6,7,8-hexahydro-1H-benzo[4,5]thieno[2,3-b]pyrrolo[3,2-e]pyridin-4(2H)-one was prepared as a yellow solid according to Synthetic Scheme 2: mp=259-261° C.; ¹H NMR (500 MHz, DMSO-d₆) δ 7.93 (br d, J=7.5 Hz, 2H), 7.45 (br s, 2H), 7.19 (br s, 1H), 6.85 (s, 1H), 4.14 (br s, 1H), 4.04-4.03 (br m, 1H), 2.74-2.59 (m, 4H), 2.21 (br s, 2H), 1.76 (br s, 4H); ESI MS m/z 339 [C₁₉H₁₈N₂O₂S+H]⁺; HPLC (Method C) >99% (AUC), t_{R}=15.91 min; Chiral HPLC (Chiralpak AD, Method A) 89.1% (AUC), t_{R}=18.50 min.

### Preparation of (S)-3a-Hydroxy-6-methyl-1-(2-methylquinolin-6-yl)-3,3a-dihydro-1H-pyrrolo[2,3-b]quinolin-4(2H)-one

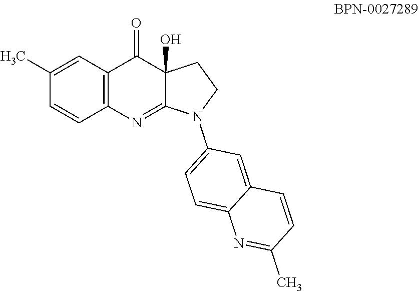 [SMILES: Cc1ccc2c(c1)C(=O)[C@]1(O)CCN(c3ccc4nc(C)ccc4c3)C1=N2]

(S)-3a-Hydroxy-6-methyl-1-(2-methylquinolin-6-yl)-3,3a-dihydro-1H-pyrrolo[2,3-b]quinolin-4(2H)-one was prepared as a yellow solid according to Synthetic Scheme 3: mp=237-239° C. decomposed; ¹H NMR (500 MHz, DMSO-d₆) δ 8.70 (dd, J=9.0, 2.5 Hz, 1H), 8.42 (d, J=2.5 Hz, 1H), 8.24 (d, J=8.5 Hz, 1H), 7.96 (d, J=8.5 Hz, 1H), 7.56 (d, J=1.5 Hz, 1H), 7.43-7.40 (m, 2H), 7.21 (d, J=8.0 Hz, 1H), 6.89 (s, 1H), 4.20-4.15 (m, 1H), 4.13-4.09 (m, 1H), 2.65 (s, 3H), 2.37-2.30 (m, 5H); ESI MS m/z 358 [C₂₂H₁₉N₃O₂+H]⁺; HPLC (Method C) 97.3% (AUC), t_{R}=11.08 min; Chiral HPLC (Chiralpak AD, Method A) 95.8% (AUC), t_{R}=17.12 min.

### Preparation of (S)-3a-Hydroxy-6-methyl-1-(quinoxalin-6-yl)-3,3a-dihydro-1H-pyrrolo[2,3-b]quinolin-4(2H)-one

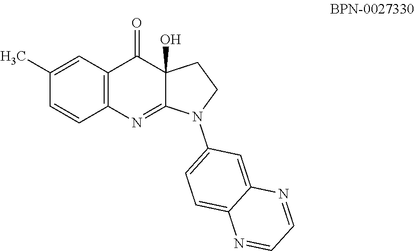 [SMILES: Cc1ccc2c(c1)C(=O)[C@]1(O)CCN(c3ccc4nccnc4c3)C1=N2]

(S)-3a-Hydroxy-6-methyl-1-(quinoxalin-6-yl)-3,3a-dihydro-1H-pyrrolo[2,3-b]quinolin-4(2H)-one was prepared as a yellow solid according to Synthetic Scheme 3: mp=151-153° C. decomposed; ¹H NMR (500 MHz, DMSO-d₆) δ 8.93 (d, J=1.5 Hz, 1H), 8.86 (d, J=2.0 Hz, 1H), 8.83 (dd, J=9.5, 2.5 Hz, 1H), 8.68 (d, J=2.5 Hz, 1H), 8.14 (d, J=9.0 Hz, 1H), 7.59 (d, J=2.0 Hz, 1H), 7.44 (dd, J=8.5, 2.0 Hz, 1H), 7.24 (d, J=8.0 Hz, 1H), 6.95 (s, 1H), 4.24-4.17 (m, 2H), 2.38-2.31 (m, 5H); ESI MS m/z 345 [C₂₀H₁₆N₄O₂+H]⁺; HPLC (Method C) >99% (AUC), t_{R}=12.39 min; Chiral HPLC (Chiralpak AD, Method A) >99% (AUC), t_{R}=20.90 min.

### Preparation of (S)-3a-Hydroxy-6,7-dimethyl-1-(2-methylquinolin-6-yl)-3,3a-dihydro-1H-pyrrolo[2,3-b]quinolin-4(2H)-one

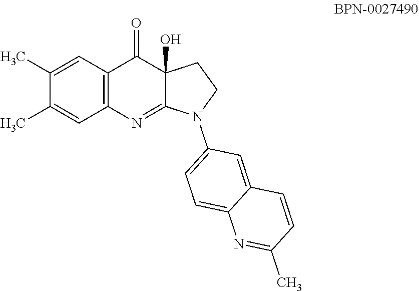 [SMILES: Cc1ccc2cc(N3CC[C@@]4(O)C(=O)c5cc(C)c(C)cc5N=C34)ccc2n1]

(S)-3a-Hydroxy-6,7-dimethyl-1-(2-methylquinolin-6-yl)-3,3a-dihydro-1H-pyrrolo[2,3-b]quinolin-4(2H)-one was prepared as a yellow solid according to Synthetic Scheme 3: mp=254-255° C. decomposed; ¹H NMR (500 MHz, DMSO-d₆) δ 8.73 (dd, J=15.5, 4.0 Hz, 1H), 8.40 (d, J=4.0 Hz, 1H), 8.25 (d, J=14.0 Hz, 1H), 7.96 (d, J=15.0 Hz, 1H), 7.51 (s, 1H), 7.42 (d, J=14.5 Hz, 1H), 7.12 (s, 1H), 6.86 (s, 1H), 4.20-4.10 (m, 2H), 2.65 (s, 3H), 2.29-2.24 (m, 8H); ESI MS m/z 372 [C₂₃H₂₁N₃O₂+H]⁺; UPLC (Method A) >99% (AUC), t_{R}=2.65 min; Chiral HPLC (Chiralpak AD, Method A) 97.0% (AUC), t_{R}=16.10 min.

### Preparation of (S)-2-Ethyl-7a-hydroxy-5-(quinolin-6-yl)-7,7a-dihydro-5H-pyrrolo[2,3-b]thieno[2,3-e]pyridin-8(6H)-one

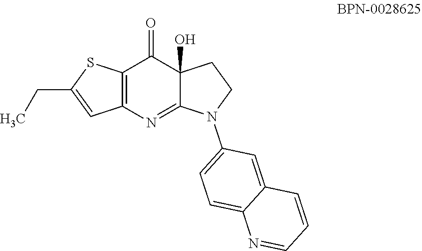 [SMILES: CCc1cc2c(s1)C(=O)[C@]1(O)CCN(c3ccc4ncccc4c3)C1=N2]

(S)-2-Ethyl-7a-hydroxy-5-(quinolin-6-yl)-7,7a-dihydro-5H-pyrrolo[2,3-b]thieno[2,3-e]pyridin-8(6H)-one was prepared as a yellow solid according to Synthetic Scheme 3: ¹H NMR (500 MHz, DMSO-d₆) δ 8.85 (dd, J=4.0, 1.5 Hz, 1H), 8.68 (dd, J=9.5, 2.5 Hz, 1H), 8.39 (d, J=2.5 Hz, 1H), 8.36 (d, J=8.0 Hz, 1H), 8.07 (d, J=9.0 Hz, 1H), 7.55 (dd, J=8.0, 4.0 Hz, 1H), 6.97 (s, 1H), 6.93 (s, 1H), 4.27-4.24 (m, 1H), 4.19-4.15 (m, 1H), 2.85 (q, J=7.5 Hz, 2H), 2.35-2.26 (m, 2H), 1.28 (t, J=7.5 Hz, 3H); ESI MS m/z 364 [C₂₀H₁₇N₃O₂S+H]⁺; UPLC (Method A) 92.2% (AUC), t_{R}=2.85 min; Chiral HPLC (Chiralpak AD, Method A) 46.3% (AUC), t_{R}=18.69 min.

### Preparation of (S)-3a-Hydroxy-5,7-dimethyl-1-(quinolin-6-yl)-3,3a-dihydro-1H-pyrrolo[2,3-b]quinolin-4(2H)-one

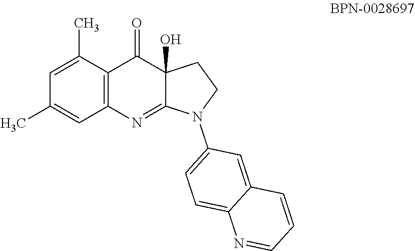 [SMILES: Cc1cc(C)c2c(c1)N=C1N(c3ccc4ncccc4c3)CC[C@@]1(O)C2=O]

(S)-3a-Hydroxy-5,7-dimethyl-1-(quinolin-6-yl)-3,3a-dihydro-1H-pyrrolo[2,3-b]quinolin-4(2H)-one was prepared as a yellow-brown solid according to Synthetic Scheme 3: mp=242-245° C. decomposed; ¹H NMR (500 MHz, DMSO-d₆) δ 8.83 (dd, J=7.0, 3.0 Hz, 1H), 8.78 (d, J=15.5, 4.0 Hz, 1H), 8.46 (d, J=4.5 Hz, 1H), 8.37 (d, J=11.5 Hz, 1H), 8.06 (d, J=15.5 Hz, 1H), 7.53 (dd, J=13.5, 7.0 Hz, 1H), 7.02 (s, 1H), 6.84 (s, 1H), 6.78 (s, 1H), 4.19-4.10 (m, 2H), 2.38-2.22 (m, 5H), 3 protons obscured by solvent; ESI MS m/z 358 [C₂₂H₁₉N₃O₂+H]⁺; UPLC (Method A) 96.8% (AUC), t_{R}=2.76 min; Chiral HPLC (Chiralpak AD, Method A) 94.2% (AUC), t_{R}=16.59 min.

### Preparation of (S)-1-(2-Ethylquinolin-6-yl)-3a-hydroxy-6,7-dimethyl-3,3a-dihydro-1H-pyrrolo[2,3-b]quinolin-4(2H)-one

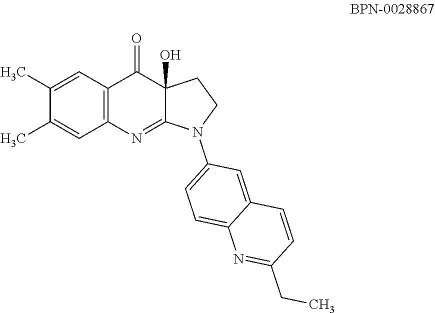 [SMILES: CCc1ccc2cc(N3CC[C@@]4(O)C(=O)c5cc(C)c(C)cc5N=C34)ccc2n1]

(S)-1-(2-Ethylquinolin-6-yl)-3a-hydroxy-6,7-dimethyl-3,3a-dihydro-1H-pyrrolo[2,3-b]quinolin-4(2H)-one was prepared as a yellow solid according to Synthetic Scheme 6: mp=241-242° C.; ¹H NMR (500 MHz, DMSO-d₆) δ 8.73 (dd, J=9.0, 2.5 Hz, 1H), 8.40 (d, J=3.0 Hz, 1H), 8.26 (d, J=8.5 Hz, 1H), 7.97 (d, J=9.0 Hz, 1H), 7.51 (s, 1H), 7.45 (d, J=8.5 Hz, 1H), 7.14 (s, 1H), 6.84 (s, 1H), 4.19-4.08 (m, 2H), 2.93 (q, J=7.5 Hz, 2H), 2.32-2.30 (m, 5H), 2.24 (s, 3H), 1.33 (t, J=7.5 Hz, 3H); ESI MS m/z 386 [C₂₄H₂₃N₃O₂+H]⁺; UPLC (Method A) 99.0% (AUC), t_{R}=2.86 min; Chiral HPLC (Chiralpak AD, Method A) 88.2% (AUC), t_{R}=16.96 min.

### Preparation of (S)-1-(2,3-Dimethylquinolin-6-yl)-3a-hydroxy-6,7-dimethyl-3,3a-dihydro-1H-pyrrolo[2,3-b]quinolin-4(2H)-one

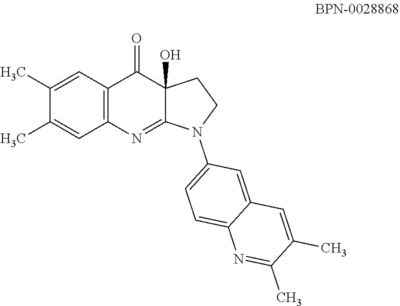

(S)-1-(2,3-Dimethylquinolin-6-yl)-3a-hydroxy-6,7-dimethyl-3,3a-dihydro-1H-pyrrolo[2,3-b]quinolin-4(2H)-one was prepared as a yellow solid according to Synthetic Scheme 6: mp=253-254° C.; ¹H NMR (500 MHz, DMSO-d₆) δ 8.57 (dd, J=9.5, 2.5 Hz, 1H), 8.39 (d, J=2.5 Hz, 1H), 8.04 (s, 1H), 7.92 (d, J=9.5 Hz, 1H), 7.51 (s, 1H), 7.13 (s, 1H), 6.82 (s, 1H), 4.18-4.13 (m, 1H), 4.11-4.07 (m, 1H), 2.61 (s, 3H), 2.44 (s, 3H), 2.31-2.28 (m, 5H), 2.24 (s, 3H); ESI MS m/z 386 [C₂₄H₂₃N₃O₂+H]⁺; UPLC (Method A) >99% (AUC), t_{R}=2.78 min; Chiral HPLC (Chiralpak AD, Method A) 88.5% (AUC), t_{R}=17.13 min.

### Preparation of (S)-1-(3-((Dimethylamino)methyl)quinolin-6-yl)-3a-hydroxy-6,7-dimethyl-1,2,3,3a-tetrahydro-4H-pyrrolo[2,3-b]quinolin-4-one

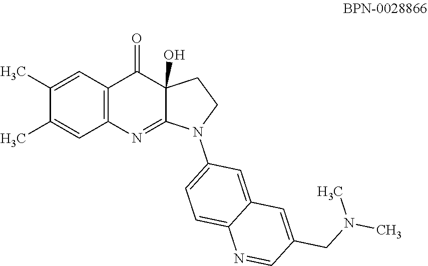

(S)-1-(3-((Dimethylamino)methyl)quinolin-6-yl)-3a-hydroxy-6,7-dimethyl-1,2,3,3a-tetrahydro-4H-pyrrolo[2,3-b]quinolin-4-one was prepared as an orange solid according to Synthetic Scheme 6: ¹H NMR (300 MHz, DMSO-d₆) δ 8.76 (dd, J=9.6, 2.1 Hz, 1H), 8.42 (d, J=2.4 Hz, 1H), 8.33-8.30 (m, 1H), 8.01 (d, J=9.3 Hz, 1H), 7.61 (d, J=8.7 Hz, 1H), 7.51 (s, 1H), 7.14 (s, 1H), 6.86 (s, 1H), 4.16-4.10 (m, 2H), 3.68 (s, 2H), 2.51-2.24 (m, 14H); ESI MS m/z 415 [C₂₅H₂₆N₄O₂+H]⁺; UPLC (Method A) 96.0% (AUC), t_{R}=2.64 min; Chiral HPLC (Chiralpak AD, Method A) 82.8% (AUC), t_{R}=17.32 min.

### Preparation of (S)-1-(2-((Dimethylamino)methyl)quinolin-6-yl)-3a-hydroxy-6,7-dimethyl-1,2,3,3a-tetrahydro-4H-pyrrolo[2,3-b]quinolin-4-one

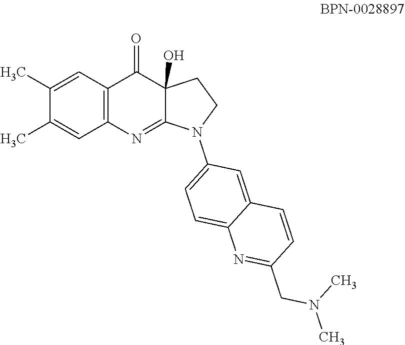

(S)-1-(3-((Dimethylamino)methyl)quinolin-6-yl)-3a-hydroxy-6,7-dimethyl-1,2,3,3a-tetrahydro-4H-pyrrolo[2,3-b]quinolin-4-one was prepared as an orange solid according to Synthetic Scheme 6: ¹H NMR (300 MHz, DMSO-d₆) δ 8.76 (dd, J=9.0, 2.4 Hz, 1H), 8.43-8.41 (m, 1H), 8.33-8.30 (m, 1H), 8.01 (d, J=9.0 Hz, 1H), 7.61 (d, J=8.4 Hz, 1H), 7.51 (s, 1H), 7.14 (s, 1H), 6.86 (s, 1H), 4.16-4.10 (m, 2H), 3.69 (s, 2H), 2.35-2.23 (m, 14H); ESI MS m/z 415 [C₂₅H₂₆N₄O₂+H]⁺; UPLC (Method A) 98.8% (AUC), t_{R}=2.77 min; Chiral HPLC (Chiralpak AD, Method A) 52.4% (AUC), t_{R}=17.67 min.

### Preparation of (S)-1-(Benzofuran-5-yl)-3a-hydroxy-6-methyl-1,2,3,3a-tetrahydro-4H-pyrrolo[2,3-b]quinolin-4-one

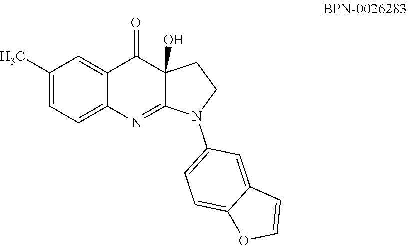 [SMILES: Cc1ccc2c(c1)C(=O)[C@]1(O)CCN(c3ccc4occc4c3)C1=N2]

(S)-1-(Benzofuran-5-yl)-3a-hydroxy-6-methyl-1,2,3,3a-tetrahydro-4H-pyrrolo[2,3-b]quinolin-4-one was prepared as a yellow solid according to Synthetic Scheme 3: mp=213-214° C.; ¹H NMR (300 MHz, DMSO-d₆) δ 8.29 (d, J=2.1 Hz, 1H), 8.06 (d, J=2.1 Hz, 1H), 8.03 (d, J=2.4 Hz, 1H), 7.65 (d, J=9.0 Hz, 1H), 7.53 (d, J=1.5 Hz, 1H), 7.36 (dd, J=8.4, 1.5 Hz, 1H), 7.10 (d, J=8.4 Hz, 1H), 7.02 (dd, J=2.1, 0.9 Hz, 1H), 6.85 (s, 1H), 4.19-4.10 (m, 1H), 4.02-3.96 (m, 1H), 2.30 (s, 3H), 2.29-2.27 (m, 2H); ESI MS m/z 333 [C₂₀H₁₆N₂O₃+H]⁺; HPLC (Method B) 97.8% (AUC), t_{R}=9.42 min; Chiral HPLC (Chiralpak AD, Method A) 96.7% (AUC), t_{R}=17.19 min.

### Preparation of (S)-4-(3a-Hydroxy-6-methyl-4-oxo-2,3,3a,4-tetrahydro-1H-pyrrolo[2,3-b]quinolin-1-yl)-2-methylbenzonitrile

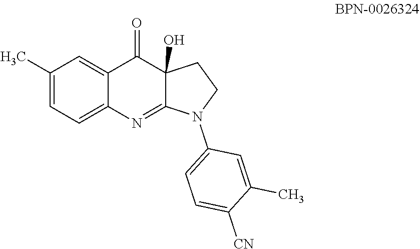

(S)-4-(3a-Hydroxy-6-methyl-4-oxo-2,3,3a,4-tetrahydro-1H-pyrrolo[2,3-b]quinolin-1-yl)-2-methylbenzonitrile was prepared as a yellow solid according to Synthetic Scheme 3: mp=229-231° C.; ¹H NMR (500 MHz, DMSO-d₆) δ 8.27 (dd, J=9.0, 2.5 Hz, 1H), 8.11 (d, J=2.0 Hz, 1H), 7.79 (d, J=4.0 Hz, 1H), 7.56 (d, J=1.5 Hz, 1H), 7.42 (dd, J=8.0, 1.5 Hz, 1H), 7.21 (d, J=8.0 Hz, 1H), 6.90 (s, 1H), 4.04-4.01 (m, 2H), 2.53 (s, 3H), 2.32 (s, 3H), 2.28-2.26 (m, 2H); ESI MS m/z 332 [C₂₀H₁₇N₃O₂+H]⁺; HPLC (Method B) 98.4% (AUC), t_{R}=9.26 min; Chiral HPLC (Chiralpak AD, Method A) 98.9% (AUC), t_{R}=17.48 min.

### Preparation of (S)-3a-Hydroxy-1-(4-methoxyphenyl)-6-(trifluoromethyl)-1,2,3,3a-tetrahydro-4H-pyrrolo[2,3-b]quinolin-4-one

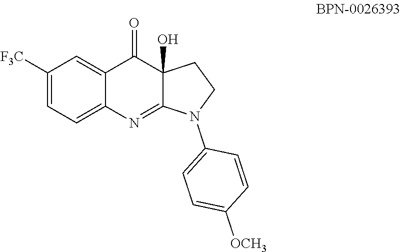 [SMILES: COc1ccc(N2CC[C@@]3(O)C(=O)c4cc(C(F)(F)F)ccc4N=C23)cc1]

(S)-3a-Hydroxy-1-(4-methoxyphenyl)-6-(trifluoromethyl)-1,2,3,3a-tetrahydro-4H-pyrrolo[2,3-b]quinolin-4-one was prepared as a yellow solid according to Synthetic Scheme 3: mp=216-220° C.; ¹H NMR (500 MHz, DMSO-d₆) δ 7.93 (dd, J=7.0, 2.5 Hz, 2H), 7.90 (d, J=2.0 Hz, 1H), 7.81 (dd, J=8.5, 2.5 Hz, 1H), 7.29 (d, J=8.0 Hz, 1H), 7.03 (dd, J=7.0, 2.0 Hz, 2H), 6.99 (s, 1H), 4.17-4.12 (m, 1H), 3.98-3.94 (m, 1H), 3.79 (s, 3H), 2.42-2.35 (m, 1H), 2.28-2.25 (m, 1H); ESI MS m/z 377 [C₁₉H₁₅F₃N₂O₃+H]⁺; HPLC (Method D) 98.6% (AUC), t_{R}=9.55 min; Chiral HPLC (Chiralpak AD, Method A) >99% (AUC), t_{R}=14.30 min.

### Preparation of (S)-2-Chloro-4-(3a-hydroxy-6-methyl-4-oxo-2,3,3a,4-tetrahydro-1H-pyrrolo[2,3-b]quinolin-1-yl)benzonitrile

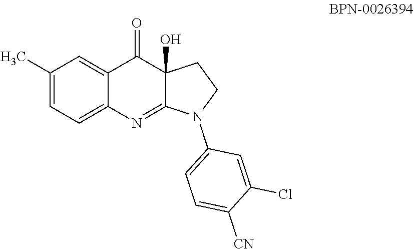 [SMILES: Cc1ccc2c(c1)C(=O)[C@]1(O)CCN(c3ccc(C#N)c(Cl)c3)C1=N2]

(S)-2-Chloro-4-(3a-hydroxy-6-methyl-4-oxo-2,3,3a,4-tetrahydro-1H-pyrrolo[2,3-b]quinolin-1-yl)benzonitrile was prepared as a yellow solid according to Synthetic Scheme 4: mp=215-220° C.; ¹H NMR (300 MHz, DMSO-d₆) δ 8.65 (d, J=2.1 Hz, 1H), 8.19 (dd, J=9.0, 2.1 Hz, 1H), 8.01 (d, J=9.0 Hz, 1H), 7.58 (s, 1H), 7.46 (d, J=8.4 Hz, 1H), 7.23 (d, J=8.1 Hz, 1H), 6.99 (s, 1H), 4.05-4.02 (m, 2H), 2.33 (s, 3H), 2.27 (apparent s, 2H); ESI MS m/z 352 [C₁₉H₁₄ClN₃O₂+H]⁺; HPLC (Method B) >99% (AUC), t_{R}=10.49 min; Chiral HPLC (Chiralpak AD, Method A) >99% (AUC), t_{R}=18.14 min.

### Preparation of (S)-3a-Hydroxy-1-(2-methylthiazol-5-yl)-6-(trifluoromethyl)-1,2,3,3a-tetrahydro-4H-pyrrolo[2,3-b]quinolin-4-one

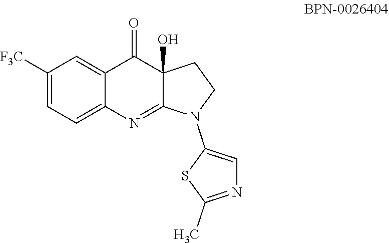 [SMILES: Cc1ncc(N2CC[C@@]3(O)C(=O)c4cc(C(F)(F)F)ccc4N=C23)s1]

(S)-3a-Hydroxy-1-(2-methylthiazol-5-yl)-6-(trifluoromethyl)-1,2,3,3a-tetrahydro-4H-pyrrolo[2,3-b]quinolin-4-one was prepared as a yellow-orange solid according to Synthetic Scheme 4: mp=233-235° C.; ¹H NMR (500 MHz, DMSO-d₆) δ 7.93 (d, J=2.5 Hz, 1H), 7.88 (dd, J=8.5, 2.5 Hz, 1H), 7.56 (s, 1H), 7.39 (d, J=8.0 Hz, 1H), 7.11 (s, 1H), 4.14-4.09 (m, 1H), 4.07-4.03 (m, 1H), 2.62 (s, 3H), 2.56-2.52 (m, 1H), 2.37-2.31 (m, 1H); ESI MS m/z 368 [C₁₆H₁₂F₃N₃O₂S+H]⁺; HPLC (Method B) >99% (AUC), t_{R}=10.32 min; Chiral HPLC (Chiralpak AD, Method A) >99% (AUC), t_{R}=13.78 min.

### Preparation of (S)-3a-Hydroxy-1-(1-methyl-1H-pyrazol-4-yl)-6-(trifluoromethyl)-1,2,3,3a-tetrahydro-4H-pyrrolo[2,3-b]quinolin-4-one

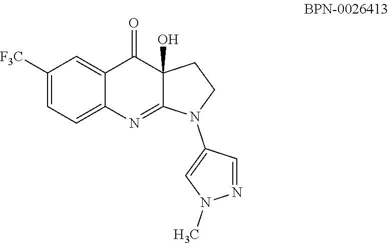 [SMILES: Cn1cc(N2CC[C@@]3(O)C(=O)c4cc(C(F)(F)F)ccc4N=C23)cn1]

(S)-3a-Hydroxy-1-(1-methyl-1H-pyrazol-4-yl)-6-(trifluoromethyl)-1,2,3,3a-tetrahydro-4H-pyrrolo[2,3-b]quinolin-4-one was prepared as a yellow-brown solid according to Synthetic Scheme 4: ¹H NMR (500 MHz, DMSO-d₆) δ 8.32 (s, 1H), 7.90-7.89 (m, 2H), 7.82 (dd, J=8.5, 2.0 Hz, 1H), 7.37 (d, J=8.5 Hz, 1H), 6.96 (s, 1H), 4.01-3.96 (m, 1H), 3.91-3.87 (m, 4H), 2.47-2.40 (m, 1H), 2.30-2.26 (m, 1H); ESI MS m/z 351 [C₁₆H₁₃F₃N₄O₂+H]⁺; Chiral HPLC (Chiralpak AD, Method A) 49.5% (AUC), t_{R}=15.24 min.

### Preparation of (S)-4-(6-Chloro-3a-hydroxy-4-oxo-2,3,3a,4-tetrahydro-1H-pyrrolo[2,3-b]quinolin-1-yl)-2-methylbenzonitrile

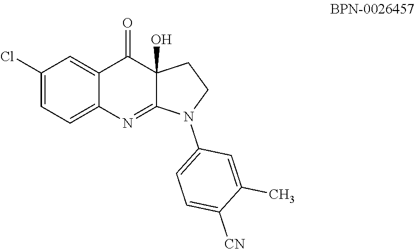 [SMILES: Cc1cc(N2CC[C@@]3(O)C(=O)c4cc(Cl)ccc4N=C23)ccc1C#N]

(S)-4-(6-Chloro-3a-hydroxy-4-oxo-2,3,3a,4-tetrahydro-1H-pyrrolo[2,3-b]quinolin-1-yl)-2-methylbenzonitrile was prepared as an orange solid according to Synthetic Scheme 3: mp=236-239° C.; ¹H NMR (500 MHz, DMSO-d₆) δ 8.24 (dd, J=9.0, 2.0 Hz, 1H), 8.12 (s, 1H), 7.81 (d, J=9.0 Hz, 1H), 7.68 (d, J=3.0 Hz, 1H), 7.62 (dd, J=8.5, 2.5 Hz, 1H), 7.33 (d, J=8.5 Hz, 1H), 7.03 (s, 1H), 4.11-4.02 (m, 2H), 2.53 (s, 3H), 2.29-2.25 (m, 2H); ESI MS m/z 352 [C₁₉H₁₄ClN₃O₂+H]⁺; HPLC (Method D) >99% (AUC), t_{R}=10.20 min; Chiral HPLC (Chiralpak AD, Method A) 96.5% (AUC), t_{R}=16.89 min.

### Preparation of (S)-1-(Benzofuran-6-yl)-3a-hydroxy-6-(trifluoromethyl)-1,2,3,3a-tetrahydro-4H-pyrrolo[2,3-b]quinolin-4-one

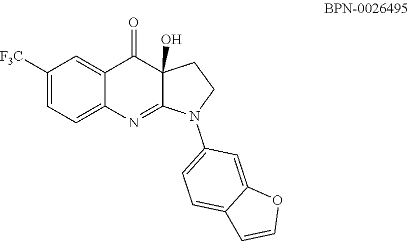 [SMILES: O=C1c2cc(C(F)(F)F)ccc2N=C2N(c3ccc4ccoc4c3)CC[C@@]12O]

(S)-1-(Benzofuran-6-yl)-3a-hydroxy-6-(trifluoromethyl)-1,2,3,3a-tetrahydro-4H-pyrrolo[2,3-b]quinolin-4-one was prepared as a yellow solid according to Synthetic Scheme 3: mp=232-234° C.; ¹H NMR (300 MHz, DMSO-d₆) δ 8.49 (s, 1H), 8.03 (d, J=2.1 Hz, 1H), 7.94 (s, 1H), 7.91-7.84 (m, 2H), 7.72 (d, J=8.7 Hz, 1H), 7.39 (d, J=8.4 Hz, 1H), 7.08 (s, 1H), 6.98 (dd, J=2.1, 0.9 Hz, 1H), 4.27-4.21 (m, 1H), 4.11-4.05 (m, 1H), 2.34-2.27 (m, 2H); ESI MS m/z 387 [C₂₀H₁₃F₃N₂O₃+H]⁺; HPLC (Method E) >99% (AUC), t_{R}=9.56 min; Chiral HPLC (Chiralpak AD, Method A) >99% (AUC), t_{R}=14.72 min.

### Preparation of (S)-3a-Hydroxy-1-(2-methoxypyridin-4-yl)-6-(trifluoromethyl)-1,2,3,3a-tetrahydro-4H-pyrrolo[2,3-b]quinolin-4-one

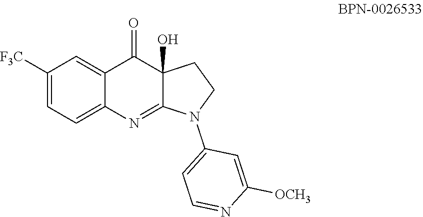 [SMILES: COc1cc(N2CC[C@@]3(O)C(=O)c4cc(C(F)(F)F)ccc4N=C23)ccn1]

(S)-3a-Hydroxy-1-(2-methoxypyridin-4-yl)-6-(trifluoromethyl)-1,2,3,3a-tetrahydro-4H-pyrrolo[2,3-b]quinolin-4-one was prepared as a yellow solid according to Synthetic Scheme 4: ¹H NMR (500 MHz, DMSO-d₆) δ 8.17 (d, J=6.0 Hz, 1H), 7.96 (s, 1H), 7.92 (dd, J=8.0, 2.5 Hz, 1H), 7.75 (dd, J=6.0, 2.0 Hz, 1H), 7.58 (d, J=2.0 Hz, 1H), 7.49 (d, J=8.5 Hz, 1H), 7.11 (s, 1H), 4.08-4.05 (m, 2H), 3.88 (s, 3H), 2.44-2.37 (m, 1H), 2.30-2.25 (m, 1H); ESI MS m/z 378 [C₁₈H₁₄F₃N₃O₃+H]⁺; HPLC (Method B) 93.3% (AUC), t_{R}=9.81 min; Chiral HPLC (Chiralpak AD, Method A) 47.7% (AUC), t_{R}=12.01 min.

### Preparation of (S)-1-(4-Chlorophenyl)-3a-hydroxy-6-(trifluoromethyl)-1,2,3,3a-tetrahydro-4H-pyrrolo[2,3-b]quinolin-4-one

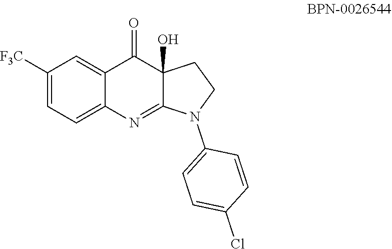 [SMILES: O=C1c2cc(C(F)(F)F)ccc2N=C2N(c3ccc(Cl)cc3)CC[C@@]12O]

(S)-1-(4-Chlorophenyl)-3a-hydroxy-6-(trifluoromethyl)-1,2,3,3a-tetrahydro-4H-pyrrolo[2,3-b]quinolin-4-one was prepared as a yellow-brown solid according to Synthetic Scheme 3: mp=204-207° C.; ¹H NMR (500 MHz, DMSO-d₆) δ 8.12 (dd, J=7.0, 2.5 Hz, 2H), 7.93 (d, J=2.0 Hz, 1H), 7.86 (dd, J=8.5, 2.5 Hz, 1H), 7.52 (dd, J=7.0, 2.0 Hz, 2H), 7.37 (d, J=8.5 Hz, 1H), 7.05 (s, 1H), 4.17-4.12 (m, 1H), 4.03-3.99 (m, 1H), 2.46-2.39 (m, 1H), 2.30-2.26 (m, 1H); ESI MS m/z 381 [C₁₈H₁₂ClF₃N₂O₂+H]⁺; HPLC (Method B) 97.9% (AUC), t_{R}=10.34 min; Chiral HPLC (Chiralpak AD, Method A) 93.0% (AUC), t_{R}=12.86 min.

### Preparation of (S)-6-Fluoro-3a-hydroxy-7-methyl-1-(p-tolyl)-1,2,3,3a-tetrahydro-4H-pyrrolo[2,3-b]quinolin-4-one

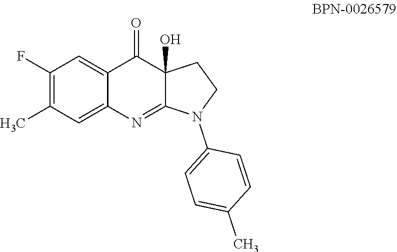 [SMILES: Cc1ccc(N2CC[C@@]3(O)C(=O)c4cc(F)c(C)cc4N=C23)cc1]

(S)-6-Fluoro-3a-hydroxy-7-methyl-1-(p-tolyl)-1,2,3,3a-tetrahydro-4H-pyrrolo[2,3-b]quinolin-4-one was prepared as a yellow solid according to Synthetic Scheme 3: mp=217-220° C.; ¹H NMR (500 MHz, DMSO-d₆) δ 7.94 (d, J=8.5 Hz, 2H), 7.35 (d, J=9.0 Hz, 1H), 7.23 (d, J=8.5 Hz, 2H), 7.14 (d, J=7.0 Hz, 1H), 6.85 (s, 1H), 4.08-4.03 (m, 1H), 3.94-3.91 (m, 1H), 2.31 (s, 3H), 2.28 (s, 3H), 2.26-2.24 (m, 2H); ESI MS m/z 325 [C₁₉H₁₇FN₂O₂+H]⁺; HPLC (Method C) 95.9% (AUC), t_{R}=12.90 min; Chiral HPLC (Chiralpak AD, Method A) 98.5% (AUC), t_{R}=13.82 min.

### Preparation of (S)-6-Fluoro-3a-hydroxy-1-(4-methoxyphenyl)-7-methyl-1,2,3,3a-tetrahydro-4H-pyrrolo[2,3-b]quinolin-4-one

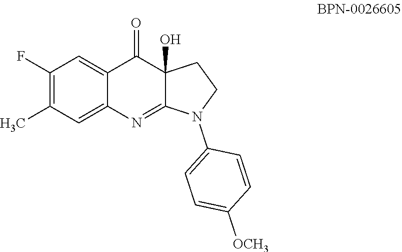 [SMILES: COc1ccc(N2CC[C@@]3(O)C(=O)c4cc(F)c(C)cc4N=C23)cc1]

(S)-6-Fluoro-3a-hydroxy-1-(4-methoxyphenyl)-7-methyl-1,2,3,3a-tetrahydro-4H-pyrrolo[2,3-b]quinolin-4-one was prepared as a yellow solid according to Synthetic Scheme 3: mp=220-222° C.; ¹H NMR (300 MHz, DMSO-d₆) δ 7.96 (d, J=9.0 Hz, 2H), 7.35 (d, J=9.6 Hz, 1H), 7.11 (d, J=6.9 Hz, 1H), 7.00 (d, J=9.0 Hz, 2H), 6.85 (s, 1H), 4.10-4.01 (m, 1H), 3.93-3.88 (m, 1H), 3.77 (s, 3H), 2.27 (s, 3H), 2.23 (apparent s, 2H); ESI MS m/z 341 [C₁₉H₁₇FN₂O₃+H]⁺; HPLC (Method B) >99% (AUC), t_{R}=8.57 min; Chiral HPLC (Chiralpak AD, Method A) >99% (AUC), t_{R}=16.69 min.

### Preparation of (S)-1-(3-Chloro-4-methylphenyl)-6-fluoro-3a-hydroxy-7-methyl-1,2,3,3a-tetrahydro-4H-pyrrolo[2,3-b]quinolin-4-one

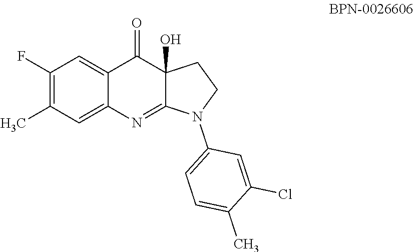 [SMILES: Cc1cc2c(cc1F)C(=O)[C@]1(O)CCN(c3ccc(C)c(Cl)c3)C1=N2]

(S)-1-(3-Chloro-4-methylphenyl)-6-fluoro-3a-hydroxy-7-methyl-1,2,3,3a-tetrahydro-4H-pyrrolo[2,3-b]quinolin-4-one was prepared as a yellow solid according to Synthetic Scheme 3: mp=213-215° C.; ¹H NMR (300 MHz, DMSO-d₆) δ 8.37 (d, J=2.4 Hz, 1H), 7.81 (dd, J=8.4, 2.4 Hz, 1H), 7.39 (dd, J=8.1, 2.4 Hz, 2H), 7.18 (d, J=7.2 Hz, 1H), 6.92 (s, 1H), 4.09-3.92 (m, 2H), 2.33 (s, 3H), 2.30 (s, 3H), 2.27-2.24 (m, 2H); ESI MS m/z 359 [C₁₉H₁₆ClFN₂O₂+H]⁺; HPLC (Method B) >99% (AUC), t_{R}=9.59 min; Chiral HPLC (Chiralpak AD, Method A) 98.6% (AUC), t_{R}=12.35 min.

### Preparation of (S)-4-(6-Fluoro-3a-hydroxy-7-methyl-4-oxo-2,3,3a,4-tetrahydro-1H-pyrrolo[2,3-b]quinolin-1-yl)-2-methylbenzonitrile

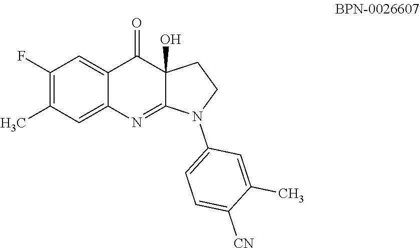

(S)-4-(6-Fluoro-3a-hydroxy-7-methyl-4-oxo-2,3,3a,4-tetrahydro-1H-pyrrolo[2,3-b]quinolin-1-yl)-2-methylbenzonitrile was prepared as a yellow solid according to Synthetic Scheme 3: mp=229-231° C.; ¹H NMR (300 MHz, DMSO-d₆) δ 8.25 (dd, J=8.4, 1.8 Hz, 1H), 8.11 (d, J=2.1 Hz, 1H), 7.80 (d, J=8.7 Hz, 1H), 7.41 (d, J=9.3 Hz, 1H), 7.28 (d, J=6.9 Hz, 1H), 6.97 (s, 1H), 4.07-4.02 (m, 2H), 2.53 (s, 3H), 2.31 (s, 3H), 2.27 (apparent s, 2H); ESI MS m/z 350 [C₂₀H₁₆FN₃O₂+H]⁺; HPLC (Method B) 97.8% (AUC), t_{R}=9.58 min; Chiral HPLC (Chiralpak AD, Method A) 96.1% (AUC), t_{R}=16.29 min.

### Preparation of provide (S)-4-(3a-Hydroxy-4-oxo-6-(trifluoromethyl)-2,3,3a,4-tetrahydro-1H-pyrrolo[2,3-b]quinolin-1-yl)benzonitrile

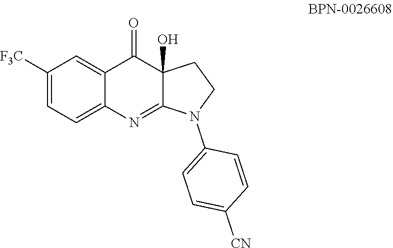 [SMILES: N#Cc1ccc(N2CC[C@@]3(O)C(=O)c4cc(C(F)(F)F)ccc4N=C23)cc1]

(S)-4-(3a-Hydroxy-4-oxo-6-(trifluoromethyl)-2,3,3a,4-tetrahydro-1H-pyrrolo[2,3-b]quinolin-1-yl)benzonitrile was prepared as a yellow solid according to Synthetic Scheme 3: mp=233-235° C.; ¹H NMR (500 MHz, DMSO-d₆) δ 8.33 (dd, J=7.0, 2.0 Hz, 2H), 7.96 (d, J=2.0 Hz, 1H), 7.93-7.90 (m, 3H), 7.46 (d, J=8.5 Hz, 1H), 7.12 (s, 1H), 4.18-4.13 (m, 1H), 4.10-4.06 (m, 1H), 2.44-2.39 (m, 1H), 2.32-2.28 (m, 1H); ESI MS m/z 372 [C₁₉H₁₂F₃N₃O₂+H]⁺; HPLC (Method B) >99% (AUC), t_{R}=10.66 min; Chiral HPLC (Chiralpak AD, Method A) 95.3% (AUC), t_{R}=16.42 min.

### Preparation of (S)-6-Fluoro-3a-hydroxy-1-(3-methoxyphenyl)-7-methyl-1,2,3,3a-tetrahydro-4H-pyrrolo[2,3-b]quinolin-4-one

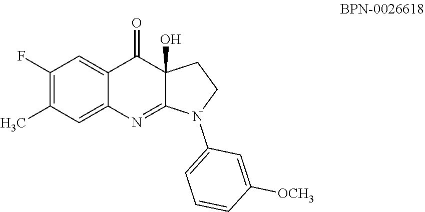 [SMILES: COc1cccc(N2CC[C@@]3(O)C(=O)c4cc(F)c(C)cc4N=C23)c1]

(S)-6-Fluoro-3a-hydroxy-1-(3-methoxyphenyl)-7-methyl-1,2,3,3a-tetrahydro-4H-pyrrolo[2,3-b]quinolin-4-one was prepared as a yellow solid according to Synthetic Scheme 3: mp=182-183° C.; ¹H NMR (500 MHz, DMSO-d₆) δ 7.96 (t, J=2.0 Hz, 1H), 7.47 (dd, J=8.0, 1.5 Hz, 1H), 7.37 (d, J=9.0 Hz, 1H), 7.32 (t, J=8.0 Hz, 1H), 7.18 (d, J=6.5 Hz, 1H), 6.87 (s, 1H), 6.74 (dd, J=8.0, 2.0 Hz, 1H), 4.08-4.03 (m, 1H), 3.98-3.95 (m, 1H), 3.80 (s, 3H), 2.29 (s, 3H), 2.28-2.24 (m, 2H); ESI MS m/z 341 [C₁₉H₁₇FN₂O₃+H]⁺; HPLC (Method B) >99% (AUC), t_{R}=8.53 min; Chiral HPLC (Chiralpak AD, Method A) >99% (AUC), t_{R}=14.78 min.

### Preparation of (S)-1-(Benzofuran-6-yl)-6-fluoro-3a-hydroxy-7-methyl-1,2,3,3a-tetrahydro-4H-pyrrolo[2,3-b]quinolin-4-one

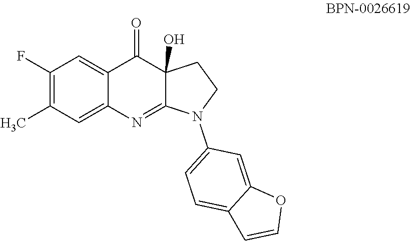 [SMILES: Cc1cc2c(cc1F)C(=O)[C@]1(O)CCN(c3ccc4ccoc4c3)C1=N2]

(S)-1-(Benzofuran-6-yl)-6-fluoro-3a-hydroxy-7-methyl-1,2,3,3a-tetrahydro-4H-pyrrolo[2,3-b]quinolin-4-one was prepared as a brown-orange solid according to Synthetic Scheme 3: mp=211-214° C.; ¹H NMR (500 MHz, DMSO-d₆) δ 8.54 (s, 1H), 7.99 (d, J=2.0 Hz, 1H), 7.87 (dd, J=8.5, 2.0 Hz, 1H), 7.68 (d, J=8.5 Hz, 1H), 7.38 (d, J=9.0 Hz, 1H), 7.22 (d, J=6.5 Hz, 1H), 6.95 (dd, J=2.0, 1.0 Hz, 1H), 6.90 (s, 1H), 4.18-4.13 (m, 1H), 4.05-4.02 (m, 1H), 2.30-2.26 (m, 5H); ESI MS m/z 351 [C₂₀H₁₅FN₂O₃+H]⁺; HPLC (Method B) >99% (AUC), t_{R}=8.89 min; Chiral HPLC (Chiralpak AD, Method A) >99% (AUC), t_{R}=17.52 min.

### Preparation of (S)-6-Fluoro-3a-hydroxy-7-methyl-1-(m-tolyl)-1,2,3,3a-tetrahydro-4H-pyrrolo[2,3-b]quinolin-4-one

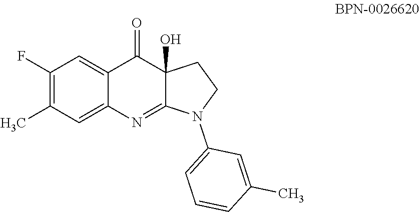

(S)-6-Fluoro-3a-hydroxy-7-methyl-1-(m-tolyl)-1,2,3,3a-tetrahydro-4H-pyrrolo[2,3-b]quinolin-4-one was prepared as a yellow solid according to Synthetic Scheme 3: mp=201-203° C.; ¹H NMR (500 MHz, DMSO-d₆) δ 7.92 (d, J=6.0 Hz, 1H), 7.83 (s, 1H), 7.36 (d, J=9.0 Hz, 1H), 7.30 (t, J=8.0 Hz, 1H), 7.16 (d, J=6.5 Hz, 1H), 6.98 (d, J=7.0 Hz, 1H), 6.86 (s, 1H), 4.08-4.03 (m, 1H), 3.97-3.93 (m, 1H), 2.36 (s, 3H), 2.28 (apparent d, J=1.0 Hz, 3H), 2.27-2.24 (m, 2H); ESI MS m/z 325 [C₁₉H₁₇FN₂O₂+H]⁺; HPLC (Method B) >99% (AUC), t_{R}=8.80 min; Chiral HPLC (Chiralpak AD, Method A) >99% (AUC), t_{R}=12.23 min.

### Preparation of (S)-4-(6-Fluoro-3a-hydroxy-7-methyl-4-oxo-2,3,3a,4-tetrahydro-1H-pyrrolo[2,3-b]quinolin-1-yl)benzonitrile

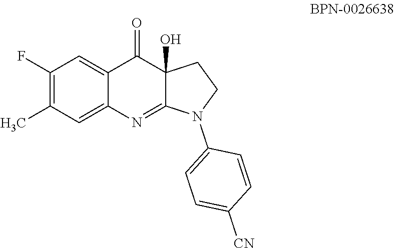 [SMILES: Cc1cc2c(cc1F)C(=O)[C@]1(O)CCN(c3ccc(C#N)cc3)C1=N2]

(S)-4-(6-Fluoro-3a-hydroxy-7-methyl-4-oxo-2,3,3a,4-tetrahydro-1H-pyrrolo[2,3-b]quinolin-1-yl)benzonitrile was prepared as a yellow solid according to Synthetic Scheme 4: ¹H NMR (500 MHz, DMSO-d₆) δ 8.31 (d, J=9.0 Hz, 2H), 7.88 (d, J=9.0 Hz, 2H), 7.42 (d, J=9.0 Hz, 1H), 7.27 (d, J=6.5 Hz, 1H), 6.98 (broad s, 1H), 4.07-4.03 (m, 2H), 2.31-2.27 (m, 5H); ESI MS m/z 336 [C₁₉H₁₄FN₃O₂+H]⁺; HPLC (Method B) >99% (AUC), t_{R}=8.24 min; Chiral SFC (Chiralcel OJ-H, Method B) >99% (AUC), t_{R}=5.29 min.

### Preparation of (S)-6,7-Dichloro-3a-hydroxy-1-phenyl-1,2,3,3a-tetrahydro-4H-pyrrolo[2,3-b]quinolin-4-one

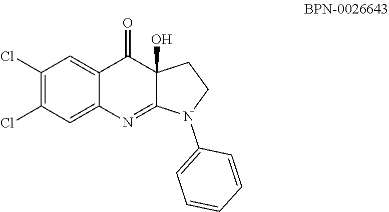 [SMILES: O=C1c2cc(Cl)c(Cl)cc2N=C2N(c3ccccc3)CC[C@@]12O]

(S)-6,7-Dichloro-3a-hydroxy-1-phenyl-1,2,3,3a-tetrahydro-4H-pyrrolo[2,3-b]quinolin-4-one was prepared as a yellow solid according to Synthetic Scheme 3: mp=202-204° C.; ¹H NMR (300 MHz, DMSO-d₆) δ 8.05 (dd, J=8.7, 1.2 Hz, 2H), 7.81 (s, 1H), 7.47-7.42 (m, 3H), 7.20 (t, J=7.5 Hz, 1H), 7.04 (s, 1H), 4.18-4.10 (m, 1H), 4.02-3.96 (m, 1H), 2.42-2.32 (m, 1H), 2.28-2.22 (m, 1H); ESI MS m/z 347 [C₁₇H₁₂Cl₂N₂O₂+H]⁺; HPLC (Method B) 97.4% (AUC), t_{R}=9.10 min; Chiral HPLC (Chiralpak AD, Method A) >99% (AUC), t_{R}=12.93 min.

### Preparation of (S)-3a-Hydroxy-6-methyl-1-phenyl-7-(trifluoromethyl)-1,2,3,3a-tetrahydro-4H-pyrrolo[2,3-b]quinolin-4-one

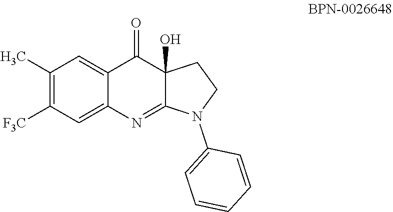 [SMILES: Cc1cc2c(cc1C(F)(F)F)N=C1N(c3ccccc3)CC[C@@]1(O)C2=O]

(S)-3a-Hydroxy-6-methyl-1-phenyl-7-(trifluoromethyl)-1,2,3,3a-tetrahydro-4H-pyrrolo[2,3-b]quinolin-4-one was prepared as a yellow solid according to Synthetic Scheme 3: mp=205-209° C.; ¹H NMR (300 MHz, DMSO-d₆) δ 8.08 (d, J=7.8 Hz, 2H), 7.73 (s, 1H), 7.48-7.43 (m, 3H), 7.18 (t, J=7.5 Hz, 1H), 6.99 (s, 1H), 4.13-4.08 (m, 1H), 4.03-3.96 (m, 1H), 2.43 (s, 3H), 2.38-2.29 (m, 2H); ESI MS m/z 361 [C₁₉H₁₅F₃N₂O₂+H]⁺; HPLC (Method B) 98.3% (AUC), t_{R}=9.29 min; Chiral SFC (Chiralcel OJ, Method A) 91.3% (AUC), t_{R}=4.16 min.

### Preparation of (S)-6-Fluoro-3a-hydroxy-7-methyl-1-(2-methylthiazol-5-yl)-1,2,3,3a-tetrahydro-4H-pyrrolo[2,3-b]quinolin-4-one

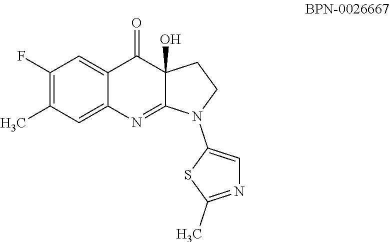 [SMILES: Cc1ncc(N2CC[C@@]3(O)C(=O)c4cc(F)c(C)cc4N=C23)s1]

(S)-6-Fluoro-3a-hydroxy-7-methyl-1-(2-methylthiazol-5-yl)-1,2,3,3a-tetrahydro-4H-pyrrolo[2,3-b]quinolin-4-one was prepared as a yellow solid according to Synthetic Scheme 4: ¹H NMR (500 MHz, DMSO-d₆) δ 7.47 (s, 1H), 7.38 (d, J=9.0 Hz, 1H), 7.18 (d, J=6.5 Hz, 1H), 6.97 (s, 1H), 4.07-3.92 (m, 2H), 2.60 (s, 3H), 2.44-2.41 (m, 1H), 2.31-2.28 (m, 4H); ESI MS m/z 332 [C₁₆H₁₄FN₃O₂S+H]⁺; HPLC (Method B) >99% (AUC), t_{R}=9.16 min; Chiral HPLC (Chiralpak AD, Method A) 98.6% (AUC), t_{R}=13.72 min.

### Preparation of (S)-6-Fluoro-3a-hydroxy-7-methyl-1-(2-methylbenzofuran-5-yl)-1,2,3,3a-tetrahydro-4H-pyrrolo[2,3-b]quinolin-4-one

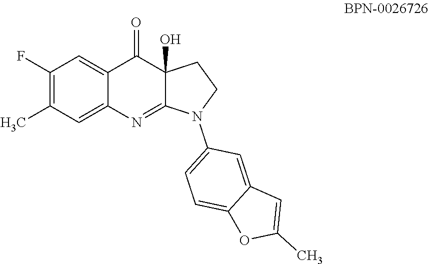

(S)-6-Fluoro-3a-hydroxy-7-methyl-1-(2-methylbenzofuran-5-yl)-1,2,3,3a-tetrahydro-4H-pyrrolo[2,3-b]quinolin-4-one was prepared as a yellow solid according to Synthetic Scheme 4: mp=203-205° C.; ¹H NMR (500 MHz, DMSO-d₆) δ 8.12 (d, J=2.5 Hz, 1H), 7.91 (dd, J=9.0, 2.5 Hz, 1H), 7.52 (d, J=9.0 Hz, 1H), 7.36 (d, J=9.5 Hz, 1H), 7.13 (d, J=7.0 Hz, 1H), 6.86 (s, 1H), 6.62 (s, 1H), 4.17-4.12 (m, 1H), 3.99-3.95 (m, 1H), 2.46 (apparent d, J=1.0 Hz, 3H), 2.28-2.26 (m, 5H); ESI MS m/z 365 [C₂₁H₁₇FN₂O₃+H]⁺; HPLC (Method B) >99% (AUC), t_{R}=9.12 min; Chiral HPLC (Chiralpak AD, Method A) 95.8% (AUC), t_{R}=15.34 min.

### Preparation of (S)-6-Fluoro-3a-hydroxy-1-(2-methoxypyridin-4-yl)-7-methyl-1,2,3,3a-tetrahydro-4H-pyrrolo[2,3-b]quinolin-4-one

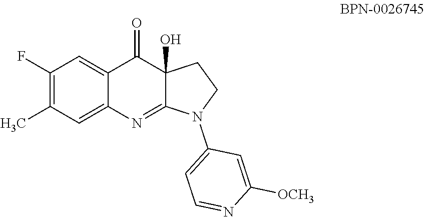 [SMILES: COc1cc(N2CC[C@@]3(O)C(=O)c4cc(F)c(C)cc4N=C23)ccn1]

(S)-6-Fluoro-3a-hydroxy-1-(2-methoxypyridin-4-yl)-7-methyl-1,2,3,3a-tetrahydro-4H-pyrrolo[2,3-b]quinolin-4-one was prepared as a yellow solid according to Synthetic Scheme 4: mp=211-215° C.; ¹H NMR (500 MHz, DMSO-d₆) δ 8.13 (d, J=6.0 Hz, 1H), 7.68 (dd, J=6.0, 2.0 Hz, 1H), 7.59 (d, J=2.0 Hz, 1H), 7.41 (d, J=9.0 Hz, 1H), 7.29 (d, J=7.0 Hz, 1H), 6.96 (s, 1H), 4.00-3.97 (m, 2H), 3.87 (s, 3H), 2.36 (s, 3H), 2.31-2.23 (m, 2H); ESI MS m/z 342 [C₁₈H₁₆FN₃O₃+H]⁺; HPLC (Method B) >99% (AUC), t_{R}=8.95 min; Chiral HPLC (Chiralpak AD, Method A) 97.7% (AUC), t_{R}=14.01 min.

### Preparation of (S)-6-Fluoro-3a-hydroxy-7-methyl-1-(1-methyl-1H-pyrazol-4-yl)-1,2,3,3a-tetrahydro-4H-pyrrolo[2,3-b]quinolin-4-one

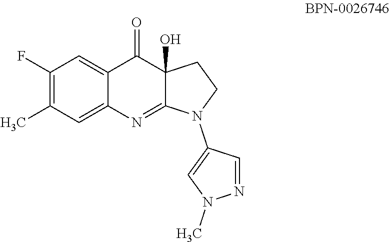 [SMILES: Cc1cc2c(cc1F)C(=O)[C@]1(O)CCN(c3cnn(C)c3)C1=N2]

(S)-6-Fluoro-3a-hydroxy-7-methyl-1-(1-methyl-1H-pyrazol-4-yl)-1,2,3,3a-tetrahydro-4H-pyrrolo[2,3-b]quinolin-4-one was prepared as a yellow solid according to Synthetic Scheme 4: mp=234-238° C.; ¹H NMR (500 MHz, DMSO-d₆) δ 8.25 (s, 1H), 7.83 (s, 1H), 7.32 (d, J=9.5 Hz, 1H), 7.15 (d, J=7.0 Hz, 1H), 6.80 (s, 1H), 3.92-3.91 (m, 1H), 3.87 (s, 3H), 3.83-3.81 (m, 1H), 2.36-2.35 (m, 1H), 2.31 (s, 3H), 2.28-2.25 (m, 1H); ESI MS m/z 315 [C₁₆H₁₅FN₄O₂+H]⁺; HPLC (Method B) >99% (AUC), t_{R}=7.31 min; Chiral HPLC (Chiralpak AD, Method A) 98.3% (AUC), t_{R}=16.79 min.

### Preparation of (S)-6-Fluoro-3a-hydroxy-7-methyl-1-(thiophen-2-yl)-1,2,3,3a-tetrahydro-4H-pyrrolo[2,3-b]quinolin-4-one

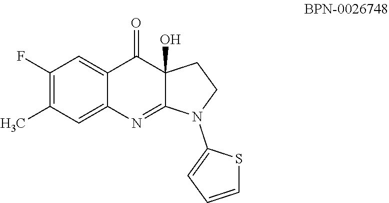 [SMILES: Cc1cc2c(cc1F)C(=O)[C@]1(O)CCN(c3cccs3)C1=N2]

(S)-6-Fluoro-3a-hydroxy-7-methyl-1-(thiophen-2-yl)-1,2,3,3a-tetrahydro-4H-pyrrolo[2,3-b]quinolin-4-one was prepared as a red-orange solid according to Synthetic Scheme 4: mp=210-215° C.; ¹H NMR (500 MHz, DMSO-d₆) δ 7.37 (d, J=9.5 Hz, 1H), 7.18-7.15 (m, 2H), 6.96 (dd, J=5.5, 4.0 Hz, 1H), 6.94 (s, 1H), 6.85 (dd, J=4.0, 1.5 Hz, 1H), 4.09-3.99 (m, 2H), 2.44-2.37 (m, 1H), 2.30-2.27 (m, 4H); ESI MS m/z 317 [C₁₆H₁₃FN₂O₂S+H]⁺; HPLC (Method B) >99% (AUC), t_{R}=8.89 min; Chiral HPLC (Chiralpak AD, Method A) 96.7% (AUC), t_{R}=15.19 min.

### Preparation of (S)-3a-Hydroxy-1-phenyl-1,2,3,3a-tetrahydro-4H-benzo[4,5]thieno[2,3-b]pyrrolo[3,2-e]pyridin-4-one

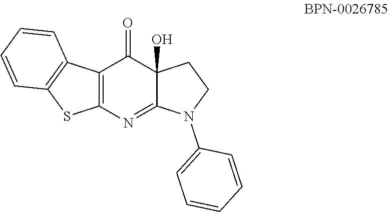 [SMILES: O=C1c2c(sc3ccccc23)N=C2N(c3ccccc3)CC[C@@]12O]

(S)-3a-Hydroxy-1-phenyl-1,2,3,3a-tetrahydro-4H-benzo[4,5]thieno[2,3-b]pyrrolo[3,2-e]pyridin-4-one was prepared as a yellow solid according to Synthetic Scheme 3: mp=247-248° C.; ¹H NMR (500 MHz, DMSO-d₆) δ 8.19 (d, J=7.5 Hz, 1H), 7.96 (dd, J=8.5, 7.5 Hz, 2H), 7.84 (d, J=7.5 Hz, 1H), 7.52-7.49 (m, 2H), 7.42-7.39 (m, 1H), 7.29-7.25 (m, 2H), 7.06 (s, 1H), 4.30-4.25 (m, 1H), 4.12 (apparent t, J=7.5 Hz, 1H), 2.36-2.30 (m, 2H); ESI MS m/z 335 [C₁₉H₁₄N₂O₂S+H]⁺; HPLC (Method B) >99% (AUC), t_{R}=10.70 min; Chiral HPLC (Chiralpak AD, Method A) >99% (AUC), t_{R}=21.14 min.

### Preparation of (S)-1-(3-Chloro-4-methylphenyl)-3a-hydroxy-6-methyl-7-(trifluoromethyl)-1,2,3,3a-tetrahydro-4H-pyrrolo[2,3-b]quinolin-4-one

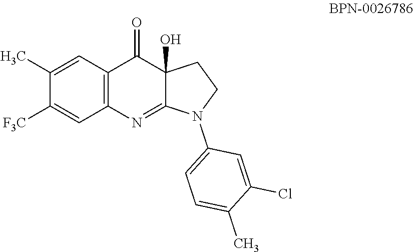 [SMILES: Cc1ccc(N2CC[C@@]3(O)C(=O)c4cc(C)c(C(F)(F)F)cc4N=C23)cc1Cl]

(S)-1-(3-Chloro-4-methylphenyl)-3a-hydroxy-6-methyl-7-(trifluoromethyl)-1,2,3,3a-tetrahydro-4H-pyrrolo[2,3-b]quinolin-4-one was prepared as an orange solid according to Synthetic Scheme 3: mp=212-217° C.; ¹H NMR (300 MHz, DMSO-d₆) δ 8.28 (d, J=2.4 Hz, 1H), 7.89 (dd, J=8.4, 2.1 Hz, 1H), 7.74 (s, 1H), 7.41 (t, J=4.8 Hz, 2H), 7.01 (s, 1H), 4.13-3.95 (m, 2H), 2.44 (s, 3H), 2.34 (s, 3H), 2.28-2.26 (m, 2H); ESI MS m/z 409 [C₂₀H₁₆ClF₃N₂O₂+H]⁺; HPLC (Method B) >99% (AUC), t_{R}=10.44 min; Chiral HPLC (Chiralpak AD, Method A) >99% (AUC), t_{R}=9.82 min.

### Preparation of (S)-3a-Hydroxy-6-methyl-1-(p-tolyl)-7-(trifluoromethyl)-1,2,3,3a-tetrahydro-4H-pyrrolo[2,3-b]quinolin-4-one

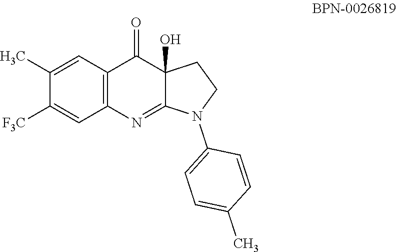 [SMILES: Cc1ccc(N2CC[C@@]3(O)C(=O)c4cc(C)c(C(F)(F)F)cc4N=C23)cc1]

(S)-3a-Hydroxy-6-methyl-1-(p-tolyl)-7-(trifluoromethyl)-1,2,3,3a-tetrahydro-4H-pyrrolo[2,3-b]quinolin-4-one was prepared as an orange-yellow solid according to Synthetic Scheme 3: mp=202-205° C.; ¹H NMR (500 MHz, DMSO-d₆) δ 7.93 (d, J=8.5 Hz, 2H), 7.71 (s, 1H), 7.39 (s, 1H), 7.25 (d, J=8.5 Hz, 2H), 6.94 (s, 1H), 4.12-4.07 (m, 1H), 3.96 (apparent t, J=7.5 Hz, 1H) 2.42 (s, 3H), 2.36-2.34 (m, 1H), 2.32 (s, 3H), 2.27-2.23 (m, 1H); ESI MS m/z 375 [C₂₀H₁₇F₃N₂O₂+H]⁺; HPLC (Method C) >99% (AUC), t_{R}=14.19 min; Chiral HPLC (Chiralpak AD, Method A) >99% (AUC), t_{R}=10.94 min.

### Preparation of (S)-3a-Hydroxy-6-methyl-1-(m-tolyl)-7-(trifluoromethyl)-1,2,3,3a-tetrahydro-4H-pyrrolo[2,3-b]quinolin-4-one

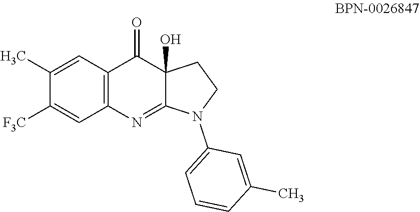

(S)-3a-Hydroxy-6-methyl-1-(m-tolyl)-7-(trifluoromethyl)-1,2,3,3a-tetrahydro-4H-pyrrolo[2,3-b]quinolin-4-one was prepared as a yellow solid according to Synthetic Scheme 3: mp=181-185° C.; ¹H NMR (300 MHz, DMSO-d₆) δ 7.95 (d, J=8.4 Hz, 1H), 7.80 (s, 1H), 7.73 (s, 1H), 7.42 (s, 1H), 7.33 (t, J=7.8 Hz, 1H), 7.00 (d, J=7.5 Hz, 1H), 6.98 (s, 1H), 4.15-4.06 (m, 1H), 3.98 (apparent t, J=9.0 Hz, 1H), 2.43 (s, 3H), 2.37 (s, 3H), 2.30-2.22 (m, 2H); ESI MS m/z 375 [C₂₀H₁₇F₃N₂O₂+H]⁺; HPLC (Method B) >99% (AUC), t_{R}=9.63 min; Chiral HPLC (Chiralpak AD, Method A) >99% (AUC), t_{R}=9.40 min.

### Preparation of (S)-3a-Hydroxy-1-(4-methoxyphenyl)-6-methyl-7-(trifluoromethyl)-1,2,3,3a-tetrahydro-4H-pyrrolo[2,3-b]quinolin-4-one

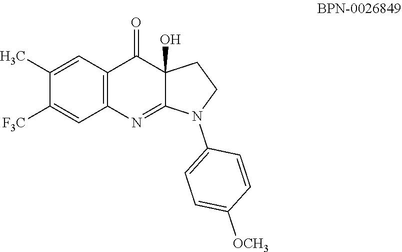 [SMILES: COc1ccc(N2CC[C@@]3(O)C(=O)c4cc(C)c(C(F)(F)F)cc4N=C23)cc1]

(S)-3a-Hydroxy-1-(4-methoxyphenyl)-6-methyl-7-(trifluoromethyl)-1,2,3,3a-tetrahydro-4H-pyrrolo[2,3-b]quinolin-4-one was prepared as a yellow solid according to Synthetic Scheme 3: mp=185-188° C.; ¹H NMR (500 MHz, DMSO-d₆) δ 7.95 (dd, J=7.0, 2.5 Hz, 2H), 7.70 (s, 1H), 7.36 (s, 1H), 7.01 (dd, J=7.0, 2.0 Hz, 2H), 6.93 (s, 1H), 4.10-4.08 (m, 1H), 3.94 (apparent t, J=9.0 Hz, 1H), 3.78 (s, 3H), 2.42 (s, 3H), 2.36-2.33 (m, 1H), 2.27-2.23 (m, 1H); ESI MS m/z 391 [C₂₀H₁₇F₃N₂O₃+H]⁺; HPLC (Method B) >99% (AUC), t_{R}=9.83 min; Chiral HPLC (Chiralpak AD, Method A) >99% (AUC), t_{R}=13.62 min.

### Preparation of (S)-5-(3-Chloro-4-methylphenyl)-7a-hydroxy-2-methyl-5,6,7,7a-tetrahydro-8H-pyrrolo[2,3-b]thieno[2,3-e]pyridin-8-one

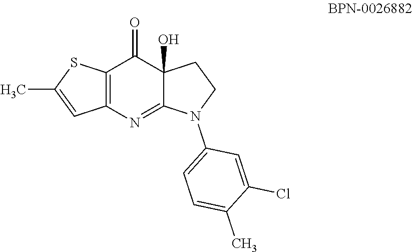 [SMILES: Cc1cc2c(s1)C(=O)[C@]1(O)CCN(c3ccc(C)c(Cl)c3)C1=N2]

(S)-5-(3-Chloro-4-methylphenyl)-7a-hydroxy-2-methyl-5,6,7,7a-tetrahydro-8H-pyrrolo[2,3-b]thieno[2,3-e]pyridin-8-one was prepared as a yellow solid according to Synthetic Scheme 3: mp=210-212° C.; ¹H NMR (500 MHz, DMSO-d₆) δ 8.25 (apparent d, J=2.0 Hz, 1H), 7.75 (dd, J=8.0, 2.0 Hz, 1H), 7.40 (d, J=8.5 Hz, 1H), 6.90 (s, 1H), 6.84 (s, 1H), 4.11-4.09 (m, 1H), 3.99 (apparent t, J=9.5 Hz, 1H), 2.33 (s, 3H), 2.24-2.18 (m, 2H), 3H obscured by solvent peak; ESI MS m/z 347 [C₁₇H₁₅ClN₂O₂S+H]⁺; HPLC (Method B) >99% (AUC), t_{R}=9.74 min; Chiral HPLC (Chiralpak AD, Method A) >99% (AUC), t_{R}=16.62 min.

### Preparation of (S)-3a-Hydroxy-1-(2-methoxypyridin-4-yl)-6-methyl-7-(trifluoromethyl)-1,2,3,3a-tetrahydro-4H-pyrrolo[2,3-b]quinolin-4-one

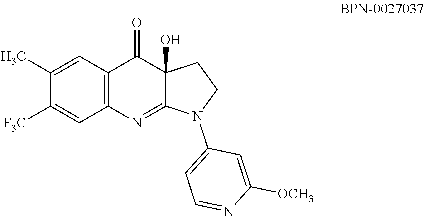 [SMILES: COc1cc(N2CC[C@@]3(O)C(=O)c4cc(C)c(C(F)(F)F)cc4N=C23)ccn1]

(S)-3a-Hydroxy-1-(2-methoxypyridin-4-yl)-6-methyl-7-(trifluoromethyl)-1,2,3,3a-tetrahydro-4H-pyrrolo[2,3-b]quinolin-4-one was prepared as a yellow solid according to Synthetic Scheme 4: ¹H NMR (500 MHz, DMSO-d₆) δ 8.15 (d, J=6.0 Hz, 1H), 7.80-7.77 (m, 2H), 7.53-7.51 (m, 2H), 7.05 (s, 1H), 4.03-4.01 (m, 2H), 3.87 (s, 3H), 2.46 (s, 3H), 2.28-2.23 (m, 2H); ESI MS m/z 392 [C₁₉H₁₆F₃N₃O₃+H]⁺; HPLC (Method B) >99% (AUC), t_{R}=11.12 min; Chiral HPLC (Chiralpak AD, Method A) 69.1% (AUC), t_{R}=11.35 min.

### Preparation of (S)-4-(3a-Hydroxy-6-methyl-4-oxo-7-(trifluoromethyl)-2,3,3a,4-tetrahydro-1H-pyrrolo[2,3-b]quinolin-1-yl)benzonitrile

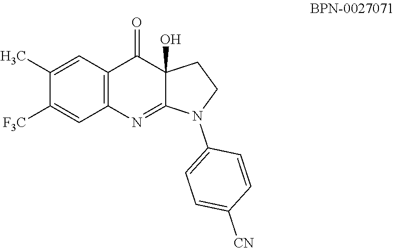 [SMILES: Cc1cc2c(cc1C(F)(F)F)N=C1N(c3ccc(C#N)cc3)CC[C@@]1(O)C2=O]

(S)-4-(3a-Hydroxy-6-methyl-4-oxo-7-(trifluoromethyl)-2,3,3a,4-tetrahydro-1H-pyrrolo[2,3-b]quinolin-1-yl)benzonitrile was prepared as a yellow solid according to Synthetic Scheme 6: mp=225-228° C.; ¹H NMR (500 MHz, DMSO-d₆) δ 8.33 (d, J=9.0 Hz, 2H), 7.89 (d, J=9.0 Hz, 2H), 7.77 (s, 1H), 7.53 (s, 1H), 7.05 (s, 1H), 4.11-4.06 (m, 2H), 2.45 (s, 3H), 2.43-2.38 (m, 1H), 2.30-2.27 (m, 1H); ESI MS m/z 386 [C₂₀H₁₄F₃N₃O₂+H]⁺; HPLC (Method C) >99% (AUC), t_{R}=15.95 min; Chiral HPLC (Chiralpak AD, Method A) 45.6% (AUC), t_{R}=16.30 min.

### Preparation of (S)-4-(7a-Hydroxy-2-methyl-8-oxo-6,7,7a,8-tetrahydro-5H-pyrrolo[2,3-b]thieno[2,3-e]pyridin-5-yl)-2-methylbenzonitrile

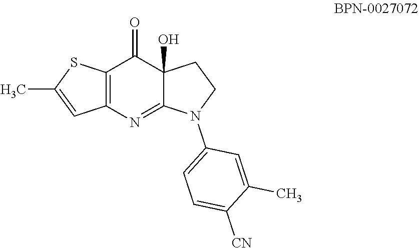

(S)-4-(7a-Hydroxy-2-methyl-8-oxo-6,7,7a,8-tetrahydro-5H-pyrrolo[2,3-b]thieno[2,3-e]pyridin-5-yl)-2-methylbenzonitrile was prepared as a yellow-brown solid according to Synthetic Scheme 3: mp=223-224° C.; ¹H NMR (500 MHz, DMSO-d₆) δ 8.19 (dd, J=8.5, 2.5 Hz, 1H), 8.02 (d, J=2.0 Hz, 1H), 7.81 (d, J=8.5 Hz, 1H), 6.97 (s, 1H), 6.90 (apparent d, J=1.0 Hz, 1H), 4.12-4.05 (m, 2H), 2.52 (s, 3H), 2.49 (s, 3H), 2.27-2.22 (m, 2H); ESI MS m/z 338 [C₁₈H₁₅N₃O₂S+H]⁺; HPLC (Method B) >99% (AUC), t_{R}=8.67 min; Chiral HPLC (Chiralpak AD, Method A) 96.3% (AUC), t_{R}=19.63 min.

### Preparation of (S)-5-(3-Bromophenyl)-7a-hydroxy-2-methyl-5,6,7,7a-tetrahydro-8H-pyrrolo[2,3-b]thieno[2,3-e]pyridin-8-one

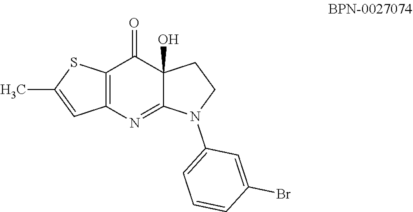 [SMILES: Cc1cc2c(s1)C(=O)[C@]1(O)CCN(c3cccc(Br)c3)C1=N2]

(S)-5-(3-Bromophenyl)-7a-hydroxy-2-methyl-5,6,7,7a-tetrahydro-8H-pyrrolo[2,3-b]thieno[2,3-e]pyridin-8-one was prepared as a yellow-orange solid according to Synthetic Scheme 3: mp=193-197° C.; ¹H NMR (300 MHz, DMSO-d₆) δ 8.42 (s, 1H), 7.90 (dd, J=7.2, 4.8 Hz, 1H), 7.43-7.38 (m, 2H), 6.94 (s, 1H), 6.90 (apparent d, J=0.9 Hz, 1H), 4.16-3.98 (m, 2H), 2.27-2.14 (m, 2H), 3H obscured by solvent peak; ESI MS m/z 377 [C₁₆H₁₃BrN₂O₂S+H]⁺; HPLC (Method B) 97.4% (AUC), t_{R}=8.27 min; Chiral HPLC (Chiralpak AD, Method A) >99% (AUC), t_{R}=16.25 min.

### Preparation of (S)-1-(4-Chlorophenyl)-3a-hydroxy-6-methyl-7-(trifluoromethyl)-1,2,3,3a-tetrahydro-4H-pyrrolo[2,3-b]quinolin-4-one

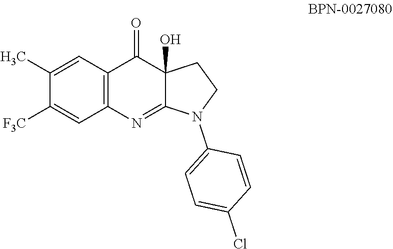 [SMILES: Cc1cc2c(cc1C(F)(F)F)N=C1N(c3ccc(Cl)cc3)CC[C@@]1(O)C2=O]

(S)-1-(4-Chlorophenyl)-3a-hydroxy-6-methyl-7-(trifluoromethyl)-1,2,3,3a-tetrahydro-4H-pyrrolo[2,3-b]quinolin-4-one was prepared as a yellow solid according to Synthetic Scheme 3: mp=223-226° C.; ¹H NMR (500 MHz, DMSO-d₆) δ 8.13 (apparent dt, J=9.0, 3.5 Hz, 2H), 7.34 (s, 1H), 7.50 (apparent dt, J=9.0, 3.5 Hz, 2H), 7.44 (s, 1H), 6.99 (s, 1H), 4.11-4.07 (m, 1H), 3.99 (apparent t, J=9.5 Hz, 1H), 2.43 (s, 3H), 2.40-2.34 (m, 1H), 2.28-2.24 (m, 1H); ESI MS m/z 395 [C₁₉H₁₄ClF₃N₂O₂+H]⁺; HPLC (Method C) >99% (AUC), t_{R}=15.90 min; Chiral HPLC (Chiralpak AD, Method A) >99% (AUC), t_{R}=12.18 min.

### Preparation of (S)-1-(Benzofuran-6-yl)-3a-hydroxy-6-methyl-7-(trifluoromethyl)-1,2,3,3a-tetrahydro-4H-pyrrolo[2,3-b]quinolin-4-one

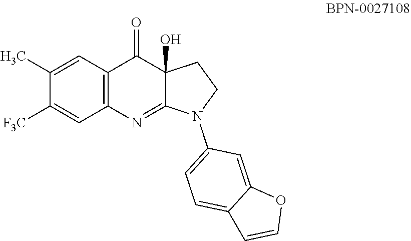 [SMILES: Cc1cc2c(cc1C(F)(F)F)N=C1N(c3ccc4ccoc4c3)CC[C@@]1(O)C2=O]

(S)-1-(Benzofuran-6-yl)-3a-hydroxy-6-methyl-7-(trifluoromethyl)-1,2,3,3a-tetrahydro-4H-pyrrolo[2,3-b]quinolin-4-one was prepared as a bright orange solid according to Synthetic Scheme 3: mp=206-209° C.; ¹H NMR (500 MHz, DMSO-d₆) δ 8.47 (s, 1H), 8.01 (d, J=2.5 Hz, 1H), 7.92 (dd, J=8.5, 2.0 Hz, 1H), 7.74 (s, 1H), 7.70 (d, J=8.5 Hz, 1H), 7.45 (s, 1H), 6.99 (s, 1H), 6.97 (apparent dd, J=2.0, 1.0 Hz, 1H), 4.22-4.17 (m, 1H), 4.06 (apparent t, J=9.0 Hz, 1H), 2.43 (s, 3H), 2.41-2.38 (m, 1H), 2.30-2.27 (m, 1H); ESI MS m/z 401 [C₂₁H₁₅F₃N₂O₃+H]⁺; HPLC (Method B) >99% (AUC), t_{R}=8.96 min; Chiral HPLC (Chiralpak AD, Method A) >99% (AUC), t_{R}=14.11 min.

### Preparation of (S)-3a-Hydroxy-6,7-dimethyl-1-(2-methylbenzo[d]oxazol-5-yl)-1,2,3,3a-tetrahydro-4H-pyrrolo[2,3-b]quinolin-4-one

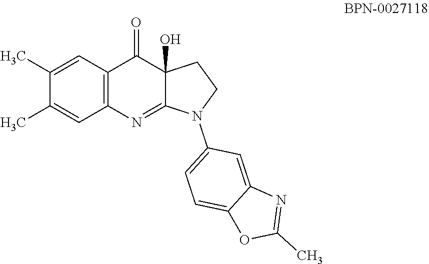

(S)-3a-Hydroxy-6,7-dimethyl-1-(2-methylbenzo[d]oxazol-5-yl)-1,2,3,3a-tetrahydro-4H-pyrrolo[2,3-b]quinolin-4-one was prepared as a yellow solid according to Synthetic Scheme 4: ¹H NMR (500 MHz, DMSO-d₆) δ 8.44 (apparent d, J=2.0 Hz, 1H), 8.01 (dd, J=9.0, 2.5 Hz, 1H), 7.68 (d, J=9.0 Hz, 1H), 7.49 (s, 1H), 7.05 (s, 1H), 6.78 (s, 1H), 4.16-4.11 (m, 1H), 4.05-3.98 (m, 1H), 2.62 (s, 3H), 2.27-2.26 (m, 5H), 2.22 (s, 3H); ESI MS m/z 362 [C₂₁H₁₉N₃O₃+H]⁺; HPLC (Method B) >99% (AUC), t_{R}=7.83 min; Chiral HPLC (Chiralpak AD, Method A) 84.2% (AUC), t_{R}=23.32 min.

### Preparation of (S)-2-Chloro-4-(7a-hydroxy-2-methyl-8-oxo-6,7,7a,8-tetrahydro-5H-pyrrolo[2,3-b]thieno[2,3-e]pyridin-5-yl)benzonitrile

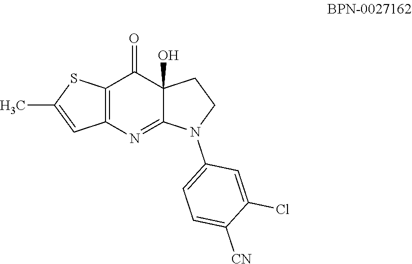 [SMILES: Cc1cc2c(s1)C(=O)[C@]1(O)CCN(c3ccc(C#N)c(Cl)c3)C1=N2]

(S)-2-Chloro-4-(7a-hydroxy-2-methyl-8-oxo-6,7,7a,8-tetrahydro-5H-pyrrolo[2,3-b]thieno[2,3-e]pyridin-5-yl)benzonitrile was prepared as a yellow solid according to Synthetic Scheme 3: mp=247-250° C.; ¹H NMR (500 MHz, DMSO-d₆) δ 8.55 (apparent d, J=2.0 Hz, 1H), 8.13 (dd, J=9.0, 2.0 Hz, 1H), 8.01 (d, J=9.0 Hz, 1H), 7.04 (s, 1H), 6.95 (apparent d, J=1.0 Hz, 1H), 4.14-4.08 (m, 2H), 2.52 (s, 3H), 2.29-2.19 (m, 2H); ESI MS m/z 358 [C₁₇H₁₂ClN₃O₂S+H]⁺; HPLC (Method B) >99% (AUC), t_{R}=9.82 min; Chiral HPLC (Chiralpak AD, Method A) 77.2% (AUC), t_{R}=20.56 min.

### Preparation of (S)-7a-Hydroxy-2-methyl-5-(quinolin-6-yl)-5,6,7,7a-tetrahydro-8H-pyrrolo[2,3-b]thieno[2,3-e]pyridin-8-one

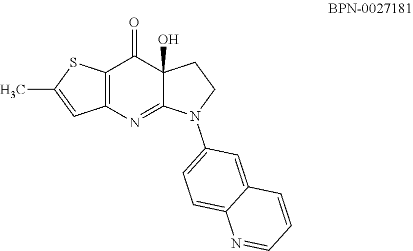 [SMILES: Cc1cc2c(s1)C(=O)[C@]1(O)CCN(c3ccc4ncccc4c3)C1=N2]

(S)-7a-Hydroxy-2-methyl-5-(quinolin-6-yl)-5,6,7,7a-tetrahydro-8H-pyrrolo[2,3-b]thieno[2,3-e]pyridin-8-one was prepared as a yellow solid according to Synthetic Scheme 3: mp=247-249° C.; ¹H NMR (500 MHz, DMSO-d₆) δ 8.86 (dd, J=4.5, 2.0 Hz, 1H), 8.67 (dd, J=9.0, 2.5 Hz, 1H), 8.38 (d, J=2.0 Hz, 1H), 8.36 (d, J=7.0 Hz, 1H), 8.07 (d, J=9.5 Hz, 1H), 7.55 (dd, J=8.0, 4.0 Hz, 1H), 6.97 (s, 1H), 6.88 (apparent d, J=0.5 Hz, 1H), 4.29-4.24 (m, 1H), 4.17 (apparent t, J=7.5 Hz, 1H), 2.52 (s, 3H), 2.32-2.27 (m, 2H); ESI MS m/z 350 [C₁₉H₁₅N₃O₂S+H]⁺; HPLC (Method B) >99% (AUC), t_{R}=7.18 min; Chiral HPLC (Chiralpak AD, Method A) 97.5% (AUC), t_{R}=21.48 min.

### Preparation of (S)-1-(Benzo[d]thiazol-6-yl)-6-fluoro-3a-hydroxy-7-methyl-1,2,3,3a-tetrahydro-4H-pyrrolo[2,3-b]quinolin-4-one

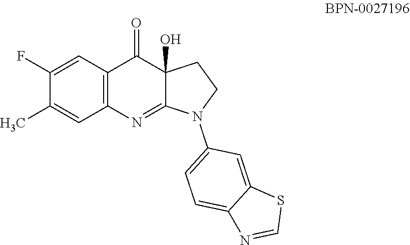 [SMILES: Cc1cc2c(cc1F)C(=O)[C@]1(O)CCN(c3ccc4ncsc4c3)C1=N2]

(S)-1-(Benzo[d]thiazol-6-yl)-6-fluoro-3a-hydroxy-7-methyl-1,2,3,3a-tetrahydro-4H-pyrrolo[2,3-b]quinolin-4-one was prepared as a yellow solid according to Synthetic Scheme 6: mp=130-133° C.; ¹H NMR (500 MHz, DMSO-d₆) δ 9.33 (s, 1H), 8.90 (d, J=2.5 Hz, 1H), 8.32 (dd, J=9.0, 2.0 Hz, 1H), 8.12 (d, J=9.0 Hz, 1H), 7.39 (d, J=9.0 Hz, 1H), 7.26 (d, J=6.5 Hz, 1H), 6.93 (s, 1H), 4.20-4.14 (m, 1H), 4.07 (apparent t, J=8.0 Hz, 1H), 2.34-2.27 (m, 5H); ESI MS m/z 368 [C₁₉H₁₄FN₃O₂S+H]⁺; HPLC (Method C) >99% (AUC), t_{R}=12.76 min; Chiral HPLC (Chiralpak AD, Method A) 75.2% (AUC), t_{R}=20.73 min.

### Preparation of (S)-6-Fluoro-3a-hydroxy-7-methyl-1-(quinolin-6-yl)-3,3a-dihydro-1H-pyrrolo[2,3-b]quinolin-4(2H)-one

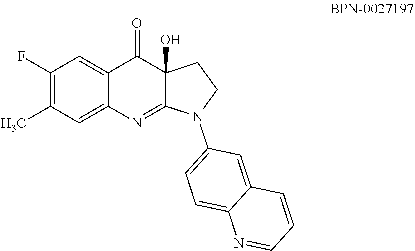 [SMILES: Cc1cc2c(cc1F)C(=O)[C@]1(O)CCN(c3ccc4ncccc4c3)C1=N2]

(S)-6-Fluoro-3a-hydroxy-7-methyl-1-(quinolin-6-yl)-3,3a-dihydro-1H-pyrrolo[2,3-b]quinolin-4(2H)-one was prepared as a yellow solid according to Synthetic Scheme 6: ¹H NMR (500 MHz, DMSO-d₆) δ 8.84 (dd, J=4.5, 2.0 Hz, 1H), 8.76 (dd, J=9.0, 2.5 Hz, 1H), 8.46 (d, J=2.5 Hz, 1H), 8.37 (d, J=8.5 Hz, 1H), 8.06 (d, J=9.0 Hz, 1H), 7.54 (dd, J=8.5, 4.5 Hz, 1H), 7.41 (d, J=9.5 Hz, 1H), 7.28 (d, J=6.5 Hz, 1H), 6.96 (s, 1H), 4.21-4.14 (m, 2H), 2.37-2.31 (m, 5H); ESI MS m/z 362 [C₂₁H₁₆FN₃O₂+H]⁺; HPLC (Method C) >99% (AUC), t_{R}=12.21 min; Chiral HPLC (Chiralpak AD, Method A) 58.8% (AUC), t_{R}=19.16 min.

### Preparation of (S)-6-Fluoro-3a-hydroxy-7-methyl-1-(2-methylbenzo[d]oxazol-5-yl)-1,2,3,3a-tetrahydro-4H-pyrrolo[2,3-b]quinolin-4-one

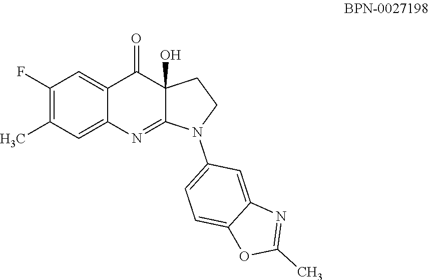 [SMILES: Cc1nc2cc(N3CC[C@@]4(O)C(=O)c5cc(F)c(C)cc5N=C34)ccc2o1]

(S)-6-Fluoro-3a-hydroxy-7-methyl-1-(2-methylbenzo[d]oxazol-5-yl)-1,2,3,3a-tetrahydro-4H-pyrrolo[2,3-b]quinolin-4-one was prepared as a yellow solid according to Synthetic Scheme 4: ¹H NMR (500 MHz, DMSO-d₆) δ 8.42 (d, J=2.0 Hz, 1H), 7.99 (dd, J=9.0, 2.0 Hz, 1H), 7.69 (d, J=9.0 Hz, 1H), 7.37 (d, J=9.0 Hz, 1H), 7.18 (d, J=7.5 Hz, 1H), 6.89 (s, 1H), 4.16-4.13 (m, 1H), 4.01 (apparent t, J=7.5 Hz, 1H), 2.63 (s, 3H), 2.31-2.26 (m, 5H); ESI MS m/z 366 [C₂₀H₁₆FN₃O₃+H]⁺; HPLC (Method C) >99% (AUC), t_{R}=12.02 min; Chiral HPLC (Chiralpak AD, Method A) 72.8% (AUC), t_{R}=20.60 min.

### Preparation of (S)-3a-Hydroxy-1-(quinolin-6-yl)-6-(trifluoromethyl)-1,2,3,3a-tetrahydro-4H-pyrrolo[2,3-b]quinolin-4-one

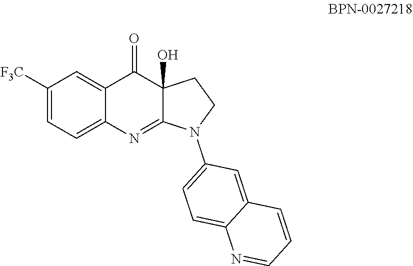 [SMILES: O=C1c2cc(C(F)(F)F)ccc2N=C2N(c3ccc4ncccc4c3)CC[C@@]12O]

(S)-3a-Hydroxy-1-(quinolin-6-yl)-6-(trifluoromethyl)-1,2,3,3a-tetrahydro-4H-pyrrolo[2,3-b]quinolin-4-one was prepared as a yellow solid according to Synthetic Scheme 6: mp=258-260° C.; ¹H NMR (300 MHz, DMSO-d₆) δ 8.87 (dd, J=3.9, 2.4 Hz, 1H), 8.73 (dd, J=9.0, 2.7 Hz, 1H), 8.51 (s, 1H), 8.41 (d, J=6.9 Hz, 1H), 8.10 (d, J=9.3 Hz, 1H), 7.96 (s, 1H), 7.91 (d, J=8.4 Hz, 1H), 7.56 (dd, J=8.4, 4.5 Hz, 1H), 7.47 (d, J=8.7 Hz, 1H), 7.13 (s, 1H), 4.30-4.16 (m, 2H), 2.74-2.63 (m, 1H), 2.17-1.98 (m, 1H); ESI MS m/z 398 [C₂₁H₁₄F₃N₃O₂+H]⁺; HPLC (Method C) >99% (AUC), t_{R}=13.48 min; Chiral HPLC (Chiralpak AD, Method A) 49.1% (AUC), t_{R}=16.74 min.

### Preparation of (S)-1-(Benzo[d]thiazol-6-yl)-3a-hydroxy-6-(trifluoromethyl)-1,2,3,3a-tetrahydro-4H-pyrrolo[2,3-b]quinolin-4-one

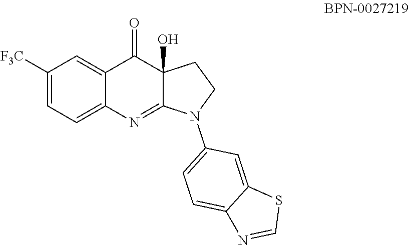 [SMILES: O=C1c2cc(C(F)(F)F)ccc2N=C2N(c3ccc4ncsc4c3)CC[C@@]12O]

(S)-1-(Benzo[d]thiazol-6-yl)-3a-hydroxy-6-(trifluoromethyl)-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 9.37 (s, 1H), 8.87 (d, J=2.5 Hz, 1H), 8.32 (dd, J=9.0, 2.5 Hz, 1H), 8.15 (d, J=9.0 Hz, 1H), 7.94 (d, J=2.0 Hz, 1H), 7.87 (dd, J=8.5, 2.5 Hz, 1H), 7.44 (d, J=8.5 Hz, 1H), 7.09 (s, 1H), 4.28-4.23 (m, 1H), 4.12 (apparent t, J=9.5 Hz, 1H), 2.34-2.31 (m, 2H); ESI MS m/z 404 [C₁₉H₁₂F₃N₃O₂S+H]⁺; HPLC (Method C) >99% (AUC), t_{R}=13.22 min; Chiral HPLC (Chiralpak AD, Method A) 50.0% (AUC), t_{R}=16.78 min.

### Preparation of (S)-3a-Hydroxy-6,7-dimethyl-1-(quinazolin-6-yl)-1,2,3,3a-tetrahydro-4H-pyrrolo[2,3-b]quinolin-4-one

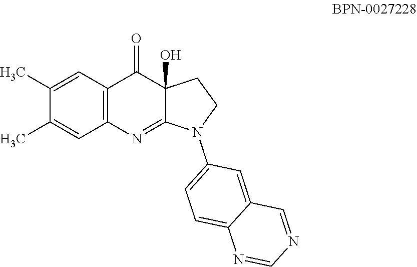 [SMILES: Cc1cc2c(cc1C)C(=O)[C@]1(O)CCN(c3ccc4ncncc4c3)C1=N2]

(S)-3a-Hydroxy-6,7-dimethyl-1-(quinazolin-6-yl)-1,2,3,3a-tetrahydro-4H-pyrrolo[2,3-b]quinolin-4-one was prepared as a yellow solid according to Synthetic Scheme 6: mp=275-276° C.; ¹H NMR (500 MHz, DMSO-d₆) δ 9.61 (s, 1H), 9.23 (s, 1H), 9.04 (dd, J=9.0, 2.5 Hz, 1H), 8.65 (d, J=2.5 Hz, 1H), 8.08 (d, J=9.5 Hz, 1H), 7.53 (s, 1H), 7.19 (s, 1H), 6.89 (s, 1H), 4.23-4.13 (m, 2H), 2.34-2.32 (m, 2H), 2.31 (s, 3H), 2.25 (s, 3H); ESI MS m/z 359 [C₂₁H₁₈N₄O₂+H]⁺; HPLC (Method C) >99% (AUC), t_{R}=11.18 min; Chiral HPLC (Chiralpak AD, Method A) 70.8% (AUC), t_{R}=20.49 min.

### Preparation of (S)-3a-Hydroxy-1-(3-methylisoxazol-5-yl)-6-(trifluoromethyl)-1,2,3,3a-tetrahydro-4H-pyrrolo[2,3-b]quinolin-4-one

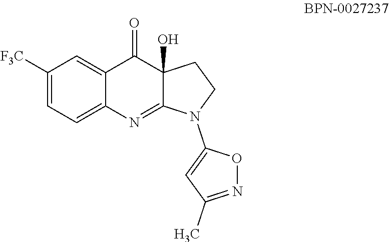 [SMILES: Cc1cc(N2CC[C@@]3(O)C(=O)c4cc(C(F)(F)F)ccc4N=C23)on1]

(S)-3a-Hydroxy-1-(3-methylisoxazol-5-yl)-6-(trifluoromethyl)-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 7.97-7.94 (m, 2H), 7.52 (d, J=8.0 Hz, 1H), 7.18 (s, 1H), 7.00 (s, 1H), 4.17-4.07 (m, 2H), 2.32-2.29 (m, 2H), 2.27 (s, 3H); ESI MS m/z 352 [C₁₆H₁₂F₃N₃O₃+H]⁺; HPLC (Method F) 98.2% (AUC), t_{R}=15.70 min; Chiral HPLC (Chiralpak AD, Method B) 59.1% (AUC), t_{R}=12.14 min.

### Preparation of (S)-1-(Cinnolin-6-yl)-3a-hydroxy-6,7-dimethyl-1,2,3,3a-tetrahydro-4H-pyrrolo[2,3-b]quinolin-4-one

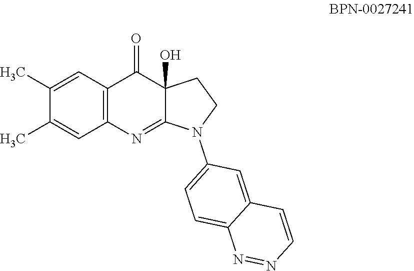 [SMILES: Cc1cc2c(cc1C)C(=O)[C@]1(O)CCN(c3ccc4nnccc4c3)C1=N2]

(S)-1-(Cinnolin-6-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=274-276° C.; ¹H NMR (500 MHz, DMSO-d₆) δ 9.28 (d, J=6.0 Hz, 1H), 9.03 (dd, J=9.5, 2.5 Hz, 1H), 8.49 (dd, J=5.0, 2.5 Hz, 2H), 8.17 (d, J=6.0 Hz, 1H), 7.55 (s, 1H), 7.22 (s, 1H), 6.92 (s, 1H), 4.19-4.16 (m, 2H), 2.36-2.32 (m, 5H), 2.26 (s, 3H); ESI MS m/z 359 [C₂₁H₁₈N₄O₂+H]⁺; HPLC (Method C) >99% (AUC), t_{R}=12.43 min; Chiral HPLC (Chiralpak AD, Method A) 54.2% (AUC), t_{R}=16.90 min.

### Preparation of (S)-3a-Hydroxy-1-(2-methoxyquinolin-6-yl)-6,7-dimethyl-1,2,3,3a-tetrahydro-4H-pyrrolo[2,3-b]quinolin-4-one

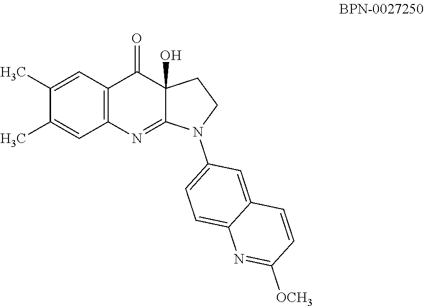 [SMILES: COc1ccc2cc(N3CC[C@@]4(O)C(=O)c5cc(C)c(C)cc5N=C34)ccc2n1]

(S)-3a-Hydroxy-1-(2-methoxyquinolin-6-yl)-6,7-dimethyl-1,2,3,3a-tetrahydro-4H-pyrrolo[2,3-b]quinolin-4-one was prepared as a yellow solid according to Synthetic Scheme 6: mp=233-237° C.; ¹H NMR (500 MHz, DMSO-d₆) δ 8.65 (dd, J=9.5, 2.5 Hz, 1H), 8.38 (d, J=2.5 Hz, 1H), 8.25 (d, J=9.0 Hz, 1H), 7.83 (d, J=9.5 Hz, 1H), 7.50 (s, 1H), 7.11 (s, 1H), 7.04 (d, J=9.0 Hz, 1H), 6.81 (s, 1H), 4.18-4.13 (m, 1H), 4.09-4.04 (m, 1H), 3.99 (s, 3H), 2.31-2.29 (m, 5H), 2.23 (s, 3H); ESI MS m/z 388 [C₂₃H₂₁N₃O₃+H]⁺; HPLC (Method C) 95.9% (AUC), t_{R}=14.47 min; Chiral HPLC (Chiralpak AD, Method A) 78.1% (AUC), t_{R}=18.25 min.

### Preparation of (S)-3a-Hydroxy-1-(2-methoxyquinolin-6-yl)-6-methyl-1,2,3,3a-tetrahydro-4H-pyrrolo[2,3-b]quinolin-4-one

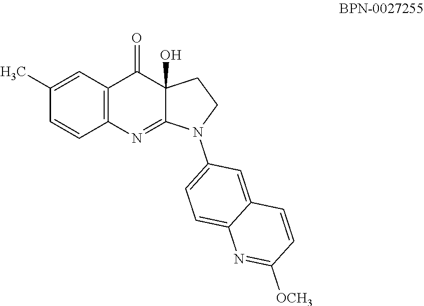 [SMILES: COc1ccc2cc(N3CC[C@@]4(O)C(=O)c5cc(C)ccc5N=C34)ccc2n1]

(S)-3a-Hydroxy-1-(2-methoxyquinolin-6-yl)-6-methyl-1,2,3,3a-tetrahydro-4H-pyrrolo[2,3-b]quinolin-4-one was prepared as a yellow solid according to Synthetic Scheme 6: mp=230-234° C.; ¹H NMR (500 MHz, DMSO-d₆) δ 8.62 (dd, J=9.0, 2.5 Hz, 1H), 8.40 (d, J=2.5 Hz, 1H), 8.25 (d, J=8.5 Hz, 1H), 7.83 (d, J=9.0 Hz, 1H), 7.55 (d, J=1.5 Hz, 1H), 7.40 (dd, J=8.5, 2.5 Hz, 1H), 7.19 (d, J=8.0 Hz, 1H), 7.04 (d, J=9.0 Hz, 1H), 6.87 (s, 1H), 4.19-4.14 (m, 1H), 4.10-4.07 (m, 1H), 3.99 (s, 3H), 2.32-2.30 (m, 5H); ESI MS m/z 374 [C₂₂H₁₉N₃O₃+H]⁺; HPLC (Method C) >99% (AUC), t_{R}=13.28 min; Chiral HPLC (Chiralpak AD, Method A) 71.5% (AUC), t_{R}=17.22 min.

### Preparation of (S)-3a-Hydroxy-6-methyl-1-(3-methylisoxazol-5-yl)-7-(trifluoromethyl)-1,2,3,3a-tetrahydro-4H-pyrrolo[2,3-b]quinolin-4-one

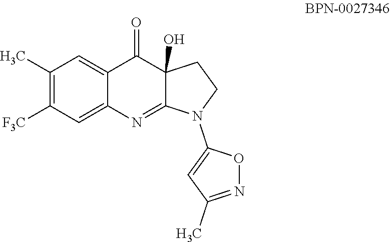 [SMILES: Cc1cc(N2CC[C@@]3(O)C(=O)c4cc(C)c(C(F)(F)F)cc4N=C23)on1]

(S)-3a-Hydroxy-6-methyl-1-(3-methylisoxazol-5-yl)-7-(trifluoromethyl)-1,2,3,3a-tetrahydro-4H-pyrrolo[2,3-b]quinolin-4-one was prepared as a yellow solid according to Synthetic Scheme 6: ¹H NMR (500 MHz, DMSO-d₆) δ 7.78 (s, 1H), 7.59 (s, 1H), 7.12 (s, 1H), 6.69 (s, 1H), 4.15-4.11 (m, 1H), 4.09-4.03 (m, 1H), 2.46 (s, 3H), 2.30-2.28 (m, 2H), 2.26 (s, 3H); ESI MS m/z 366 [C₁₇H₁₄F₃N₃O₃+H]⁺; HPLC (Method C) 98.3% (AUC), t_{R}=18.85 min; Chiral HPLC (Chiralpak AD, Method A) 49.5% (AUC), t_{R}=15.32 min.

### Preparation of (S)-3a-Hydroxy-6-methyl-1-(3-methylbenzo[d]isoxazol-6-yl)-1,2,3,3a-tetrahydro-4H-pyrrolo[2,3-b]quinolin-4-one

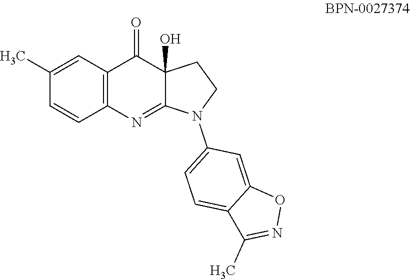 [SMILES: Cc1ccc2c(c1)C(=O)[C@]1(O)CCN(c3ccc4c(C)noc4c3)C1=N2]

(S)-3a-Hydroxy-6-methyl-1-(3-methylbenzo[d]isoxazol-6-yl)-1,2,3,3a-tetrahydro-4H-pyrrolo[2,3-b]quinolin-4-one was prepared as a yellow solid according to Synthetic Scheme 6: mp=249-251° C.; ¹H NMR (500 MHz, DMSO-d₆) δ 8.62 (d, J=1.5 Hz, 1H), 8.08 (dd, J=9.0, 2.0 Hz, 1H), 7.86 (d, J=9.0 Hz, 1H), 7.57 (d, J=1.5 Hz, 1H), 7.42 (dd, J=8.0, 1.5 Hz, 1H), 7.23 (d, J=8.0 Hz, 1H), 6.91 (s, 1H), 4.17-4.02 (m, 2H), 2.55 (s, 3H), 2.33 (s, 3H), 2.31-2.29 (m, 2H); ESI MS m/z 348 [C₂₀H₁₇N₃O₃+H]⁺; HPLC (Method C) >99% (AUC), t_{R}=12.92 min; Chiral HPLC (Chiralpak AD, Method A) 68.8% (AUC), t_{R}=19.68 min.

### Preparation of (S)-3a-Hydroxy-6-methyl-1-(pyrazolo[1,5-a]pyridin-5-yl)-1,2,3,3a-tetrahydro-4H-pyrrolo[2,3-b]quinolin-4-one

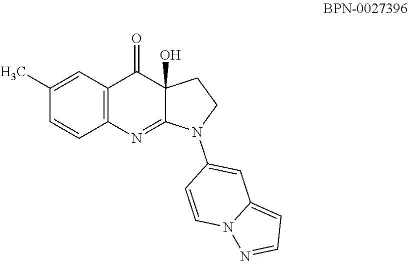 [SMILES: Cc1ccc2c(c1)C(=O)[C@]1(O)CCN(c3ccn4nccc4c3)C1=N2]

(S)-3a-Hydroxy-6-methyl-1-(pyrazolo[1,5-a]pyridin-5-yl)-1,2,3,3a-tetrahydro-4H-pyrrolo[2,3-b]quinolin-4-one was prepared as a yellow solid according to Synthetic Scheme 6: mp=242-243° C.; ¹H NMR (500 MHz, DMSO-d₆) δ 8.69 (d, J=8.0 Hz, 1H), 8.16 (d, J=2.5 Hz, 1H), 8.00 (dd, J=8.0, 2.5 Hz, 1H), 7.96 (d, J=2.0 Hz, 1H), 7.56 (d, J=2.0 Hz, 1H), 7.42 (dd, J=8.5, 2.0 Hz, 1H), 7.21 (d, J=8.0 Hz, 1H), 6.91 (s, 1H), 6.56 (dd, J=2.0, 0.5 Hz, 1H), 4.18-4.13 (m, 1H), 4.09-4.05 (m, 2H), 2.36 (s, 3H), 2.30-2.28 (m, 2H); ESI MS m/z 333 [C₁₉H₁₆N₄O₂+H]⁺; UPLC (Method A) 95.5% (AUC), t_{R}=2.80 min; Chiral HPLC (Chiralpak AD, Method A) 74.1% (AUC), t_{R}=21.26 min.

### Preparation of (S)-3a-Hydroxy-1-(imidazo[1,5-a]pyridin-6-yl)-6-methyl-1,2,3,3a-tetrahydro-4H-pyrrolo[2,3-b]quinolin-4-one

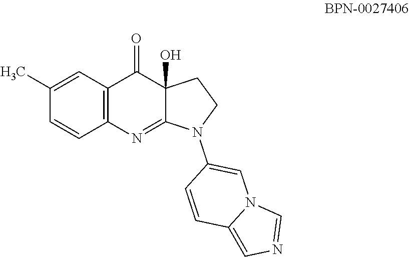 [SMILES: Cc1ccc2c(c1)C(=O)[C@]1(O)CCN(c3ccc4cncn4c3)C1=N2]

(S)-3a-Hydroxy-1-(imidazo[1,5-a]pyridin-6-yl)-6-methyl-1,2,3,3a-tetrahydro-4H-pyrrolo[2,3-b]quinolin-4-one was prepared as a yellow solid according to Synthetic Scheme 6: mp=215-219° C. dec.; ¹H NMR (500 MHz, DMSO-d₆) δ 9.26 (s, 1H), 8.45 (s, 1H), 7.62 (d, J=10.0 Hz, 1H), 7.54-7.50 (m, 2H), 7.40 (d, J=8.0 Hz, 1H), 7.37 (s, 1H), 7.18 (d, J=8.0 Hz, 1H), 6.87 (s, 1H), 4.08-4.02 (m, 1H), 3.96 (apparent t, J=8.0 Hz, 1H), 2.31 (s, 3H), 2.28-2.25 (m, 2H); ESI MS m/z 333 [C₁₉H₁₆N₄O₂+H]⁺; UPLC (Method A) >99% (AUC), t_{R}=2.40 min; Chiral HPLC (Chiralpak AD, Method A) 84.0% (AUC), t_{R}=20.09 min.

### Preparation of (S)-3a-Hydroxy-6-methyl-1-(thieno[3,2-b]pyridin-2-yl)-1,2,3,3a-tetrahydro-4H-pyrrolo[2,3-b]quinolin-4-one

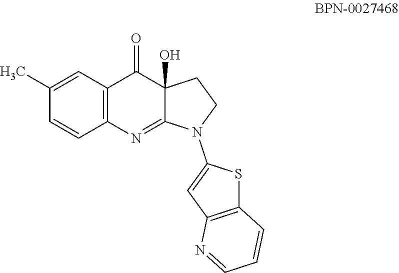 [SMILES: Cc1ccc2c(c1)C(=O)[C@]1(O)CCN(c3cc4ncccc4s3)C1=N2]

(S)-3a-Hydroxy-6-methyl-1-(thieno[3,2-b]pyridin-2-yl)-1,2,3,3a-tetrahydro-4H-pyrrolo[2,3-b]quinolin-4-one was prepared as an orange solid according to Synthetic Scheme 6: mp=260-262° C.; ¹H NMR (300 MHz, DMSO-d₆) δ 8.57 (d, J=3.3 Hz, 1H), 8.36 (d, J=7.5 Hz, 1H), 7.58 (s, 1H), 7.45 (d, J=7.8 Hz, 1H), 7.25-7.20 (m, 2H), 7.16 (s, 1H), 7.05 (s, 1H), 4.17-4.15 (m, 2H), 2.38-2.28 (m, 5H); ESI MS m/z 350 [C₁₉H₁₅N₃O₂S+H]⁺; UPLC (Method A) >99% (AUC), t_{R}=3.30 min; Chiral HPLC (Chiralpak AD, Method A) 62.6% (AUC), t_{R}=17.28 min.

### Preparation of (S)-3a-Hydroxy-6-methyl-1-(thieno[2,3-b]pyridin-2-yl)-1,2,3,3a-tetrahydro-4H-pyrrolo[2,3-b]quinolin-4-one

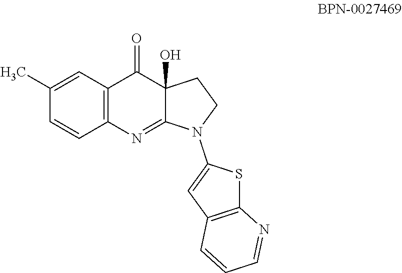 [SMILES: Cc1ccc2c(c1)C(=O)[C@]1(O)CCN(c3cc4cccnc4s3)C1=N2]

(S)-3a-Hydroxy-6-methyl-1-(thieno[2,3-b]pyridin-2-yl)-1,2,3,3a-tetrahydro-4H-pyrrolo[2,3-b]quinolin-4-one was prepared as a yellow solid according to Synthetic Scheme 6: mp=255-257° C.; ¹H NMR (300 MHz, DMSO-d₆) δ 8.42 (dd, J=4.5, 1.5 Hz, 1H), 8.08 (dd, J=8.1, 1.5 Hz, 1H), 7.57 (s, 1H), 7.45 (d, J=7.8 Hz, 1H), 7.39 (dd, J=7.8, 4.5 Hz, 1H), 7.25 (d, J=7.8 Hz, 1H), 7.02 (d, J=7.8 Hz, 2H), 4.14-4.06 (m, 2H), 2.36-2.33 (m, 5H); ESI MS m/z 350 [C₁₉H₁₅N₃O₂S+H]⁺; UPLC (Method A) >99% (AUC), t_{R}=3.71 min; Chiral HPLC (Chiralpak AD, Method A) 61.0% (AUC), t_{R}=19.64 min.

### Preparation of (S)-3a-Hydroxy-6,7-dimethyl-1-(pyrazolo[1,5-a]pyridin-5-yl)-1,2,3,3a-tetrahydro-4H-pyrrolo[2,3-b]quinolin-4-one

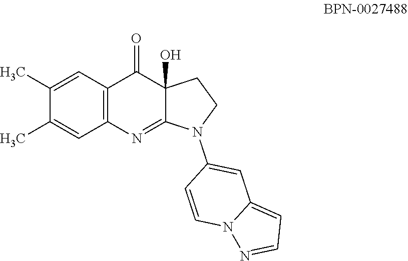 [SMILES: Cc1cc2c(cc1C)C(=O)[C@]1(O)CCN(c3ccn4nccc4c3)C1=N2]

(S)-3a-Hydroxy-6,7-dimethyl-1-(pyrazolo[1,5-a]pyridin-5-yl)-1,2,3,3a-tetrahydro-4H-pyrrolo[2,3-b]quinolin-4-one was prepared as a yellow solid according to Synthetic Scheme 6: mp=261-264° C.; ¹H NMR (300 MHz, DMSO-d₆) δ 8.70 (d, J=7.8 Hz, 1H), 8.17 (d, J=2.1 Hz, 1H), 8.01 (dd, J=7.5, 2.1 Hz, 1H), 7.97 (d, J=2.1 Hz, 1H), 7.52 (s, 1H), 7.13 (s, 1H), 6.88 (s, 1H), 6.56 (d, J=2.1 Hz, 1H), 4.08-3.99 (m, 2H), 2.29 (s, 3H), 2.26 (apparent s, 2H), 2.24 (s, 3H); ESI MS m/z 347 [C₂₀H₁₈N₄O₂+H]⁺; UPLC (Method A) 98.7% (AUC), t_{R}=3.02 min; Chiral HPLC (Chiralpak AD, Method A) 67.3% (AUC), t_{R}=18.70 min.

### Preparation of (S)-3a-Hydroxy-6,7-dimethyl-1-(thieno[2,3-b]pyridin-5-yl)-1,2,3,3a-tetrahydro-4H-pyrrolo[2,3-b]quinolin-4-one

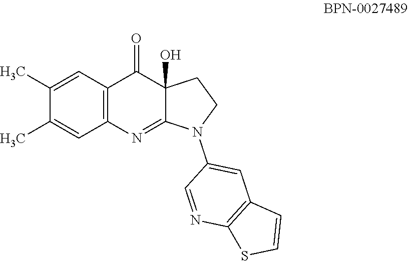 [SMILES: Cc1cc2c(cc1C)C(=O)[C@]1(O)CCN(c3cnc4sccc4c3)C1=N2]

(S)-3a-Hydroxy-6,7-dimethyl-1-(thieno[2,3-b]pyridin-5-yl)-1,2,3,3a-tetrahydro-4H-pyrrolo[2,3-b]quinolin-4-one was prepared as a yellow solid according to Synthetic Scheme 6: mp=237-241° C.; ¹H NMR (300 MHz, DMSO-d₆) δ 9.32 (d, J=2.1 Hz, 1H), 8.97 (d, J=2.1 Hz, 1H), 7.94 (d, J=6.0 Hz, 1H), 7.50 (d, J=5.7 Hz, 2H), 7.11 (s, 1H), 6.88 (s, 1H), 4.21-4.07 (m, 2H), 2.32-2.28 (m, 5H), 2.23 (s, 3H); ESI MS m/z 364 [C₂₀H₁₇N₃O₂S+H]⁺; UPLC (Method A) >99% (AUC), t_{R}=3.16 min; Chiral HPLC (Chiralpak AD, Method A) 85.0% (AUC), t_{R}=18.22 min.

### Preparation of (S)-3a-Hydroxy-6,7-dimethyl-1-(2-methylbenzo[d]thiazol-5-yl)-1,2,3,3a-tetrahydro-4H-pyrrolo[2,3-b]quinolin-4-one

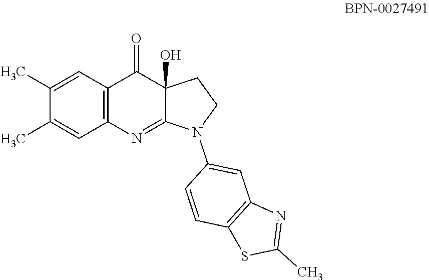

(S)-3a-Hydroxy-6,7-dimethyl-1-(2-methylbenzo[d]thiazol-5-yl)-1,2,3,3a-tetrahydro-4H-pyrrolo[2,3-b]quinolin-4-one was prepared as a yellow solid according to Synthetic Scheme 6: mp=233-235° C.; ¹H NMR (300 MHz, DMSO-d₆) δ 8.75 (d, J=2.1 Hz, 1H), 8.12 (dd, J=9.0, 2.1 Hz, 1H), 8.04 (d, J=9.0 Hz, 1H), 7.50 (s, 1H), 7.10 (s, 1H), 6.83 (s, 1H), 4.19-4.02 (m, 2H), 2.82 (s, 3H), 2.29 (apparent s, 5H), 2.23 (s, 3H); ESI MS m/z 378 [C₂₁H₁₉N₃O₂S+H]⁺; UPLC (Method A) 96.9% (AUC), t_{R}=3.19 min; Chiral HPLC (Chiralpak AD, Method A) 84.9% (AUC), t_{R}=21.69 min.

### Preparation of (S)-6-(3a-Hydroxy-6,7-dimethyl-4-oxo-2,3,3a,4-tetrahydro-1H-pyrrolo[2,3-b]quinolin-1-yl)quinoline-2-carbonitrile

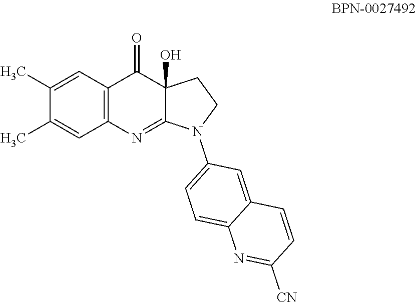 [SMILES: Cc1cc2c(cc1C)C(=O)[C@]1(O)CCN(c3ccc4nc(C#N)ccc4c3)C1=N2]

(S)-6-(3a-Hydroxy-6,7-dimethyl-4-oxo-2,3,3a,4-tetrahydro-1H-pyrrolo[2,3-b]quinolin-1-yl)quinoline-2-carbonitrile was prepared as a yellow-orange solid according to Synthetic Scheme 6: mp=272-274° C.; ¹H NMR (300 MHz, DMSO-d₆) δ 9.04 (dd, J=9.6, 2.4 Hz, 1H), 8.63 (d, J=8.4 Hz, 1H), 8.58 (d, J=2.4 Hz, 1H), 8.19 (d, J=9.3 Hz, 1H), 8.02 (d, J=8.7 Hz, 1H), 7.54 (s, 1H), 7.21 (s, 1H), 6.93 (s, 1H), 4.18-4.14 (m, 2H), 2.33-2.31 (m, 5H), 2.25 (s, 3H); ESI MS m/z 383 [C₂₃H₁₈N₄O₂+H]⁺; UPLC (Method A) >99% (AUC), t_{R}=3.46 min; Chiral HPLC (Chiralpak AD, Method A) 89.5% (AUC), t_{R}=21.66 min.

### Preparation of (S)-1-(Benzo[d][1,2,3]thiadiazol-6-yl)-3a-hydroxy-6,7-dimethyl-1,2,3,3a-tetrahydro-4H-pyrrolo[2,3-b]quinolin-4-one

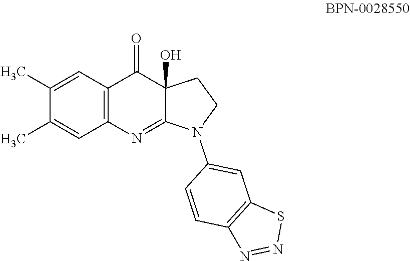 [SMILES: Cc1cc2c(cc1C)C(=O)[C@]1(O)CCN(c3ccc4nnsc4c3)C1=N2]

(S)-1-(Benzo[d][1,2,3]thiadiazol-6-yl)-3a-hydroxy-6,7-dimethyl-1,2,3,3a-tetrahydro-4H-pyrrolo[2,3-b]quinolin-4-one was prepared as a yellow solid according to Synthetic Scheme 6: ¹H NMR (300 MHz, DMSO-d₆) δ 9.10 (d, J=1.8 Hz, 1H), 8.74-8.64 (m, 2H), 7.54 (s, 1H), 7.22 (s, 1H), 6.93 (s, 1H), 4.18-4.13 (m, 2H), 2.31 (apparent s, 5H), 2.25 (s, 3H); ESI MS m/z 365 [C₁₉H₁₆N₄O₂S+H]⁺; UPLC (Method A) 96.2% (AUC), t_{R}=3.36 min; Chiral HPLC (Chiralpak AD, Method A) 50.7% (AUC), t_{R}=18.41 min.

### Preparation of (S)-1-(3,4-Dihydro-2H-benzo[b][1,4]oxazin-7-yl)-3a-hydroxy-6,7-dimethyl-1,2,3,3a-tetrahydro-4H-pyrrolo[2,3-b]quinolin-4-one

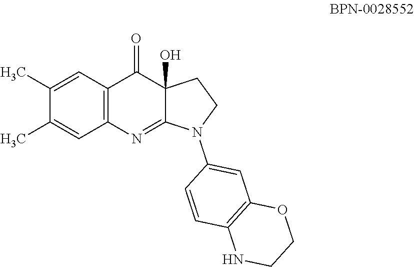 [SMILES: Cc1cc2c(cc1C)C(=O)[C@]1(O)CCN(c3ccc4c(c3)OCCN4)C1=N2]

(S)-1-(3,4-Dihydro-2H-benzo[b][1,4]oxazin-7-yl)-3a-hydroxy-6,7-dimethyl-1,2,3,3a-tetrahydro-4H-pyrrolo[2,3-b]quinolin-4-one was prepared as a red-orange solid according to Synthetic Scheme 4: ¹H NMR (300 MHz, DMSO-d₆) δ 7.57 (d, J=2.4 Hz, 1H), 7.44 (s, 1H), 7.21 (dd, J=8.4, 2.7 Hz, 1H), 6.96 (s, 1H), 6.62 (s, 1H), 6.58 (d, J=8.7 Hz, 1H), 5.73 (s, 1H), 4.14-4.13 (m, 2H), 3.99-3.79 (m, 2H), 3.30 (apparent s, 2H), 2.26 (s, 3H), 2.25-2.20 (m, 5H); ESI MS m/z 364 [C₂₁H₂₁N₃O₃+H]⁺; UPLC (Method A) 96.7% (AUC), t_{R}=3.16 min; Chiral HPLC (Chiralpak AD, Method A) 71.1% (AUC), t_{R}=23.14 min.

### Preparation of (S)-3a-Hydroxy-6,7-dimethyl-1-(2-methylpyridin-4-yl)-1,2,3,3a-tetrahydro-4H-pyrrolo[2,3-b]quinolin-4-one

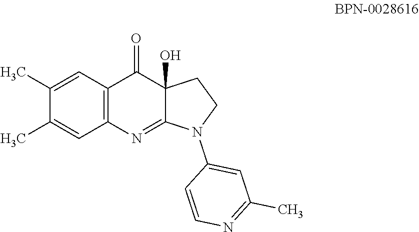

(S)-3a-Hydroxy-6,7-dimethyl-1-(2-methylpyridin-4-yl)-1,2,3,3a-tetrahydro-4H-pyrrolo[2,3-b]quinolin-4-one was prepared as a yellow solid according to Synthetic Scheme 6: mp=265-267° C.; ¹H NMR (300 MHz, DMSO-d₆) δ 8.38 (d, J=6.0 Hz, 1H), 8.03 (dd, J=6.0, 2.4 Hz, 1H), 7.85 (d, J=2.1 Hz, 1H), 7.52 (s, 1H), 7.16 (s, 1H), 6.87 (s, 1H), 4.04-3.94 (m, 2H), 2.30 (s, 3H), 2.28-2.24 (m, 1H), 2.21 (apparent s, 4H), 3H obscured by solvent peak; ESI MS m/z 322 [C₁₉H₁₉N₃O₂+H]⁺; UPLC (Method A) 95.1% (AUC), t_{R}=3.17 min; Chiral HPLC (Chiralpak AD, Method A) 50.8% (AUC), t_{R}=14.81 min.

### Preparation of (S)-3a-Hydroxy-1-(6-methoxypyridin-3-yl)-6,7-dimethyl-1,2,3,3a-tetrahydro-4H-pyrrolo[2,3-b]quinolin-4-one

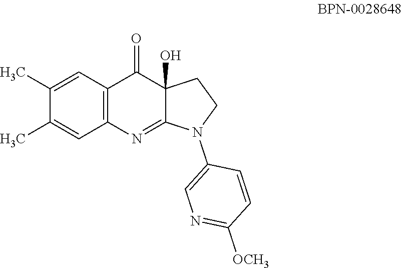 [SMILES: COc1ccc(N2CC[C@@]3(O)C(=O)c4cc(C)c(C)cc4N=C23)cn1]

(S)-3a-Hydroxy-1-(6-methoxypyridin-3-yl)-6,7-dimethyl-1,2,3,3a-tetrahydro-4H-pyrrolo[2,3-b]quinolin-4-one was prepared as a yellow solid according to Synthetic Scheme 6: mp=213-216° C.; ¹H NMR (300 MHz, DMSO-d₆) δ 8.74 (d, J=3.0 Hz, 1H), 8.53 (dd, J=9.0, 2.7 Hz, 1H), 7.48 (s, 1H), 7.02 (s, 1H), 6.91 (d, J=9.3 Hz, 1H), 6.80 (s, 1H), 4.09-3.91 (m, 2H), 3.87 (s, 3H), 2.28-2.26 (m, 5H), 2.21 (s, 3H); ESI MS m/z 338 [C₁₉H₁₉N₃O₃+H]⁺; UPLC (Method A) >99% (AUC), t_{R}=2.97 min; Chiral HPLC (Chiralpak AD, Method A) 82.7% (AUC), t_{R}=15.44 min.

### Preparation of (S)-1-(5-Chloro-6-(hydroxymethyl)pyridin-3-yl)-3a-hydroxy-6,7-dimethyl-1,2,3,3a-tetrahydro-4H-pyrrolo[2,3-b]quinolin-4-one

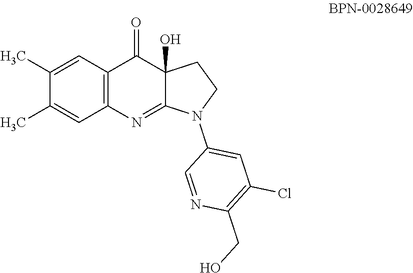 [SMILES: Cc1cc2c(cc1C)C(=O)[C@]1(O)CCN(c3cnc(CO)c(Cl)c3)C1=N2]

(S)-1-(5-Chloro-6-(hydroxymethyl)pyridin-3-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=242-245° C.; ¹H NMR (300 MHz, DMSO-d₆) δ 9.10 (d, J=2.1 Hz, 1H), 8.83 (d, J=2.4 Hz, 1H), 7.52 (s, 1H), 7.10 (s, 1H), 6.89 (s, 1H), 5.24 (t, J=6.0 Hz, 1H), 4.64 (apparent t, J=5.7 Hz, 2H), 4.12-4.03 (m, 2H), 2.30-2.27 (m, 5H), 2.23 (s, 3H); ESI MS m/z 372 [C₁₉H₁₈ClN₃O₃+H]⁺; UPLC (Method A) >99% (AUC), t_{R}=2.98 min; Chiral HPLC (Chiralpak AD, Method A) 62.9% (AUC), t_{R}=17.17 min.

### Preparation of (S)-3a-Hydroxy-6,7-dimethyl-1-(2-(methylamino)pyridin-4-yl)-1,2,3,3a-tetrahydro-4H-pyrrolo[2,3-b]quinolin-4-one

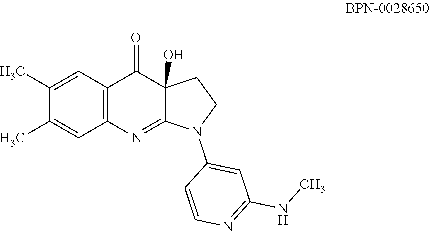

(S)-3a-Hydroxy-6,7-dimethyl-1-(2-(methylamino)pyridin-4-yl)-1,2,3,3a-tetrahydro-4H-pyrrolo[2,3-b]quinolin-4-one was prepared as a yellow solid according to Synthetic Scheme 6: ¹H NMR (300 MHz, DMSO-d₆) δ 7.93 (d, J=6.0 Hz, 1H), 7.51 (s, 1H), 7.29 (s, 1H), 7.22 (d, J=5.7 Hz, 1H), 7.11 (s, 1H), 6.83 (s, 1H), 6.61-6.40 (m, 1H), 3.92 (apparent t, J=6.0 Hz, 2H), 2.79 (d, J=4.8 Hz, 3H), 2.29 (s, 3H), 2.24-2.18 (m, 5H); ESI MS m/z 337 [C₁₉H₂₀N₄O₂+H]⁺; UPLC (Method A) 95.5% (AUC), t_{R}=3.22 min; Chiral HPLC (Chiralpak AD, Method A) 70.0% (AUC), t_{R}=18.22 min.

### Preparation of (S)-3a-Hydroxy-1-(6-methoxy-5-methylpyridin-3-yl)-6,7-dimethyl-1,2,3,3a-tetrahydro-4H-pyrrolo[2,3-b]quinolin-4-one

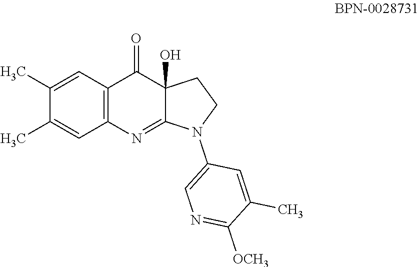

(S)-3a-Hydroxy-1-(6-methoxy-5-methylpyridin-3-yl)-6,7-dimethyl-1,2,3,3a-tetrahydro-4H-pyrrolo[2,3-b]quinolin-4-one was prepared as a yellow solid according to Synthetic Scheme 6: mp=195-199° C.; ¹H NMR (300 MHz, DMSO-d₆) δ 8.65 (d, J=2.4 Hz, 1H), 8.29 (s, 1H), 7.47 (s, 1H), 7.03 (s, 1H), 6.78 (s, 1H), 4.07-3.93 (m, 2H), 3.89 (s, 3H), 2.26 (apparent s, 5H), 2.21 (s, 6H); ESI MS m/z 352 [C₂₀H₂₁N₃O₃+H]⁺; UPLC (Method A) >99% (AUC), t_{R}=3.26 min; Chiral HPLC (Chiralpak AD, Method A) 81.1% (AUC), t_{R}=13.45 min.

### Preparation of (S)-3a-Hydroxy-1-(6-(hydroxymethyl)-5-methylpyridin-3-yl)-6,7-dimethyl-1,2,3,3a-tetrahydro-4H-pyrrolo[2,3-b]quinolin-4-one

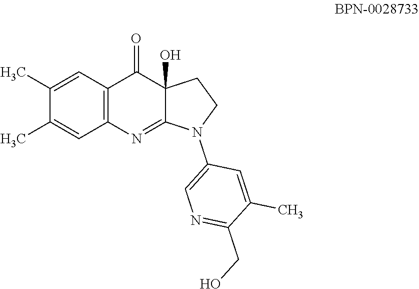

(S)-3a-Hydroxy-1-(6-(hydroxymethyl)-5-methylpyridin-3-yl)-6,7-dimethyl-1,2,3,3a-tetrahydro-4H-pyrrolo[2,3-b]quinolin-4-one was prepared as a yellow solid according to Synthetic Scheme 6: mp=218-222° C.; ¹H NMR (300 MHz, DMSO-d₆) δ 9.07 (d, J=2.7 Hz, 1H), 8.28 (s, 1H), 7.50 (s, 1H), 7.07 (s, 1H), 6.83 (s, 1H), 5.02 (t, J=5.4 Hz, 1H), 4.57 (d, J=5.4 Hz, 2H), 4.09-3.98 (m, 2H), 2.39 (s, 3H), 2.28 (apparent s, 5H), 2.23 (s, 3H); ESI MS m/z 352 [C₂₀H₂₁N₃O₃+H]⁺; UPLC (Method A) 98.3% (AUC), t_{R}=2.89 min; Chiral HPLC (Chiralpak AD, Method A) 74.8% (AUC), t_{R}=18.63 min.

### Preparation of (S)-5-(3a-Hydroxy-6,7-dimethyl-4-oxo-2,3,3a,4-tetrahydro-1H-pyrrolo[2,3-b]quinolin-1-yl)-3-methylpicolinonitrile

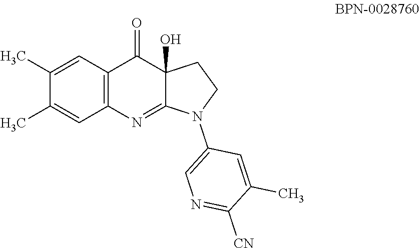

(S)-5-(3a-Hydroxy-6,7-dimethyl-4-oxo-2,3,3a,4-tetrahydro-1H-pyrrolo[2,3-b]quinolin-1-yl)-3-methylpicolinonitrile was prepared as a yellow solid according to Synthetic Scheme 6: mp=249-253° C.; ¹H NMR (300 MHz, DMSO-d₆) δ 9.41 (d, J=2.4 Hz, 1H), 8.58 (d, J=2.1 Hz, 1H), 7.54 (s, 1H), 7.19 (s, 1H), 6.94 (s, 1H), 4.05 (apparent t, J=6.3 Hz, 2H), 2.34 (s, 3H), 2.30-2.27 (m, 5H), 2.25 (s, 3H); ESI MS m/z 347 [C₂₀H₁₈N₄O₂+H]⁺; UPLC (Method A) 95.0% (AUC), t_{R}=3.91 min; Chiral HPLC (Chiralpak AD, Method A) 40.3% (AUC), t_{R}=17.33 min.

### Preparation of (S)-7a-Hydroxy-2-methyl-5-(2-methylquinolin-6-yl)-5,6,7,7a-tetrahydro-8H-pyrrolo[2,3-b]thieno[2,3-e]pyridin-8-one

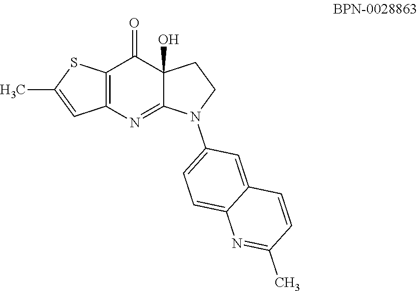 [SMILES: Cc1ccc2cc(N3CC[C@@]4(O)C(=O)c5sc(C)cc5N=C34)ccc2n1]

(S)-7a-Hydroxy-2-methyl-5-(2-methylquinolin-6-yl)-5,6,7,7a-tetrahydro-8H-pyrrolo[2,3-b]thieno[2,3-e]pyridin-8-one was prepared as an orange solid according to Synthetic Scheme 3: mp=244-247° C.; ¹H NMR (300 MHz, DMSO-d₆) δ 8.61 (dd, J=9.3, 2.7 Hz, 1H), 8.33 (d, J=2.7 Hz, 1H), 8.24 (d, J=8.4 Hz, 1H), 7.97 (d, J=9.3 Hz, 1H), 7.44 (d, J=8.4 Hz, 1H), 6.97 (s, 1H), 6.88 (d, J=1.2 Hz, 1H), 4.29-4.12 (m, 2H), 2.66 (s, 3H), 2.36-2.25 (m, 2H), 3H obscured by solvent peak; ESI MS m/z 364 [C₂₀H₁₇N₃O₂S+H]⁺; UPLC (Method A) 98.8% (AUC), t_{R}=2.47 min; Chiral HPLC (Chiralpak AD, Method A) 91.2% (AUC), t_{R}=19.24 min.

### Preparation of (S)-1-(2-Ethylpyridin-4-yl)-3a-hydroxy-6,7-dimethyl-1,2,3,3a-tetrahydro-4H-pyrrolo[2,3-b]quinolin-4-one

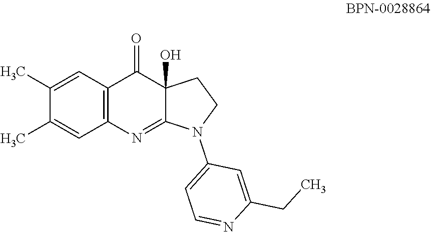 [SMILES: CCc1cc(N2CC[C@@]3(O)C(=O)c4cc(C)c(C)cc4N=C23)ccn1]

(S)-1-(2-Ethylpyridin-4-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=269-270° C.; ¹H NMR (300 MHz, DMSO-d₆) δ 8.41 (d, J=5.7 Hz, 1H), 7.99 (dd, J=5.7, 2.4 Hz, 1H), 7.91 (d, J=1.8 Hz, 1H), 7.52 (s, 1H), 7.15 (s, 1H), 6.87 (s, 1H), 4.04-3.95 (m, 2H), 2.76 (q, J=7.5 Hz, 2H), 2.30 (s, 3H), 2.28-2.24 (m, 5H), 1.26 (t, J=7.5 Hz, 3H); ESI MS m/z 336 [C₂₀H₂₁N₃O₂+H]⁺; UPLC (Method A) >99% (AUC), t_{R}=3.31 min; Chiral HPLC (Chiralpak AD, Method A) 65.4% (AUC), t_{R}=13.92 min.

### Preparation of (S)-1-(2-Ethylbenzo[d]thiazol-5-yl)-3a-hydroxy-6,7-dimethyl-1,2,3,3a-tetrahydro-4H-pyrrolo[2,3-b]quinolin-4-one

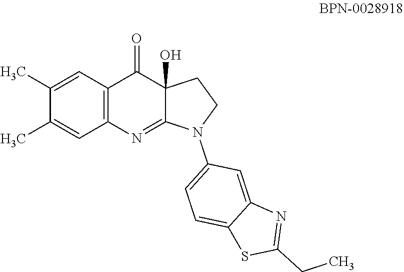 [SMILES: CCc1nc2cc(N3CC[C@@]4(O)C(=O)c5cc(C)c(C)cc5N=C34)ccc2s1]

(S)-1-(2-Ethylbenzo[d]thiazol-5-yl)-3a-hydroxy-6,7-dimethyl-1,2,3,3a-tetrahydro-4H-pyrrolo[2,3-b]quinolin-4-one was prepared as a yellow solid according to Synthetic Scheme 6: mp=232-235° C.; ¹H NMR (300 MHz, DMSO-d₆) δ 8.73 (d, J=2.1 Hz, 1H), 8.16 (dd, J=9.0, 2.4 Hz, 1H), 8.06 (d, J=8.7 Hz, 1H), 7.50 (s, 1H), 7.10 (s, 1H), 6.83 (s, 1H), 4.19-4.02 (m, 2H), 3.15 (q, J=7.2 Hz, 2H), 2.29 (m, 5H), 2.23 (s, 3H), 1.40 (t, J=7.5 Hz, 3H); ESI MS m/z 392 [C₂₂H₂₁N₃O₂S+H]⁺; UPLC (Method A) >99% (AUC), t_{R}=3.45 min; Chiral HPLC (Chiralpak AD, Method A) 97.2% (AUC), t_{R}=22.48 min.

### Preparation of (S)-3a-Hydroxy-1-(2-isopropylbenzo[d]thiazol-5-yl)-6,7-dimethyl-1,2,3,3a-tetrahydro-4H-pyrrolo[2,3-b]quinolin-4-one

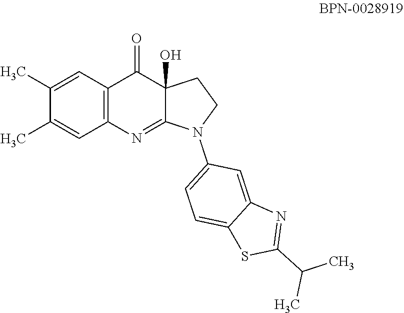 [SMILES: Cc1cc2c(cc1C)C(=O)[C@]1(O)CCN(c3ccc4sc(C(C)C)nc4c3)C1=N2]

(S)-3a-Hydroxy-1-(2-isopropylbenzo[d]thiazol-5-yl)-6,7-dimethyl-1,2,3,3a-tetrahydro-4H-pyrrolo[2,3-b]quinolin-4-one was prepared as a yellow solid according to Synthetic Scheme 6: ¹H NMR (300 MHz, DMSO-d₆) δ 8.71 (d, J=2.1 Hz, 1H), 8.20 (dd, J=8.7, 2.1 Hz, 1H), 8.07 (d, J=8.7 Hz, 1H), 7.50 (s, 1H), 7.10 (s, 1H), 6.83 (s, 1H), 4.16-4.02 (m, 2H), 3.50-3.38 (m, 1H), 2.29 (m, 5H), 2.23 (s, 3H), 1.44 (s, 3H), 1.42 (s, 3H); ESI MS m/z 406 [C₂₃H₂₃N₃O₂S+H]⁺; UPLC (Method A) >99% (AUC), t_{R}=3.72 min; Chiral HPLC (Chiralpak AD, Method A) 95.4% (AUC), t_{R}=20.82 min.

### Preparation of (S)-3a-Hydroxy-6,7-dimethyl-1-(5,6,7,8-tetrahydroquinolin-3-yl)-1,2,3,3a-tetrahydro-4H-pyrrolo[2,3-b]quinolin-4-one

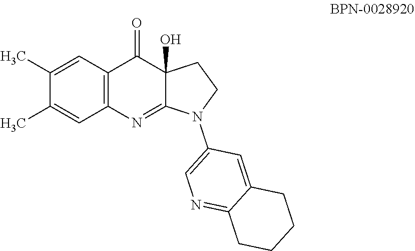 [SMILES: Cc1cc2c(cc1C)C(=O)[C@]1(O)CCN(c3cnc4c(c3)CCCC4)C1=N2]

(S)-3a-Hydroxy-6,7-dimethyl-1-(5,6,7,8-tetrahydroquinolin-3-yl)-1,2,3,3a-tetrahydro-4H-pyrrolo[2,3-b]quinolin-4-one was prepared as a yellow solid according to Synthetic Scheme 6: mp=243-248° C.; ¹H NMR (300 MHz, DMSO-d₆) δ 9.00 (d, J=2.7 Hz, 1H), 8.13 (d, J=2.4 Hz, 1H), 7.49 (s, 1H), 7.03 (s, 1H), 6.81 (s, 1H), 4.06-3.91 (m, 2H), 2.82-2.78 (m, 4H), 2.33 (s, 5H), 2.22 (s, 3H), 1.84-1.76 (m, 4H); ESI MS m/z 362 [C₂₂H₂₃N₃O₂+H]⁺; UPLC (Method A) >99% (AUC), t_{R}=3.23 min; Chiral HPLC (Chiralpak AD, Method A) 97.5% (AUC), t_{R}=15.36 min.

### Preparation of (S)-1-(2,3-Dihydrobenzo[b][1,4]dioxin-6-yl)-3a-hydroxy-6,7-dimethyl-1,2,3,3a-tetrahydro-4H-pyrrolo[2,3-b]quinolin-4-one

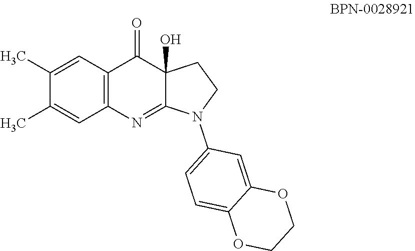 [SMILES: Cc1cc2c(cc1C)C(=O)[C@]1(O)CCN(c3ccc4c(c3)OCCO4)C1=N2]

(S)-1-(2,3-Dihydrobenzo[b][1,4]dioxin-6-yl)-3a-hydroxy-6,7-dimethyl-1,2,3,3a-tetrahydro-4H-pyrrolo[2,3-b]quinolin-4-one was prepared as a yellow-orange solid according to Synthetic Scheme 6: mp=213-217° C.; ¹H NMR (300 MHz, DMSO-d₆) δ 7.80 (d, J=2.7 Hz, 1H), 7.46 (s, 1H), 7.39 (dd, J=9.0, 2.7 Hz, 1H), 7.00 (s, 1H), 6.89 (d, J=9.0 Hz, 1H), 6.74 (s, 1H), 4.27-4.26 (m, 4H), 4.00-3.85 (m, 2H), 2.28 (s, 5H), 2.21 (s, 3H); ESI MS m/z 365 [C₂₁H₂₀N₂O₄+H]⁺; UPLC (Method A) >99% (AUC), t_{R}=3.26 min; Chiral HPLC (Chiralpak AD, Method A) 95.9% (AUC), t_{R}=22.57 min.

### Preparation of (S)-1-(3,4-Dihydro-2H-benzo[b][1,4]thiazin-6-yl)-3a-hydroxy-6,7-dimethyl-1,2,3,3a-tetrahydro-4H-pyrrolo[2,3-b]quinolin-4-one

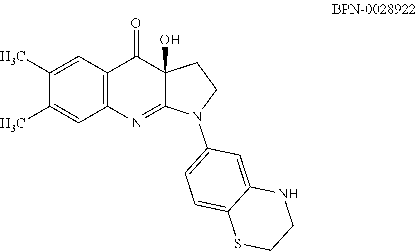 [SMILES: Cc1cc2c(cc1C)C(=O)[C@]1(O)CCN(c3ccc4c(c3)NCCS4)C1=N2]

(S)-1-(3,4-Dihydro-2H-benzo[b][1,4]thiazin-6-yl)-3a-hydroxy-6,7-dimethyl-1,2,3,3a-tetrahydro-4H-pyrrolo[2,3-b]quinolin-4-one was prepared as an orange solid according to Synthetic Scheme 6: ¹H NMR (300 MHz, DMSO-d₆) δ 7.46 (s, 1H), 7.36 (d, J=2.4 Hz, 1H), 7.12 (dd, J=8.4, 2.1 Hz, 1H), 7.02 (s, 1H), 6.86 (d, J=8.4 Hz, 1H), 6.72 (s, 1H), 6.18 (s, 1H), 3.95-3.82 (m, 2H), 3.53-3.46 (m, 2H), 2.99-2.96 (m, 2H), 2.27 (s, 3H), 2.21 (s, 5H); ESI MS m/z 380 [C₂₁H₂₁N₃O₂S+H]⁺; UPLC (Method A) 98.5% (AUC), t_{R}=3.50 min; Chiral HPLC (Chiralpak AD, Method A) 95.3% (AUC), t_{R}=22.99 min.

### Preparation of (S)-3a-Hydroxy-1-(2-methoxypyridin-4-yl)-6-methyl-3,3a-dihydro-1H-pyrrolo[2,3-b]quinolin-4(2H)-one

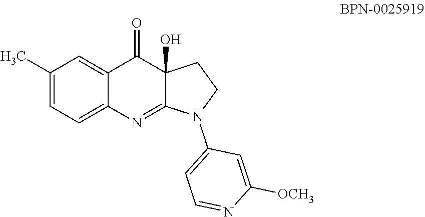 [SMILES: COc1cc(N2CC[C@@]3(O)C(=O)c4cc(C)ccc4N=C23)ccn1]

(S)-3a-Hydroxy-1-(2-methoxypyridin-4-yl)-6-methyl-3,3a-dihydro-1H-pyrrolo[2,3-b]quinolin-4(2H)-one was prepared as a yellow solid according to Synthetic Scheme 4: mp=229-230° C.; ¹H NMR (500 MHz, CDCl₃) δ 8.14 (d, J=6.0 Hz, 1H), 7.70 (d, J=1.6 Hz, 1H), 7.62 (dd, J=6.0, 1.9 Hz, 1H), 7.42-7.39 (m, 2H), 7.30 (d, J=8.1 Hz, 1H), 4.13-4.08 (m, 1H), 3.98-3.92 (m, 4H), 2.50 (dd, J=14.0, 5.9 Hz, 1H), 2.36-2.27 (m, 4H), OH proton is missing; ESI MS m/z 324 [C₁₈H₁₇N₃O₃+H]⁺; HPLC (Method C) 95.0% (AUC), t_{R}=12.46 min; Chiral HPLC (Chiralpak AD, Method A) 48.1% (AUC), t_{R}=15.26 min.

### Preparation of (S)-1-(Benzo[b]thiophen-6-yl)-3a-hydroxy-6-methyl-3,3a-dihydro-1H-pyrrolo[2,3-b]quinolin-4(2H)-one

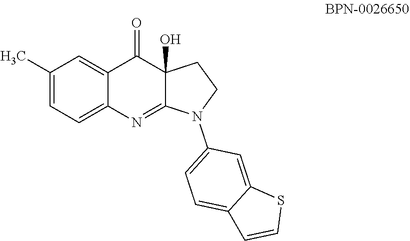 [SMILES: Cc1ccc2c(c1)C(=O)[C@]1(O)CCN(c3ccc4ccsc4c3)C1=N2]

(S)-1-(Benzo[b]thiophen-6-yl)-3a-hydroxy-6-methyl-3,3a-dihydro-1H-pyrrolo[2,3-b]quinolin-4(2H)-one was prepared as a yellow solid according to Synthetic Scheme 5: mp=217-218° C.; ¹H NMR (500 MHz, DMSO-d₆) δ 8.74 (d, J=1.9 Hz, 1H), 8.16 (dd, J=8.8, 2.1 Hz, 1H), 7.91 (d, J=8.8 Hz, 1H), 7.70 (d, J=5.5 Hz, 1H), 7.54 (d, J=1.3 Hz, 1H), 7.44-7.43 (m, 1H), 7.39-7.37 (m, 1H), 7.17 (d, J=8.0 Hz, 1H), 6.85 (s, 1H), 4.17-4.12 (m, 1H), 4.07-4.03 (m, 1H), 2.31-2.28 (m, 5H); ESI MS m/z 349 [C₂₀H₁₆N₂O₂S+H]⁺; HPLC (Method C) >99% (AUC), t_{R}=12.99 min; Chiral HPLC (Chiralpak AD, Method A) >99% (AUC), t_{R}=17.48 min.

### Preparation of (S)-1-(Benzo[b]thiophen-5-yl)-3a-hydroxy-6-methyl-3,3a-dihydro-1H-pyrrolo[2,3-b]quinolin-4(2H)-one

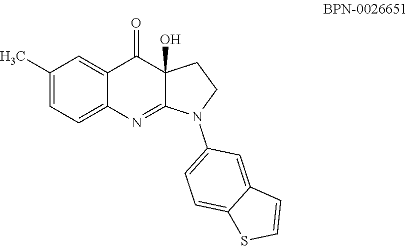 [SMILES: Cc1ccc2c(c1)C(=O)[C@]1(O)CCN(c3ccc4sccc4c3)C1=N2]

(S)-1-(Benzo[b]thiophen-5-yl)-3a-hydroxy-6-methyl-3,3a-dihydro-1H-pyrrolo[2,3-b]quinolin-4(2H)-one was prepared as an orange solid according to Synthetic Scheme 5: mp=215-216° C.; ¹H NMR (500 MHz, DMSO-d₆) δ 8.50 (d, J=2.1 Hz, 1H), 8.20 (dd, J=8.9, 2.1 Hz, 1H), 8.03 (d, J=8.9 Hz, 1H), 7.80 (d, J=5.4 Hz, 1H), 7.54 (d, J=1.5 Hz, 1H), 7.49 (d, J=5.4 Hz, 1H), 7.38 (d, J=8.1, 1.8 Hz, 1H), 7.14 (d, J=8.1 Hz, 1H), 6.85 (s, 1H), 4.18-4.12 (m, 1H), 4.05-4.02 (m, 1H), 2.32-2.29 (m, 5H); ESI MS m/z 349 [C₂₀H₁₆N₂O₂S+H]⁺; HPLC (Method C) >99% (AUC), t_{R}=13.35 min; Chiral HPLC (Chiralpak AD, Method A) 96.3% (AUC), t_{R}=21.69 min.

### Preparation of (S)-1-(Benzo[d]thiazol-5-yl)-3a-hydroxy-6-methyl-3,3a-dihydro-1H-pyrrolo[2,3-b]quinolin-4(2H)-one

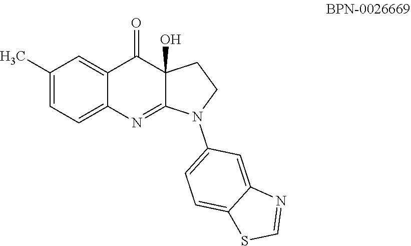 [SMILES: Cc1ccc2c(c1)C(=O)[C@]1(O)CCN(c3ccc4scnc4c3)C1=N2]

(S)-1-(Benzo[d]thiazol-5-yl)-3a-hydroxy-6-methyl-3,3a-dihydro-1H-pyrrolo[2,3-b]quinolin-4(2H)-one was prepared as an orange-yellow solid according to Synthetic Scheme 4: mp=224-225° C.; ¹H NMR (500 MHz, DMSO-d₆) δ 9.42 (s, 1H), 8.83 (d, J=2.1 Hz, 1H), 8.28 (dd, J=8.9, 2.2 Hz, 1H), 8.19 (d, J=8.9 Hz, 1H), 7.55 (d, J=1.6 Hz, 1H), 7.41-7.39 (m, 1H), 7.17 (d, J=8.1 Hz, 1H), 6.89 (s, 1H), 4.20-4.15 (m, 1H), 4.10-4.06 (m, 1H), 2.34-2.27 (m, 5H); ESI MS m/z 350 [C₁₉H₁₅N₃O₂S+H]⁺; HPLC (Method C) >99% (AUC), t_{R}=11.40 min; Chiral HPLC (Chiralpak AD, Method A) >99% (AUC), t_{R}=26.92 min.

### Preparation of (S)-3a-Hydroxy-6,7-dimethyl-1-(2-methylbenzofuran-5-yl)-3,3a-dihydro-1H-pyrrolo[2,3-b]quinolin-4(2H)-one

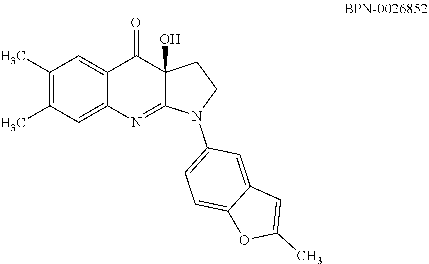

(S)-3a-Hydroxy-6,7-dimethyl-1-(2-methylbenzofuran-5-yl)-3,3a-dihydro-1H-pyrrolo[2,3-b]quinolin-4(2H)-one was prepared as a yellow solid according to Synthetic Scheme 5: mp=191-192° C.; ¹H NMR (500 MHz, DMSO-d₆) δ 8.14 (d, J=2.3 Hz, 1H), 7.94 (dd, J=9.0, 2.3 Hz, 1H), 7.52 (d, J=9.0 Hz, 1H), 7.47 (s, 1H), 7.01 (s, 1H), 6.74 (s, 1H), 6.62 (apparent t, J=0.9 Hz, 1H), 4.14-4.09 (m, 1H), 3.98-3.94 (m, 1H), 2.46 (d, J=0.8 Hz, 3H), 2.26-2.25 (m, 5H), 2.21 (s, 3H); ESI MS m/z 361 [C₂₂H₂₀N₂O₃+H]⁺; HPLC (Method A) 96.6% (AUC), t_{R}=13.64 min; Chiral HPLC (Chiralpak AD, Method A) 81.8% (AUC), t_{R}=17.48 min.

### Preparation of (S)-1-(Benzo[d]thiazol-6-yl)-3a-hydroxy-6-methyl-3,3a-dihydro-1H-pyrrolo[2,3-b]quinolin-4(2H)-one

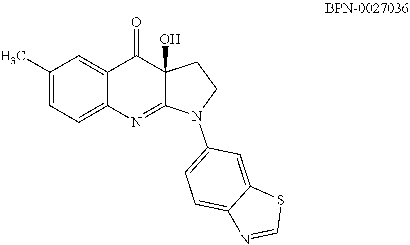 [SMILES: Cc1ccc2c(c1)C(=O)[C@]1(O)CCN(c3ccc4ncsc4c3)C1=N2]

(S)-1-(Benzo[d]thiazol-6-yl)-3a-hydroxy-6-methyl-3,3a-dihydro-1H-pyrrolo[2,3-b]quinolin-4(2H)-one was prepared as a yellow solid according to Synthetic Scheme 6: mp=225-226° C.; ¹H NMR (500 MHz, DMSO-d₆) δ 9.32 (s, 1H), 8.88 (d, J=2.2 Hz, 1H), 8.35 (dd, J=9.0, 2.3 Hz, 1H), 8.12 (d, J=9.0 Hz, 1H), 7.55 (d, J=1.7 Hz, 1H), 7.40 (dd, J=8.2, 2.1 Hz, 1H), 7.20 (d, J=8.1 Hz, 1H), 6.88 (s, 1H), 4.18-4.13 (m, 1H), 4.08-4.04 (m, 1H), 2.33-2.29 (m, 5H); ESI MS m/z 350 [C₁₉H₁₅N₃O₂S+H]⁺; HPLC (Method C) 97.0% (AUC), t_{R}=11.57 min; Chiral HPLC (Chiralpak AD, Method A) >99% (AUC), t_{R}=23.11 min.

### Preparation of (S)-4a-Hydroxy-1-methyl-7-phenyl-4a,5,6,7-tetrahydropyrazolo[3,4-b]pyrrolo[3,2-e]pyridin-4(1)-one

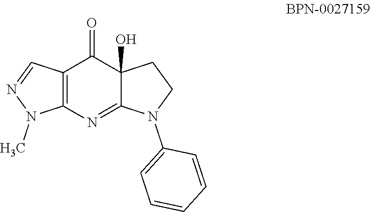 [SMILES: Cn1ncc2c1N=C1N(c3ccccc3)CC[C@@]1(O)C2=O]

(S)-4a-Hydroxy-1-methyl-7-phenyl-4a,5,6,7-tetrahydropyrazolo[3,4-b]pyrrolo[3,2-e]pyridin-4(1H)-one was prepared as a yellow solid according to Synthetic Scheme 2: mp=244-245° C.; ¹H NMR (500 MHz, DMSO-d₆) δ 8.01 (d, J=7.9 Hz, 2H), 7.72 (s, 1H), 7.49-7.46 (m, 2H), 7.23 (apparent t, J=7.4 Hz, 1H), 6.84 (s, 1H), 4.22-4.17 (m, 1H), 4.07-4.04 (m, 1H), 3.73 (s, 3H), 2.26-2.14 (m, 2H); ESI MS m/z 283 [C₁₅H₁₄N₄O₂+H]⁺; HPLC (Method C) >99% (AUC), t_{R}=12.99 min; Chiral HPLC (Chiralpak AD, Method A) >99% (AUC), t_{R}=22.19 min.

### Preparation of (S)-1-(3-Chloro-4-(hydroxymethyl)phenyl)-3a-hydroxy-6-methyl-3,3a-dihydro-1H-pyrrolo[2,3-b]quinolin-4(2H)-one

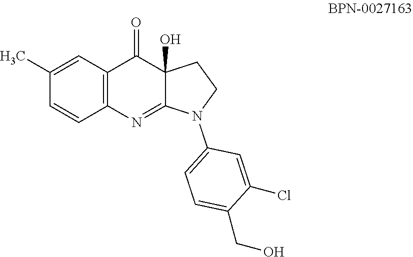 [SMILES: Cc1ccc2c(c1)C(=O)[C@]1(O)CCN(c3ccc(CO)c(Cl)c3)C1=N2]

(S)-1-(3-Chloro-4-(hydroxymethyl)phenyl)-3a-hydroxy-6-methyl-3,3a-dihydro-1H-pyrrolo[2,3-b]quinolin-4(2H)-one was prepared as a yellow solid according to Synthetic Scheme 6: mp=199-200° C.; ¹H NMR (500 MHz, DMSO-d₆) δ 8.40 (d, J=2.3 Hz, 1H), 7.88 (dd, J=8.6, 2.3 Hz, 1H), 7.56-7.54 (m, 2H), 7.40-7.38 (m, 1H), 7.13 (d, J=8.1 Hz, 1H), 6.85 (s, 1H), 5.34 (apparent t, J=5.6 Hz, 1H), 4.56 (d, J=5.6 Hz, 2H), 4.06-4.01 (m, 1H), 3.99-3.95 (m, 1H), 2.31 (s, 3H), 2.29-2.24 (m, 2H); ESI MS m/z 357 [C₁₉H₁₇ClN₂O₃+H]⁺; HPLC (Method C) 98.9% (AUC), t_{R}=11.91 min; Chiral HPLC (Chiralpak AD, Method A) 92.4% (AUC), t_{R}=20.92 min.

### Preparation of (S)-3a-Hydroxy-6,7-dimethyl-1-(2-methylbenzo[d]oxazol-6-yl)-3,3a-dihydro-1H-pyrrolo[2,3-b]quinolin-4(2H)-one

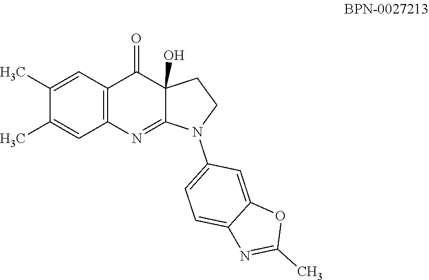

(S)-3a-Hydroxy-6,7-dimethyl-1-(2-methylbenzo[d]oxazol-6-yl)-3,3a-dihydro-1H-pyrrolo[2,3-b]quinolin-4(2H)-one was prepared as a yellow solid according to Synthetic Scheme 6: mp=205-209° C.; ¹H NMR (500 MHz, DMSO-d₆) δ 8.66 (d, J=2.0 Hz, 1H), 7.90 (dd, J=8.8, 2.1 Hz, 1H), 7.67 (d, J=8.7 Hz, 1H), 7.50 (s, 1H), 7.09 (s, 1H), 6.80 (s, 1H), 4.14-4.09 (m, 1H), 4.04-3.99 (m, 1H), 2.62 (s, 3H), 2.28-2.25 (m, 5H), 2.23 (s, 3H); ESI MS m/z 362 [C₂₁H₁₉N₃O₃+H]⁺; HPLC (Method C) 90.8% (AUC), t_{R}=12.58 min; Chiral HPLC (Chiralpak AD, Method A) 79.2% (AUC), t_{R}=18.93 min.

### Preparation of (S)-3a-Hydroxy-1-(2-(hydroxymethyl)quinolin-6-yl)-6-methyl-3,3a-dihydro-1H-pyrrolo[2,3-b]quinolin-4(2H)-one

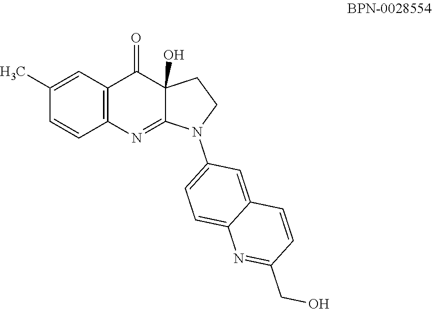 [SMILES: Cc1ccc2c(c1)C(=O)[C@]1(O)CCN(c3ccc4nc(CO)ccc4c3)C1=N2]

(S)-3a-Hydroxy-1-(2-(hydroxymethyl)quinolin-6-yl)-6-methyl-3,3a-dihydro-1H-pyrrolo[2,3-b]quinolin-4(2H)-one was prepared as a yellow solid according to Synthetic Scheme 6: mp=223-224° C.; ¹H NMR (300 MHz, DMSO-d₆) δ 8.75 (dd, J=9.3, 2.6 Hz, 1H), 8.46 (d, J=2.4 Hz, 1H), 8.36 (d, J=8.5 Hz, 1H), 8.00 (d, J=9.3 Hz, 1H), 7.66 (d, J=8.5 Hz, 1H), 7.56 (d, J=2.1 Hz, 1H), 7.43-7.40 (m, 1H), 7.22 (d, J=8.1 Hz, 1H), 6.91 (s, 1H),), 5.55 (apparent t, J=5.9 Hz, 1H), 4.72 (d, J=5.9 Hz, 2H), 4.19-4.12 (m, 2H), 2.36-2.27 (m, 5H); ESI MS m/z 374 [C₂₂H₁₉N₃O₃+H]⁺; UPLC (Method A) 97.9% (AUC), t_{R}=2.47 min; Chiral HPLC (Chiralpak AD, Method A) 91.6% (AUC), t_{R}=19.66 min.

### Preparation of (S)-3a-Hydroxy-1-(2-(hydroxymethyl)benzofuran-5-yl)-6,7-dimethyl-3,3a-dihydro-1H-pyrrolo[2,3-b]quinolin-4(2H)-one

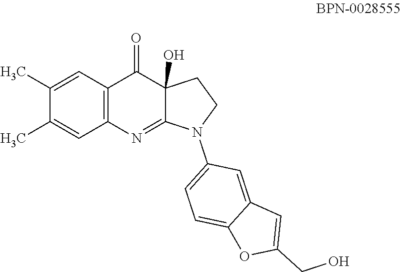 [SMILES: Cc1cc2c(cc1C)C(=O)[C@]1(O)CCN(c3ccc4oc(CO)cc4c3)C1=N2]

(S)-3a-Hydroxy-1-(2-(hydroxymethyl)benzofuran-5-yl)-6,7-dimethyl-3,3a-dihydro-1H-pyrrolo[2,3-b]quinolin-4(2H)-one was prepared as a yellow-orange solid according to Synthetic Scheme 6: mp=210-211° C.; ¹H NMR (300 MHz, DMSO-d₆) δ 8.21 (d, J=2.2 Hz, 1H), 8.03 (dd, J=9.0, 2.3 Hz, 1H), 7.58 (d, J=9.0 Hz, 1H), 7.48 (s, 1H), 7.03 (s, 1H), 6.80 (d, J=0.7 Hz, 1H), 6.78 (s, 1H), 5.52-5.48 (m, 1H), 4.58 (d, J=5.7 Hz, 2H), 4.17-4.09 (m, 1H), 4.00-3.94 (m, 1H), 2.26-2.22 (m, 8H); ESI MS m/z 377 [C₂₂H₂₀N₂₀₄+H]⁺; UPLC (Method A) 97.4% (AUC), t_{R}=3.05 min; Chiral HPLC (Chiralpak AD, Method A) 64.2% (AUC), t_{R}=24.05 min.

All patents and publications referred to herein are incorporated by reference herein to the same extent as if each individual publication was specifically and individually indicated to be incorporated by reference in its entirety.

### DOCUMENTS CITED

1. Childress, A. R.; Mozley, P. D.; McElgin, W.; Fitzgerald, J.; Reivich, M.; O'Brien, C. P., Limbic activation during cue-induced cocaine craving. The American journal of psychiatry 1999, 156 (1), 11-8.
2. Si, J.; Ge, Y.; Zhuang, S.; Gong, R., Inhibiting nonmuscle myosin II impedes inflammatory infiltration and ameliorates progressive renal disease. Lab Invest 2010, 90 (3), 448-58.
3. Doller, A.; Badawi, A.; Schmid, T.; Brauss, T.; Pleli, T.; zu Heringdorf, D. M.; Piiper, A.; Pfeilschifter, J.; Eberhardt, W., The cytoskeletal inhibitors latrunculin A and blebbistatin exert antitumorigenic properties in human hepatocellular carcinoma cells by interfering with intracellular HuR trafficking. Exp Cell Res 2015, 330 (1), 66-80.
4. Duxbury, M. S.; Ashley, S. W.; Whang, E. E., Inhibition of pancreatic adenocarcinoma cellular invasiveness by blebbistatin: a novel myosin II inhibitor. Biochem Biophys Res Commun 2004, 313 (4), 992-7.
5. Derycke, L.; Stove, C.; Vercoutter-Edouart, A. S.; De Wever, O.; Dolle, L.; Colpaert, N.; Depypere, H.; Michalski, J. C.; Bracke, M., The role of non-muscle myosin IIA in aggregation and invasion of human MCF-7 breast cancer cells. Int J Dev Biol 2011, 55 (7-9), 835-40.
6. Poincloux, R.; Collin, O.; Lizarraga, F.; Romao, M.; Debray, M.; Piel, M.; Chavrier, P., Contractility of the cell rear drives invasion of breast tumor cells in 3D Matrigel. Proc Natl Acad Sci USA 2011, 108 (5), 1943-8.
7. Tomii, S.; Akashi, T.; Ando, N.; Tamura, T.; Sakurai, A.; Terada, A.; Furukawa, A.; Suzuki, Y.; Kayamori, K.; Sakamoto, K.; Ishibashi, H.; Eishi, Y., Cortical Actin Alteration at the Matrix-Side Cytoplasm in Lung Adenocarcinoma Cells and Its Significance in Invasion. Pathobiology 2017, 84 (4), 171-183.
8. Beadle, C.; Assanah, M. C.; Monzo, P.; Vallee, R.; Rosenfeld, S. S.; Canoll, P., The role of myosin II in glioma invasion of the brain. Mol Biol Cell 2008, 19 (8), 3357-68.
9. Ivkovic, S.; Beadle, C.; Noticewala, S.; Massey, S. C.; Swanson, K. R.; Toro, L. N.; Bresnick, A. R.; Canoll, P.; Rosenfeld, S. S., Direct inhibition of myosin II effectively blocks glioma invasion in the presence of multiple motogens. Mol Biol Cell 2012, 23 (4), 533-42.
10. Wigton, E. J.; Thompson, S. B.; Long, R. A.; Jacobelli, J., Myosin-IIA regulates leukemia engraftment and brain infiltration in a mouse model of acute lymphoblastic leukemia. J Leukoc Biol 2016, 100 (1), 143-53.
11. Picariello, H.; Kenchappa, R.; Rai, V.; Crish, J.; Dovas, A.; Pogoda, K.; McMahon, M.; Bell, E.; Chandrasekharan, U.; Luu, A.; West, R.; Lammerding, J.; Canoll, P.; Odde, D.; Janmey, P.; Egelhoff, T.; Rosenfeld, S. S., Myosin IIA Suppresses Glioblastoma Development In a Mechanically-Sensitive Manner. Proceedings of the National Academy of Sciences of the United States of America 2019, (submitted).
12. Chen, P.; Yin, J.; Guo, Y. M.; Xiao, H.; Wang, X. H.; DiSanto, M. E.; Zhang, X. H., The expression and functional activities of smooth muscle myosin and non-muscle myosin isoforms in rat prostate. J Cell Mol Med 2018, 22 (1), 576-588.
13. Zhai, K.; Tang, Y.; Zhang, Y.; Li, F.; Wang, Y.; Cao, Z.; Yu, J.; Kou, J.; Yu, B., NMMHC IIA inhibition impedes tissue factor expression and venous thrombosis via Akt/GSK3beta-NF-kappaB signalling pathways in the endothelium. Thromb Haemost 2015, 114 (1), 173-85.
14. Zhang, Y.; Li, L.; Zhao, Y.; Han, H.; Hu, Y.; Liang, D.; Yu, B.; Kou, J., The Myosin II Inhibitor, Blebbistatin, Ameliorates FeCl3-induced Arterial Thrombosis via the GSK3beta-NF-kappaB Pathway. Int J Biol Sci 2017, 13 (5), 630-639.
15. Feghhi, S.; Tooley, W. W.; Sniadecki, N. J., Nonmuscle Myosin IIA Regulates Platelet Contractile Forces Through Rho Kinase and Myosin Light-Chain Kinase. J Biomech Eng 2016, 138 (10).
16. Wang, Y.; Xu, Y.; Liu, Q.; Zhang, Y.; Gao, Z.; Yin, M.; Jiang, N.; Cao, G.; Yu, B.; Cao, Z.; Kou, J., Myosin IIA-related Actomyosin Contractility Mediates Oxidative Stress-induced Neuronal Apoptosis. Front Mol Neurosci 2017, 10, 75.
17. Wang, J. F.; Zarbin, M. A.; Sugino, I.; Whitehead, I.; Townes-Anderson, E., RhoA Signaling and Synaptic Damage Occur within Hours in a Live Pig Model of Retinal Detachment. Investigative Ophthalmology & Visual Science 2016, 57 (12).
18. Southern, B. D.; Grove, L. M.; Rahaman, S. O.; Abraham, S.; Scheraga, R. G.; Niese, K. A.; Sun, H.; Herzog, E. L.; Liu, F.; Tschumperlin, D. J.; Egelhoff, T. T.; Rosenfeld, S. S.; Olman, M. A., Matrix-driven Myosin II Mediates the Pro-fibrotic Fibroblast Phenotype. J Biol Chem 2016, 291 (12), 6083-95.
19. Liu, Z.; van Grunsven, L. A.; Van Rossen, E.; Schroyen, B.; Timmermans, J. P.; Geerts, A.; Reynaert, H., Blebbistatin inhibits contraction and accelerates migration in mouse hepatic stellate cells. Br J Pharmacol 2010, 159 (2), 304-15.
20. Atluri, K.; De Jesus, A. M.; Chinnathambi, S.; Brouillette, M. J.; Martin, J. A.; Salem, A. K.; Sander, E. A., Blebbistatin-Loaded Poly(D,L-lactide-co-glycolide) Particles For Treating Arthrofibrosis. Acs Biomaterials Science & Engineering 2016, 2 (7), 1097-1107.
21. Bond, J. E.; Bergeron, A.; Thurlow, P.; Selim, M. A.; Bowers, E. V.; Kuang, A.; Levinson, H., Angiotensin-II mediates nonmuscle myosin II activation and expression and contributes to human keloid disease progression. Mol Med 2011, 17 (11-12), 1196-203.
22. Bond, J. E.; Ho, T. Q.; Selim, M. A.; Hunter, C. L.; Bowers, E. V.; Levinson, H., Temporal spatial expression and function of non-muscle myosin II isoforms IIA and IIB in scar remodeling. Lab Invest 2011, 91 (4), 499-508.
23. Arora, P. D.; Wang, Y.; Bresnick, A.; Janmey, P. A.; McCulloch, C. A., Flightless I interacts with NMMIIA to promote cell extension formation, which enables collagen remodeling. Mol Biol Cell 2015, 26 (12), 2279-97.
24. Kubo, T.; Yamaguchi, A.; Iwata, N.; Yamashita, T., The therapeutic effects of Rho-ROCK inhibitors on CNS disorders. Ther Clin Risk Manag 2008, 4 (3), 605-15.
25. Yoshimoto, T.; Fujita, T.; Kajiya, M.; Ouhara, K.; Matsuda, S.; Komatsuzawa, H.; Shiba, H.; Kurihara, H., Aggregatibacter actinomycetemcomitans outer membrane protein 29 (Omp29) induces TGF-beta-regulated apoptosis signal in human gingival epithelial cells via fibronectin/integrinbeta1/FAK cascade. Cell Microbiol 2016, 18 (12), 1723-1738.
26. Zhang, M.; Rao, P. V., Blebbistatin, a novel inhibitor of myosin II ATPase activity, increases aqueous humor outflow facility in perfused enucleated porcine eyes. Invest Ophthalmol Vis Sci 2005, 46 (11), 4130-8.
27. Epstein, D. L.; Rowlette, L. L.; Roberts, B. C., Acto-myosin drug effects and aqueous outflow function. Invest Ophthalmol Vis Sci 1999, 40 (1), 74-81.
28. Jiang, L.; Hu, J.; Feng, J.; Han, D.; Yang, C., Substrate stiffness of endothelial cells directs LFA-1/ICAM-1 interaction: A physical trigger of immune-related diseases?Clin Hemorheol Microcirc 2016, 61 (4), 633-43.
29. Antoine, T. E.; Shukla, D., Inhibition of myosin light chain kinase can be targeted for the development of new therapies against herpes simplex virus type-1 infection. Antivir Ther 2014, 19 (1), 15-29.
30. Arii, J.; Goto, H.; Suenaga, T.; Oyama, M.; Kozuka-Hata, H.; Imai, T.; Minowa, A.; Akashi, H.; Arase, H.; Kawaoka, Y.; Kawaguchi, Y., Non-muscle myosin IIA is a functional entry receptor for herpes simplex virus-1. Nature 2010, 467 (7317), 859-62.
31. Kumakura, M.; Kawaguchi, A.; Nagata, K., Actin-myosin network is required for proper assembly of influenza virus particles. Virology 2015, 476, 141-150.
32. Sun, Y.; Qi, Y.; Liu, C.; Gao, W.; Chen, P.; Fu, L.; Peng, B.; Wang, H.; Jing, Z.; Zhong, G.; Li, W., Nonmuscle myosin heavy chain IIA is a critical factor contributing to the efficiency of early infection of severe fever with thrombocytopenia syndrome virus. J Virol 2014, 88 (1), 237-48.
33. Cymerys, J.; Slonska, A.; Skwarska, J.; Banbura, M. W., Function of myosin during entry and egress of equid herpesvirus type 1 in primary murine neurons. Acta Virol 2016, 60 (4), 410-416.
34. Michael, S. K.; Surks, H. K.; Wang, Y.; Zhu, Y.; Blanton, R.; Jamnongjit, M.; Aronovitz, M.; Baur, W.; Ohtani, K.; Wilkerson, M. K.; Bonev, A. D.; Nelson, M. T.; Karas, R. H.; Mendelsohn, M. E., High blood pressure arising from a defect in vascular function. Proc Natl Acad Sci USA 2008, 105 (18), 6702-7.
35. Xiao, J. W.; Zhu, X. Y.; Wang, Q. G.; Zhang, D. Z.; Cui, C. S.; Zhang, P.; Chen, H. Y.; Meng, L. L., Acute effects of Rho-kinase inhibitor fasudil on pulmonary arterial hypertension in patients with congenital heart defects. Circ J 2015, 79 (6), 1342-8.
36. Sirigu, S.; Hartman, J. J.; Planelles-Herrero, V. J.; Ropars, V.; Clancy, S.; Wang, X.; Chuang, G.; Qian, X.; Lu, P. P.; Barrett, E.; Rudolph, K.; Royer, C.; Morgan, B. P.; Stura, E. A.; Malik, F. I.; Houdusse, A. M., Highly selective inhibition of myosin motors provides the basis of potential therapeutic application. Proc Natl Acad Sci USA 2016, 113 (47), E7448-E7455.
37. Brozovich, F. V.; Nicholson, C. J.; Degen, C. V.; Gao, Y. Z.; Aggarwal, M.; Morgan, K. G., Mechanisms of Vascular Smooth Muscle Contraction and the Basis for Pharmacologic Treatment of Smooth Muscle Disorders. Pharmacol Rev 2016, 68 (2), 476-532.
38. Zhang, X. H.; Aydin, M.; Kuppam, D.; Melman, A.; Disanto, M. E., In vitro and in vivo relaxation of corpus cavernosum smooth muscle by the selective myosin II inhibitor, blebbistatin. J Sex Med 2009, 6 (10), 2661-71.
39. Zhang, X.; Kuppam, D. S.; Melman, A.; DiSanto, M. E., In vitro and in vivo relaxation of urinary bladder smooth muscle by the selective myosin II inhibitor, blebbistatin. BJU Int 2011, 107 (2), 310-7.
40. Kampourakis, T.; Zhang, X.; Sun, Y. B.; Irving, M., Omecamtiv mercabil and blebbistatin modulate cardiac contractility by perturbing the regulatory state of the myosin filament. J Physiol 2018, 596 (1), 31-46.
41. Maher, C.; Underwood, M.; Buchbinder, R., Non-specific low back pain. Lancet 2017, 389 (10070), 736-747.
42. Hirayama, J.; Yamagata, M.; Ogata, S.; Shimizu, K.; Ikeda, Y.; Takahashi, K., Relationship between low-back pain, muscle spasm and pressure pain thresholds in patients with lumbar disc herniation. Eur Spine J 2006, 15 (1), 41-7.
43. Foley, P. L.; Vesterinen, H. M.; Laird, B. J.; Sena, E. S.; Colvin, L. A.; Chandran, S.; MacLeod, M. R.; Fallon, M. T., Prevalence and natural history of pain in adults with multiple sclerosis: systematic review and meta-analysis. Pain 2013, 154 (5), 632-42.
44. Mameniskiene, R.; Wolf, P., Epilepsia partialis continua: A review. Seizure 2017, 44, 74-80.
45. Espay, A. J.; Schmithorst, V. J.; Szaflarski, J. P., Chronic isolated hemifacial spasm as a manifestation of epilepsia partialis continua. Epilepsy Behav 2008, 12 (2), 332-6.
46. Pacik, P. T., Understanding and treating vaginismus: a multimodal approach. Int Urogynecol J 2014, 25 (12), 1613-20.
47. Ochala, J.; Sun, Y. B., Novel myosin-based therapies for congenital cardiac and skeletal myopathies. J Med Genet 2016, 53 (10), 651-4.
48. Mohamed, R. M. P.; Kumar, J.; Yap, E.; Mohamed, I. N.; Sidi, H.; Adam, R. L.; Das, S., Try to Remember: Interplay between Memory and Substance Use Disorder. Current Drug Targets 2019, 20 (2), 158-165.
49. Soeter, M.; Kindt, M., An Abrupt Transformation of Phobic Behavior After a Post-Retrieval Amnesic Agent. Biol Psychiatry 2015, 78 (12), 880-6.
50. Brunet, A.; Saumier, D.; Liu, A.; Streiner, D. L.; Tremblay, J.; Pitman, R. K., Reduction of PTSD Symptoms With Pre-Reactivation Propranolol Therapy: A Randomized Controlled Trial. Am J Psychiatry 2018, 175 (5), 427-433.
51. Young, E. J.; Blouin, A. M.; Briggs, S. B.; Sillivan, S. E.; Lin, L.; Cameron, M. D.; Rumbaugh, G.; Miller, C. A., Nonmuscle myosin IIB as a therapeutic target for the prevention of relapse to methamphetamine use. Molecular Psychiatry 2016, 21 (5), 615-623.
52. Young, E. J.; Briggs, S. B.; Rumbaugh, G.; Miller, C. A., Nonmuscle myosin II inhibition disrupts methamphetamine-associated memory in females and adolescents. Neurobiol Learn Mem 2017, 139, 109-116.
53. Grant, J. E.; Chamberlain, S. R., Expanding the definition of addiction: DSM-5 vs. ICD-11. CNS Spectr 2016, 21 (4), 300-3.
54. Kasai, H.; Fukuda, M.; Watanabe, S.; Hayashi-Takagi, A.; Noguchi, J., Structural dynamics of dendritic spines in memory and cognition. Trends in neurosciences 2010, 33 (3), 121-9.
55. Lai, C. S. W.; Franke, T. F.; Gan, W.-B., Opposite effects of fear conditioning and extinction on dendritic spine remodelling. Nature 2012, 483 (7387), 87-91.
56. Yang, G.; Pan, F.; Gan, W.-B., Stably maintained dendritic spines are associated with lifelong memories. Nature 2009, 462 (7275), 920-924.
57. Kasai, H.; Matsuzaki, M.; Noguchi, J.; Yasumatsu, N.; Nakahara, H., Structure-stability-function relationships of dendritic spines. Trends in Neurosciences 2003, 26 (7), 360-368.
58. Smart, F. M.; Halpain, S., Regulation of dendritic spine stability. Hippocampus 2000, 10 (5), 542-554.
59. Rex, C. S.; Gavin, C. F.; Rubio, M. D.; Kramar, E. A.; Chen, L. Y.; Jia, Y.; Huganir, R. L.; Muzyczka, N.; Gall, C. M.; Miller, C. A.; Lynch, G.; Rumbaugh, G., Myosin IIb regulates actin dynamics during synaptic plasticity and memory formation. Neuron 2010, 67 (4), 603-17.
60. Mantzur, L.; Joels, G.; Lamprecht, R., Actin polymerization in lateral amygdala is essential for fear memory formation. Neurobiology of learning and memory 2009, 91 (1), 85-8.
61. Rehberg, K.; Bergado-Acosta, J. R.; Koch, J. C.; Stork, O., Disruption of fear memory consolidation and reconsolidation by actin filament arrest in the basolateral amygdala. Neurobiology of learning and memory 2010, 94 (2), 117-26.
62. Fischer, A.; Sananbenesi, F.; Schrick, C.; Spiess, J.; Radulovic, J., Distinct roles of hippocampal de novo protein synthesis and actin rearrangement in extinction of contextual fear. The Journal of neuroscience: the official journal of the Society for Neuroscience 2004, 24 (8), 1962-6.
63. Gavin, C. F.; Rubio, M. D.; Young, E.; Miller, C.; Rumbaugh, G., Myosin II motor activity in the lateral amygdala is required for fear memory consolidation. Learn Mem 2012, 19 (1), 9-14.
64. Star, E. N.; Kwiatkowski, D. J.; Murthy, V. N., Rapid turnover of actin in dendritic spines and its regulation by activity. Nature neuroscience 2002, 5 (3), 239-46.
65. Young, E. J.; Aceti, M.; Griggs, E. M.; Fuchs, R. A.; Zigmond, Z.; Rumbaugh, G.; Miller, C. A., Selective, retrieval-independent disruption of methamphetamine-associated memory by actin depolymerization. Biological psychiatry 2014, 75 (2), 96-104.
66. Krucker, T.; Siggins, G. R.; Halpain, S., Dynamic actin filaments are required for stable long-term potentiation (LTP) in area CA1 of the hippocampus. Proc. Nat. Acad. Sci. 2000, 97 (12), 6856-6861.
67. Honkura, N.; Matsuzaki, M.; Noguchi, J.; Ellis-Davies, G. C.; Kasai, H., The subspine organization of actin fibers regulates the structure and plasticity of dendritic spines. Neuron 2008, 57 (5), 719-29.
68. Kaech, S.; Fischer, M.; Doll, T.; Matus, A., Isoform specificity in the relationship of actin to dendritic spines. J Neurosci 1997, 17 (24), 9565-72.
69. Kramar, E. A., Integrin-driven actin polymerization consolidates long-term potentiation. Proc. Nat. Acad. Sci. 2006, 103 (14), 5579-5584.
70. Straight, A. F.; Cheung, A.; Limouze, J.; Chen, I.; Westwood, N. J.; Sellers, J. R.; Mitchison, T. J., Dissecting temporal and spatial control of cytokinesis with a myosin II Inhibitor. Science 2003, 299 (5613), 1743-7.
71. Kovacs, M.; Toth, J.; Hetenyi, C.; Malnasi-Csizmadia, A.; Sellers, J. R., Mechanism of blebbistatin inhibition of myosin II. The Journal of biological chemistry 2004, 279 (34), 35557-63.
72. Limouze, J.; Straight, A. F.; Mitchison, T.; Sellers, J. R., Specificity of blebbistatin, an inhibitor of myosin II. Journal of muscle research and cell motility 2004, 25 (4-5), 337-41.
73. Sakamoto, T.; Limouze, J.; Combs, C. A.; Straight, A. F.; Sellers, J. R., Blebbistatin, a myosin II inhibitor, is photoinactivated by blue light. Biochemistry 2005, 44 (2), 584-8.
74. Young, E. J.; Blouin, A. M.; Briggs, S. B.; Sillivan, S. E.; Lin, L.; Cameron, M. D.; Rumbaugh, G.; Miller, C. A., Nonmuscle myosin IIB as a therapeutic target for the prevention of relapse to methamphetamine use. Molecular psychiatry 2015.
75. Briggs, S. B.; Blouin, A. M.; Young, E. J.; Rumbaugh, G.; Miller, C. A., Memory disrupting effects of nonmuscle myosin II inhibition depend on the class of abused drug and brain region. Learn Mem 2017, 24 (2), 70-75.
76. Briggs, S. B.; Hafenbreidel, M.; Young, E. J.; Rumbaugh, G.; Miller, C. A., The role of nonmuscle myosin II in polydrug memories and memory reconsolidation. Learn Mem 2018, 25 (9), 391-398.
77. Allingham, J. S.; Smith, R.; Rayment, I., The structural basis of blebbistatin inhibition and specificity for myosin II. Nat. Struct. Mol. Biol. 2005, 12 (4), 378-379.
78. Lucas-Lopez, C.; Allingham, J. S.; Lebl, T.; Lawson, C. P.; Brenk, R.; Sellers, J. R.; Rayment, I.; Westwood, N. J., The small molecule tool (S)-(−)-blebbistatin: novel insights of relevance to myosin inhibitor design. Org. Biomol. Chem. 2008, 6 (12), 2076-2084.
79. Eddinger, T. J.; Meer, D. P.; Miner, A. S.; Meehl, J.; Rovner, A. S.; Ratz, P. H., Potent inhibition of arterial smooth muscle tonic contractions by the selective myosin II inhibitor, blebbistatin. J. Pharmacol. Exp. Ther. 2007, 320 (2), 865-870.
80. Borlak, J.; Zwadlo, C., The Myosin ATPase Inhibitor 2,3-Butanedione monoxime Dictates Transcriptional Activation of Ion Channels and Ca²⁺-Handling Proteins. Mol. Pharmacol. 2004, 66 (3), 708-717.
81. Cheung, A.; Dantzig, J. A.; Hollingworth, S.; Baylor, S. M.; Goldman, Y. E.; Mitchison, T. J.; Straight, A. F., A small-molecule inhibitor of skeletal muscle myosin II. Nature Cell Biol. 2002, 4 (83-88).
82. Lucas-Lopez, C.; Patterson, S.; Blum, T.; Straight, A. F.; Toth, J.; Slawin, A. M. Z.; Mitchison, T. J.; Sellers, J. R.; Westwood, N. J., Absolute Stereochemical Assignment and Fluorescence Tuning of the Small Molecule Tool, (−)-Blebbistatin. Eur. J. Org. Chem. 2005, 1736-1740.
83. Kepiro, M.; Varkuti, B. H.; Vegner, L.; Voros, G.; Hegyi, G.; Varga, M.; Malnasi-Csizmadia, A., para-Nitroblebbistatin, the Non-Cytotoxic and Photostable Myosin II Inhibitor. Angew. Chem. Int. Ed. Engl. 2014, 53 (31), 8211-8215.
84. Kepiro, M.; Varkuti, B. H.; Bodor, A.; Hegyi, G.; Drahos, L.; Kovacs, M.; Malnasi-Csizmadia, A., Azidoblebbistatin, a photoreactive myosin inhibitor. Proc. Nat. Acad. Sci. 2012, 109 (24), 9402-9407.
85. Kovacs, M.; Toth, J.; Hetenyi, C.; Malnasi-Csizmadia, A.; Sellers, J. R., Mechanism of blebbistatin inhibition of myosin II. J. Biol. Chem. 2004, 279 (34), 35557-35563.
86. Zhang, X.; Kuppam, D. S. R.; Melman, A.; DiSanto, M. E., In vitro and in vivo relaxation of urinary bladder smooth muscle by the selective myosin II inhibitor, blebbistatin. BJUI 2010, 107, 310-317.
87. Zhang, X. H.; Aydin, M.; Kuppam, D.; Melman, A.; Disanto, M. E., In vitro and in vivo relaxation of corpus cavernosum smooth muscle by the selective myosin II inhibitor, blebbistatin. J. Sex Med. 2009, 6 (10), 2661-2671.
88. Limouze, J.; Straight, A. F.; Mitchison, T.; Sellers, J. R., Specificity of blebbistatin, an inhibitor of myosin II. J. Muscle Res. Cell Motil. 2004, 25 (4-5), 337-341.
89. Ponsaerts, R.; D′Hondt, C.; Bultynck, G.; Srinivas, S. P.; Vereecke, J.; Himpens, B., The myosin II ATPase inhibitor blebbistatin prevents thrombin-induced inhibition of intercellular calcium wave propagation in corneal endothelial cells. Invest. Ophthalmol. Vis. Sci. 2008, 49 (11), 4816-4827.
90. Zhang, M.; Rao, P. V., Blebbistatin, a novel inhibitor of myosin II ATPase activity, increases aqueous humor outflow facility in perfused enucleated porcine eyes. Invest. Ophthalmol. Vis. Sci. 2005, 46 (11), 4130-4138.
91. Villanueva, J.; Torres, V.; Torregrosa-Hetland, C. J.; Garcia-Martinez, V.; Lopez-Font, I.; Viniegra, S.; Gutierrez, L. M., F-actin-myosin II inhibitors affect chromaffin granule plasma membrane distance and fusion kinetics by retraction of the cytoskeletal cortex. J. Mol. Neurosci. 2012, 48 (2), 328-338.
92. Yumoto, M.; Watanabe, M., Blebbistatin, a myosin II inhibitor, suppresses Ca(2+)-induced and “sensitized”-contraction of skinned tracheal muscles from guinea pig. J. Smooth Muscle Res. 2013, 49, 89-98.
93. Watanabe, M.; Yumoto, M.; Tanaka, H.; Wang, H. H.; Katayama, T.; Yoshiyama, S.; Black, J.; Thatcher, S. E.; Kohama, K., Blebbistatin, a myosin II inhibitor, suppresses contraction and disrupts contractile filaments organization of skinned taenia cecum from guinea pig. Am. J. Physiol. Cell Physiol. 2010, 298 (5), C1118-26.
94. Bond, J. E.; Ho, T. Q.; Selim, M. A.; Hunter, C. L.; Bowers, E. V.; Levinson, H., Temporal spatial expression and function of non-muscle myosin II isoforms IIA and IIB in scar remodeling. Lab. Invest. 2011, 91 (4), 499-508.

All patents and publications referred to herein are incorporated by reference herein to the same extent as if each individual publication was specifically and individually indicated to be incorporated by reference in its entirety.

1.-5. (canceled)

6. A compound according to the formula: 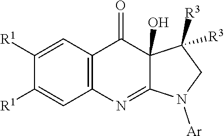
or a pharmaceutically acceptable salt thereof, wherein:
R¹ is independently at each occurrence H, (C₁-C₄) alkyl, —CF₃, or halo;
Ar is a monocyclic aryl, a monocyclic heteroaryl, or a bicyclic heteroaryl substituted with 1, 2 or 3 R², or a bicyclic aryl substituted with 0, 1, 2 or 3 R²; wherein
when Ar is a monocyclic aryl, then R² is independently at each occurrence hydroxymethyl, R²NCH₂—, or nitro; and
when Ar is a bicyclic aryl or heteroaryl or a monocyclic heteroaryl, then R² is independently at each occurrence (C₁-C₄) alkyl, (C₁-C₄) alkoxyl, (C₁-C₄) alkoxycarbonyl, (C₁-C₄)haloalkyl, hydroxymethyl, R²NCH₂—, cyano, nitro, halo, or —CF₃;
R is at each occurrence H or (C₁-C₄) alkyl; and
R³ is independently at each occurrence H or —CH₃;
provided that the compound is not:

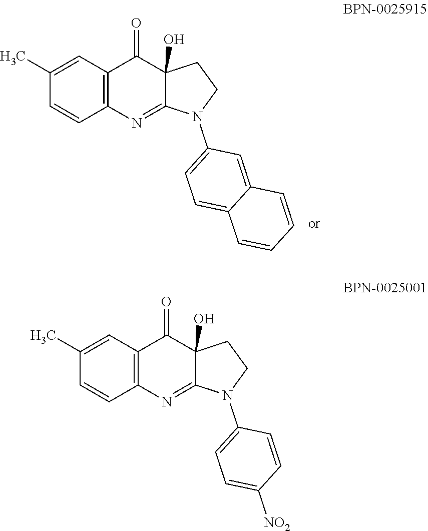 [SMILES: Cc1ccc2c(c1)C(=O)[C@]1(O)CCN(c3ccc4ccccc4c3)C1=N2 ; Cc1ccc2c(c1)C(=O)[C@]1(O)CCN(c3ccc([N+](=O)[O-])cc3)C1=N2]

7. The compound of claim 6, wherein R¹ is independently at each occurrence H, methyl, —CF₃, or fluoro.

8. The compound of claim 7, wherein R′ is independently at each occurrence H or methyl.

9. The compound of claim 8, wherein each R¹ is methyl.

10. The compound of claim 8, wherein one R¹ is methyl and the other R¹ is H.

11. The compound of claim 6, wherein Ar is a pyrazolyl, thiophenyl, isoquinolinyl, benzoxazolyl, quinolinyl, quinazolinyl, isoxazolyl, cinnolinyl, quinoxalinyl, benzisoxazolyl, benzothiadiazolyl, pyrazolopyridinyl, imidazopyridinyl, thieopyridinyl, dihydrobenzoxazinyl, triazolopyridinyl, dihydropyridoxazinyl, tetrahydrobenzoxazepinyl, dihydrobenzodioxinyl, dihydrobenzothiazinyl, tetrahydroquinolinyl, tetrahydronaphthyl, or chromanyl ring system, any of which is substituted with 1, 2, or 3 R².

12. The compound of claim 6, wherein Ar is a bicyclic heteroaryl substituted with 1, 2 or 3 R².

13. The compound of claim 12, wherein Ar is a bicyclic heteroaryl substituted with one R².

14. The compound of claim 13, wherein R² is (C₁-C₄) alkyl, (C₁-C₄) alkoxyl, cyano, or R²NCH₂—.

15. The compound of claim 14, wherein R² is (C₁-C₄) alkyl.

16. The compound of claim 15, wherein R² is methyl.

17. The compound of claim 6, wherein R³ is H at each occurrence.

18. The compound of claim 6, wherein the compound is selected from the group consisting of: 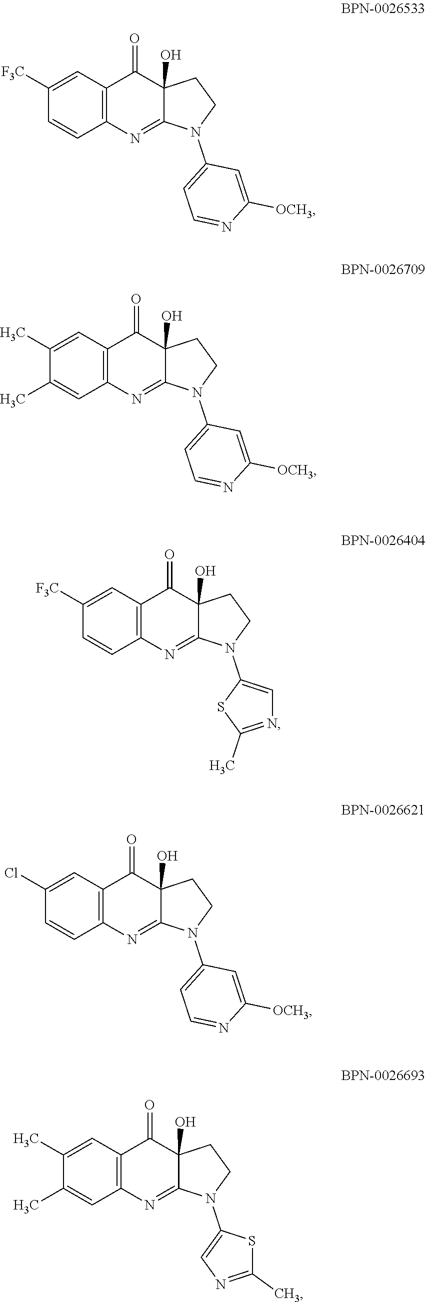 [SMILES: COc1cc(N2CC[C@@]3(O)C(=O)c4cc(C(F)(F)F)ccc4N=C23)ccn1 ; COc1cc(N2CC[C@@]3(O)C(=O)c4cc(C)c(C)cc4N=C23)ccn1 ; Cc1ncc(N2CC[C@@]3(O)C(=O)c4cc(C(F)(F)F)ccc4N=C23)s1 ; COc1cc(N2CC[C@@]3(O)C(=O)c4cc(Cl)ccc4N=C23)ccn1 ; Cc1ncc(N2CC[C@@]3(O)C(=O)c4cc(C)c(C)cc4N=C23)s1] 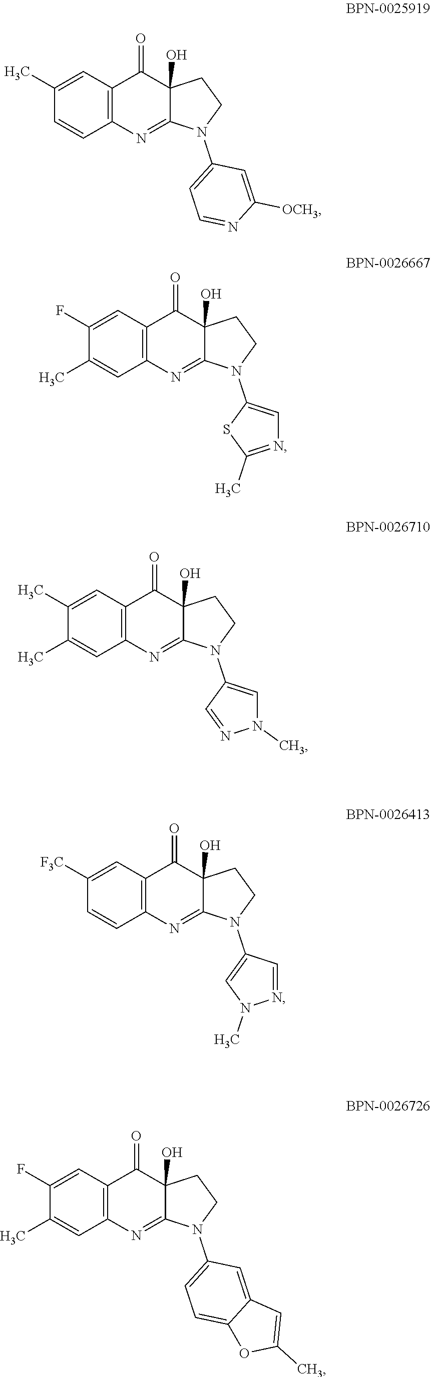 [SMILES: COc1cc(N2CC[C@@]3(O)C(=O)c4cc(C)ccc4N=C23)ccn1 ; Cc1ncc(N2CC[C@@]3(O)C(=O)c4cc(F)c(C)cc4N=C23)s1 ; Cc1cc2c(cc1C)C(=O)[C@]1(O)CCN(c3cnn(C)c3)C1=N2 ; Cn1cc(N2CC[C@@]3(O)C(=O)c4cc(C(F)(F)F)ccc4N=C23)cn1 ; Cc1cc2cc(N3CC[C@@]4(O)C(=O)c5cc(F)c(C)cc5N=C34)ccc2o1] 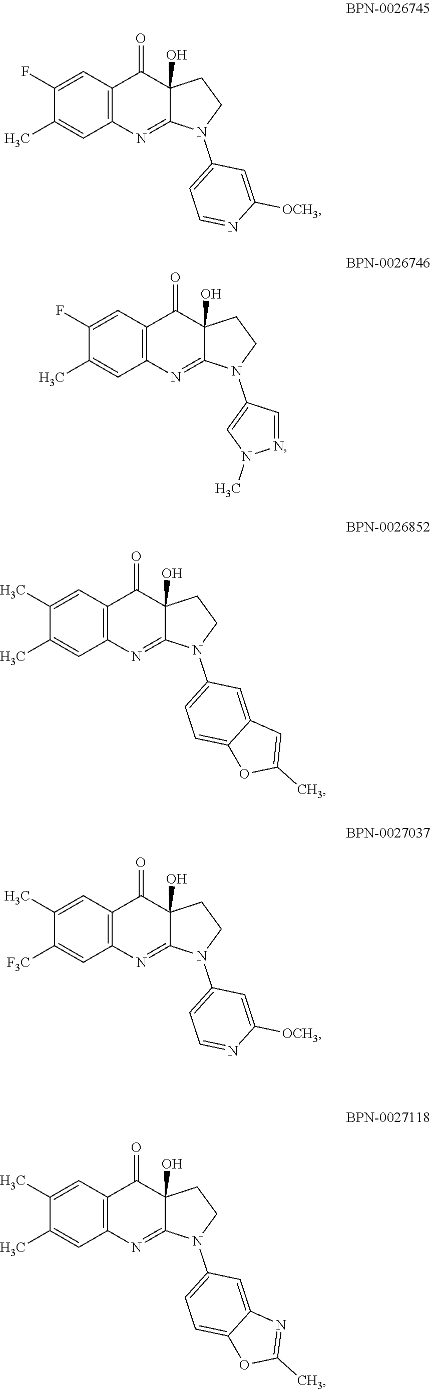 [SMILES: COc1cc(N2CC[C@@]3(O)C(=O)c4cc(F)c(C)cc4N=C23)ccn1 ; Cc1cc2c(cc1F)C(=O)[C@]1(O)CCN(c3cnn(C)c3)C1=N2 ; Cc1cc2cc(N3CC[C@@]4(O)C(=O)c5cc(C)c(C)cc5N=C34)ccc2o1 ; COc1cc(N2CC[C@@]3(O)C(=O)c4cc(C)c(C(F)(F)F)cc4N=C23)ccn1 ; Cc1nc2cc(N3CC[C@@]4(O)C(=O)c5cc(C)c(C)cc5N=C34)ccc2o1] 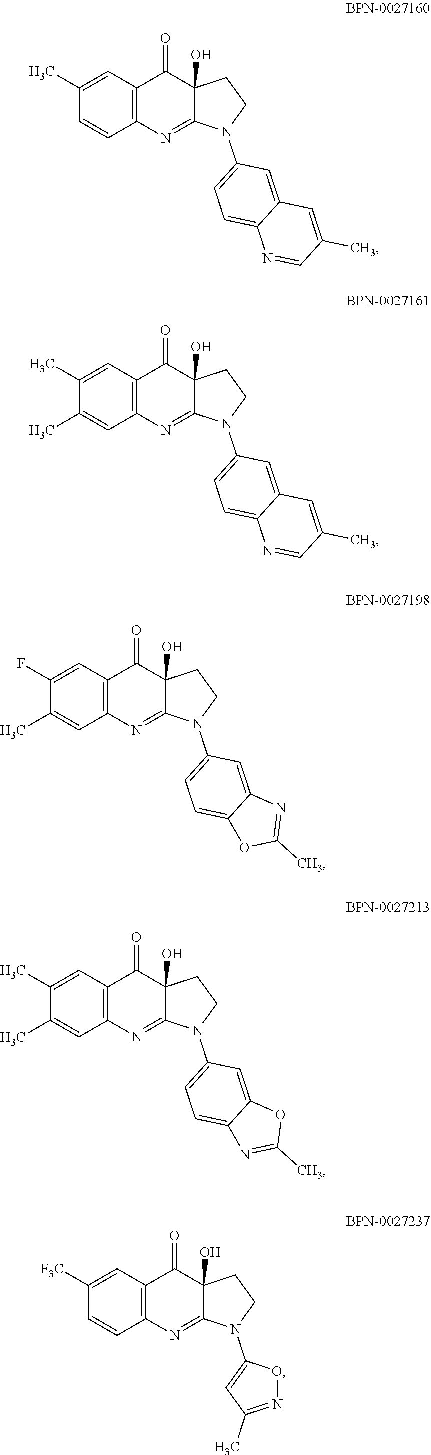 [SMILES: Cc1ccc2c(c1)C(=O)[C@]1(O)CCN(c3ccc4ncc(C)cc4c3)C1=N2 ; Cc1cnc2ccc(N3CC[C@@]4(O)C(=O)c5cc(C)c(C)cc5N=C34)cc2c1 ; Cc1nc2cc(N3CC[C@@]4(O)C(=O)c5cc(F)c(C)cc5N=C34)ccc2o1 ; Cc1nc2ccc(N3CC[C@@]4(O)C(=O)c5cc(C)c(C)cc5N=C34)cc2o1 ; Cc1cc(N2CC[C@@]3(O)C(=O)c4cc(C(F)(F)F)ccc4N=C23)on1] 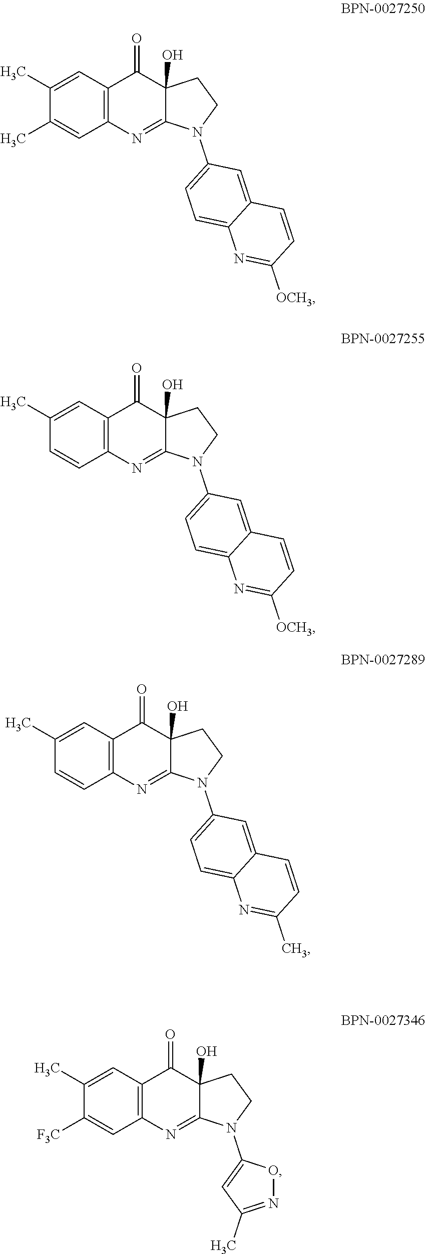 [SMILES: COc1ccc2cc(N3CC[C@@]4(O)C(=O)c5cc(C)c(C)cc5N=C34)ccc2n1 ; COc1ccc2cc(N3CC[C@@]4(O)C(=O)c5cc(C)ccc5N=C34)ccc2n1 ; Cc1ccc2c(c1)C(=O)[C@]1(O)CCN(c3ccc4nc(C)ccc4c3)C1=N2 ; Cc1cc(N2CC[C@@]3(O)C(=O)c4cc(C)c(C(F)(F)F)cc4N=C23)on1] 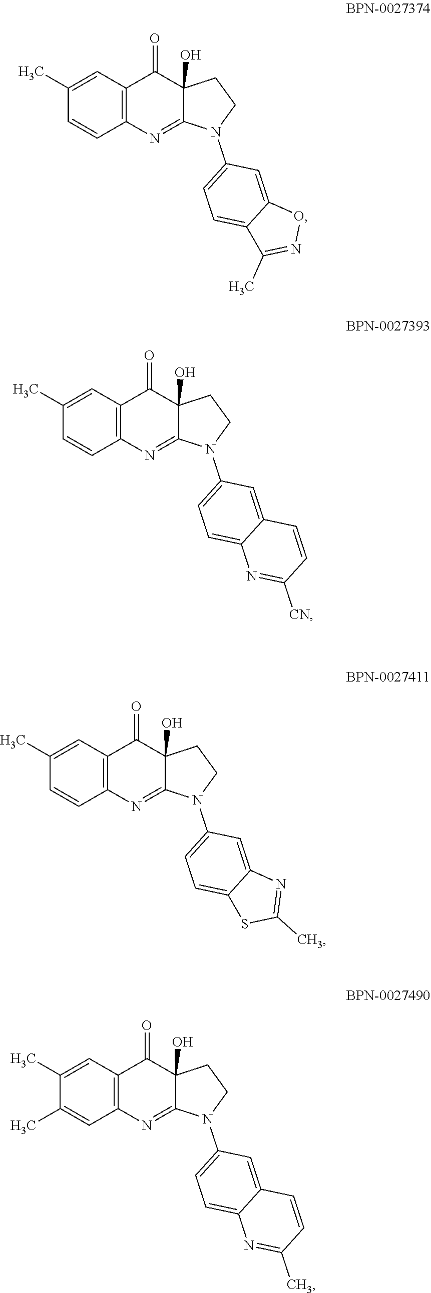 [SMILES: Cc1ccc2c(c1)C(=O)[C@]1(O)CCN(c3ccc4c(C)noc4c3)C1=N2 ; Cc1ccc2c(c1)C(=O)[C@]1(O)CCN(c3ccc4nc(C#N)ccc4c3)C1=N2 ; Cc1ccc2c(c1)C(=O)[C@]1(O)CCN(c3ccc4sc(C)nc4c3)C1=N2 ; Cc1ccc2cc(N3CC[C@@]4(O)C(=O)c5cc(C)c(C)cc5N=C34)ccc2n1] 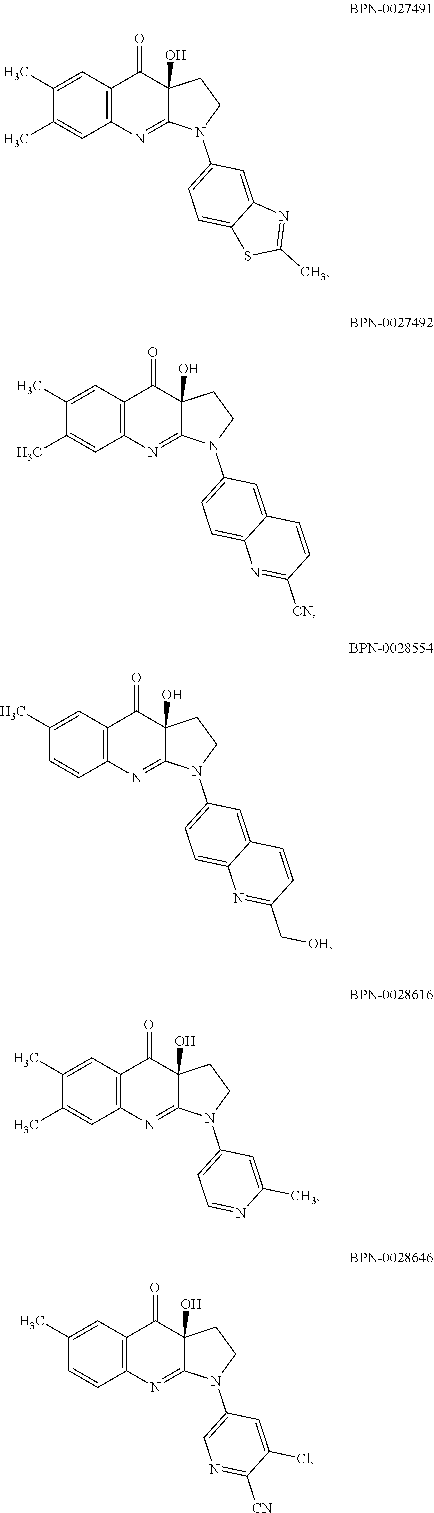 [SMILES: Cc1nc2cc(N3CC[C@@]4(O)C(=O)c5cc(C)c(C)cc5N=C34)ccc2s1 ; Cc1cc2c(cc1C)C(=O)[C@]1(O)CCN(c3ccc4nc(C#N)ccc4c3)C1=N2 ; Cc1ccc2c(c1)C(=O)[C@]1(O)CCN(c3ccc4nc(CO)ccc4c3)C1=N2 ; Cc1cc(N2CC[C@@]3(O)C(=O)c4cc(C)c(C)cc4N=C23)ccn1 ; Cc1ccc2c(c1)C(=O)[C@]1(O)CCN(c3cnc(C#N)c(Cl)c3)C1=N2] 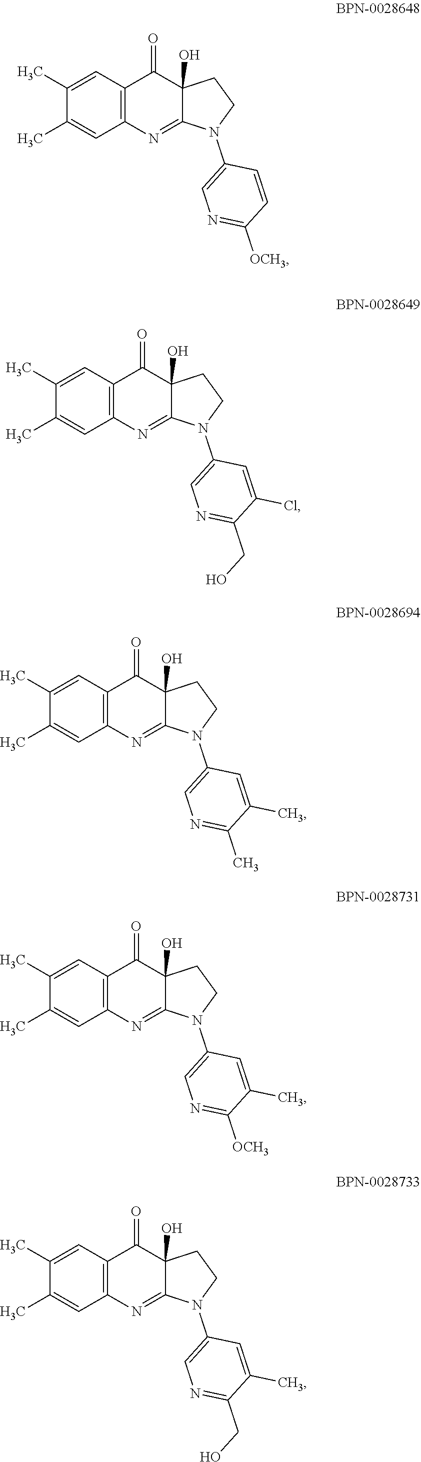 [SMILES: COc1ccc(N2CC[C@@]3(O)C(=O)c4cc(C)c(C)cc4N=C23)cn1 ; Cc1cc2c(cc1C)C(=O)[C@]1(O)CCN(c3cnc(CO)c(Cl)c3)C1=N2 ; Cc1cc2c(cc1C)C(=O)[C@]1(O)CCN(c3cnc(C)c(C)c3)C1=N2 ; COc1ncc(N2CC[C@@]3(O)C(=O)c4cc(C)c(C)cc4N=C23)cc1C ; Cc1cc2c(cc1C)C(=O)[C@]1(O)CCN(c3cnc(CO)c(C)c3)C1=N2] 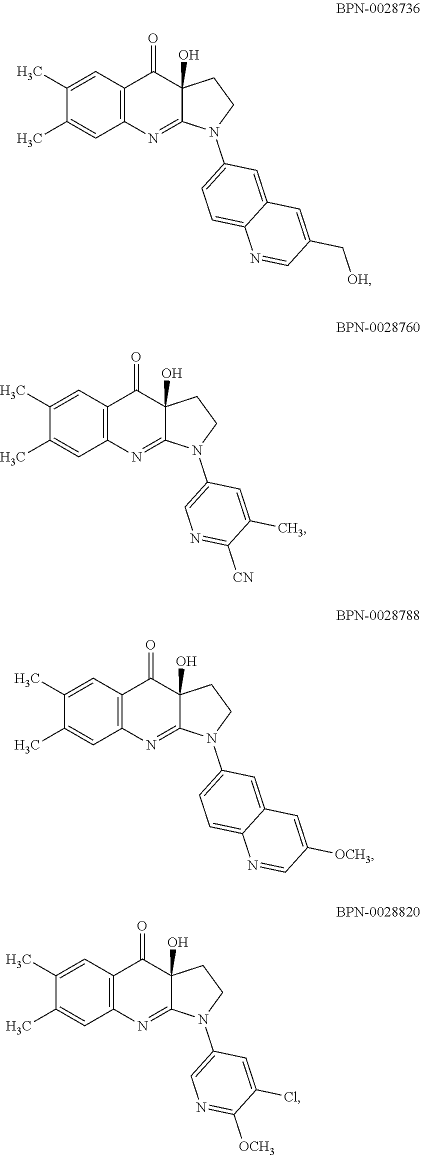 [SMILES: Cc1cc2c(cc1C)C(=O)[C@]1(O)CCN(c3ccc4ncc(CO)cc4c3)C1=N2 ; Cc1cc2c(cc1C)C(=O)[C@]1(O)CCN(c3cnc(C#N)c(C)c3)C1=N2 ; COc1cnc2ccc(N3CC[C@@]4(O)C(=O)c5cc(C)c(C)cc5N=C34)cc2c1 ; COc1ncc(N2CC[C@@]3(O)C(=O)c4cc(C)c(C)cc4N=C23)cc1Cl] 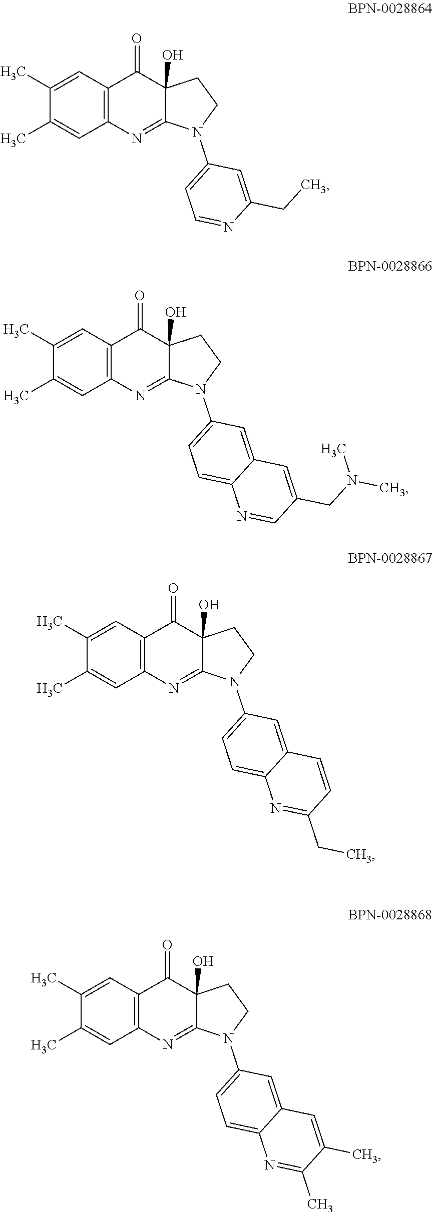 [SMILES: CCc1cc(N2CC[C@@]3(O)C(=O)c4cc(C)c(C)cc4N=C23)ccn1 ; Cc1cc2c(cc1C)C(=O)[C@]1(O)CCN(c3ccc4ncc(CN(C)C)cc4c3)C1=N2 ; CCc1ccc2cc(N3CC[C@@]4(O)C(=O)c5cc(C)c(C)cc5N=C34)ccc2n1 ; Cc1cc2c(cc1C)C(=O)[C@]1(O)CCN(c3ccc4nc(C)c(C)cc4c3)C1=N2] 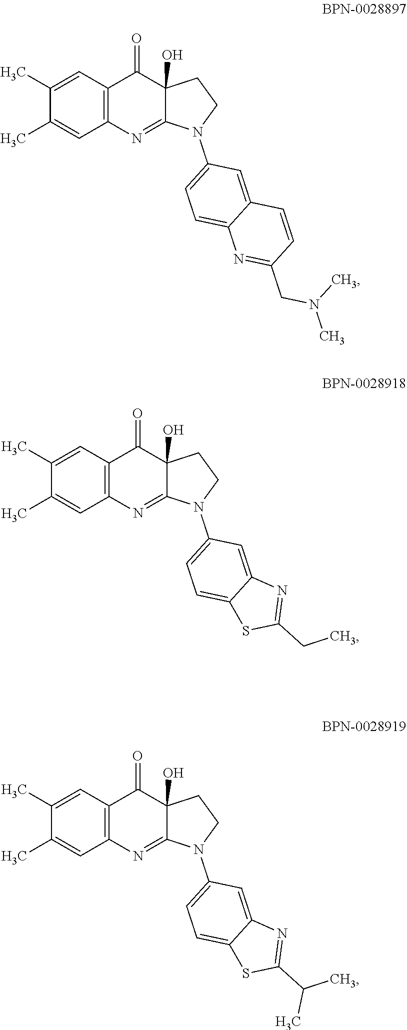 [SMILES: Cc1cc2c(cc1C)C(=O)[C@]1(O)CCN(c3ccc4nc(CN(C)C)ccc4c3)C1=N2 ; CCc1nc2cc(N3CC[C@@]4(O)C(=O)c5cc(C)c(C)cc5N=C34)ccc2s1 ; Cc1cc2c(cc1C)C(=O)[C@]1(O)CCN(c3ccc4sc(C(C)C)nc4c3)C1=N2]
and pharmaceutically acceptable salts thereof.

19. The compound of claim 6, wherein the compound is selected from the group 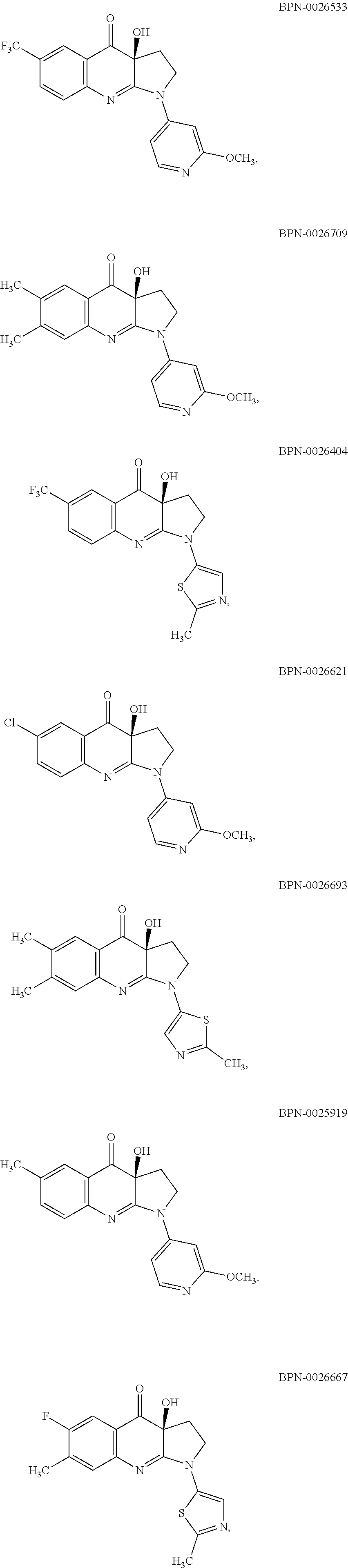
and pharmaceutically acceptable salts thereof.

20. The compound of claim 6, wherein the compound is selected from the group 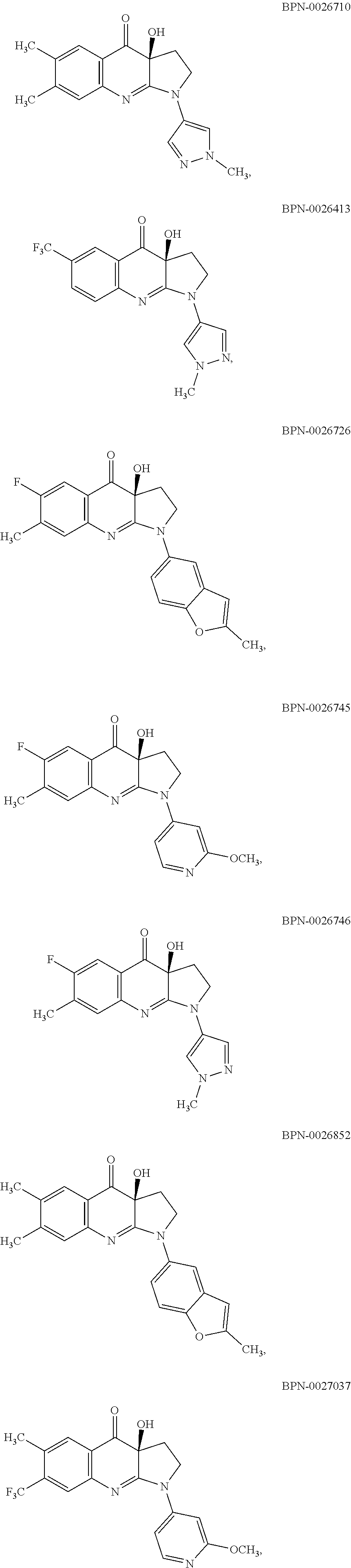
and pharmaceutically acceptable salts thereof.

21. The compound of claim 6, wherein the compound is selected from the group 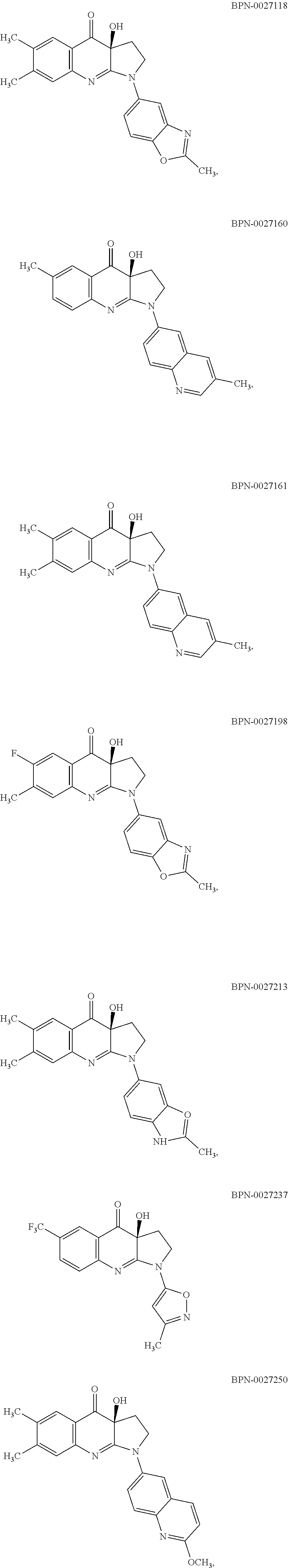
and pharmaceutically acceptable salts thereof.

22. The compound of claim 6, wherein the compound is selected from the group 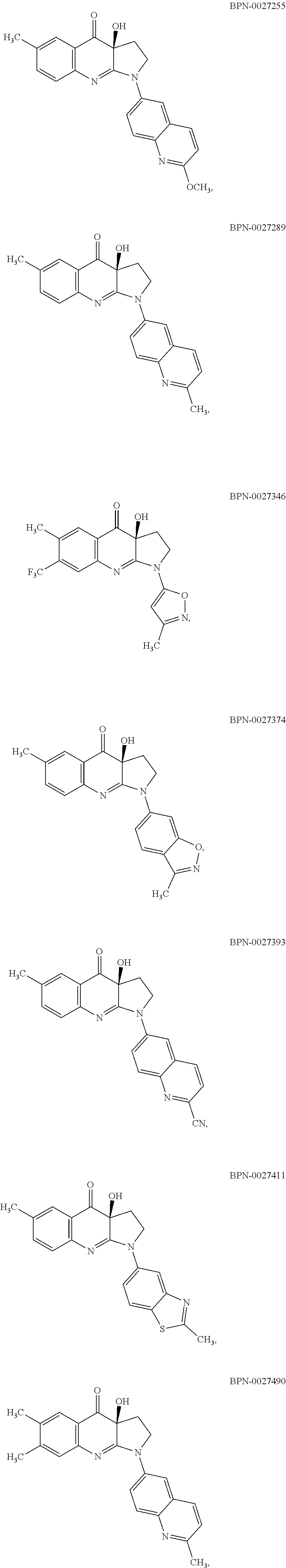
and pharmaceutically acceptable salts thereof.

23. The compound of claim 6, wherein the compound is selected from the group 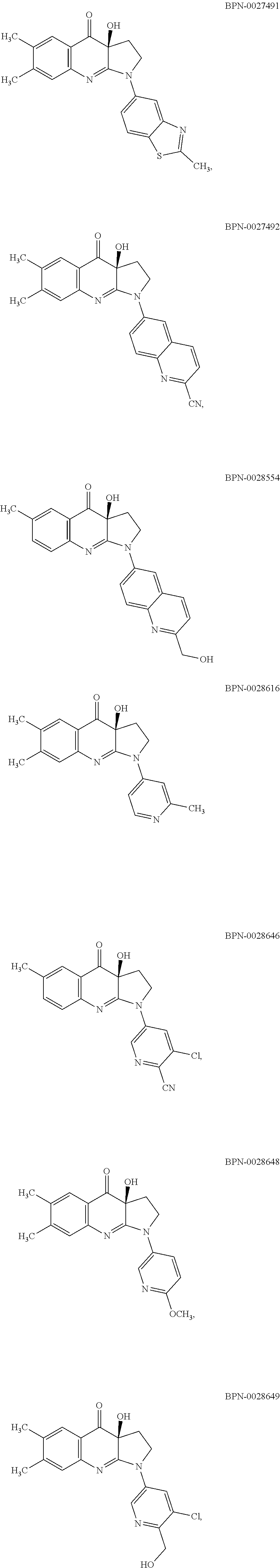
and pharmaceutically acceptable salts thereof.

24. The compound of claim 6, wherein the compound is selected from the group 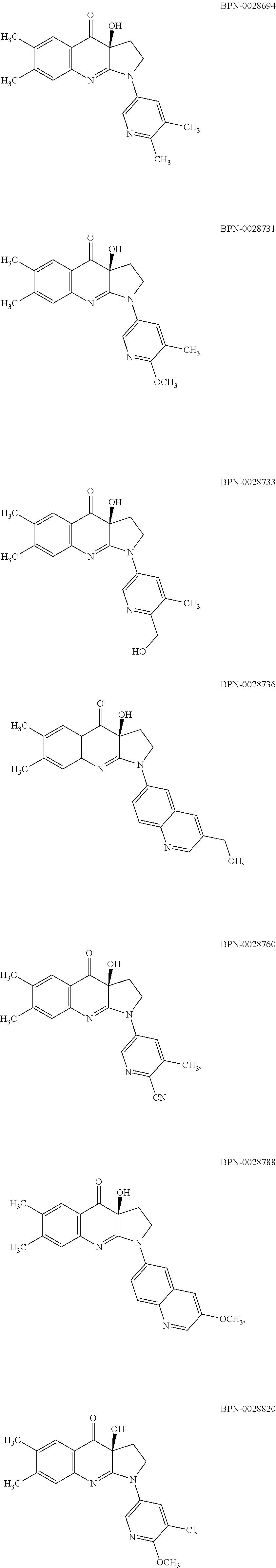
and pharmaceutically acceptable salts thereof.

25. The compound of claim 6, wherein the compound is selected from the group 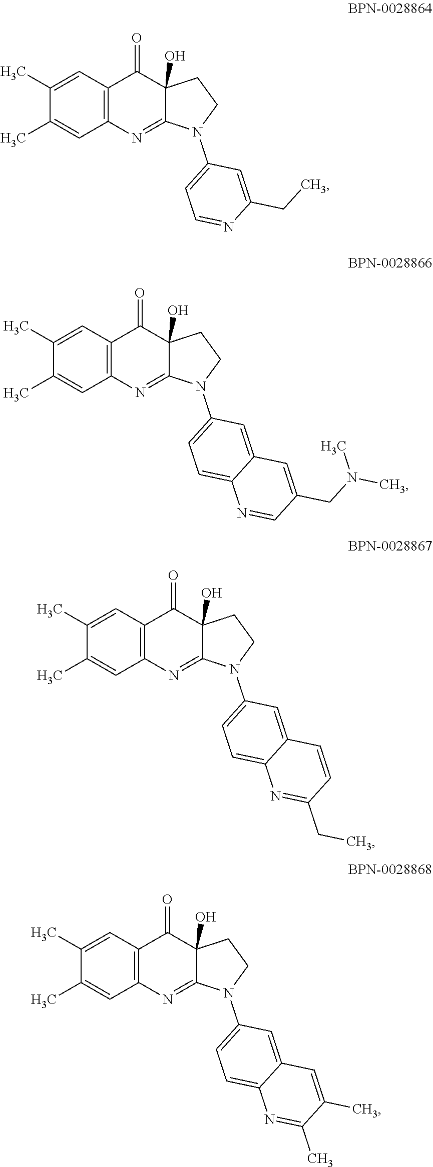 [SMILES: CCc1cc(N2CC[C@@]3(O)C(=O)c4cc(C)c(C)cc4N=C23)ccn1 ; Cc1cc2c(cc1C)C(=O)[C@]1(O)CCN(c3ccc4ncc(CN(C)C)cc4c3)C1=N2 ; CCc1ccc2cc(N3CC[C@@]4(O)C(=O)c5cc(C)c(C)cc5N=C34)ccc2n1 ; Cc1cc2c(cc1C)C(=O)[C@]1(O)CCN(c3ccc4nc(C)c(C)cc4c3)C1=N2] 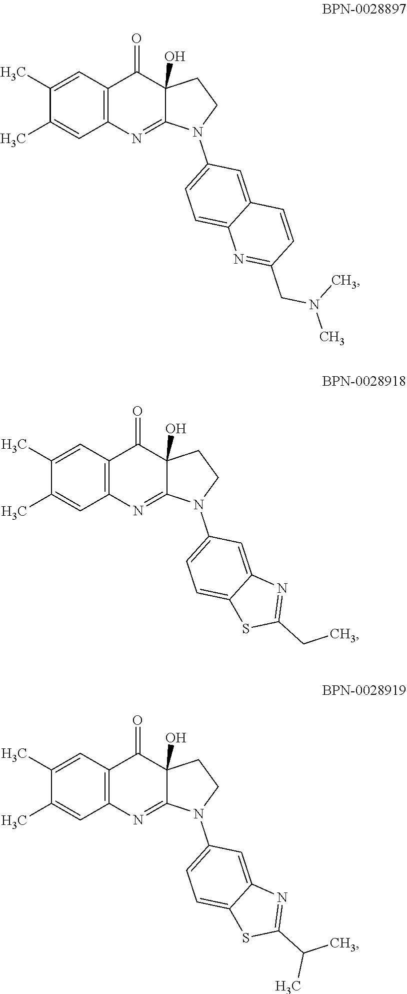 [SMILES: Cc1cc2c(cc1C)C(=O)[C@]1(O)CCN(c3ccc4nc(CN(C)C)ccc4c3)C1=N2 ; CCc1nc2cc(N3CC[C@@]4(O)C(=O)c5cc(C)c(C)cc5N=C34)ccc2s1 ; Cc1cc2c(cc1C)C(=O)[C@]1(O)CCN(c3ccc4sc(C(C)C)nc4c3)C1=N2]
and pharmaceutically acceptable salts thereof.
